(19) United States
(12) Patent Application Publication
(10) Pub. No.: US 2024/0173433 A1
(43) Pub. Date: May 30, 2024

(54) PROGRAMMABLE NUCLEASES AND METHODS OF USE

(71) Applicant: Mammoth Biosciences, Inc., Brisbane, CA (US)

(72) Inventors: Benjamin Julius RAUCH, San Francisco, CA (US); Aaron Deloughery, San Francisco, CA (US); William Douglass Wright, Fairfield, CA (US); David Paez-Espino, Concord, CA (US)

(21) Appl. No.: 18/481,182

(22) Filed: Oct. 4, 2023

Related U.S. Application Data
(63) Continuation of application No. PCT/US22/24896, filed on Apr. 14, 2022.
(60) Provisional application No. 63/175,524, filed on Apr. 15, 2021.
(60) Provisional application No. 63/286,489, filed on Dec. 6, 2021.
(60) Provisional application No. 63/315,743, filed on Mar. 2, 2022.

(51) Int. Cl. A61K 48/00; C12N 9/22; C12N 15/11; C12N 15/86

(52) U.S. Cl. CPC A61K 48/005; A61K 48/0033; C12N 9/22; C12N 15/111; C12N 15/86; C07K 2319/00; C12N 2310/20; C12N 2750/14143

(57) ABSTRACT

The present disclosure provides compositions and methods of use for Type V CRISPR/Cas proteins. Type V CRISPR/Cas proteins may be configured to bind and modify nucleic acids in a sequence specific manner.

### CROSS REFERENCE TO RELATED APPLICATIONS

This application is a continuation of International Patent Application No. PCT/US22/24896, filed Apr. 14, 2022, which claims the benefit of U.S. Provisional Application No. 63/175,524, filed Apr. 15, 2021, U.S. Provisional Application No. 63/286,489, filed Dec. 6, 2021, and U.S. Provisional Application No. 63/315,743, filed Mar. 2, 2022, the disclosures of which are incorporated herein by reference in their entirety.

### SEQUENCE LISTING

The instant application contains a Sequence Listing which has been submitted electronically. The Sequence Listing titled 203477-740301_US_SL.xml, which was created on Oct. 4, 2023 and is 565,142 bytes in size, is hereby incorporated by reference in its entirety.

### BACKGROUND OF THE INVENTION

There is a long-standing need in the life sciences for rapid and accurate nucleic acid modification and detection. Samples often contain low concentrations of nucleic acids interspersed among complex mixtures of other biomolecules. Present systems and methods of modifying and detecting nucleic acids may not be suitable in such instances or may require intensive sample preparation. Programmable nucleases are proteins that bind and cleave nucleic acids in a sequence-specific manner. A programmable nuclease may bind a target region of a nucleic acid and cleave the nucleic acid within the target region or at a position adjacent to the target region. In some instances, a programmable nuclease is activated when it binds a target region of a nucleic acid to cleave regions of the nucleic acid that are near, but not adjacent to the target region.

A programmable nuclease, such as a CRISPR-associated (Cas) protein, may be coupled to a guide nucleic acid that imparts activity or sequence selectivity to the programmable nuclease. In general, guide nucleic acids comprise a CRISPR RNA (crRNA) that is at least partially complementary to a target nucleic acid. In some cases, guide nucleic acids comprise a trans-activating crRNA (tracrRNA), at least a portion of which interacts with the programmable nuclease. In some cases, a tracrRNA or intermediary RNA is provided separately from the guide nucleic acid. The tracrRNA may hybridize to a portion of the guide nucleic acid that does not hybridize to the target nucleic acid.

A programmable nuclease may cleave a target nucleic acid. The target nucleic acid may be single stranded RNA (ssRNA), double stranded DNA (dsDNA) or single-stranded DNA (ssDNA). A programmable nuclease may provide cis cleavage activity, trans cleavage activity, nickase activity, or a combination thereof. Cis cleavage activity is cleavage of a target nucleic acid that is hybridized to a guide RNA (crRNA or sgRNA), wherein cleavage occurs within or directly adjacent to the region of the target nucleic acid that is hybridized to guideRNA. Trans cleavage activity (also referred to as transcollateral cleavage) is cleavage of ssDNA or ssRNA that is near, but not hybridized to the guide RNA. Trans cleavage activity is triggered by the hybridization of guide RNA to the target nucleic acid. Nickase activity is the selective cleavage of one strand of a dsDNA molecule. While certain programmable nucleases may be used to edit and detect nucleic acid molecules in a sequence specific manner, challenging biological sample conditions (e.g., high viscosity, metal chelating) may limit their accuracy and effectiveness. There is thus a need for systems and methods that employ programmable nucleases having specificity and efficiency across a wide range of sample conditions.

### SUMMARY OF THE INVENTION

The present disclosure provides compositions, systems, and methods comprising a Type V CRISPR/Cas protein and uses thereof. The present disclosure also provides for compositions comprising a nucleic acid encoding a Type V CRISPR/Cas protein. A Type V CRISPR/Cas protein is a programmable nuclease, which when coupled to a guide nucleic acid that is at least partially complementary to the nucleobase sequence of a target nucleic acid, binds and modifies (e.g., cleaves, nicks) the target nucleic acid.

Various compositions, systems, and methods of the present disclosure leverage cis cleavage activity, transcollateral cleavage activity, and nickase activity of Type V CRISPR/Cas proteins for the modification and detection of nucleic acids.

### Certain Embodiments

Provided herein is a composition comprising a programmable nuclease comprising an amino acid sequence that is at least 70%, at least 75%, at least 80%, at least 85%, at least 90%, at least 95%, or 100% identical to any one of SEQ ID NOs: 1-5, and an engineered guide nucleic acid that binds to the programmable nuclease.

Also provided herein is a composition comprising a multimeric complex of a first programmable nuclease comprising an amino acid sequence that is at least 70%, at least 75%, at least 80%, at least 85%, at least 90%, at least 95%, or 100% identical to any one of SEQ ID NOs: 1-5, a second programmable nuclease, and an engineered guide nucleic acid that binds to at least one of the first programmable nuclease and the second programmable nuclease. In some embodiments, the multimeric complex is a dimer, and the amino acid sequence of the second programmable nuclease is at least 90%, at least 95%, or 100% identical to the amino acid sequence of the first programmable nuclease. In some embodiments, the amino acid sequence of the second programmable nuclease is at least 70%, at least 75%, at least 80%, at least 85%, at least 90%, at least 95%, or 100% identical to any one of SEQ ID NOs: 6-198. In some embodiments, the amino acid sequence of the second programmable nuclease is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, or less than 15% identical to the amino acid sequence of the first programmable nuclease. In some embodiments, the first programmable nuclease and the second programmable nuclease are non-covalently coupled. In some embodiments, the first programmable nuclease and second programmable nuclease comprise different tertiary protein conformations in a solution. In some embodiments, the engineered guide nucleic acid comprises a crRNA, a tracrRNA, or a combination thereof. In some embodiments, the engineered guide nucleic acid comprises a nucleotide sequence that is at least 80%, at least 85%, at least 90% or at least 95% identical to SEQ ID NO: 255. In some embodiments, the engineered guide nucleic acid comprises a nucleotide sequence that is at least 80%, at least 85%, at least 90% or at least 95% identical to SEQ ID NO: 256. In some embodiments, the engineered guide nucleic acid comprises a nucleotide sequence that is at least 80%, at least 85%, at least 90% or at least 95% identical to SEQ ID NO: 257. In some embodiments, the crRNA comprises a nucleotide sequence that is at least 80%, at least 85%, at least 90% or at least 95% identical to SEQ ID NO: 258. In some embodiments, the crRNA comprises a nucleotide sequence that is at least 80%, at least 85%, at least 90% or at least 95% identical to SEQ ID NO: 259. In some embodiments, the crRNA comprises a nucleotide sequence that is at least 80%, at least 85%, at least 90% or at least 95% identical to SEQ ID NO: 260. In some embodiments, the crRNA comprises a nucleotide sequence that is at least 80%, at least 85%, at least 90% or at least 95% identical to SEQ ID NO: 261. In some embodiments, the crRNA comprises a nucleotide sequence that is at least 80%, at least 85%, at least 90% or at least 95% identical to SEQ ID NO: 262. In some cases, the engineered guide nucleic acid is a single guide RNA (sgRNA).

In some embodiments, the composition provides cis-cleavage activity on a target nucleic acid. In some embodiments, the composition provides transcollateral cleavage activity on a target nucleic acid. In some embodiments, the transcollateral cleavage activity cleaves a single strand of a nucleic acid in a sequence non-specific manner. In some embodiments, the programmable nuclease provides nickase activity on a target nucleic acid.

In some embodiments, at least one programmable nuclease of the composition comprises no greater than 540 amino acids. In some embodiments, the at least one programmable nuclease comprises no greater than 500 amino acids. In some embodiments, the at least one programmable nuclease comprises no greater than 480 amino acids. In some embodiments, at least one programmable nuclease of the composition comprises at least three partial RuvC domains, and wherein the at least three partial RuvC domains are not contiguous with respect to the primary amino acid sequence of the programmable nuclease. In some embodiments, the at least one programmable nuclease of the composition comprises a single RuvC domain. In some embodiments, at least one programmable nuclease of the composition recognizes a protospacer adjacent motif (PAM) sequence directly adjacent to a target sequence of a target nucleic acid. In some embodiments, the target nucleic acid is a double stranded DNA molecule comprising a target strand and a non-target strand, wherein at least a portion of the engineered guide nucleic acid is complementary to the target sequence on the target strand, and wherein the PAM is directly adjacent to the 5′ end of the target sequence on the non-target strand of the double stranded DNA molecule. In some embodiments, the PAM sequence comprises 5′-NTTR-3′, wherein N is any base, T is thymine, and R is a purine. In some embodiments, the PAM sequence comprises 5′-TTTR-3′, wherein T is thymine and R is a purine. In some embodiments, the PAM sequence comprises 5′-TTTG-3′, wherein T is thymine, and G is a Guanine. In some embodiments, the PAM sequence comprises 5′-GTTG-3′, wherein T is thymine, and G is a Guanine. In some embodiments, the PAM sequence comprises 5′-NTTR-3′, wherein N is any base, T is thymine, and R is a purine, and the programmable nuclease comprises an amino acid sequence that is at least 70%, at least 75%, at least 80%, at least 85%, at least 90%, at least 95%, or 100% identical to SEQ ID NO: 2. In some embodiments, the PAM sequence comprises 5′-TTTR-3′, wherein T is thymine and R is a purine, and the programmable nuclease comprises an amino acid sequence that is at least 70%, at least 75%, at least 80%, at least 85%, at least 90%, at least 95%, or 100% identical to any one of SEQ ID NO: 1 and 3-5. In some embodiments, the engineered guide nucleic acid comprises at least about 140 linked nucleosides. In some embodiments, the engineered guide nucleic acid comprises at least about 160 linked nucleosides. In some embodiments, at least one programmable nuclease of the composition comprises at least two recognition domains. In some embodiments, the at least two recognition domains comprise a first recognition domain and a second recognition domain that identify and direct nuclease binding to PAM sequences of different strands of a double-stranded target nucleic acid. In some embodiments, the multimeric complex comprises a first recognition domain and a second recognition domain that identify and direct nuclease binding to PAM sequences of different strands of a double-stranded target nucleic acid. In some embodiments, at least one programmable nuclease comprises a zinc finger domain.

In some embodiments, at least one programmable nuclease comprises an amino acid sequence that is at least at least 70%, at least 75%, at least 80%, at least 85%, at least 90%, at least 95%, or 100% identical to SEQ ID NO: 1. In some embodiments, at least one programmable nuclease consists of an amino acid sequence that is at least at least 70%, at least 75%, at least 80%, at least 85%, at least 90%, at least 95%, or 100% identical to SEQ ID NO: 1. In some embodiments, at least one programmable nuclease comprises an amino acid sequence that is at least at least 70%, at least 75%, at least 80%, at least 85%, at least 90%, at least 95%, or 100% identical to SEQ ID NO: 2. In some embodiments, at least one programmable nuclease consists of an amino acid sequence that is at least at least 70%, at least 75%, at least 80%, at least 85%, at least 90%, at least 95%, or 100% identical to SEQ ID NO: 2. In some embodiments, at least one programmable nuclease comprises an amino acid sequence that is at least at least 70%, at least 75%, at least 80%, at least 85%, at least 90%, at least 95%, or 100% identical to SEQ ID NO: 3. In some embodiments, at least one programmable nuclease consists of an amino acid sequence that is at least at least 70%, at least 75%, at least 80%, at least 85%, at least 90%, at least 95%, or 100% identical to SEQ ID NO: 3. In some embodiments, at least one programmable nuclease comprises an amino acid sequence that is at least at least 70%, at least 75%, at least 80%, at least 85%, at least 90%, at least 95%, or 100% identical to SEQ ID NO: 4.

In some embodiments, at least one programmable nuclease consists of an amino acid sequence that is at least at least 70%, at least 75%, at least 80%, at least 85%, at least 90%, at least 95%, or 100% identical to SEQ ID NO: 4. In some embodiments, at least one programmable nuclease comprises an amino acid sequence that is at least at least 70%, at least 75%, at least 80%, at least 85%, at least 90%, at least 95%, or 100% identical to SEQ ID NO: 5. In some embodiments, the at least one programmable nuclease consists of an amino acid sequence that is at least at least 70%, at least 75%, at least 80%, at least 85%, at least 90%, at least 95%, or 100% identical to SEQ ID NO: 5.

In some embodiments, provided herein is a system for detecting a target nucleic acid comprising the composition described herein in a solution, wherein the solution comprises at least one of a buffering agent, a salt, a crowding agent, a detergent, a reducing agent, a competitor, and a reporter nucleic acid. In some embodiments, the pH of the solution is selected from at least about 6.0, at least about 6.5, at least about 7.0, at least about 7.5, at least about 8.0, at least about 8.5, or at least about 9. In some embodiments, the salt is selected from a magnesium salt, a potassium salt, a sodium salt and a calcium salt. In some embodiments, the concentration of the salt in the solution is selected from at least about 1 mM, at least about 3 mM, at least about 5 mM, at least about 7 mM, at least about 9 mM, at least about 11 mM, at least about 13 mM, or at least about 15 mM. In some embodiments, the reporter nucleic acid comprises at least one of a fluorophore and a quencher. In some embodiments, the reporter nucleic acid is in the form of single stranded DNA.

Provided herein is a method of detecting a target nucleic acid in a sample, comprising: (a) contacting the sample with: a programmable nuclease or a multimeric complex thereof, wherein the programmable nuclease comprises an amino acid sequence that is at least 70%, at least 75%, at least 80%, at least 85%, at least 90%, at least 95%, or 100% identical to any one of SEQ ID NOs: 1-5, an engineered guide nucleic acid, wherein at least a portion of the engineered guide nucleic acid is complementary to at least a portion of the target nucleic acid, and a reporter nucleic acid that is cleaved in the presence of the programmable nuclease, the engineered guide nucleic acid, and the target nucleic acid, and detecting a signal produced by cleavage of the reporter nucleic acid, thereby detecting the target nucleic acid in the sample. In some embodiments, the multimeric complex is a dimer, wherein the programmable nuclease is a first programmable nuclease and the multimeric complex comprises a second programmable nuclease, and wherein the amino acid sequence of the second programmable nuclease is at least 90%, at least 95%, or 100% identical to the amino acid sequence of the first programmable nuclease. In some embodiments, the multimeric complex, wherein the programmable nuclease is a first programmable nuclease and the multimeric complex comprises a second programmable nuclease that is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, or less than 15% identical to the amino acid sequence of the first programmable nuclease. In some embodiments, the first programmable nuclease and the second programmable nuclease are non-covalently coupled. In some embodiments, the first programmable nuclease and second programmable nuclease comprise different tertiary protein conformations in a solution. In some embodiments, the engineered guide nucleic acid comprises a crRNA, a tracrRNA, or a combination thereof. In some embodiments, the engineered guide nucleic acid comprises a nucleotide sequence that is at least 80%, at least 85%, at least 90% or at least 95% identical to SEQ ID NO: 255. In some embodiments, the engineered guide nucleic acid comprises a nucleotide sequence that is at least 80%, at least 85%, at least 90% or at least 95% identical to SEQ ID NO: 256. In some embodiments, the engineered guide nucleic acid comprises a nucleotide sequence that is at least 80%, at least 85%, at least 90% or at least 95% identical to SEQ ID NO: 257. In some embodiments, the crRNA comprises a nucleotide sequence that is at least 80%, at least 85%, at least 90% or at least 95% identical to SEQ ID NO: 258, SEQ ID NO: 259, SEQ ID NO: 260, SEQ ID NO: 261, or SEQ ID NO: 262. In some cases, the engineered guide nucleic acid is a single guide RNA (sgRNA). In some embodiments, the composition provides cis-cleavage activity on a target nucleic acid. In some embodiments, the composition provides transcollateral cleavage activity on a target nucleic acid. In some embodiments, the transcollateral cleavage activity cleaves a single strand of a nucleic acid in a sequence non-specific manner. In some embodiments, the at least one programmable nuclease of the composition comprises no greater than 540 amino acids. In some embodiments, the at least one programmable nuclease comprises no greater than 500 amino acids. In some embodiments, the at least one programmable nuclease comprises no greater than 480 amino acids. In some embodiments, the at least one programmable nuclease of the composition comprises at least three partial RuvC domains, and wherein the at least three partial RuvC domains are not contiguous with respect to the primary amino acid sequence of the programmable nuclease. In some embodiments, the at least one programmable nuclease of the composition comprises a single RuvC domain. In some embodiments, the at least one programmable nuclease of the composition recognizes a protospacer adjacent motif (PAM) sequence directly adjacent to a target sequence of a target nucleic acid. In some embodiments, the PAM sequence comprises 5′-NTTR-3′, wherein N is any base, T is thymine, and R is a purine. In some embodiments, the PAM sequence comprises 5′-TTTR-3′, wherein T is thymine and R is a purine. In some embodiments, the PAM sequence comprises 5′-TTTG-3′, wherein T is thymine, and G is a Guanine. In some embodiments, the PAM sequence comprises 5′-GTTG-3′, wherein T is thymine, and G is a Guanine. In some embodiments, the PAM sequence comprises 5′-NTTR-3′, wherein N is any base, T is thymine, and R is a purine, and the programmable nuclease comprises an amino acid sequence that is at least 70%, at least 75%, at least 80%, at least 85%, at least 90%, at least 95%, or 100% identical to SEQ ID NO: 2. In some embodiments, the PAM sequence comprises 5′-TTTR-3′, wherein T is thymine and R is a purine, and the programmable nuclease comprises an amino acid sequence that is at least 70%, at least 75%, at least 80%, at least 85%, at least 90%, at least 95%, or 100% identical to any one of SEQ ID NO: 1 and 3-5. In some embodiments, the engineered guide nucleic acid comprises at least about 140 linked nucleosides. In some embodiments, the engineered guide nucleic acid comprises at least about 160 linked nucleosides. In some embodiments, the at least one programmable nuclease of the composition comprises at least two recognition domains. In some embodiments, the at least two recognition domains comprise a first recognition domain and a second recognition domain that identify and direct nuclease binding to PAM sequences of different strands of a double-stranded target nucleic acid. In some embodiments, the multimeric complex comprises a first recognition domain and a second recognition domain that identify and direct nuclease binding to PAM sequences of different strands of a double-stranded target nucleic acid. In some embodiments, the at least one programmable nuclease comprises a zinc finger domain. In some embodiments, the amino acid sequence of at least one programmable nuclease is at least 70%, at least 75%, at least 80%, at least 85%, at least 90%, at least 95%, or 100% identical to any one of SEQ ID NOs: 1-5. In some embodiments, the reporter nucleic acid comprises at least one of a fluorophore and a quencher.

Provided herein is a method of generating a recombinant cell, the method comprising delivering a guide nucleic acid and a programmable nuclease to a target cell, wherein the programmable nuclease comprises an amino acid sequence that is at least 70%, at least 75%, at least 80%, at least 85%, at least 90%, at least 95%, or 100% identical to any one of SEQ ID NOs: 1-5, thereby generating the recombinant cell from the target cell. In some embodiments, the method comprises delivering a nucleic acid encoding the programmable nuclease. In some embodiments, the nucleic acid encodes the guide nucleic acid. In some embodiments, the programmable nuclease is capable of forming a dimer. In some embodiments, the nucleic acid encodes multiple programmable nucleases, and wherein the multiple programmable nucleases are capable of forming a multimeric complex. In some embodiments of the method, delivering comprises electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof. In some embodiments of the method, delivering the guide nucleic acid and programmable nuclease generates a double-stranded break in the genome of the target cell, and optionally, wherein the method comprises detecting the double-stranded break. In some embodiments of the method, the method comprises repairing the double-stranded break, and wherein the repair results in an indel in the genome of the target cell. In some embodiments of the method, the method comprises delivering a donor nucleic acid to the target cell. In some embodiments of the method, the donor nucleic acid is incorporated into the genome of the target cell, and optionally wherein the method comprises detecting the incorporation of the donor nucleic acid in the genome of the target cell. In some embodiments of the method, the target cell is a eukaryotic cell. In some embodiments, the target cell is a mammalian cell. In some embodiments, the target cell is a prokaryotic cell. Provided herein is a recombinant cell produced by the any one of the embodiments of the method described here. Provided herein is a population of cells generated from the recombinant cell derived using any of the methods here.

Provided herein is a method of modifying a target nucleic acid, comprising contacting the target nucleic acid with the composition described here, thereby modifying the target nucleic acid. In some embodiments, the method comprises contacting the target nucleic acid with a donor sequence. In some embodiments, the target nucleic acid is located in a target cell and contacting the target nucleic acid comprises delivering the composition to the target cell. In some embodiments, delivering the composition to the target cell occurs in vitro. In some embodiments, delivering the composition to the target cell occurs in vivo.

Provided herein is a composition comprising at least one nucleic acid that encodes: a programmable nuclease, wherein the programmable nuclease comprises an amino acid sequence that is at least 70%, at least 75%, at least 80%, at least 85%, at least 90%, at least 95%, or 100% identical to any one of SEQ ID NOs: 1-5, and an engineered guide nucleic acid that binds to the programmable nuclease. In some embodiments, the nucleic acid encoding the programmable nuclease and the engineered guide nucleic acid are linked by at least one internucleoside linkage. In some embodiments, the amino acid sequence of the programmable nuclease is at least 70%, at least 75%, at least 80%, at least 85%, at least 90%, at least 95%, or 100% identical to SEQ ID NO: 1. In some embodiments, the amino acid sequence of the programmable nuclease is at least 70%, at least 75%, at least 80%, at least 85%, at least 90%, at least 95%, or 100% identical to SEQ ID NO: 2. In some embodiments, the amino acid sequence of the programmable nuclease is at least 70%, at least 75%, at least 80%, at least 85%, at least 90%, at least 95%, or 100% identical to SEQ ID NO: 3. In some embodiments, the amino acid sequence of the programmable nuclease is at least 70%, at least 75%, at least 80%, at least 85%, at least 90%, at least 95%, or 100% identical to SEQ ID NO: 4. In some embodiments, the amino acid sequence of the programmable nuclease is at least 70%, at least 75%, at least 80%, at least 85%, at least 90%, at least 95%, or 100% identical to SEQ ID NO: 5.

Provided here in is a composition comprising a programmable nuclease, a crRNA, and a tracrRNA, wherein the amino acid sequence of the programmable nuclease is at least 80%, at least 85%, at least 90%, or at least 95% identical to the SEQ ID NO: 8, wherein the nucleobase sequence of the crRNA is at least 80%, at least 85%, at least 90% or at least 95% identical to SEQ ID NO: 233, and wherein the nucleobase sequence of the tracrRNA is at least 80%, at least 85%, at least 90% or at least 95% identical to any one of SEQ ID NOs: 220-222. Provided here in is a composition comprising a programmable nuclease, a crRNA, and a tracrRNA, wherein the amino acid sequence of the programmable nuclease is at least 80%, at least 85%, at least 90%, or at least 95% identical to the SEQ ID NO: 6, wherein the nucleobase sequence of the crRNA is at least 80%, at least 85%, at least 90% or at least 95% identical to SEQ ID NO: 233, and wherein the nucleobase sequence of the tracrRNA is at least 80%, at least 85%, at least 90% or at least 95% identical to any one of SEQ ID NOs: 221-222. Provided herein is a composition comprising a programmable nuclease, a crRNA, and a tracrRNA, wherein the amino acid sequence of the programmable nuclease is at least 80%, at least 85%, at least 90%, or at least 95% identical to the SEQ ID NO: 1, wherein the nucleobase sequence of the crRNA is at least 80%, at least 85%, at least 90% or at least 95% identical to any one of SEQ ID NOs: 233-234, and wherein the nucleobase sequence of the tracrRNA is at least 80%, at least 85%, at least 90% or at least 95% identical to any one of SEQ ID NOs: 221, or 223-224. Provided here in is a composition comprising a programmable nuclease, a crRNA, and a tracrRNA, wherein the amino acid sequence of the programmable nuclease is at least 80%, at least 85%, at least 90%, or at least 95% identical to the SEQ ID NO: 2, wherein the nucleobase sequence of the crRNA is at least 80%, at least 85%, at least 90% or at least 95% identical to any one of SEQ ID NOs: 233 or 235, and wherein the nucleobase sequence of the tracrRNA is at least 80%, at least 85%, at least 90% or at least 95% identical to any one of SEQ ID NOs: 221, or 225-226. Provided here in is a composition comprising a programmable nuclease, a crRNA, and a tracrRNA, wherein the amino acid sequence of the programmable nuclease is at least 80%, at least 85%, at least 90%, or at least 95% identical to the SEQ ID NO: 3, wherein the nucleobase sequence of the crRNA is at least 80%, at least 85%, at least 90% or at least 95% identical to any one of SEQ ID NOs: 233 or 237, and wherein the nucleobase sequence of the tracrRNA is at least 80%, at least 85%, at least 90% or at least 95% identical to any one of SEQ ID NOs: 221, or 227-228. Provided here in is a composition comprising a programmable nuclease, a crRNA, and a tracrRNA, wherein the amino acid sequence of the programmable nuclease is at least 80%, at least 85%, at least 90%, or at least 95% identical to the SEQ ID NO: 4, wherein the nucleobase sequence of the crRNA is at least 80%, at least 85%, at least 90% or at least 95% identical to any one of SEQ ID NOs: 233 or 236, and wherein the nucleobase sequence of the tracrRNA is at least 80%, at least 85%, at least 90% or at least 95% identical to any one of SEQ ID NOs: 221, or 229-230. Provided here in is a composition comprising a programmable nuclease, a crRNA, and a tracrRNA, wherein the amino acid sequence of the programmable nuclease is at least 80%, at least 85%, at least 90%, or at least 95% identical to the SEQ ID NO: 5, wherein the nucleobase sequence of the crRNA is at least 80%, at least 85%, at least 90% or at least 95% identical to any one of SEQ ID NOs: 233 or 238, and wherein the nucleobase sequence of the tracrRNA is at least 80%, at least 85%, at least 90% or at least 95% identical to any one of SEQ ID NOs: 221, or 231-232. Provided here in is a composition comprising a programmable nuclease, a crRNA, and a tracrRNA, wherein the amino acid sequence of the programmable nuclease is at least 80%, at least 85%, at least 90%, or at least 95% identical to the SEQ ID NO: 2, wherein the nucleobase sequence of the crRNA is at least 80%, at least 85%, at least 90% or at least 95% identical to any one of SEQ ID NOs: 233 or 235, and wherein the nucleobase sequence of the tracrRNA is at least 80%, at least 85%, at least 90% or at least 95% identical to any one of SEQ ID NOs: 221, or 225-226. In some embodiments, the crRNA and the tracrRNA are linked.

Provided herein is a composition comprising a programmable nuclease, or a nucleic acid encoding the programmable nuclease, and a sgRNA, or a DNA molecule encoding the sgRNA, wherein the amino acid sequence of the programmable nuclease is at least 80%, at least 85%, at least 90%, or at least 95% identical to the SEQ ID NO: 1, and wherein the sgRNA comprises a nucleotide sequence that is at least 80%, at least 85%, at least 90% or at least 95% identical to SEQ ID NO: 255.

Provided herein is a composition comprising a programmable nuclease, or a nucleic acid encoding the programmable nuclease, and a sgRNA, or a DNA molecule encoding the sgRNA, wherein the amino acid sequence of the programmable nuclease is at least 80%, at least 85%, at least 90%, or at least 95% identical to any one of SEQ ID NO: 2, 4, and 5, and wherein the sgRNA comprises a nucleotide sequence that is at least 80%, at least 85%, at least 90% or at least 95% identical to SEQ ID NO: 256.

Provided herein is a composition comprising a programmable nuclease, or a nucleic acid encoding the programmable nuclease, and a sgRNA, or a DNA molecule encoding the sgRNA, wherein the amino acid sequence of the programmable nuclease is at least 80%, at least 85%, at least 90%, or at least 95% identical to the SEQ ID NO: 3, and wherein the sgRNA comprises a nucleotide sequence that is at least 80%, at least 85%, at least 90% or at least 95% identical to SEQ ID NO: 257.

Provided herein is a composition comprising a programmable nuclease, or a nucleic acid encoding the programmable nuclease, and a crRNA, or a DNA molecule encoding the crRNA, wherein the amino acid sequence of the programmable nuclease is at least 80%, at least 85%, at least 90%, or at least 95% identical to the SEQ ID NO: 1, and wherein the crRNA comprises a nucleotide sequence that is at least 80%, at least 85%, at least 90% or at least 95% identical to SEQ ID NO: 258.

Provided herein is a composition comprising a programmable nuclease, or a nucleic acid encoding the programmable nuclease, and a crRNA, or a DNA molecule encoding the crRNA, wherein the amino acid sequence of the programmable nuclease is at least 80%, at least 85%, at least 90%, or at least 95% identical to the SEQ ID NO: 2, and wherein the crRNA comprises a nucleotide sequence that is at least 80%, at least 85%, at least 90% or at least 95% identical to SEQ ID NO: 259.

Provided herein is a composition comprising a programmable nuclease, or a nucleic acid encoding the programmable nuclease, and a crRNA, or a DNA molecule encoding the crRNA, wherein the amino acid sequence of the programmable nuclease is at least 80%, at least 85%, at least 90%, or at least 95% identical to the SEQ ID NO: 3, and wherein the crRNA comprises a nucleotide sequence that is at least 80%, at least 85%, at least 90% or at least 95% identical to SEQ ID NO: 260.

Provided herein is a composition comprising a programmable nuclease, or a nucleic acid encoding the programmable nuclease, and a crRNA, or a DNA molecule encoding the crRNA, wherein the amino acid sequence of the programmable nuclease is at least 80%, at least 85%, at least 90%, or at least 95% identical to the SEQ ID NO: 4, and wherein the crRNA comprises a nucleotide sequence that is at least 80%, at least 85%, at least 90% or at least 95% identical to SEQ ID NO: 261.

Provided herein is a composition comprising a programmable nuclease, or a nucleic acid encoding the programmable nuclease, and a crRNA, or a DNA molecule encoding the crRNA, wherein the amino acid sequence of the programmable nuclease is at least 80%, at least 85%, at least 90%, or at least 95% identical to the SEQ ID NO: 5, and wherein the crRNA comprises a nucleotide sequence that is at least 80%, at least 85%, at least 90% or at least 95% identical to SEQ ID NO: 262.

In some embodiments of the compositions above, the nucleic acid encoding the programmable nuclease is an expression vector. In some embodiments, the expression vector is an adeno associated viral vector. In some embodiments, the expression vector encodes the crRNA or sgRNA. In some embodiments, the nucleic acid encoding the programmable nuclease is a messenger RNA. In some embodiments, the compositions further comprise a lipid or a lipid nanoparticle encapsulating the programmable nuclease, the sgRNA or crRNA, the nucleic acid encoding the programmable nuclease and/or the DNA molecule encoding the sgRNA or crRNA.

In some embodiments, the compositions above present in a pharmaceutical composition.

In some embodiments, provided herein is a method of detecting a target nucleic acid using the embodiments herein. In some embodiments, provided herein is a cell comprising any of the compositions described here.

Also provided herein are compositions comprising a programmable nuclease comprising an amino acid sequence as described herein, or a nucleic acid encoding the programmable nuclease. In some embodiments, the programmable nuclease is at least 70%, at least 75%, at least 80%, at least 85%, at least 90%, at least 95%, or 100% identical to any one of SEQ ID NOs: 1-5. In some embodiments, a composition can comprise an engineered guide nucleic acid, or a DNA molecule encoding the engineered guide nucleic acid. In some embodiments, the programmable nuclease binds to at least a portion of the engineered guide nucleic acid. In some embodiments, the engineered guide nucleic acid comprises a crRNA, a tracrRNA, or a combination thereof. In some embodiments, the engineered guide nucleic acid comprises a single guide RNA (sgRNA). In some embodiments, the engineered guide nucleic acid comprises a nucleotide sequence that is at least 80%, at least 85%, at least 90% or at least 95% identical to SEQ ID NO: 255. In some embodiments, the engineered guide nucleic acid comprises a nucleotide sequence that is at least 80%, at least 85%, at least 90% or at least 95% identical to SEQ ID NO: 256. In some embodiments, the engineered guide nucleic acid comprises a nucleotide sequence that is at least 80%, at least 85%, at least 90% or at least 95% identical to SEQ ID NO: 257. In some embodiments, the engineered guide nucleic acid comprises a nucleotide sequence that is at least 80%, at least 85%, at least 90% or at least 95% identical to SEQ ID NO: 258. In some embodiments, the engineered guide nucleic acid comprises a nucleotide sequence that is at least 80%, at least 85%, at least 90% or at least 95% identical to SEQ ID NO: 259. In some embodiments, the engineered guide nucleic acid comprises a nucleotide sequence that is at least 80%, at least 85%, at least 90% or at least 95% identical to SEQ ID NO: 260. In some embodiments, the engineered guide nucleic acid comprises a nucleotide sequence that is at least 80%, at least 85%, at least 90% or at least 95% identical to SEQ ID NO: 261. In some embodiments, the engineered guide nucleic acid comprises a nucleotide sequence that is at least 80%, at least 85%, at least 90% or at least 95% identical to SEQ ID NO: 262. In some embodiments, the composition provides cis-cleavage activity on a target nucleic acid. In some embodiments, the composition provides trans cleavage activity on a target nucleic acid. In some embodiments, the programmable nuclease comprises no greater than 540 amino acids. In some embodiments, the programmable nuclease comprises no greater than 500 amino acids. In some embodiments, the programmable nuclease comprises no greater than 480 amino acids. In some embodiments, the programmable nuclease comprises at least three partial RuvC domains. In some embodiments, the at least three partial RuvC domains are not contiguous with respect to the primary amino acid sequence of the programmable nuclease. In some embodiments, the programmable nuclease comprises a single RuvC domain. In some embodiments, the programmable nuclease recognizes a protospacer adjacent motif (PAM) sequence directly adjacent to a target sequence of a target nucleic acid. In some embodiments, the PAM sequence comprises 5′-NTTR-3′, wherein N is any base, T is thymine, and R is a purine. In some embodiments, the PAM sequence comprises 5′-TTTR-3′, wherein T is thymine, and R is a purine. In some embodiments, the PAM sequence comprises 5′-TTTG-3′, wherein T is thymine, and G is a Guanine. In some embodiments, the PAM sequence comprises 5′-GTTG-3′, wherein T is thymine, and G is a Guanine.

Provided herein are compositions comprising a programmable nuclease, or a nucleic acid encoding the programmable nuclease, and an engineered guide nucleic acid, or a DNA molecule encoding the engineered guide nucleic acid, wherein the programmable nuclease comprises an amino acid sequence that is at least 80%, at least 85%, at least 90%, or at least 95% identical to the SEQ ID NO: 1, and wherein the engineered guide nucleic acid comprises a nucleotide sequence that is at least 80%, at least 85%, at least 90% or at least 95% identical to SEQ ID NO: 255. In some embodiments, the engineered guide nucleic acid is a sgRNA.

Provided herein are compositions comprising a programmable nuclease, or a nucleic acid encoding the programmable nuclease, and an engineered guide nucleic acid, or a DNA molecule encoding the an engineered guide nucleic acid, wherein the programmable nuclease comprises an amino acid sequence that is at least 80%, at least 85%, at least 90%, or at least 95% identical to any one of SEQ ID NOs: 2, 4, and 5, and wherein the engineered guide nucleic acid comprises a nucleotide sequence that is at least 80%, at least 85%, at least 90% or at least 95% identical to SEQ ID NO: 256. In some embodiments, the engineered guide nucleic acid is a sgRNA.

Provided herein are compositions comprising a programmable nuclease, or a nucleic acid encoding the programmable nuclease, and an engineered guide nucleic acid, or a DNA molecule encoding the engineered guide nucleic acid, wherein the programmable nuclease comprises an amino acid sequence that is at least 80%, at least 85%, at least 90%, or at least 95% identical to the SEQ ID NO: 3, and wherein the engineered guide nucleic acid comprises a nucleotide sequence that is at least 80%, at least 85%, at least 90% or at least 95% identical to SEQ ID NO: 257. In some embodiments, the engineered guide nucleic acid is a sgRNA.

Provided herein are compositions comprising a programmable nuclease, or a nucleic acid encoding the programmable nuclease, and an engineered guide nucleic acid, or a DNA molecule encoding the engineered guide nucleic acid, wherein the programmable nuclease comprises an amino acid sequence that is at least 80%, at least 85%, at least 90%, or at least 95% identical to the SEQ ID NO: 1, and wherein the engineered guide nucleic acid comprises a nucleotide sequence that is at least 80%, at least 85%, at least 90% or at least 95% identical to 258. In some embodiments, the engineered guide nucleic acid is a crRNA.

Provided herein are compositions comprising a programmable nuclease, or a nucleic acid encoding the programmable nuclease, and an engineered guide nucleic acid, or a DNA molecule encoding the engineered guide nucleic acid, wherein the programmable nuclease comprises an amino acid sequence that is at least 80%, at least 85%, at least 90%, or at least 95% identical to the SEQ ID NO: 2, and wherein the engineered guide nucleic acid comprises a nucleotide sequence that is at least 80%, at least 85%, at least 90% or at least 95% identical to SEQ ID NO: 259. In some embodiments, the engineered guide nucleic acid is a crRNA.

Provided herein are compositions comprising a programmable nuclease, or a nucleic acid encoding the programmable nuclease, and an engineered guide nucleic acid, or a DNA molecule encoding the engineered guide nucleic acid, wherein the programmable nuclease comprises an amino acid sequence that is at least 80%, at least 85%, at least 90%, or at least 95% identical to the SEQ ID NO: 3, and wherein the engineered guide nucleic acid comprises a nucleotide sequence that is at least 80%, at least 85%, at least 90% or at least 95% identical to SEQ ID NO: 260. In some embodiments, the engineered guide nucleic acid is a crRNA.

Provided herein are compositions comprising a programmable nuclease, or a nucleic acid encoding the programmable nuclease, and an engineered guide nucleic acid, or a DNA molecule encoding the engineered guide nucleic acid, wherein the programmable nuclease comprises an amino acid sequence that is at least 80%, at least 85%, at least 90%, or at least 95% identical to the SEQ ID NO: 4, and wherein the engineered guide nucleic acid comprises a nucleotide sequence that is at least 80%, at least 85%, at least 90% or at least 95% identical to SEQ ID NO: 261. In some embodiments, the engineered guide nucleic acid is a crRNA.

Provided herein are compositions comprising a programmable nuclease, or a nucleic acid encoding the programmable nuclease, and an engineered guide nucleic acid, or a DNA molecule encoding the engineered guide nucleic acid, wherein the programmable nuclease comprises an amino acid sequence that is at least 80%, at least 85%, at least 90%, or at least 95% identical to the SEQ ID NO: 5, and wherein the engineered guide nucleic acid comprises a nucleotide sequence that is at least 80%, at least 85%, at least 90% or at least 95% identical to SEQ ID NO: 262. In some embodiments, the engineered guide nucleic acid is a crRNA. In some embodiments, the programmable nuclease comprises at least one amino acid change relative to a wildtype counterpart that reduces a nucleic acid-cleaving activity of the programmable nuclease relative to that of a wildtype counterpart.

In some embodiments, the compositions above comprise a fusion partner fused to the programmable nuclease, or a nucleic acid encoding the fusion partner. In some embodiments, the fusion partner comprises a base editing enzyme. In some embodiments, the fusion partner comprises a transcription activator domain or a transcription repressor domain. In some embodiments, the fusion partner comprises a nuclear localization signal. In some embodiments, the fusion partner comprises a heterologous moiety. In some embodiments, the heterologous moiety comprises a protein purification tag.

In some embodiments, in the compositions described above, the nucleic acid encoding the programmable nuclease is an expression vector. In some embodiments, the expression vector is an adeno associated viral vector. In some embodiments, the expression vector encodes the engineered guide nucleic acid. In some embodiments, in the compositions described above, the nucleic acid encoding the programmable nuclease is a messenger RNA. In some embodiments, the composition further comprises a lipid or a lipid nanoparticle. In some embodiments, the engineered guide RNA comprises at least 10 contiguous nucleotides that are complementary to a eukaryotic sequence.

In some embodiments, the compositions above are present in a pharmaceutical composition. In some embodiments, a method of treating a disease or syndrome comprises administering the pharmaceutical composition.

Provided herein are systems comprising a programmable nuclease comprising an amino acid sequence that is at least 70%, at least 75%, at least 80%, at least 85%, at least 90%, at least 95%, or 100% identical to any one of SEQ ID NOs: 1-5, and an engineered guide nucleic acid that binds to the programmable nuclease.

Provided herein are systems for detecting a target nucleic acid comprising the compositions described above in a solution, wherein the solution comprises at least one of a buffering agent, a salt, a crowding agent, a detergent, a reducing agent, a competitor, and a reporter nucleic acid.

Provided herein are methods of detecting a target nucleic acid in a sample, comprising: (a) contacting the sample with: (i) a composition described above, or system described above, (ii) a reporter nucleic acid that is cleaved in the presence of the programmable nuclease, the engineered guide nucleic acid, and the target nucleic acid, and (iii) detecting a signal produced by cleavage of the reporter nucleic acid, thereby detecting the target nucleic acid in the sample.

Provided herein are methods of generating a recombinant cell, the method comprising delivering to a target cell a composition described above, or system described above, thereby generating the recombinant cell from the target cell.

Provided herein are cells comprising the compositions described above.

Provided herein are recombinant cells produced by the methods described above. In some embodiments, a population of cells is generated from a recombinant cell.

Provided herein are methods of modifying a target nucleic acid, comprising contacting the target nucleic acid with the composition described above, or a system described above, thereby modifying the target nucleic acid.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

It is to be understood that both the foregoing general description and the following detailed description are exemplary and explanatory only and are not restrictive of the disclosure. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

Unless otherwise indicated, all technical terms used herein have the same meaning as commonly understood by one of ordinary skill in the art to which this invention belongs. Unless otherwise indicated or obvious from context, the following terms have the following meanings:

As used in this specification and the appended claims, the singular forms “a,” “an,” and “the” include plural references unless the context clearly dictates otherwise. Any reference to “or” herein is intended to encompass “and/or” unless otherwise stated.

As used herein, the term “about” in reference to a number or range of numbers is understood to mean the stated number and numbers +/−10% thereof, or 10% below the lower listed limit and 10% above the higher listed limit for the values listed for a range.

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, the terms “individual,” “subject,” and “patient” are used interchangeably and include any member of the animal kingdom, including humans.

As used herein, the terms “percent identity” and “% identity” refer to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs may be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The terms “nucleotide” and “nucleoside” when used in the context of a nucleic acid molecule having multiple residues are used interchangeably and mean the sugar and base of the residue contained in the nucleic acid molecule. The term “nucleobase” when used in the context of a nucleic acid molecule can refer to the base of the residue contained in the nucleic acid molecule, for example, the base of a nucleotide or a nucleoside.

Disclosed herein are non-naturally occurring compositions and systems comprising at least one of an engineered Cas protein and an engineered guide nucleic acid, which may simply be referred to herein as a Cas protein and a guide nucleic acid, respectively. Also disclosed herein are non-naturally occurring compositions and systems comprising a nucleic acid encoding the engineered Cas protein, and an engineered guide nucleic acid, or a nucleic acid encoding the engineered guide nucleic acid. In general, an engineered Cas protein and an engineered guide nucleic acid refer to a Cas protein and a guide nucleic acid, respectively, that are not found in nature. In some instances, systems and compositions comprise at least one non-naturally occurring component. For example, compositions and systems may comprise a guide nucleic acid, wherein the sequence of the guide nucleic acid is different or modified from that of a naturally-occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, composition and systems may comprise a guide nucleic acid and a Cas protein that do not naturally occur together. Conversely, and for clarity, a Cas protein or guide nucleic acid that is “natural,” “naturally-occurring,” or “found in nature” includes Cas proteins and guide nucleic acids from cells or organisms that have not been genetically modified by a human or machine.

In some embodiments, guide nucleic acid comprises a nucleic acid comprising: a first nucleotide sequence that hybridizes to a target nucleic acid; and a second nucleotide sequence that is capable of being connected to a programmable nuclease by, for example, being non-covalently bound by a programmable nuclease or hybridized to a separate nucleic acid molecule that is bound by a programmable nuclease. The first sequence may be referred to herein as a spacer sequence. The second sequence may be referred to herein as a repeat sequence. In some instances, the first sequence is located 5′ of the second nucleotide sequence. In some instances, the first sequence is located 3′ of the second nucleotide sequence. In some instances, the guide nucleic acid comprises a non-natural nucleobase sequence. In some instances, the non-natural sequence is a nucleobase sequence that is not found in nature. The non-natural sequence may comprise a portion of a naturally-occurring sequence, wherein the portion of the naturally-occurring sequence is not present in nature absent the remainder of the naturally-occurring sequence. In some instances, the guide nucleic acid comprises two naturally-occurring sequences arranged in an order or proximity that is not observed in nature. In some instances, compositions and systems comprise a ribonucleotide complex comprising a CRISPR/Cas programmable nuclease and a guide nucleic acid that do not occur together in nature. Engineered guide nucleic acids may comprise a first sequence and a second sequence that do not occur naturally together. For example, an engineered guide nucleic acid may comprise a sequence of a naturally-occurring repeat region and a spacer region that is complementary to a naturally-occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, an engineered guide nucleic acid may comprise a naturally occurring crRNA and tracrRNA coupled by a linker sequence.

In some embodiments, CRISPR RNA or crRNA is a type of guide nucleic acid, wherein the nucleic acid is RNA comprising a first sequence, often referred to herein as a spacer sequence, that hybridizes to a target sequence of a target nucleic acid, and a second sequence that is capable of being connected to an programmable nuclease by either a) hybridization to a portion of a tracrRNA or b) being non-covalently bound by a programmable nuclease. In some embodiments, the crRNA is covalently linked to an additional nucleic acid (e.g., a tracrRNA) that is bound by the programmable nuclease. In some embodiments, the crRNA and a tracrRNA are in a dual guide system and are not linked by a covalent bond. In such a dual guide system, the crRNA can be connected to the programmable nuclease by hybridization to a portion of the tracrRNA, and the tracrRNA includes a separate portion that is bound by the programmable nuclease.

In some instances, compositions and systems described herein comprise an engineered Cas protein that is similar to a naturally occurring Cas protein. The engineered Cas protein may lack a portion of the naturally occurring Cas protein. The Cas protein may comprise a mutation relative to the naturally-occurring Cas protein, wherein the mutation is not found in nature. The Cas protein may also comprise at least one additional amino acid relative to the naturally-occurring Cas protein. For example, the Cas protein may comprise an addition of a nuclear localization signal relative to the natural occurring Cas protein. In certain embodiments, the nucleotide sequence encoding the Cas protein is codon optimized (e.g., for expression in a eukaryotic cell) relative to the naturally occurring sequence.

In some instances, compositions and systems provided herein comprise a multi-vector system encoding a Cas protein and a guide nucleic acid described herein, wherein the guide nucleic acid and the Cas protein are encoded by the same or different vectors. In some embodiments, the engineered guide and the engineered Cas protein are encoded by different vectors of the system.

In some instances, compositions and systems provided herein further comprise a modified host cell comprising one or more Cas protein, engineered guide nucleic acids, and/or nucleic acids encoding the same.

### I. Programmable Nucleases

Disclosed herein are programmable nucleases, or nucleic acids encoding the programmable nucleases, and uses thereof, e.g., detection and editing of target nucleic acids. In some instances, programmable nucleases comprise a Type V CRISPR/Cas protein. In some instances, Type V CRISPR/Cas proteins comprise nucleic acid cleavage activity. In some instances, Type V CRISPR/Cas proteins cleave or nick single-stranded nucleic acids, double, stranded nucleic acids, or a combination thereof. In some cases, Type V CRISPR/Cas proteins cleave single-stranded nucleic acids. In some cases, Type V CRISPR/Cas proteins cleave double-stranded nucleic acids. In some cases, Type V CRISPR/Cas proteins nick double-stranded nucleic acids. Typically, guide RNAs of Type V CRISPR/Cas proteins hybridize to ssDNA or dsDNA. However, the trans cleavage activity of Type V CRISPR/Cas protein is typically directed towards ssDNA.

In some cases, the Type V CRISPR/Cas protein comprises a catalytically inactive nuclease domain. In some cases, the Type V CRISPR/Cas protein comprises a catalytically inactive nuclease domain. A catalytically inactive domain of a Type V CRISPR/Cas protein may comprise at least 1, at least 2, at least 3, at least 4, or at least 5 mutations relative to a wild type nuclease domain of the Type V CRISPR/Cas protein. Said mutations may be present within a cleaving or active site of the nuclease.

The Type V CRISPR/Cas protein may be a Cas14 protein. The Type V CRISPR/Cas protein may be a Cas12 protein. The Type V CRISPR/Cas protein may be a CasΦ protein. The Type V CRISPR/Cas protein may be a CasY protein. In some instances, Type V CRISPR/Cas proteins comprise an amino acid sequence that is at least 70%, at least 75%, at least 80%, at least 85%, at least 90%, at least 92%, at least 95%, at least 97%, at least 99%, or 100% identical to any one of SEQ ID NOs: 1-198, as presented in TABLES 1-5. In some instances, the Type V CRISPR/Cas protein comprises an amino acid sequence that is at least 70%, at least 75%, at least 80%, at least 85%, at least 90%, at least 92%, at least 95%, at least 97%, at least 99%, or 100% identical to any one of SEQ ID NOs: 1-5.

In some instances, the Type V CRISPR/Cas protein has been modified (also referred to as an engineered protein). In some instances, an engineered protein comprises an amino acid sequence that is at 70%, at least 75%, at least 80%, at least 85%, at least 90%, at least 92%, at least 95%, at least 97%, at least 99%, or 100% identical to any one of SEQ ID NOS: 1-198. A Type V CRISPR/Cas protein disclosed herein or a variant thereof may comprise a nuclear localization signal (NLS). In some cases, an NLS comprises an entity (e.g., peptide) that facilitates localization of a nucleic acid, protein, or small molecule to the nucleus, when present in a cell that contains a nuclear compartment. An NLS can be located at or near the amino terminus (N-terminus) of the programmable nucleases disclosed herein. An NLS can be located at or near the carboxy terminus (C-terminus) of the programmable nucleases disclosed herein. In some embodiments, a vector encodes the programmable nucleases described herein, wherein the vector or vector systems disclosed herein comprises one or more NLSs, such as about or more than about 1, 2, 3, 4, 5, 6, 7, 8, 9, 10, or more NLSs. In some embodiments, a programmable nuclease described herein comprises 1, 2, 3, 4, 5, 6, 7, 8, 9, 10, or more NLSs at or near the N-terminus, 1, 2, 3, 4, 5, 6, 7, 8, 9, 10, or more NLSs at or near the C-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In some cases, the NLS may comprise a sequence of KRPAATKKAGQAKKKKEF (SEQ ID NO: 199).

In some embodiments, programmable nucleases described herein, such as Type V CRISPR/Cas proteins, are encoded by a codon optimized nucleic acid. In some embodiments, a nucleic acid sequence encoding a programmable nuclease described herein, such as a Type V CRISPR/Cas protein, is codon optimized. This type of optimization can entail a mutation of a programmable nuclease encoding nucleotide sequence to mimic the codon preferences of the intended host organism or cell while encoding the same polypeptide. Thus, the codons can be changed, but the encoded protein remains unchanged. For example, if the intended target cell was a human cell, a human codon-optimized programmable nuclease-encoding nucleotide sequence could be used. As another non-limiting example, if the intended host cell were a mouse cell, then a mouse codon-optimized programmable nuclease—encoding nucleotide sequence could be generated. As another non-limiting example, if the intended host cell were a eukaryotic cell, then a eukaryote codon-optimized programmable nuclease nucleotide sequence could be generated. As another non-limiting example, if the intended host cell were a prokaryotic cell, then a prokaryote codon-optimized programmable nuclease-encoding nucleotide sequence could be generated. Codon usage tables are readily available, for example, at the “Codon Usage Database” available at www.kazusa.or.jp/codon.

Type V CRISPR/Cas proteins may be codon optimized for expression in a specific cell, for example, a bacterial cell, a plant cell, a eukaryotic cell, an animal cell, a mammalian cell, or a human cell. In some embodiments, the Type V CRISPR/Cas protein is codon optimized for a human cell.

When describing a mutation that changes an amino acid residue or a nucleotide as described herein, such a change or changes can include, for example, deletions, insertions, and/or substitutions. The mutation can refer to a change in structure of an amino acid residue or nucleotide relative to the starting or reference residue or nucleotide. A mutation of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. A mutation of a nucleotide includes, for example, changing one naturally occurring base for a different naturally occurring base, such as changing an adenine to a thymine or a guanine to a cytosine or an adenine to a cytosine or a guanine to a thymine. A mutation of a nucleotide base may result in a structural and/or functional alteration of the encoding peptide, polypeptide or protein by changing the encoded amino acid residue of the peptide, polypeptide or protein. A mutation of a nucleotide base may not result in an alteration of the amino acid sequence or function of encoded peptide, polypeptide or protein, also known as a silent mutation.

When a conservative substitution is described herein, such a substitution refers to the replacement of one amino acid for another such that the replacement takes place within a family of amino acids that are related in their side chains. Alternatively, a non-conservative substitution, when described herein, refers to the replacement of one amino acid residue for another such that the replaced residue is going from one family of amino acids to a different family of residues. Genetically encoded amino acids can be divided into four families: (1) acidic (negatively charged)=Asp (D), Glu (G); (2) basic (positively charged)=Lys (K), Arg (R), His (H); (3) non-polar (hydrophobic)=Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic=Ala (A), Val (V), Leu (L), Ile (I), Met (M), Phe (F); and (ii) moderately hydrophobic=Gly (G), Pro (P), Cys (C), Tyr (Y), Trp (W); and (4) uncharged polar=Asn (N), Gln (Q), Ser (S), Thr (T). In alternative fashion, the amino acid repertoire can be grouped as (1) acidic (negatively charged)=Asp (D), Glu (G); (2) basic (positively charged)=Lys (K), Arg (R), His (H), and (3) aliphatic=Gly (G), Ala (A), Val (V), Leu (L), Ile (I), Ser (S), Thr (T), with Ser (S) and Thr (T) optionally being grouped separately as aliphatic-hydroxyl; (4) aromatic=Phe (F), Tyr (Y), Trp (W); (5) amide=Asn (N), Glu (Q); and (6) sulfur-containing=Cys (C) and Met (M) (see, for example, Biochemistry, 4th ed., Ed. by L. Stryer, WH Freeman and Co., 1995, which is incorporated by reference herein in its entirety).

It is understood that when describing coding sequences of polypeptides described herein, said coding sequences do not necessarily require a codon encoding a N-terminal Methionine (M) or a Valine (V) as described for the programmable nucleases described herein. One skilled in the art would understand that a start codon could be replaced or substituted with a start codon that encodes for an amino acid residue sufficient for initiating translation in a host cell.

In some cases, compositions comprise a Type V CRISPR/Cas protein and a cell. In some embodiments, compositions comprise a cell that expresses a Type V CRISPR/Cas protein. In some cases, compositions comprise a nucleic acid encoding a Type V CRISPR/Cas protein and a cell. In some embodiments, compositions comprise a cell expressing a nucleic acid encoding a Type V CRISPR/Cas protein. In some instances, the cell is a prokaryotic cell. In some instances, the cell is a eukaryotic cell. In some instances, the cell is a mammalian cell.

Programmable nucleases of the present disclosure may be produced in vitro or by eukaryotic cells or by prokaryotic cells. Programmable nucleases can be further processed by unfolding, e.g. heat denaturation, dithiothreitol reduction, etc. and may be further refolded, using any suitable method. Programmable nucleases of the present disclosure of the present disclosure may be synthesized, using any suitable method.

In some embodiments, programmable nucleases described herein can be isolated and purified for use in compositions, systems, and/or methods described herein. Methods described here can include the step of isolating programmable nucleases described herein. Compositions and/or systems described herein can further comprise a purification tag that can be attached to a programmable nuclease, or a nucleic acid encoding for a purification tag that can be attached to a nucleic acid encoding for a programmable nuclease as described herein. A purification tag, as used herein, can be an amino acid sequence which can attach or bind with high affinity to a separation substrate and assist in isolating the protein of interest from its environment, which can be its biological source, such as a cell lysate. Attachment of the purification tag can be at the N or C terminus of the programmable nuclease. Furthermore, an amino acid sequence recognized by a protease or a nucleic acid encoding for an amino acid sequence recognized by a protease, such as TEV protease or the HRV3C protease can be inserted between the purification tag and the programmable nuclease, such that biochemical cleavage of the sequence with the protease after initial purification liberates the purification tag. Purification and/or isolation can be through high performance liquid chromatography (HPLC), exclusion chromatography, gel electrophoresis, affinity chromatography, or other purification technique. In some embodiments, purification tags can be a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like.

For example, in some embodiments, programmable nucleases described herein are isolated from cell lysate. In some embodiments, the compositions described herein can comprise 20% or more by weight, 75% or more by weight, 95% or more by weight, or 99.5% or more by weight of a programmable nuclease, related to the method of preparation of compositions described herein and its purification thereof, wherein percentages can be upon total protein content in relation to contaminants. Thus, in some cases, a programmable nuclease described herein is at least 80% pure, at least 85% pure, at least 90% pure, at least 95% pure, at least 98% pure, or at least 99% pure (e.g., free of contaminants, non-engineered polypeptide proteins or other macromolecules, etc.).

### A. Cas14 Proteins

In some instances, the TypeV CRISPR/Cas protein comprises a Cas14 protein. Cas14 proteins may comprise a bilobed structure with distinct amino-terminal and carboxy-terminal domains. The amino- and carboxy-terminal domains may be connected by a flexible linker. The flexible linker may affect the relative conformations of the amino- and carboxyl-terminal domains. The flexible linker may be short, for example less than 10 amino acids, less than 8 amino acids, less than 6 amino acids, less than 5 amino acids, or less than 4 amino acids in length. The flexible linker may be sufficiently long to enable different conformations of the amino- and carboxy-terminal domains among two Cas14 proteins of a Cas14 dimer complex (e.g., the relative orientations of the amino- and carboxy-terminal domains differ between two Cas14 proteins of a Cas14 homodimer complex). The linker domain may comprise a mutation which affects the relative conformations of the amino- and carboxyl-terminal domains. The linker may comprise a mutation which affects Cas14 dimerization. For example, a linker mutation may enhance the stability of a Cas14 dimer.

In some instances, the amino-terminal domain of a Cas14 protein comprises a wedge domain, a recognition domain, a zinc finger domain, or any combination thereof. The wedge domain may comprise a multi-strand β-barrel structure. A multi-strand β-barrel structure may comprise an oligonucleotide/oligosaccharide-binding fold that is structurally comparable to those of some Cas12 proteins. The recognition domain and the zinc finger domain may each (individually or collectively) be inserted between β-barrel strands of the wedge domain. The recognition domain may comprise a 4-α-helix structure, structurally comparable but shorter than those found in some Cas12 proteins. The recognition domain may comprise a binding affinity for a guide nucleic acid or for a guide nucleic acid-target nucleic acid heteroduplex. In some cases, a REC lobe may comprise a binding affinity for a PAM sequence in the target nucleic acid. The amino-terminal may comprise a wedge domain, a recognition domain, and a zinc finger domain. The carboxy-terminal may comprise a RuvC domain, a zinc finger domain, or any combination thereof. The carboxy-terminal may comprise one RuvC and one zinc finger domain.

Cas14 proteins may comprise a RuvC domain or a partial RuvC domain. The RuvC domain may be defined by a single, contiguous sequence, or a set of partial RuvC domains that are not contiguous with respect to the primary amino acid sequence of the Cas14 protein. In some instances, a partial RuvC domain does not have any substrate binding activity or catalytic activity on its own. A Cas14 protein of the present disclosure may include multiple partial RuvC domains, which may combine to generate a RuvC domain with substrate binding or catalytic activity. For example, a Cas14 may include 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. A Cas14 protein may comprise a linker loop connecting a carboxy terminal domain of the Cas14 protein with the amino terminal domain of the Cas 14 protein, and wherein the carboxy terminal domain comprises one or more RuvC domains and the amino terminal domain comprises a recognition domain.

Cas14 proteins may comprise a zinc finger domain. In some instances, a carboxy terminal domain of a Cas14 protein comprises a zinc finger domain. In some instances, an amino terminal domain of a Cas14 protein comprises a zinc finger domain. In some instances, the amino terminal domain comprises a wedge domain (e.g., a multi-β-barrel wedge structure), a zinc finger domain, or any combination thereof. In some cases, the carboxy terminal domain comprises the RuvC domains and a zinc finger domain, and the amino terminal domain comprises a recognition domain, a wedge domain, and a zinc finger domain.

Cas14 proteins may be relatively small compared to many other Cas proteins, making them suitable for nucleic acid detection or gene editing. For instance, a Cas14 protein may be less likely to adsorb to a surface or another biological species due to its small size. The smaller nature of these proteins also allows for them to be more easily packaged as a reagent in a system or assay, and delivered with higher efficiency as compared to other larger Cas proteins. In some cases, a Cas14 protein is 400 to 800 amino acid residues long, 400 to 600 amino acid residues long, 440 to 580 amino acid residues long, 460 to 560 amino acid residues long, 460 to 540 amino acid residues long, 460 to 500 amino acid residues long, 400 to 500 amino acid residues long, or 500 to 600 amino acid residues long. In some cases, a Cas14 protein is less than about 550 amino acid residues long. In some cases, a Cas14 protein is less than about 500 amino acid residues long.

In some instances, a Cas14 protein may function as an endonuclease that catalyzes cleavage at a specific position within a target nucleic acid. In some instances, a Cas14 protein is capable of catalyzing non-sequence-specific cleavage of a single stranded nucleic acid. In some cases, a Cas14 protein is activated to perform trans cleavage activity after binding of a guide nucleic acid with a target nucleic acid. This trans cleavage activity is also referred to as “collateral” or “transcollateral” cleavage. Trans cleavage activity may be non-specific cleavage of nearby single-stranded nucleic acid by the activated programmable nuclease, such as trans cleavage of detector nucleic acids with a detection moiety.

A Cas14 protein may comprise an amino acid sequence that is at least 70%, at least 75%, at least 80%, at least 85%, at least 90%, at least 95%, at least 98%, at least 99%, or 100% identical to any one of SEQ ID NOs: 1-5. SEQ ID NOs: 1-5 are provided in TABLE 1. In some cases, the amino acid sequence of the Cas14 protein is at least 70%, at least 75%, at least 80%, at least 85%, at least 90%, at least 95%, at least 97%, or at least 98%, at least 99%, or 100% identical to any one of SEQ ID NOs: 1-5. The amino acid sequence of the Cas14 protein may be at least 70%, at least 75%, at least 80%, at least 85%, at least 90%, at least 95%, at least 97%, or at least 98%, at least 99%, or 100% identical to SEQ ID NO: 1. The amino acid sequence of the Cas14 protein may be at least 70%, at least 75%, at least 80%, at least 85%, at least 90%, at least 95%, at least 97%, or at least 98%, at least 99%, or 100% identical to SEQ ID NO: 2. The amino acid sequence of the Cas14 protein may be at least 70%, at least 75%, at least 80%, at least 85%, at least 90%, at least 95%, at least 97%, or at least 98%, at least 99%, or 100% identical to SEQ ID NO: 3. The amino acid sequence of the Cas14 protein may be at least 70%, at least 75%, at least 80%, at least 85%, at least 90%, at least 95%, at least 97%, or at least 98%, at least 99%, or 100% identical to SEQ ID NO: 4. The amino acid sequence of the Cas14 protein may be at least 70%, at least 75%, at least 80%, at least 85%, at least 90%, at least 95%, at least 97%, or at least 98%, at least 99%, or 100% identical to SEQ ID NO: 5.

TABLE 1
Select Cas14 Protein Sequences

| SEQ ID | Enzyme | |
|---|---|---|
| NO | Name | Sequence |
| SEQ ID | 280852 | MAGKKKDKDVINKTLSVRIIRPRYSDDIEKEISDEKAKRKQDGKTGE |
| NO: 1 | | LDRAFFSELKSRNPDIITNDELFPLFTEIQKNLTEIYNKSISLLYMKLIV |
| | | EEEGGSTASALSAGPYKECKARFNSYISLGLRQKIQSNFRRKELKGFQ |
| | | VSLPTAKSDRFPIPFCHQVENGKGGFKVYETGDDFIFEVPLIKYTATN |
| | | KKSTSGKNYTKVQLNNPPVPMNVPLLLSTMRRRQTKKGMQWNKD |
| | | EGTNAELRRVMSGEYKVSYAEIIRRTRFGKHDDWFVNFSIKFKNKTD |
| | | ELNQNVRGGIDIGVSNPLVCAVINGLDRYIVANNDIMAFNERAMAR |
| | | RRTLLRKNRFKRSGHGAKNKLEPITVLTEKNERFRKSILQRWAREVA |
| | | EFFKRTSASVVNMEDLSGITEREDFFSTKLRTTWNYRLMQTTIENKL |
| | | KEYGIAVNYISPKYTSQTCHSCGKRNDYFTFSYRSENNYPPFECKEC |
| | | NKVKCNADFNAAKNIALKVVL |
| SEQ ID | 292901 | MRISKTLSLRIVRPFYTPEVEAGIKAEKDKREAQGQTRSLDAKFFNEL |
| NO: 2 | | KKKHSEIILSSEFYSLLSEVQRQLTSIYNHAMSNLYHKIIVEGEKTSTS |
| | | KALSNIGYDECKAIFPSYMALGLRQKIQSNFRRRDLKNFRMAVPTAK |
| | | SDKFPIPIYRQVDGSKGGFKISENDGKDFIVELPLVDYVAEEVKTAKG |
| | | RFTKINISKPPKIKNIPVILSTLRRRQSGQWFSDDGTNAEIRRVISGEYK |
| | | VSWIEIVRRTRFGKHDDWFVNMVIKYDKPEEGLDSKVVGGIDVGVS |
| | | SPLVCALNNSLDRYFVKSSDIIAFNKRAMARRRTLLRQNKYKRSGH |
| | | GSKNKLEPITVLTEKNERFKKSIMQRWAKEVAEFFRGKGASVVRME |
| | | ELSGLKEKDNFFSSYLRMYWNYGQLQQIIENKLKEYGIKVNYVSPK |
| | | DTSKKCHSCTHINEFFTFEYRQKNNFPLFKCEKCGVECSADYNAAKN |
| | | MAIA |
| SEQ ID | 294601 | MKDYIRKTLSLRILRPYYGEEIEKEIAAAKKKSQAEGGDGALDNKFW |
| NO: 3 | | DRLKAEHPEIISSREFYDLLDAIQRETTLYYNRAISKLYHSLIVEREQV |
| | | STAKALSAGPYHEFREKFNAYISLGLREKIQSNFRRKELARYQVALP |
| | | TAKSDTFPIPIYKGFDKNGKGGFKVREIENGDFVIDLPLMAYHRVGG |
| | | KAGREYIELDRPPAVLNVPVILSTSRRRANKTWFRDEGTDAEIRRVM |
| | | AGEYKVSWVEILQRKRFGKPYGGWYVNFTIKYQPRDYGLDPKVKG |
| | | GIDIGLSSPLVCAVTNSLARLTIRDNDLVAFNRKAMARRRTLLRQNR |
| | | YKRSGHGSANKLKPIEALTEKNELYRKAIMRRWAREAADFFRQHRA |
| | | ATVNMEDLTGIKDREDYFSQMLRCYWNYSQLQTMLENKLKEYGIA |
| | | VKYIEPKDTSKTCHSCGHVNEYFDFNYRSAHKFPMFKCEKCGVECG |
| | | ADYNAARNIAQA |
| SEQ ID | 295351 | VKISKTLSLRIIRPYYTPEVESAIKAEKDKREAQGQTRNLDAKFFNEL |
| NO: 4 | | KKKHPQIILSGEFYSLLFEMQRQLTSIYNRAMSSLYHKIIVEGEKTSTS |
| | | KALSDIGYDECKSVFPSYIALGLRQKIQSNFRRKELKGFRMAVPTAK |
| | | SDKFPIPIYKQVDDGKGGFKISENKEGDFIVELPLVEYTAEDVKTAKG |
| | | KFTKINISKPPKIKNIPVILSTLRRKQSGQWFSDEGTNAEIRRVISGEYK |
| | | VSWIEVVRRTRFGKHDDWFLNIVIKYDKTEDGLDPEVVGGIDVGVS |
| | | TPLVCAVNNSLDRYFVKSSDIIAFKKRAMARRRTLLRQNRFKRSGHG |
| | | SKSKLEPITILTEKNERFKKSIMQRWAKEVAEFFKGERASVVQMEEL |
| | | SGLKEKDNFFGSYLRMYWNYGQLQQIIENKLKEYGIKVNYVSPKDT |
| | | SKKCHSCGYINEFFTFEFRQKNNFPLFKCKKCGVECNADYNAAKNIA |
| | | IA |
| SEQ ID | 298142 | VPITKTISLRILRPYYPPEIEAKIKAEKEKRKENGDTGSLNSSYYRELK |
| NO: 5 | | KEYPSIIINDEFFPLLSEMQRNITSIYNRTISHLYHRLIIKKESISTAKAL |
| | | SEGPYRDFKSTFNSYIALGLRQKVQSNFRKKDLMAFKIALPTAKSDK |
| | | FPIPIYMQTNFKIKESPDSDFIIELPLVEYIAKETKGKNKMFTKVEILSP |
| | | PKVKNIPVILSTRRRKESGQWFSDEGTNAEIRRIISGEYKVSWIEIVKR |
| | | TRFGKHDWFVNMVISFEESQEGLDPDVIGGIDIGVSKPLICAINNSLD |
| | | RYIVKGDDIIAFNRRALSRRRSLLRRNRLKRSGHGSRNKLEPITVLTE |
| | | KNERFKKSIMQRWAKEVAEFFKSKRASIVQMEELTGIKEREDFFSKT |
| | | LRMYWNYGQLQKTVENKLREYGIEVRYASPKDTSRRCHSCGHIND |
| | | YFTFEFRQQNNFPLFKCMNCGIECSADYNAARNIAIAR |

### B. Engineered Proteins

In some instances, a programmable nuclease disclosed herein is an engineered protein. The engineered protein is not identical to a naturally-occurring protein. Such an engineered protein can include one or more mutations, including an insertion, deletion or substitution (e.g., conservative or non-conservative substitution). An engineered protein, in some embodiments, includes at least one mutation relative to a reference protein (e.g., a naturally naturally-occurring protein). In some embodiments, an engineered protein includes at least 1, at least 2, at least 3, at least 4, at least 5, at least 6, at least 7, at least 8, at least 9, at least 10, at least 15, at least 20, at least 25 or at least 30 mutations relative to a reference protein (e.g., a naturally naturally-occurring protein). In some embodiments, an engineered protein includes no more than 10, 20, 30, 40, or 50 mutations relative to a reference protein (e.g., a naturally naturally-occurring protein). The engineered protein may provide enhanced nuclease or nickase activity as compared to a naturally occurring nuclease or nickase. By way of non-limiting example, some engineered proteins exhibit optimal activity at lower salinity and viscosity than the protoplasm of their bacterial cell of origin. Also by way of non-limiting example, bacteria often comprise protoplasmic salt concentrations greater than 250 mM and room temperature intracellular viscosities above 2 centipoise, whereas engineered proteins exhibit optimal activity (e.g., cis-cleavage activity) at salt concentrations below 150 mM and viscosities below 1.5 centipoise. The present disclosure leverages these dependencies by providing engineered proteins in solutions optimized for their activity and stability.

Compositions and systems described herein may comprise an engineered protein in a solution comprising a room temperature viscosity of less than about 15 centipoise, less than about 12 centipoise, less than about 10 centipoise, less than about 8 centipoise, less than about 6 centipoise, less than about 5 centipoise, less than about 4 centipoise, less than about 3 centipoise, less than about 2 centipoise, or less than about 1.5 centipoise. Compositions and systems may comprise an engineered protein in a solution comprising an ionic strength of less than about 500 mM, less than about 400 mM, less than about 300 mM, less than about 250 mM, less than about 200 mM, less than about 150 mM, less than about 100 mM, less than about 80 mM, less than about 60 mM, or less than about 50 mM. Compositions and systems may comprise an engineered protein and an assay excipient, which may stabilize a reagent or product, prevent aggregation or precipitation, or enhance or stabilize a detectable signal (e.g., a fluorescent signal). Examples of assay excipients include, but are not limited to, saccharides and saccharide derivatives (e.g., sodium carboxymethyl cellulose and cellulose acetate), detergents, glycols, polyols, esters, buffering agents, alginic acid, and organic solvents (e.g., DMSO).

An engineered protein may comprise a modified form of a wild type counterpart protein. The modified form of the wild type counterpart may comprise an amino acid change (e.g., deletion, insertion, or substitution) that reduces the nucleic acid-cleaving activity of the programmable nuclease. For example, a nuclease domain (e.g., RuvC domain) of a Type V CRISPR/Cas protein may be deleted or mutated so that it is no longer functional or comprises reduced nuclease activity. The modified form of the programmable nuclease may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. Engineered proteins may have no substantial nucleic acid-cleaving activity. Engineered proteins may be enzymatically inactive or “dead,” that is it may bind to a nucleic acid but not cleave it. An enzymatically inactive protein may comprise an enzymatically inactive domain (e.g. inactive nuclease domain). Enzymatically inactive may refer to an activity less than 1%, less than 2%, less than 3%, less than 4%, less than 5%, less than 6%, less than 7%, less than 8%, less than 9%, or less than 10% activity compared to the wild-type counterpart. A dead protein may associate with a guide nucleic acid to activate or repress transcription of a target nucleic acid sequence. In some embodiments, the enzymatically inactive protein is fused with a protein comprising recombinase activity. In some embodiments, the enzymatically inactive protein is fused with a recombinase. In certain embodiments, the recombinase is a site-specific recombinase. In some embodiments, described herein is a programmed nuclease comprising reduced nuclease activity or no nuclease activity and fused with a recombinase, wherein the recombinase can be a site-specific recombinase. Such polypeptides can be used for site-directed transgene insertion. Examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the site-recombinase is an integrase. In certain embodiments, the site-recombinase is an integrase selected from the group consisting of Bxb1, wBeta, BL3, phiR4, A118, TG1, MR11, phi370, SPBc, TP901-1, phiRV, FC1, K38, phiBT1 and phiC31. In some embodiments, a programmable nuclease comprises a protein, polypeptide, or peptide that non-covalently binds to a guide nucleic acid to form a complex that contacts a target nucleic acid, wherein at least a portion of the guide nucleic acid hybridizes to a target sequence of the target nucleic acid. A complex between a programmable nuclease and a guide nucleic acid can include multiple programmable nucleases or a single programmable nuclease. In some instances, the programmable nuclease modifies the target nucleic acid when the complex contacts the target nucleic acid. In some instances, the programmable nuclease does not modify the target nucleic acid, but it is fused to a fusion partner protein that modifies the target nucleic acid when the complex contacts the target nucleic acid. A non-limiting example of a programmable nuclease modifying a target nucleic acid is cleaving of a phosphodiester bond of the target nucleic acid. Additional examples of modifications a programmable nuclease can make to target nucleic acids are described herein and throughout. A programmable nuclease may be brought into proximity of a target nucleic acid in the presence of a guide nucleic acid when the guide nucleic acid includes a nucleotide sequence that is complementary with a target sequence in the target nucleic acid. The ability of a programmable nuclease to modify a target nucleic acid may be dependent upon the programmable nuclease being bound to a guide nucleic acid and the guide nucleic acid being hybridized to a target nucleic acid. A programmable nuclease may also recognize a protospacer adjacent motif (PAM) sequence present in the target nucleic acid, which may direct the modification activity of the programmable nuclease. A programmable nuclease may modify a nucleic acid by cis cleavage or trans cleavage. The modification of the target nucleic acid generated by a programmable nuclease may, as a non-limiting example, result in modulation of the expression of the nucleic acid (e.g., increasing or decreasing expression of the nucleic acid) or modulation of the activity of a translation product of the target nucleic acid (e.g., inactivation of a protein binding to an RNA molecule or hybridization). A programmable nuclease may be a CRISPR-associated (“Cas”) protein. A programmable nuclease may function as a single protein, including a single protein that is capable of binding to a guide nucleic acid and modifying a target nucleic acid. Alternatively, a programmable nuclease may function as part of a multiprotein complex, including, for example, a complex having two or more programmable nucleases, including two or more of the same programmable nucleases (e.g., dimer or multimer). A programmable nuclease, when functioning in a multiprotein complex, may have only one functional activity (e.g., binding to a guide nucleic acid), while other programmable nucleases present in the multiprotein complex are capable of the other functional activity (e.g., modifying a target nucleic acid). A programmable nuclease may be a modified programmable nuclease having reduced modification activity (e.g., a catalytically defective programmable nuclease) or no modification activity (e.g., a catalytically inactive programmable nuclease). Accordingly, a programmable nuclease as used herein encompasses a modified or programmable nuclease that does not have nuclease activity.

### C. Fusion Proteins

In some embodiments, a fusion programmable nuclease, a fusion protein, a fusion polypeptide comprise a protein comprising at least two heterologous polypeptides. Often a fusion programmable nuclease comprises a programmable nuclease and a fusion partner protein. In general, the fusion partner protein is not a programmable nuclease. In some cases, a fusion partner protein or a fusion partner comprises a polypeptide or peptide that is fused to a programmable nuclease. The fusion partner generally imparts some function to the fusion protein that is not provided by the programmable nuclease. The fusion partner may provide a detectable signal. The fusion partner may modify a target nucleic acid, including changing a nucleobase of the target nucleic acid and making a chemical modification to one or more nucleotides of the target nucleic acid. The fusion partner may be capable of modulating the expression of a target nucleic acid. The fusion partner may inhibit, reduce, activate or increase expression of a target nucleic acid via additional proteins or nucleic acid modifications to the target sequence.

In some instances, a programmable nuclease is a fusion protein, wherein the fusion protein comprises a Type V CRISPR/Cas protein (e.g., a Cas12 or Cas14 protein) and a fusion partner protein. In some instances, the Type V CRISPR/Cas protein comprises an amino acid sequence that is at least 70%, at least 75%, at least 80%, at least 85%, at least 90%, at least 95%, or 100% identical to any one of SEQ ID NOs: 1-5. In some instances the amino acid of the Type V CRISPR/Cas protein is at least 70%, at least 75%, at least 80%, at least 85%, at least 90%, at least 95%, or 100% identical to any one of SEQ ID NOs: 1-5. Unless otherwise indicated, reference to Cas proteins (e.g., Cas14 proteins and Cas 12 proteins) throughout the present disclosure include fusion proteins thereof.

A fusion partner protein is also simply referred to herein as a fusion partner. In some cases, the fusion partner promotes the formation of a multimeric complex of the Type V CRISPR/Cas protein. In some instances, the fusion partner inhibits the formation of a multimeric complex of the Type V CRISPR/Cas protein. By way of non-limiting example, the fusion protein may comprise a Cas14 protein, and a fusion partner comprising a Calcineurin A tag, wherein the fusion protein dimerizes in the presence of Tacrolimus (FK506). Also by way of non-limiting example, the fusion protein may comprise a Cas14 protein and a SpyTag configured to dimerize or associate with another programmable nuclease in a multimeric complex.

In some instances, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some instances, fusion partners that increase or decrease transcription include a transcription activator domain or a transcription repressor domain, respectively.

In some embodiments, a fusion protein and/or a fusion partner can comprise a prime editing enzyme. In some embodiments, a prime editing enzyme is a protein, a polypeptide or a fragment thereof that is capable of catalyzing the modification (insertion, deletion, or base-to-base conversion) of a target nucleotide or nucleotide sequence in a nucleic acid. A prime editing enzyme capable of catalyzing such a reaction includes a reverse transcriptase. A prime editing enzyme may require a prime editing guide RNA (pegRNA) to catalyze the modification. Such a pegRNA can be capable of identifying the nucleotide or nucleotide sequence in the target nucleic acid to be edited and encoding the new genetic information that replaces the targeted nucleotide or nucleotide sequence in the nucleic acid. A prime editing enzyme may require a prime editing guide RNA (pegRNA) and a single guide RNA to catalyze the modification. In some embodiments, such a prime editing enzyme is an M-MLV RT enzyme or a mutant thereof. In some embodiments, the M-MLV RT enzyme comprises at least one mutation selected from D200N, L603W, T330P, T306K, and W313F relative to wildtype M-MLV RT enzyme. In some embodiments, the M-MLV RT enzyme comprises a sequence that is at least 75%, at least 80%, at least 85%, at least 90%, at least 95%, at least 99% or 100% identical to SEQ ID NO: 283.

In some cases, the fusion partner modulates transcription (e.g., inhibits transcription, increases transcription) of a target nucleic acid. In some cases, the fusion partner is a protein (or a domain from a protein) that inhibits transcription, also referred to as a transcriptional repressor. Transcriptional repressors may inhibit transcription via recruitment of transcription inhibitor proteins, modification of target DNA such as methylation, recruitment of a DNA modifier, modulation of histones associated with target DNA, recruitment of a histone modifier such as those that modify acetylation and/or methylation of histones, or a combination thereof. In some cases, the fusion partner is a protein (or a domain from a protein) that increases transcription, also referred to as a transcription activator. Transcriptional activators may promote transcription via recruitment of transcription activator proteins, modification of target DNA such as demethylation, recruitment of a DNA modifier, modulation of histones associated with target DNA, recruitment of a histone modifier such as those that modify acetylation and/or methylation of histones, or a combination thereof. In some cases, the fusion partner is a reverse transcriptase. In some cases, the fusion partner is a base editor. In general, a base editor comprises a deaminase that when fused with a Cas protein changes a nucleobase to a different nucleobase, e.g., cytosine to thymine or guanine to adenine. In some instances, the base editor comprises a deaminase.

In some cases, fusion proteins are targeted by a guide nucleic acid (guide RNA) to a specific location in the target nucleic acid and exert locus-specific regulation such as blocking RNA polymerase binding to a promoter (which selectively inhibits transcription activator function), and/or modifying the local chromatin status (e.g., when a fusion sequence is used that modifies the target nucleic acid or modifies a protein associated with the target nucleic acid). In some cases, the modifications are transient (e.g., transcription repression or activation). In some cases, the modifications are inheritable. For instance, epigenetic modifications made to a target nucleic acid, or to proteins associated with the target nucleic acid, e.g., nucleosomal histones, in a cell, are observed in cells produced by proliferation of the cell.

i. Modifying Target Nucleic Acid

In some cases, fusion partners provide enzymatic activity that modifies a target nucleic acid. Such enzymatic activities include, but are not limited to, nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, and glycosylase activity. In some cases nuclease activity comprises the enzymatic activity of an enzyme which allows the enzyme to cleave the phosphodiester bonds between the nucleotide subunits of nucleic acids. In some case, an enzyme with nuclease activity can comprise a nuclease.

Disclosed herein are compositions and methods for modifying a target nucleic acid. The target nucleic acid may be a gene or a portion thereof. Methods and compositions may modify a coding portion of a gene, a non-coding portion of a gene, or a combination thereof. Modifying at least one gene using the compositions and methods described herein may reduce or increase expression of one or more genes. In some embodiments, compositions and methods reduce expression of one or more genes by at least 10%, at least 20%, at least 30%, at least 40%, at least 50%, at least 60%, at least 70%, at least 80%, at least 90%, or at least 95%. In some embodiments, compositions and methods remove all expression of a gene, also referred to as genetic knock out. In some embodiments, compositions and methods increase expression of one or more genes by at least 10%, at least 20%, at least 30%, at least 40%, at least 50%, at least 60%, at least 70%, at least 80%, at least 90%, or at least 100%.

In some instances, compositions and methods use Cas proteins that are fused to a heterologous protein. Heterologous proteins include, but are not limited to, transcriptional activators, transcriptional repressors, deaminases, methyltransferases, acetyltransferases, and other nucleic acid modifying proteins. In some cases, Cas proteins need not be fused to a partner protein to accomplish the required protein (expression) modification.

In some cases, fusion partners have enzymatic activity that modifies the target nucleic acid. The target nucleic acid may comprise or consist of a ssRNA, dsRNA, ssDNA, or a dsDNA. Examples of enzymatic activity that modifies the target nucleic acid include, but are not limited to: nuclease activity such as that provided by a restriction enzyme (e.g., FokI nuclease); methyltransferase activity such as that provided by a methyltransferase (e.g., HhaJ DNA m5c-methyltransferase (M.HhaJ), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase); transposase activity, recombinase activity such as that provided by a recombinase (e.g., catalytic domain of Gin recombinase); as well as polymerase activity, ligase activity, helicase activity, photolyase activity, and glycosylase activity.

Non-limiting examples of fusion partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins. It is understood that a fusion protein may include the entire protein or in some cases may include a fragment of the protein (e.g., a functional domain). In some instances, the functional domain interacts with or binds ssRNA, including intramolecular and/or intermolecular secondary structures thereof, e.g., hairpins, stem-loops, etc.). The functional domain may interact transiently or irreversibly, directly or indirectly. In some cases, a functional domain comprises a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity. Fusion proteins may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for repressing translation (e.g., Ago2 and Ago4); proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); proteins and protein domains responsible for RNA localization (e.g., from IMP1, ZBP1, She2p, She3p, and Bicaudal-D); proteins and protein domains responsible for nuclear retention of RNA (e.g., Rrp6); proteins and protein domains responsible for nuclear export of RNA (e.g., TAP, NXF1, THO, TREX, REF, and Aly); proteins and protein domains responsible for repression of RNA splicing (e.g., PTB, Sam68, and hnRNP A1); proteins and protein domains responsible for stimulation of RNA splicing (e.g., Serine/Arginine-rich (SR) domains); proteins and protein domains responsible for reducing the efficiency of transcription (e.g., FUS (TLS)); and proteins and protein domains responsible for stimulating transcription (e.g., CDK7 and HIV Tat). Alternatively, the effector domain may be a domain of a protein selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable fusion partner is a PUF RNA-binding domain, which is described in more detail in WO2012068627, which is hereby incorporated by reference in its entirety.

In some instances, the fusion partner comprises an RNA splicing factor. The RNA splicing factor may be used (in whole or as fragments thereof) for modular organization, with separate sequence-specific RNA binding modules and splicing effector domains. Non-limiting examples of RNA splicing factors include members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal Glycine-rich domain. Some splicing factors may regulate alternative use of splice site (ss) by binding to regulatory sequences between the two alternative sites. For example, ASF/SF2 may recognize ESEs and promote the use of intron proximal sites, whereas hnRNP A1 may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c6-elements that are located in either the core exon region or the exon extension region (i.e., between the two alternative 5′ splice sites). For more examples, see WO2010075303, which is hereby incorporated by reference in its entirety.

ii. Base Editors

In some embodiments, fusion partners modify a nucleobase of a target nucleic acid. Fusion proteins comprising such fusion partners and a Cas programmable nuclease may be referred to as base editors. In some embodiments, base editors modify a sequence of a target nucleic acid. In some embodiments, base editors provide a nucleobase change in a DNA molecule. In some embodiments, the nucleobase change in the DNA molecule is selected from: an adenine (A) to guanine (G); cytosine (C) to thymine (T); and cytosine (C) to guanine (G). In some embodiments, base editors provide a nucleobase change in an RNA molecule. In some embodiments, the nucleobase change in the RNA molecule is selected from: adenine (A) to guanine (G); uracil (U) to cytosine (C); cytosine (C) to guanine (G); and guanine (G) to adenine (A). In some embodiments, the fusion partner is a deaminase, e.g., ADAR1/2.

Some base editors modify a nucleobase of on a single strand of DNA. In some embodiments, base editors modify a nucleobase on both strands of dsDNA. In some embodiments, upon binding to its target locus in DNA, base pairing between the guide RNA and target DNA strand leads to displacement of a small segment of single-stranded DNA in an “R-loop”. In some embodiments, DNA bases within the R-loop are modified by the deaminase enzyme. In some embodiments, DNA base editors for improved efficiency in eukaryotic cells comprise a catalytically inactive programmable nuclease that may generate a nick in the non-edited DNA strand, inducing repair of the non-edited strand using the edited strand as a template.

In some embodiments, a catalytically inactive programmable nuclease can comprise a programmable nuclease that is modified relative to a naturally-occurring programmable nuclease to have a reduced or eliminated catalytic activity relative to that of the naturally-occurring programmable nuclease, but retains its ability to interact with a guide nucleic acid. The catalytic activity that is reduced or eliminated is often a nuclease activity. The naturally-occurring programmable nuclease may be a wildtype protein. In some embodiments, the catalytically inactive programmable nuclease is referred to as a catalytically inactive variant of a programmable nuclease, e.g., a Cas programmable nuclease.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise a Cas programmable nuclease that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions comprise a base editor and a guide nucleic acid, wherein the guide nucleic acid directs the base editor to a sequence in a target gene. The target gene may be associated with a disease. In some embodiments, the guide nucleic acid directs that base editor to or near a mutation in the sequence of a target gene. The mutation may be the deletion of one more nucleotides. The mutation may be the addition of one or more nucleotides. The mutation may be the substitution of one or more nucleotides. The mutation may be the insertion, deletion or substitution of a single nucleotide, also referred to as a point mutation. The point mutation may be a SNP. The mutation may be associated with a disease. In some embodiments, the guide nucleic acid directs the base editor to bind a target sequence within the target nucleic acid that is within 0, 1, 2, 3, 4, 5, 6, 7, 8, 9, 10, 11, 12, 13, 14, 15, 16, 17, 18, 19, or 20 nucleotides of the mutation. In some embodiments, the guide nucleic acid comprises a sequence that is identical, complementary or reverse complementary to a target sequence of a target nucleic acid that comprises the mutation. In some embodiments, the guide nucleic acid comprises a sequence that is identical, complementary or reverse complementary to a target sequence of a target nucleic acid that is within 1, 2, 3, 4, 5, 6, 7, 8, 9, 10, 11, 12, 13, 14, 15, 16, 17, 18, 19, or 20 nucleotides of the mutation.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise a Cas programmable nuclease that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions comprise a base editor and a guide nucleic acid, wherein the guide nucleic acid directs the base editor to a sequence in a target gene

In some embodiments, fusion partners comprise a base editing enzyme. In some embodiments, the base editing enzyme modifies the nucleobase of a deoxyribonucleotide. In some embodiments, the base editing enzyme modifies the nucleobase of a ribonucleotide. A base editing enzyme that converts a cytosine to a guanine or thymine may be referred to as a cytosine base editing enzyme. A base editing enzyme that converts an adenine to a to a guanine may be referred to as an adenine base editing enzyme. In some embodiments, the base editing enzyme comprises a deaminase enzyme. In some embodiments, the deaminase functions as a monomer. In some embodiments, the deaminase functions as heterodimer with an additional protein. In some embodiments, base editors comprise a DNA glycosylase inhibitor. In some embodiments, base editors comprise a uracil glycosylase inhibitor (UGI) or uracil N-glycosylase (UNG). In some embodiments, base editors do not comprise a UGI. In some embodiments, base editors do not comprise a UNG. In some embodiments, base editors do not comprise a functional fragment of a UGI. A functional fragment of a UGI is a fragment of a UGI that is capable of excising a uracil residue from DNA by cleaving an N-glycosydic bond. In some cases, a functional fragment comprises a fragment of a protein that retains some function relative to the entire protein. Non-limiting examples of functions are nucleic acid binding, protein binding, nuclease activity, nickase activity, deaminase activity, demethylase activity, or acetylation activity.

In some embodiments, a base editing enzyme can comprise a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase. In some cases, a base editor can be a fusion protein comprising a base editing enzyme fused to a programmable nuclease. The base editor is functional when the programmable nuclease is coupled to a guide nucleic acid. The guide nucleic acid imparts sequence specific activity to the base editor. By way of non-limiting example, the programmable nuclease may comprise a catalytically inactive programmable nuclease. Also, by way of non-limiting example, the base editing enzyme may comprise deaminase activity. Additional base editors are described herein.

In some embodiments, the base editor is a cytidine deaminase base editor generated by ancestral sequence reconstruction as described in WO2019226953, which is hereby incorporated by reference in its entirety.

Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3:eaao4774 (2017), and Rees et al., Nat Rev Genet. 2018 December; 19(12):770-788. doi: 10.1038/s41576-018-0059-1, which are hereby incorporated by reference in their entirety.

In some embodiments, the base editor is a cytosine base editor (CBE). In general, a CBE comprises a cytosine base editing enzyme and a catalytically inactive programmable nuclease. In some embodiments, the catalytically inactive programmable nuclease is a catalytically inactive variant of a Cas programmable nuclease described herein. The CBE may convert a cytosine to a thymine. In some embodiments, the base editor is an adenine base editor (ABE). In general, an ABE comprises an adenine base editing enzyme and a catalytically inactive programmable nuclease. In some embodiments, the catalytically inactive programmable nuclease is a catalytically inactive variant of a Cas programmable nuclease described herein. The ABE generally converts an adenine to a guanine. In some embodiments, the base editor is a cytosine to guanine base editor (CGBE). In general, a CGBE converts a cytosine to a guanine.

In some embodiments, the base editor is a CBE. In some embodiments, the cytosine base editing enzyme is a cytidine deaminase. In some embodiments, the cytosine deaminase is an APOBEC1 cytosine deaminase, which accept ssDNA as a substrate but is incapable of cleaving dsDNA, fused to a catalytically inactive programmable nuclease. In some embodiments, when bound to its cognate DNA, the catalytically inactive programmable nuclease performs local denaturation of the DNA duplex to generate an R-loop in which the DNA strand not paired with the guide RNA exists as a disordered single-stranded bubble. In some embodiments, the catalytically inactive programmable nuclease generated ssDNA R-loop enables the CBE to perform efficient and localized cytosine deamination in vitro. In some examples, deamination activity is exhibited in a window of about 4 to about 10 base pairs. In some embodiments, fusion to the catalytically inactive programmable nuclease presents the target site to APOBEC1 in high effective molarity, enabling the CBE to deaminate cytosines located in a variety of different sequence motifs, with differing efficacies. In some embodiments, the CBE is capable of mediating RNA-programmed deamination of target cytosines in vitro. In some embodiments, the CBE is capable of mediating RNA-programmed deamination of target cytosines in vivo. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C⋅G-to-G⋅C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise a uracil glycosylase inhibitor (UGI) or uracil N-glycosylase (UNG). In some embodiments, base excision repair (BER) of U⋅G in DNA is initiated by a UNG, which recognizes the U⋅G mismatch and cleaves the glyosidic bond between uracil and the deoxyribose backbone of DNA. In some embodiments, BER results in the reversion of the U⋅G intermediate created by the first CBE back to a C⋅G base pair. In some embodiments, UNG may be inhibited by fusion of uracil DNA glycosylase inhibitor (UGI), in some embodiments, a small protein from bacteriophage PBS, to the C-terminus of the CBE. In some embodiments, UGI is a DNA mimic that potently inhibits both human and bacterial UNG. In some embodiments, a UGI inhibitor is any protein or polypeptide that inhibits UNG. In some embodiments, the CBE mediates efficient base editing in bacterial cells and moderately efficient editing in mammalian cells, enabling conversion of a C⋅G base pair to a T⋅A base pair through a U⋅G intermediate. In some embodiments, the CBE is modified to increase base editing efficiency while editing more than one strand of DNA.

In some embodiments, the CBE nicks the non-edited DNA strand. In some embodiments, the non-edited DNA strand nicked by the CBE biases cellular repair of the U⋅G mismatch to favor a U⋅A outcome, elevating base editing efficiency. In some embodiments, the APOBEC1-nickase-UGI fusion efficiently edits in mammalian cells, while minimizing frequency of non-target indels.

In some embodiments, the cytidine deaminase is selected from APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, or saBE4-Gam as described in WO2021163587, WO202108746, WO2021062227, and WO2020123887, which are incorporated herein by reference in their entirety.

In some embodiments, the fusion protein further comprises a non-protein uracil-DNA glcosylase inhibitor (npUGI). In some embodiments, the npUGI is selected from a group of small molecule inhibitors of uracil-DNA glycosylase (UDG), or a nucleic acid inhibitor of UDG. In some embodiments, the non-protein uracil-DNA glcosylase inhibitor (npUGI) is a small molecule derived from uracil. Examples of small molecule non-protein uracil-DNA glcosylase inhibitors, fusion proteins, and Cas-CRISPR systems comprising base editing activity are described in WO202108746, which is incorporated by reference in its entirety.

In some embodiments, the fusion partner is a deaminase, e.g., ADAR1/2, ADAR-2, or AID. In some embodiments, the base editor is an ABE. In some embodiments, the adenine base editing enzyme of the ABE is an adenosine deaminase. In some embodiments, the adenine base editing enzyme is selected from ABE8e, ABE8.20m, APOBEC3A, Anc APOBEC, and BtAPOBEC2. In some embodiments, the ABE base editor is an ABE7 base editor. In some embodiments, the deaminase or enzyme with deaminase activity is selected from ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, or ABE8.24d. In some embodiments, the adenine base editing enzyme is ABE8.1d. In some embodiments, the adenosine base editor is ABE9. Exemplary deaminases are described in US20210198330, WO2021041945, WO2021050571A1, and WO2020123887, all of which are incorporated herein by reference in their entirety. Sequences of a selection of these enzymes are provided in TABLE 2. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871. Additional examples of deaminase domains are also described in WO2018027078 and WO2017070632, which are hereby incorporated by reference in their entirety.

In some embodiments, an ABE converts an A⋅T base pair to a G⋅C base pair. In some embodiments, the ABE converts a target A⋅T base pair to G⋅C in vivo. In some embodiments, the ABE converts a target A⋅T base pair to G⋅C in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. In some embodiments, an ABE comprises an engineered adenosine deaminase enzyme capable of acting on ssDNA.

In some embodiments, a base editor comprises an adenosine deaminase variant that differs from a naturally occurring deaminase. Relative to the naturally occurring deaminase, the adenosine deaminase variant may comprise a V82S alteration, a T166R alteration, or a combination thereof. In some embodiments, the adenosine deaminase variant comprises at least one of the following alterations relative to a naturally occurring adenosine deaminase: Y147T, Y147R, Q154S, Y123H, and Q154R., which are incorporated herein by reference in their entirety.

In some embodiments, a base editor comprises a deaminase dimer. In some embodiments, a base editor is a deaminase dimer further comprising a base editing enzyme and an adenine deaminase (e.g., TadA).

TadA comprises or consists of at least a portion of the sequence:

(SEQ ID NO: 273)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
RRQEIKAQKKAQSSTD.

In some embodiments, the adenosine deaminase is a TadA monomer (e.g., Tad*7.10, TadA*8 or TadA*9). In some embodiments, the adenosine deaminase is a TadA*8 variant. Such a TadA*8 variant includes TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entiry.

In some embodiments, a base editor is a deaminase dimer comprising a base editing enzyme fused to TadA via a linker. In some embodiments the linker comprises or consists of at least a portion of the sequence:

SGGSSGGSSGSETPGTSESATPESSGGSSGGS (SEQ ID NO: 274). In some embodiments, the amino acid sequence of the linker is 70%, 75%, 80%, 85%, 90%, or 95% identical to SGGSSGGSSGSETPGTSESATPESSGGSSGGS (SEQ ID NO: 275).

In some embodiments, the amino terminus of the fusion partner protein is linked to the carboxy terminus of the programmable nuclease via the linker. In some embodiments, the carboxy terminus of the fusion partner protein is linked to the amino terminus of the programmable nuclease via the linker.

In some embodiments, the base editing enzyme is fused to TadA at the N-terminus. In some embodiments, the base editing enzyme is fused to TadA at the C-terminus. In some embodiments, the base editing enzyme is a deaminase dimer comprising an ABE. In some embodiments, the deaminase dimer comprises an adenosine deaminase. In some embodiments, the deaminase dimer comprises TadA fused to an adenine base editing enzyme selected from ABE8e, ABE8.20m, APOBEC3A, Anc APOBEC, and BtAPOBEC2. In some embodiments TadA is fused to ABE8e or a variant thereof. In some embodiments TadA is fused to ABE8e or a variant thereof at the amino-terminus (ABE8e-TadA). In some embodiments, TadA is fused to ABE8e or a variant thereof at the carboxy terminus (ABE8e-TadA).

In some embodiments, fusion partners comprise an amino acid sequence that is at least 50%, at least 55%, at least 60%, at least 65%, at least 70%, at least 75%, at least 80%, at least 85%, at least 90%, at least 95%, at least 98%, at least 99% or 100% identical to a fusion partner disclosed in TABLE 2. In some embodiments, compositions and methods comprise a fusion partner, wherein the amino acid sequence of the fusion partner is at least 50%, at least 55%, at least 60%, at least 65%, at least 70%, at least 75%, at least 80%, at least 85%, at least 90%, at least 95%, at least 98%, at least 99% or 100% identical to a fusion partner disclosed in TABLE 2.

TABLE 2
Exemplary Fusion Partner Sequences

| Nickname (typical function) | SEQ ID NO: |
|---|---|
| ABE8e (base editor) | 276 |
| ABE8e-TadA (base editor) | 277 |
| TadA-ABE8e (base editor) | 278 |
| ABE8.20m (base editor) | 279 |
| APOBEC3A (base editor) | 280 |
| Anc APOBEC (a.k.a. AncBE4Max) (base editor) | 281 |
| BtAPOBEC2 (base editor) | 282 |
| M-MLV RT or mutant thereof | 283 |
| (D200N/L603W/T330P/T306K/W313F mutations) | |
| (prime editing) | |
| TET1 (CRISPRa) | 284 |
| TET2 (CRISPRa) | 285 |
| P300 (CRISPRa) | 286 |
| VPR (CRISPRa) | 287 |
| DNMT3A (CRISPRi) | 288 |
| DNMT3L (CRISPRi) | 289 |
| VP64 (CRISPRa) | 290 |
| EZH2 (CRISPRi) | 291 |
| KRAB/KOX1 (CRISPRi) | 292 |
| ZIM3 (CRISPRi) | 293 |
| Tn5 transposase (DNA transposition; | 294 |
| targeted gene insertion) | |
| SB100X (DNA transposition; targeted gene insertion) | 295 |
| Phage encoded serine integrases/recombinase 2 | 296 |
| (DNA transposition; targeted gene insertion) | |
| Phage encoded serine integrase/recombinase 13 | 297 |
| (DNA transposition; targeted gene insertion) | |
| Human WT Exonuclease 1a (DNA transposition; | 298 |
| targeted gene insertion) | |

iii. Recombinases

In some embodiments, the fusion partners comprise a recombinase domain. In some embodiments, the recombinase is a site-specific recombinase. In some embodiments, the recombinase is a serine recombinase. Non-limiting examples of serine recombinases include, but are not limited to, gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include, but are not limited to: Bxb1, wBeta, BL3, phiR4, A118, TG1, MR11, phi370, SPBc, TP901-1, phiRV, FC1, K38, phiBT1, and phiC31. Further discussion and examples of suitable recombinase fusion partners are described in U.S. Pat. No. 10,975,392, which is incorporated herein by reference in its entirety.

In some embodiments, the fusion protein comprises a linker that links the recombinase domain to the Cas-CRISPR domain of the programmable nuclease. In some embodiments, the linker is The-Ser.

iv. Modifying Proteins

In some cases, a fusion partner provides enzymatic activity that modifies a protein (e.g., a histone) associated with a target nucleic acid. Such enzymatic activities include, but are not limited to, methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, de-ribosylation activity, myristoylation activity, and demyristoylation activity.

In some cases, the fusion partner has enzymatic activity that modifies a protein associated with a target nucleic acid. The protein may be a histone, an RNA binding protein, or a DNA binding protein. Examples of such protein modification activities include methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

v. CRISPRi Fusions

In some embodiments, fusions partners inhibit or reduce expression of a target nucleic acid. Fusion proteins comprising such fusion partners and a Cas programmable nuclease may be referred to as CRISPRi fusions. In some embodiments, fusion partners reduce expression of the target nucleic acid relative to its expression in the absence of the fusion programmable nuclease. Relative expression, including transcription and RNA levels, may be assessed, quantified, and compared, e.g., by RT-qPCR. In some embodiments, fusion partners may comprise a transcriptional repressor. Transcriptional repressors may inhibit transcription via: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

Non-limiting examples of fusion partners that decrease or inhibit transcription include, but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof.

vi. CRISPRa Fusions

In some embodiments, fusion partners activate or increase expression of a target nucleic acid. Fusion proteins comprising such fusion partners and a Cas programmable nuclease may be referred to as CRISPRa fusions. In some embodiments, fusion partners increase expression of the target nucleic acid relative to its expression in the absence of the fusion programmable nuclease. Relative expression, including transcription and RNA levels, may be assessed, quantified, and compared, e.g., by RT-qPCR. In some embodiments, fusion partners comprise a transcriptional activator. Transcriptional activators may promote transcription via: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof. Non-limiting examples of fusion partners that promote or increase transcription include, but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof.

vii. Additional Fusion Partners

In some cases, the fusion partner is a chloroplast transit peptide (CTP), also referred to as a plastid transit peptide. In some instances, this targets the fusion protein to a chloroplast. Chromosomal transgenes from bacterial sources must have a sequence encoding a CTP sequence fused to a sequence encoding an expressed protein if the expressed protein is to be compartmentalized in the plant plastid (e.g. chloroplast). The CTP is removed in a processing step during translocation into the plastid. Accordingly, localization of an exogenous protein to a chloroplast is often accomplished by means of operably linking a polynucleotide sequence encoding a CTP sequence to the 5′ region of a polynucleotide encoding the exogenous protein. In some cases, the CTP is located at the N-terminus of the fusion protein. Processing efficiency may, however, be affected by the amino acid sequence of the CTP and nearby sequences at the amino terminus (NH2 terminus) of the peptide.

In some cases, the fusion partner is an endosomal escape peptide. In some cases, an endosomal escape protein comprises the amino acid sequence GLFXALLXLLXSLWXLLLXA (SEQ ID NO: 200), wherein each X is independently selected from lysine, histidine, and arginine. In some cases, an endosomal escape protein comprises the amino acid sequence GLFHALLHLLHSLWHLLLHA (SEQ ID NO: 201). In some cases, the amino acid sequence of the endosomal escape protein is SEQ ID NO: 200 or SEQ ID NO: 201.

Further suitable fusion partners include, but are not limited to, proteins (or fragments/domains thereof) that are boundary elements (e.g., CTCF), proteins and fragments thereof that provide periphery recruitment (e.g., Lamin A, Lamin B, etc.), protein docking elements (e.g., FKBP/FRB, Pil1/Aby1, etc.).

viii. Linkers for Fusion Partners

In general, programmable nucleases and fusion partners of a fusion programmable nuclease are connected via a linker. The linker may comprise or consist of a covalent bond. The linker may comprise or consist of a chemical group. In some embodiments, the linker comprises an amino acid. In some cases, a linker comprises a bond or molecule that links a first polypeptide to a second polypeptide. In some instances, a peptide linker comprises at least two amino acids linked by an amide bond. In general, the linker connects a terminus of the programmable nuclease to a terminus of the fusion partner. In some embodiments, the carboxy terminus of the programmable nuclease is linked to the amino terminus of the fusion partner. In some embodiments, the carboxy terminus of the fusion partner is linked to the amino terminus of the programmable nuclease.

In some cases, a terminus of the Type V CRISPR/Cas protein is linked to a terminus of the fusion partner through an amide bond. In some cases, a Type V CRISPR/Cas protein is coupled to a fusion partner via a linker protein. The linker protein may have any of a variety of amino acid sequences. A linker protein may comprise a region of rigidity (e.g., beta sheet, alpha helix), a region of flexibility, or any combination thereof. In some instances, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. In some cases, a linked amino acid comprises at least two amino acids linked by an amide bond. These linkers may be produced by using synthetic, linker-encoding oligonucleotides to couple the proteins, or may be encoded by a nucleic acid sequence encoding a fusion protein (e.g., a programmable nuclease coupled to a fusion partner). Examples of linker proteins include glycine polymers (G)n, glycine-serine polymers (including, for example, (GS)n (SEQ ID NO: 299), GSGGSn (SEQ ID NO: 300), GGSGGSn (SEQ ID NO: 301), and GGGSn (SEQ ID NO: 302), where n is an integer of at least one), glycine-alanine polymers, and alanine-serine polymers. Exemplary linkers may comprise amino acid sequences including, but not limited to, GGSG (SEQ ID NO: 303), GGSGG (SEQ ID NO: 304), GSGSG (SEQ ID NO: 305), GSGGG (SEQ ID NO: 306), GGGSG (SEQ ID NO: 307), and GSSSG (SEQ ID NO: 308).

In some embodiments, linkers comprise or consist of 4 to 60, 6 to 55, 8 to 50, 10 to 45, 12 to 40, 14 to 35, 16 to 30, 18 to 25 linked amino acids. In some embodiments, linkers comprise or consist of 1 to 10, 10 to 20, 20 to 30, 30 to 40, 40 to 50, or 50 to 60 linked amino acids. In some embodiments, linkers comprise or consist of 5 to 10, 10 to 15, 15 to 20, 20 to 25, 25 to 30, 30 to 35, 35 to 40, 40 to 45, 45 to 50, 50 to 55 or 55 to 60 linked amino acids. In some embodiments, linkers comprise or consist of about 5, about 10, about 15, about 20, about 25, about 30, about 35, about 40, about 45, about 50, about 55 or about 60 amino acids.

In some embodiments, linkers comprise or consists of a non-peptide linker. Non-limiting examples of non-peptide linkers are linkers comprising polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, an alkyl linker, or a combination thereof.

In some embodiments, linkers comprise or consist of a nucleic acid. In some embodiments, the nucleic acid comprises DNA. In some embodiments, the nucleic acid comprises RNA. In some embodiments, the programmable nuclease and the fusion partner each interact with the nucleic acid, the nucleic acid thereby linking the programmable nuclease and the fusion partner. In some embodiments, the nucleic acid serves as a scaffold for both the programmable nuclease and the fusion partner to interact with, thereby linking the programmable nuclease and the fusion partner. Such nucleic acids include those described by Tadakuma et al., (2016), Progress in Molecular Biology and Translational Science, Volume 139, 2016, Pages 121-163, incorporated herein by reference.

In some embodiments, the fusion programmable nuclease or the guide nucleic acid comprises a chemical modification that allows for direct crosslinking between the guide nucleic acid or the programmable nuclease and the fusion partner. By way of non-limiting example, the chemical modification may comprise any one of a SNAP-tag, CLIP-tag, ACP-tag, Halo-tag, and an MCP-tag. In some embodiments, modifications are introduced with a Click Reaction, also known as Click Chemistry. The Click reaction may be copper dependent or copper independent.

In some embodiments, guide nucleic acids comprise an aptamer. The aptamer may serve as a linker between the programmable nuclease and the fusion partner by interacting non-covalently with both. In some embodiments, the aptamer binds a fusion partner, wherein the fusion partner is a transcriptional activator. In some embodiments, the aptamer binds a fusion partner, wherein the fusion partner is a transcriptional inhibitor. In some embodiments, the aptamer binds a fusion partner, wherein the fusion partner comprises a base editor. In some embodiments, the aptamer binds the fusion partner directly. In some embodiments, the aptamer binds the fusion partner indirectly. Aptamers may bind the fusion partner indirectly through an aptamer binding protein. By way of non-limiting example, the aptamer binding protein may be MS2 and the aptamer sequence may be ACATGAGGATCACCCATGT (SEQ ID NO: 309); the aptamer binding protein may be PP7 and the aptamer sequence may be GGAGCAGACGATATGGCGTCGCTCC (SEQ ID NO: 310); or the aptamer binding protein may be BoxB and the aptamer sequence may be GCCCTGAAGAAGGGC (SEQ ID NO: 311).

In some embodiments, fusion programmable nucleases do not comprise a linker. In some embodiments, the fusion partner is located within programmable nuclease. For example, the fusion partner may be a domain of a fusion partner protein that is internally integrated into the programmable nuclease. In other words, the fusion partner may be located between the 5′ and 3′ ends of the programmable nuclease without disrupting the ability of an RNP comprising the fusion programmable nuclease to recognize/bind a target nucleic acid. In some embodiments, the fusion partner replaces a portion of the programmable nuclease. In some embodiments, the fusion partner replaces a domain of the programmable nuclease. In some embodiments, the fusion partner does not replace a portion of the programmable nuclease.

### II. Multimeric Complexes

Compositions, systems, and methods of the present disclosure may comprise a multimeric complex or uses thereof, wherein the multimeric complex comprises multiple programmable nucleases that non-covalently interact with one another. A multimeric complex may comprise enhanced activity relative to the activity of any one of its programmable nucleases alone. For example, a multimeric complex comprising two Cas proteins may comprise greater nucleic acid binding affinity, cis-cleavage activity, and/or transcollateral cleavage activity than that of either of the Cas proteins provided in monomeric form. A multimeric complex may have an affinity for a target region of a target nucleic acid and is capable of catalytic activity (e.g., cleaving, nicking or modifying the nucleic acid) at or near the target region. Multimeric complexes may be activated when complexed with a guide nucleic acid. Multimeric complexes may be activated when complexed with a guide nucleic acid and a target nucleic acid. In some instances, the multimeric complex cleaves the target nucleic acid. In some instances, the multimeric complex nicks the target nucleic acid.

In some instances, multimeric complexes comprise at least one Type V CRISPR/Cas protein, or a fusion protein thereof, comprising an amino acid sequence with at least 70%, at least 75%, at least 80%, at least 85%, at least 90%, at least 95% or 100% identity to any one of SEQ ID NOs: 1-5. In some instances, multimeric complexes comprise at least one Type V CRISPR/Cas protein or a fusion protein thereof, wherein the amino acid sequence of the Type V CRISPR/Cas protein is at least 70%, at least 75%, at least 80%, at least 85%, at least 90%, at least 95% or 100% identical to any one of SEQ ID NOs: 1-5.

In some instances, the multimeric complex is a dimer comprising two programmable nucleases of identical amino acid sequences. In some instances, the multimeric complex comprises a first programmable nuclease and a second programmable nuclease, wherein the amino acid sequence of the first programmable nuclease is at least 90%, at least 92%, at least 94%, at least 96%, at least 98% identical, or at least 99% identical to the amino acid sequence of the second programmable nuclease.

In some instances, the multimeric complex is a heterodimeric complex comprising at least two programmable nucleases of different amino acid sequences. In some instances, the multimeric complex is a heterodimeric complex comprising a first programmable nuclease and a second programmable nuclease, wherein the amino acid sequence of the first programmable nuclease is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second programmable nuclease.

In some instances, a multimeric complex comprises at least two programmable nucleases. In some instances, a multimeric complex comprises more than two programmable nucleases. In some instances, a multimeric complex comprises two, three or four Cas14 proteins. In some instances, at least one programmable nuclease of the multimeric complex comprises an amino acid sequence with at least 70%, at least 75%, at least 80%, at least 85%, at least 90%, at least 95% or 100% identity to any one of SEQ ID NOs: 1-5. In some instances, each programmable nuclease of the multimeric complex comprises an amino acid sequence with at least 70%, at least 75%, at least 80%, at least 85%, at least 90%, at least 95% or 100% identity to any one of SEQ ID NOs: 1-5.

### A. Cas14 Dimers

In some instances, the multimeric complex is a dimer comprising two Cas14 proteins, (also referred to as a “Cas14 dimer”), wherein the amino acid sequence of the first Cas14 protein is at least 90%, at least 92%, at least 94%, at least 96%, at least 98%, at least 99%, or 100% identical to the second Cas14 protein. In some instances, dimerization promotes Cas14 activity and/or substrate or guide nucleic acid binding. A Cas14 dimer may comprise a two-lobe structure with a central channel. The Cas14 dimer may comprise enhanced activity (e.g., binding affinity or target nucleic acid cleavage kinetics) relative to a Cas14 protein of the dimer in its monomeric form. The Cas14 dimer may bind a single guide nucleic acid and single target nucleic acid. The Cas14 dimer may be capable of performing one or both of cis-cleavage activity and transcollateral cleavage activity.

In some instances, dimers comprise: a first Cas14 protein comprising at least 70%, at least 75%, at least 80%, at least 85%, at least 90%, at least 92%, at least 95%, at least 97%, at least 98%, at least 99%, or 100% sequence identity to SEQ ID NO: 1 and a second Cas14 comprising at least 70%, at least 75%, at least 80%, at least 85%, at least 90%, at least 92%, at least 95%, at least 97%, at least 98%, at least 99%, or 100% sequence identity to SEQ ID NO: 1.

In some instances, dimers comprise: a first Cas14 protein comprising at least 70%, at least 75%, at least 80%, at least 85%, at least 90%, at least 92%, at least 95%, at least 97%, at least 98%, at least 99%, or 100% sequence identity to SEQ ID NO: 2 and a second Cas14 comprising at least 70%, at least 75%, at least 80%, at least 85%, at least 90%, at least 92%, at least 95%, at least 97%, at least 98%, at least 99%, or 100% sequence identity to SEQ ID NO: 2.

In some instances, dimers comprise: a first Cas14 protein comprising at least 70%, at least 75%, at least 80%, at least 85%, at least 90%, at least 92%, at least 95%, at least 97%, at least 98%, at least 99%, or 100% sequence identity to SEQ ID NO: 3 and a second Cas14 comprising at least 70%, at least 75%, at least 80%, at least 85%, at least 90%, at least 92%, at least 95%, at least 97%, at least 98%, at least 99%, or 100% sequence identity to SEQ ID NO: 3.

In some instances, dimers comprise: a first Cas14 protein comprising at least 70%, at least 75%, at least 80%, at least 85%, at least 90%, at least 92%, at least 95%, at least 97%, at least 98%, at least 99%, or 100% sequence identity to SEQ ID NO: 4 and a second Cas14 comprising at least 70%, at least 75%, at least 80%, at least 85%, at least 90%, at least 92%, at least 95%, at least 97%, at least 98%, at least 99%, or 100% sequence identity to SEQ ID NO: 4.

In some instances, dimers comprise: a first Cas14 protein comprising at least 70%, at least 75%, at least 80%, at least 85%, at least 90%, at least 92%, at least 95%, at least 97%, at least 98%, at least 99%, or 100% sequence identity to SEQ ID NO: 5 and a second Cas14 comprising at least 70%, at least 75%, at least 80%, at least 85%, at least 90%, at least 92%, at least 95%, at least 97%, at least 98%, at least 99%, or 100% sequence identity to SEQ ID NO: 5.

A Cas14 dimer may require specific conditions (e.g., a minimum ionic strength requirement) or a substrate or cofactor (e.g., a guide nucleic acid) for dimerization. A composition of the present disclosure may therefore comprise monomeric Cas14 proteins which dimerize upon modification of a solution condition (e.g., an increase in salinity or decrease in pH) or addition of a guide nucleic acid. A Cas14 protein of the present disclosure may exhibit concentration-dependent dimerization. For example, a Cas14 protein may comprise an equilibrium constant for dimerization (e.g., in standard conditions

$$Keq = \frac{[ CAS 14 Dimer ]}{{[ Cas 14 Monomer ]}^{2}} )$$

of at least 0.0001 mM⁻¹, at least 0.0005 mM⁻¹, at least 0.001 mM⁻¹, at least 0.005 mM⁻¹, at least 0.01 mM⁻¹, at least 0.05 mM⁻¹, at least 0.1 mM⁻¹, at least 0.5 mM⁻¹, at least 1 mM⁻¹, at least 5 mM⁻¹, at least 10 mM⁻¹, at least 50 mM⁻¹, or at least 100 mM⁻¹. A Cas14 protein may comprise an equilibrium constant for dimerization that is less than about 50 mM⁻¹, less than about 10 mM⁻¹, less than about 5 mM⁻¹, less than about 1 mM⁻¹, less than about 0.5 mM⁻¹, less than about 0.1 mM⁻¹, less than about 0.05 mM⁻¹, less than about 0.01 mM⁻¹, less than about 0.005 mM⁻¹, less than about 0.001 mM⁻¹, less than about 0.0005 mM⁻¹, or less than about 0.0001 mM⁻¹.

### B. Multimeric Complexes of Cas14 Proteins

In some cases, a multimeric complex comprises a first Cas14 protein and a second Cas14 protein, wherein the amino acid sequence of the first Cas14 protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second Cas14 protein. In some instances, the first Cas14 protein binds a first nucleic acid or portion thereof and the second Cas14 protein binds a second nucleic acid or portion thereof, wherein the nucleobase sequence of the first nucleic acid is different from the nucleobase sequence of the second nucleic acid. In some instances, the first Cas14 protein binds a single-stranded nucleic acid or portion thereof and the second Cas14 protein binds a double stranded nucleic acid or portion thereof. In some cases, the multimeric complex has faster cis-cleavage kinetics than either of the monomeric forms of the first or second Cas14 proteins. In some cases, the multimeric complex has faster cis-cleavage kinetics than a dimer of the first Cas14 protein or a dimer of the second Cas14 protein. In some cases, the multimeric complex has faster transcollateral cleavage kinetics than either of the monomeric forms of the first or second Cas14 proteins. In some cases, the multimeric complex has faster transcollateral cleavage kinetics than a dimer of the first Cas14 protein or a dimer of the second Cas14 protein.

In some instances, the multimeric complex comprises a first Cas14 protein and the second Cas14 protein each comprising an amino acid sequence that is at least 70%, at least 75%, at least 80%, at least 85%, at least 90%, at least 95%, at least 98%, at least 99%, or 100% amino acid sequence identical to any one of SEQ ID NOs: 1-5. In some cases, each of the amino acid sequences of the first and second Cas14 proteins is at least 70%, at least 75%, at least 80%, at least 85%, at least 90%, at least 95%, or 100% identical to any one of SEQ ID NOs: 1-97.

In some cases, multimeric complexes comprise at least one Cas14 protein selected from a Cas14a protein, a Cas14b protein, a Cas14c protein, a Cas14d protein, a Cas14e protein, a Cas14f protein, a Cas14g protein, a Cas14h protein, and a Cas14u protein, and a combination thereof. In some cases, the amino acid sequence of the at least one Cas14 protein is at least 70%, at least 75%, at least 80%, at least 85%, at least 90%, at least 95%, or 100% identical to any one of SEQ ID NOs: 6-97.

In some instances, the amino acid sequence of the first Cas14 protein is at least 70%, at least 75%, at least 80%, at least 85%, at least 90%, at least 95%, or 100% identical to any one of SEQ ID NOs: 1-5 and the second Cas14 protein is selected from a Cas14a protein, a Cas14b protein, a Cas14c protein, a Cas14d protein, a Cas14e protein, a Cas14f protein, a Cas14g protein, a Cas14h protein, and a Cas14u protein. In some cases, the amino acid sequence of the second Cas14 protein comprises an amino acid sequence that is at least 80%, at least 85%, at least 90%, at least 95%, at least 98%, at least 99%, or 100% identical to any one of SEQ ID NOs: 6-97. In some cases, the amino acid sequence of the second Cas 14 protein consists of an amino acid sequence having at least 80%, at least 85%, at least 90%, at least 95%, at least 98%, at least 99%, or 100%, amino acid sequence identity to any one of SEQ ID NOs: 6-97.

In some cases, a multimeric complex comprises a Cas14 protein and a Type V CRISPR/Cas programmable nuclease, wherein the type V CRISPR/Cas programmable nuclease is not a Cas14 protein. In some instances, the Type V CRISPR/Cas programmable nuclease is a Cas12 protein. In some instances, the Type V CRISPR/Cas programmable nuclease is a CasΦ protein. In some instances, the Type V CRISPR/Cas programmable nuclease is a CasY protein. In some cases, the Type V CRISPR/Cas programmable nuclease lacks an HNH domain. In some embodiments, the Type V CRISPR/Cas programmable nuclease comprises a partial RuvC-like domain.

### C. Cas12

In some instances, multimeric complexes comprise a Cas14 protein and a Cas12 protein. The Cas12 protein may be a Cas12a protein (also referred to as Cpf1), a Cas12b protein, Cas12c protein, Cas12d protein, or a Cas12e protein. The Cas12c protein may be a C12c4 protein, a C12c8 protein, a C12c5 protein, a C12c10 protein, or a C12c9 protein.

In some cases, the Cas12 protein is capable of cleaving a nucleic acid via a single catalytic RuvC domain. In such cases, the Cas12 protein may cleave both strands of a double-stranded target nucleic acid molecule within the single catalytic RuvC domain. The RuvC domain may be disposed within a nuclease lobe (NUC lobe) of the protein. The RuvC domain may target complementary positions on a double stranded nucleic acid target, or may target separate positions (e.g., between different base pairs) on the target and non-target strands of the target nucleic acid.

A Cas12 protein may comprise a recognition lobe (REC lobe) which may have a binding affinity for a guide nucleic acid or for a guide nucleic acid-target nucleic acid complex. In some cases, a REC lobe comprises a binding affinity for a PAM sequence in the target nucleic acid. Some Cas12 proteins (e.g., SEQ ID NO: 98) comprise two recognition domains, which may both be disposed within the REC lobe. The two recognition domains may separately identify and direct nuclease binding to PAM sequences disposed within separate strands of the target nucleic acid. The REC lobe may be disposed between regions of a wedge (WED) domain. The REC lobe may be connected to the NUC lobe by a bridge helix. A Cas12 protein may additionally include two domains for PAM recognition termed the PAM interacting (PI) domain and the wedge (WED) domain.

In some cases, multimeric complexes comprise a Cas12 protein and a Cas14 proteins, wherein the amino acid sequence of the Cas12 protein comprises an amino acid sequence that is at least 80%, at least 85%, at least 90%, at least 95%, at least 98%, at least 99%, or 100% identical to any one of SEQ ID NOs: 98-140. In some cases, the amino acid sequence of the Cas 12 protein consists of an amino acid sequence having at least 80%, at least 85%, at least 90%, at least 95%, at least 98%, at least 99%, or 100%, amino acid sequence identity to any one of SEQ ID NOs: 98-140. In some instances, the amino acid sequence of the Cas14 protein is at least 70%, at least 75%, at least 80%, at least 85%, at least 90%, at least 95%, at least 98%, at least 99%, or 100% identical to any one of SEQ ID NOs: 1-5.

### D. CasΦ

In some instances, multimeric complexes comprise a Cas14 protein and a CasΦ protein (also referred to as CasPhi). A CasΦ protein may function as an endonuclease that catalyzes cleavage at a specific sequence in a target nucleic acid. A CasΦ protein may have a compact catalytic site in a RuvC domain that is capable of catalyzing pre-crRNA processing and nicking or cleaving of nucleic acids. The compact catalytic site may render the CasΦ protein especially advantageous for genome engineering and new functionalities for genome manipulation. A CasΦ protein may also be referred to as a Cas12J protein.

A CasΦ protein may have a molecular weight of about 65 kiloDaltons (kDa) to about 85 kDa. For example, a CasΦ protein can have a molecular weight of about 65 kDa to about 70 kDa, about 70 kDa to about 75 kDa, or about 75 kDa to about 80 kDa. For example, a CasΦ protein may have a molecular weight of from about 70 kDa to about 80 kDa.

In some instances, multimeric complexes comprise a CasΦ protein and a Cas14 protein, wherein the amino acid sequence of the CasΦ protein comprises an amino acid sequence that is at least 70%, at least 75%, at least 80%, at least 85%, at least 90%, at least 95%, at least 98%, at least 99%, or 100% identical to any one of SEQ ID NOs: 141-188. In some cases, the amino acid sequence of the CasΦ protein consists of an amino acid sequence having at least 80%, at least 85%, at least 90%, at least 95%, at least 98%, at least 99%, or 100%, amino acid sequence identity to any one of SEQ ID NOs: 141-188. In some instances, the Cas14 protein comprises an amino acid sequence that is at least 70%, at least 75%, at least 80%, at least 85%, at least 90%, at least 95%, at least 98%, at least 99%, or 100% identical to any one of SEQ ID NOs: 1-5. In some instances, the amino acid sequence of the Cas14 protein is at least 70%, at least 75%, at least 80%, at least 85%, at least 90%, at least 95%, at least 98%, at least 99%, or 100% identical to any one of SEQ ID NOs: 1-5.

### E. CasY

In some instances, multimeric complexes comprise a Cas14 protein and a CasY protein. A CasY protein may include an N-terminal domain roughly 800-1000 amino acids in length (e.g., about 815 for CasY1 and about 980 for CasY5), and a C-terminal domain that includes three partial RuvC domains (RuvC-I, RuvC-II, and RuvC-III, also referred to herein as subdomains). The sequences of the three partial RuvC domains are typically not contiguous, but form a complete RuvC domain once the protein is translated and folds. In some cases, a CasY protein does not include a localization sequence and/or a single domain with catalytic activity). In some cases, the length of the CasY protein is 750 to 1050 amino acids. In some cases, the length of the CasY protein is 750 to 1025, 750 to 1000, 750 to 950, 775 to 1050, 775 to 1025, 775 to 1000, 775 to 950, 800 to 1050, 800 to 1025, 800 to 1000, or 800 to 950 amino acids. In some cases, the CasY protein comprises a region that is N-terminal to its partial RuvC-III domain, wherein the length of the region is 750 to 1050 amino acids. In some cases, the CasY protein comprises a region that is N-terminal to its partial RuvC-III domain, wherein the length of the region is 750 to 1025, 750 to 1000, 750 to 950, 775 to 1050, 775 to 1025, 775 to 1000, 775 to 950, 800 to 1050, 800 to 1025, 800 to 1000, or 800 to 950 amino acids.

In some cases, a CasY protein may recognize a protospacer adjacent motif (PAM) having a sequence of TR, where R represents any purine (e.g., A or G). In some embodiments, a CasY protein may recognize a PAM having a sequence of TN, where N represents any nucleotide (e.g., A, C, T, U, or G). In some embodiments, a CasY protein may recognize a PAM having a sequence of TA. In some embodiments, a CasY protein may recognize a PAM having a sequence of TG. A CasY protein can be a CasY variant.

In some instances, multimeric complexes comprise a CasY protein and a Cas14 protein, wherein the amino acid sequence of the CasY protein comprises an amino acid sequence that is at least 70%, at least 75%, at least 80%, at least 85%, at least 90%, at least 95%, at least 98%, at least 99%, or 100% identical to any one of SEQ ID NOs: 189-198. In some cases, the amino acid sequence of the CasY protein consists of an amino acid sequence having at least 80%, at least 85%, at least 90%, at least 95%, at least 98%, at least 99%, or 100%, amino acid sequence identity to any one of SEQ ID NOs: 189-198. In some instances, the Cas14 protein comprises an amino acid sequence of the Cas14 protein is at least 70%, at least 75%, at least 80%, at least 85%, at least 90%, at least 95%, at least 98%, at least 99%, or 100% identical to any one of SEQ ID NOs: 1-5. In some instances, the amino acid sequence of the Cas14 protein is at least 70%, at least 75%, at least 80%, at least 85%, at least 90%, at least 95%, at least 98%, at least 99%, or 100% identical to any one of SEQ ID NOs: 1-5.

### F. Certain Multimeric Complexes

Provided herein are compositions comprising a multimeric complex, wherein the multimeric complex comprises: a Cas14 protein comprising an amino acid sequence that is at least 70%, at least 75%, at least 80%, at least 85%, at least 90%, at least 92%, at least 95%, at least 97%, at least 98%, at least 99%, or 100% identical to any one of SEQ ID NOs: 1-5, and a Type V CRISPR/Cas protein comprising an amino acid sequence that is at least 70%, at least 75%, at least 80%, at least 85%, at least 90%, at least 92%, at least 95%, at least 97%, at least 98%, at least 99%, or 100% identical to any one of SEQ ID NOs: 6-198.

In some instances, compositions comprise a multimeric complex, wherein the multimeric complex comprises: a first Cas14 protein comprising an amino acid sequence that is at least 70%, at least 75%, at least 80%, at least 85%, at least 90%, at least 92%, at least 95%, at least 97%, at least 98%, at least 99%, or 100% identical to any one of SEQ ID NOs: 1-5, and a second Cas14 protein comprising an amino acid sequence that is at least 70%, at least 75%, at least 80%, at least 85%, at least 90%, at least 92%, at least 95%, at least 97%, at least 98%, at least 99%, or 100% identical to any one of SEQ ID NOs: 6-97. In some instances, the amino acid sequence of the first Cas14 protein is at least 70%, at least 75%, at least 80%, at least 85%, at least 90%, at least 92%, at least 95%, at least 97%, at least 98%, at least 99%, or 100% identical to any one of SEQ ID NOs: 1-5.

In some instances, compositions comprise a multimeric complex, wherein the multimeric complex comprises: a Cas14 protein comprising an amino acid sequence that is at least 70%, at least 75%, at least 80%, at least 85%, at least 90%, at least 92%, at least 95%, at least 97%, at least 98%, at least 99%, or 100% identical to any one of SEQ ID NOs: 1-5, and a Cas12 protein comprising an amino acid sequence that is at least 70%, at least 75%, at least 80%, at least 85%, at least 90%, at least 92%, at least 95%, at least 97%, at least 98%, at least 99%, or 100% identical to any one of SEQ ID NOs: 98-140. In some instances, the amino acid sequence of the Cas14 protein is at least 70%, at least 75%, at least 80%, at least 85%, at least 90%, at least 92%, at least 95%, at least 97%, at least 98%, at least 99%, or 100% identical to any one of SEQ ID NOs: 1-5.

In some instances, compositions comprise a multimeric complex, wherein the multimeric complex comprises: a Cas14 protein comprising an amino acid sequence that is at least 70%, at least 75%, at least 80%, at least 85%, at least 90%, at least 92%, at least 95%, at least 97%, at least 98%, at least 99%, or 100% identical to any one of SEQ ID NOs: 1-5 and a CasPhi protein comprising an amino acid sequence that is at least 70%, at least 75%, at least 80%, at least 85%, at least 90%, at least 92%, at least 95%, at least 97%, at least 98%, at least 99%, or 100% identical to any one of SEQ ID NOs: 141-188. In some instances, the amino acid sequence of the Cas14 protein is at least 70%, at least 75%, at least 80%, at least 85%, at least 90%, at least 92%, at least 95%, at least 97%, at least 98%, at least 99%, or 100% identical to any one of SEQ ID NOs: 1-5.

In some instances, compositions comprise a multimeric complex, wherein the multimeric complex comprises: a Cas14 protein comprising an amino acid sequence that is at least 70%, at least 75%, at least 80%, at least 85%, at least 90%, at least 92%, at least 95%, at least 97%, at least 98%, at least 99%, or 100% identical to any one of SEQ ID NOs: 1-5, and a CasY protein comprising an amino acid sequence that is at least 70%, at least 75%, at least 80%, at least 85%, at least 90%, at least 92%, at least 95%, at least 97%, at least 98%, at least 99%, or 100% identical to any one of SEQ ID NOs: 189-198. In some instances, the amino acid sequence of the Cas14 protein is at least 70%, at least 75%, at least 80%, at least 85%, at least 90%, at least 92%, at least 95%, at least 97%, at least 98%, at least 99%, or 100% identical to any one of SEQ ID NOs: 1-5.

A type V CRISPR/Cas protein, a dimer thereof, or a multimeric complex thereof may cleave both strands of a target DNA molecule at different locations (thereby generating a sticky ended product) or at the complementary positions (thereby generating a blunt end product). A type V CRISPR/Cas protein, a dimer thereof, or a multimeric complex thereof may cleave a double-stranded nucleic acid to generate product nucleic acids comprising 5′ overhangs. The 5′ overhangs may be 1-4 nucleotides, 1-6 nucleotides, 2-6 nucleotides, 3-8 nucleotides, or greater than 4 nucleotides in length.

A type V CRISPR/Cas protein, a dimer thereof, or a multimeric complex thereof, may cleave each strand of a target DNA molecule with different kinetics. For example, a programmable nuclease may cleave a first strand of a target DNA molecule with faster kinetics than the second strand. In such cases, the type V CRISPR/Cas protein, the dimer thereof, or the multimeric complex thereof releases the target nucleic acid subsequent to the first cleavage and prior to the second cleavage, thereby generating a “nicked” (e.g., cleaved only on one strand) product.

### III. Systems

Disclosed herein, in some aspects, are systems for modifying a nucleic acid, comprising any one of the programmable nucleases described herein, or a multimeric complex thereof. Systems may be used to detect, modify, or edit a target nucleic acid. Systems may be used to modify the activity or expression of a target nucleic acid. In some embodiments, systems comprise a programmable nuclease described herein, a reagent, support medium, or a combination thereof. In some instances, the programmable nuclease comprises a Type V CRISPR/Cas protein, or a fusion protein thereof, described herein. In some instances, the programmable nuclease comprises an amino acid sequence that is at least 70%, at least 75%, at least 80%, at least 85%, at least 90%, at least 95%, or 100% identical to any one of SEQ ID NOs: 1-5. In some instances, the amino acid sequence of the programmable nuclease is at least 70%, at least 75%, at least 80%, at least 85%, at least 90%, at least 95%, or 100% identical to any one of SEQ ID NOs: 1-5. Such systems may be used for detecting the presence of a target nucleic acid associated with or causative of a disease, such as cancer, a genetic disorder, or an infection. In some instances, such methods and systems are useful for phenotyping, genotyping, or determining ancestry. Unless specified otherwise, systems include kits and may be referred to as kits. Unless specified otherwise, systems include devices and may also be referred to as devices. Systems described herein may be provided in the form of a companion diagnostic assay or device, a point-of-care assay or device, or an over-the-counter diagnostic assay/device.

In some embodiments, in vitro can be used to describe an event that takes places in a container for holding laboratory reagents such that it is separated from the biological source from which the material is obtained. In vitro assays can encompass cell-based assays in which living or dead cells are employed. In vitro assays can also encompass a cell-free assay in which no intact cells are employed. In some cases, in vivo can be used to describe an event that takes place in a subject's body. In some cases, ex vivo can be used to describe an event that takes place outside of a subject's body. An ex vivo assay is not performed on a subject. Rather, it is performed upon a sample separate from a subject. An example of an ex vivo assay performed on a sample is an in vitro assay.

Reagents and programmable nucleases of various systems may be provided in a reagent chamber or on the support medium. Alternatively, the reagent and/or programmable nuclease may be contacted with the reagent chamber or the support medium by the individual using the system. An exemplary reagent chamber is a test well or container. The opening of the reagent chamber may be large enough to accommodate the support medium. Optionally, the system comprises a buffer and a dropper. The buffer may be provided in a dropper bottle for ease of dispensing. The dropper may be disposable and transfer a fixed volume. The dropper may be used to place a sample into the reagent chamber or on the support medium.

### A. Certain System Components and Reagents

i. System Solutions

In general, systems comprise a solution in which the activity of a programmable nuclease occurs. Often, the solution comprises or consists essentially of a buffer. The solution or buffer may comprise a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some instances, a buffer is required for cell lysis activity or viral lysis activity.

In some cases, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. In some instances, the concentration of the buffering agent in the buffer is 1 mM to 200 mM. A buffer compatible with a programmable nuclease may comprise a buffering agent at a concentration of 10 mM to 30 mM. A buffer compatible with a programmable nuclease may comprise a buffering agent at a concentration of about 20 mM. A buffering agent may provide a pH for the buffer or the solution in which the activity of the programmable nuclease occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, 7 to 9, 7 to 9.5, 6.5 to 8, 6.5 to 9, 6.5 to 9.5, 7.5 to 8.5, 7.5 to 9, 7.5 to 9.5, or 9.5 to 10.5.

In some cases, systems comprise a solution, wherein the solution comprises at least one salt. In some instances, the at least one salt is selected from potassium acetate, magnesium acetate, sodium chloride, potassium chloride, magnesium chloride, calcium chloride, and any combination thereof. In some instances, the concentration of the at least one salt in the solution is 5 mM to 100 mM, 5 mM to 10 mM, 1 mM to 60 mM, or 1 mM to 10 mM. In some instances, the concentration of the at least one salt is about 105 mM. In some instances, the concentration of the at least one salt is about 55 mM. In some instances, the concentration of the at least one salt is about 7 mM. In some embodiments, the solution comprises potassium acetate and magnesium acetate. In some embodiments, the solution comprises sodium chloride and magnesium chloride. In some embodiments, the solution comprises potassium chloride and magnesium chloride. In some instances, the salt is a magnesium salt and the concentration of magnesium in the solution is at least 5 mM, 7 mM, at least 9 mM, at least 11 mM, at least 13 mM, or at least 15 mM. In some instances, the concentration of magnesium is less than 20 mM, less than 18 mM or less than 16 mM.

In some cases, systems comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some instances, the crowding agent is glycerol. In some instances, the concentration of the crowding agent in the solution is 0.01% (v/v) to 10% (v/v). In some instances, the concentration of the crowding agent in the solution is 0.5% (v/v) to 10% (v/v).

In some cases, systems comprise a solution, wherein the solution comprises at least one detergent. Exemplary detergents include Tween, Triton-X, and IGEPAL. A solution may comprise Tween, Triton-X, or any combination thereof. A solution may comprise Triton-X. A solution may comprise IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some cases, systems comprise a solution, wherein the solution comprises at least one reducing agent. Exemplary reducing agents comprise dithiothreitol (DTT), ß-mercaptoethanol (BME), or tris(2-carboxyethyl)phosphine (TCEP). In some instances, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some cases, systems comprise a solution, wherein the solution comprise a competitor. In general, competitors compete with the target nucleic acid or the reporter nucleic acid for cleavage by the programmable nuclease or a dimer thereof. Exemplary competitors include heparin, and imidazole, and salmon sperm DNA. In some cases, the concentration of the competitor in the solution is 1 μg/mL to 100 μg/mL. In some cases, the concentration of the competitor in the solution is 40 μg/mL to 60 μg/mL.

In some cases, systems comprise a solution, wherein the solution comprise a co-factor. In some cases, the co-factor allows a programmable nuclease or a multimeric complex thereof to perform a function, including pre-crRNA processing and/or target nucleic acid cleavage. For example, as discussed in Jiang F. and Doudna J. A. (Annu. Rev. Biophys. 2017. 46:505-29), Cas9 may use divalent metal ions as co-factors. The suitability of a cofactor for a programmable nuclease or a multimeric complex thereof may be assessed, such as by methods based on those described by Sundaresan et al. (Cell Rep. 2017 Dec. 26; 21(13): 3728-3739). In some cases, a programmable or a multimeric complex thereof forms a complex with a co-factor. In some cases, the co-factor is a divalent metal ion. In some embodiments, the divalent metal ion is selected from Mg²⁺, Mn²⁺, Zn²⁺, Ca²⁺, Cu²⁺. In some cases, the divalent metal ion is Mg²⁺. In some cases, the programmable nuclease is a Type V CRISPR/Cas protein and the co-factor is Mg²⁺.

ii. Reporters

In some instances, systems disclosed herein comprise a reporter. By way of non-limiting and illustrative example, a reporter may comprise a single stranded nucleic acid and a detection moiety (e.g., a labeled single stranded RNA reporter), wherein the nucleic acid is capable of being cleaved by a programmable nuclease (e.g., a Type V CRISPR/Cas protein as disclosed herein) or a multimeric complex thereof, releasing the detection moiety, and, generating a detectable signal. In some cases, a detectable signal comprises a signal that can be detected using optical, fluorescent, chemiluminescent, electrochemical and other detection methods known in the art. As used herein, “reporter” is used interchangeably with “reporter nucleic acid” or “reporter molecule”. The programmable nucleases disclosed herein, activated upon hybridization of a guide RNA to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded.

In some instances, reporters comprise a protein capable of generating a signal. A signal may be a calorimetric, potentiometric, amperometric, optical (e.g., fluorescent, colorimetric, etc.), or piezo-electric signal. In some cases, the reporter comprises a detection moiety. Suitable detectable labels and/or moieties that may provide a signal include, but are not limited to, an enzyme, a radioisotope, a member of a specific binding pair; a fluorophore; a fluorescent protein; a quantum dot; and the like.

In some cases, the reporter comprises a detection moiety and a quenching moiety. In some instances, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some cases, the detection moiety is at the 5′ terminus of the nucleic acid of a reporter. In some cases, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, and glucose oxidase (GO).

In some instances, the detection moiety comprises an invertase. The substrate of the invertase may be sucrose. A DNS reagent may be included in the system to produce a colorimetric change when the invertase converts sucrose to glucose. In some cases, the reporter nucleic acid and invertase are conjugated using a heterobifunctional linker via sulfo-SMCC chemistry.

Suitable fluorophores may provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Non-limiting examples of fluorophores are fluorescein amidite, 6-Fluorescein, IRDye 700, TYE 665, Alex Fluor 594, or ATTO TM 633 (NHS Ester). The fluorophore may be an infrared fluorophore. The fluorophore may emit fluorescence in the range of 500 nm and 720 nm. In some cases, the fluorophore emits fluorescence at a wavelength of 700 nm or higher. In other cases, the fluorophore emits fluorescence at about 665 nm. In some cases, the fluorophore emits fluorescence in the range of 500 nm to 520 n, 500 nm to 540 nm, 500 nm to 590 n, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some cases, the fluorophore emits fluorescence in the range 450 nm to 750 nm, 500 nm to 650 n, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the quenching moiety quenches a detection moiety that emits fluorescence in the range 450 nm to 750 nm, 500 nm to 650 nm, or 550 to 650 n. A quenching moiety may quench fluorescein amidite, 6-Fluorescein, IRDye 700, TYE 665, Alex Fluor 594, or ATTO TM 633 (NHS Ester). A quenching moiety may be Iowa Black RQ, Iowa Black FQ or IRDye QC-1 Quencher. A quenching moiety may quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

The generation of the detectable signal from the release of the detection moiety indicates that cleavage by the programmable nucleases has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively or in combination, the detection moiety comprises a protein. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal may be a colorimetric signal or a signal visible by eye. In some instances, the detectable signal may be fluorescent, electrical, chemical, electrochemical, or magnetic. In some cases, the first detection signal may be generated by binding of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some cases, the detectable signal may be generated directly by the cleavage event. Alternatively or in combination, the detectable signal may be generated indirectly by the signal event. Sometimes the detectable signal is not a fluorescent signal. In some instances, the detectable signal may be a colorimetric or color-based signal. In some cases, the detected target nucleic acid may be identified based on its spatial location on the detection region of the support medium. In some cases, the second detectable signal may be generated in a spatially distinct location than the first generated signal.

Examples of reporter nucleic acids are provided in TABLE 3.

TABLE 3
Examples of Single Stranded Nucleic Acids in a Reporter

| 5′ Detection Moiety* | Sequence (SEQ ID NO:) | 3′ Quencher* |
|---|---|---|
| /56-FAM/ | rUrUrUrUrU (SEQ ID NO: 202) | /3IABkFQ/ |
| /5IRD700/ | rUrUrUrUrU (SEQ ID NO: 202) | /3IRQC1N/ |
| /5TYE665/ | rUrUrUrUrU (SEQ ID NO: 202) | /3IAbRQSp/ |
| /5Alex594N/ | rUrUrUrUrU (SEQ ID NO: 202) | /3IAbRQSp/ |
| /5ATTO633N/ | rUrUrUrUrU (SEQ ID NO: 202) | /3IAbRQSp/ |
| /56-FAM/ | rUrUrUrUrUrUrUrU(SEQ ID NO: 203) | /3IABkFQ/ |
| /5IRD700/ | rUrUrUrUrUrUrUrU(SEQ ID NO: 203) | /3IRQC1N/ |
| /5TYE665/ | rUrUrUrUrUrUrUrU(SEQ ID NO: 203) | /3IAbRQSp/ |
| /5Alex594N/ | rUrUrUrUrUrUrUrU(SEQ ID NO: 203) | /3IAbRQSp/ |
| /5ATTO633N/ | rUrUrUrUrUrUrUrU(SEQ ID NO: 203) | /3IAbRQSp/ |
| /56-FAM/ | rUrUrUrUrUrUrUrUrUrU(SEQ ID NO: 204) | /3IABkFQ/ |
| /5IRD700/ | rUrUrUrUrUrUrUrUrUrU(SEQ ID NO: 204) | /3IRQC1N/ |
| /5TYE665/ | rUrUrUrUrUrUrUrUrUrU(SEQ ID NO: 204) | /3IAbRQSp/ |
| /5Alex594N/ | rUrUrUrUrUrUrUrUrUrU(SEQ ID NO: 204) | /3IAbRQSp/ |
| /5ATTO633N/ | rUrUrUrUrUrUrUrUrUrU(SEQ ID NO: 204) | /3IAbRQSp/ |
| /56-FAM/ | TTTTrUrUTTTT(SEQ ID NO: 205) | /3IABkFQ/ |
| /5IRD700/ | TTTTrUrUTTTT(SEQ ID NO: 205) | /3IRQC1N/ |
| /5TYE665/ | TTTTrUrUTTTT(SEQ ID NO: 205) | /3IAbRQSp/ |
| /5Alex594N/ | TTTTrUrUTTTT(SEQ ID NO: 205) | /3IAbRQSp/ |
| /5ATTO633N/ | TTTTrUrUTTTT(SEQ ID NO: 205) | /3IAbRQSp/ |
| /56-FAM/ | TTrUrUTT(SEQ ID NO: 206) | /3IABkFQ/ |
| /5IRD700/ | TTrUrUTT(SEQ ID NO: 206) | /3IRQC1N/ |
| /5TYE665/ | TTrUrUTT(SEQ ID NO: 206) | /3IAbRQSp/ |
| /5Alex594N/ | TTrUrUTT(SEQ ID NO: 206) | /3IAbRQSp/ |
| /5ATTO633N/ | TTrUrUTT(SEQ ID NO: 206) | /3IAbRQSp/ |
| /56-FAM/ | TArArUGC(SEQ ID NO: 207) | /3IABkFQ/ |
| /5IRD700/ | TArArUGC(SEQ ID NO: 207) | /3IRQC1N/ |
| /5TYE665/ | TArArUGC(SEQ ID NO: 207) | /3IAbRQSp/ |
| /5Alex594N/ | TArArUGC(SEQ ID NO: 207) | /3IAbRQSp/ |
| /5ATTO633N/ | TArArUGC(SEQ ID NO: 207) | /3IAbRQSp/ |
| /56-FAM/ | TArUrGGC(SEQ ID NO: 208) | /3IABkFQ/ |
| /5IRD700/ | TArUrGGC(SEQ ID NO: 208) | /3IRQC1N/ |
| /5TYE665/ | TArUrGGC(SEQ ID NO: 208) | /3IAbRQSp/ |
| /5Alex594N/ | TArUrGGC(SEQ ID NO: 208) | /3IAbRQSp/ |
| /5ATTO633N/ | TArUrGGC(SEQ ID NO: 208) | /3IAbRQSp/ |
| /56-FAM/ | rUrUrUrUrU(SEQ ID NO: 209) | /3IABkFQ/ |
| /5IRD700/ | rUrUrUrUrU(SEQ ID NO: 209) | /3IRQC1N/ |
| /5TYE665/ | rUrUrUrUrU(SEQ ID NO: 209) | /3IAbRQSp/ |
| /5Alex594N/ | rUrUrUrUrU(SEQ ID NO: 209) | /3IAbRQSp/ |
| /5ATTO633N/ | rUrUrUrUrU(SEQ ID NO: 209) | /3IAbRQSp/ |
| /56-FAM/ | TTATTATT (SEQ ID NO: 210) | /3IABkFQ/ |
| /56-FAM/ | TTATTATT (SEQ ID NO: 210) | /3IABkFQ/ |
| /5IRD700/ | TTATTATT (SEQ ID NO: 210) | /3IRQC1N/ |
| /5TYE665/ | TTATTATT (SEQ ID NO: 210) | /3IAbRQSp/ |
| /5Alex594N/ | TTATTATT (SEQ ID NO: 210) | /3IAbRQSp/ |
| /5ATTO633N/ | TTATTATT (SEQ ID NO: 210) | /3IAbRQSp/ |
| /56-FAM/ | TTTTTT (SEQ ID NO: 211) | /3IABkFQ/ |
| /56-FAM/ | TTTTTTTT (SEQ ID NO: 212) | /3IABkFQ/ |
| /56-FAM/ | TTTTTTTTTT (SEQ ID NO: 213) | /3IABkFQ/ |
| /56-FAM/ | TTTTTTTTTTTT (SEQ ID NO: 214) | /3IABkFQ/ |
| /56-FAM/ | TTTTTTTTTTTTTT (SEQ ID NO: 215) | /3IABkFQ/ |
| /56-FAM/ | AAAAAA (SEQ ID NO: 216) | /3IABkFQ/ |
| /56-FAM/ | CCCCCC (SEQ ID NO: 217) | /3IABkFQ |
| /56-FAM/ | GGGGGG (SEQ ID NO: 218) | /3IABkFQ/ |
| /56-FAM/ | TTATTATT (SEQ ID NO: 219) | /3IABkFQ/ |

/56-FAM/: 5′ 6-Fluorescein (Integrated DNA Technologies)
/3IABkFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester)(Integrated DNA Technologies)
/5ATTO633N/: 5′ ATTO TM 633 (NHS Ester)(Integrated DNA Technologies)
/3IRQC1N/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

In some cases, the reporter nucleic acid is a single-stranded nucleic acid sequence comprising ribonucleotides. The nucleic acid of a reporter may be a single-stranded nucleic acid sequence comprising at least one ribonucleotide. In some cases, the nucleic acid of a reporter is a single-stranded nucleic acid comprising at least one ribonucleotide residue at an internal position that functions as a cleavage site. In some cases, the nucleic acid of a reporter comprises at least 2, at least 3, at least 4, at least 5, at least 6, at least 7, at least 8, at least 9, or at least 10 ribonucleotide residues at an internal position. In some cases, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some cases, the nucleic acid of a reporter has only ribonucleotide residues. In some cases, the nucleic acid of a reporter has only deoxyribonucleotide residues. In some cases, the nucleic acid comprises nucleotides resistant to cleavage by the programmable nuclease described herein. In some cases, the nucleic acid of a reporter comprises synthetic nucleotides. In some cases, the nucleic acid of a reporter comprises at least one ribonucleotide residue and at least one non-ribonucleotide residue.

In some cases, the nucleic acid of a reporter comprises at least one uracil ribonucleotide. In some cases, the nucleic acid of a reporter comprises at least two uracil ribonucleotides. Sometimes the nucleic acid of a reporter has only uracil ribonucleotides. In some cases, the nucleic acid of a reporter comprises at least one adenine ribonucleotide. In some cases, the nucleic acid of a reporter comprises at least two adenine ribonucleotide. In some cases, the nucleic acid of a reporter has only adenine ribonucleotides. In some cases, the nucleic acid of a reporter comprises at least one cytosine ribonucleotide. In some cases, the nucleic acid of a reporter comprises at least two cytosine ribonucleotide. In some cases, the nucleic acid of a reporter comprises at least one guanine ribonucleotide. In some cases, the nucleic acid of a reporter comprises at least two guanine ribonucleotide. In some instances, a nucleic acid of a reporter comprises a single unmodified ribonucleotide. In some instances, a nucleic acid of a reporter comprises only unmodified deoxyribonucleotides.

In some cases, the nucleic acid of a reporter is 5 to 20, 5 to 15, 5 to 10, 7 to 20, 7 to 15, or 7 to 10 nucleotides in length. In some cases, the nucleic acid of a reporter is 3 to 20, 4 to 10, 5 to 10, or 5 to 8 nucleotides in length. In some cases, the nucleic acid of a reporter is 5 to 12 nucleotides in length. In some cases, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some cases, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some cases, systems comprise a plurality of reporters. The plurality of reporters may comprise a plurality of signals. In some cases, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some cases, there are 2 to 50, 3 to 40, 4 to 30, 5 to 20, or 6 to 10 different reporters.

In some instances, systems comprise a Type V CRISPR/Cas protein and a reporter nucleic acid configured to undergo transcollateral cleavage by the Type V CRISPR/Cas protein. Transcollateral cleavage of the reporter may generate a signal from reporter or alter a signal from the reporter. In some cases, the signal is an optical signal, such as a fluorescence signal or absorbance band. Transcollateral cleavage of the reporter may alter the wavelength, intensity, or polarization of the optical signal. For example, the reporter may comprise a fluorophore and a quencher, such that transcollateral cleavage of the reporter separates the fluorophore and the quencher thereby increasing a fluorescence signal from the fluorophore. Herein, detection of reporter cleavage to determine the presence of a target nucleic acid sequence may be referred to as ‘DETECTR’. In some embodiments described herein is a method of assaying for a target nucleic acid in a sample comprising contacting the target nucleic acid with a programmable nuclease, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of a programmable nuclease (e.g., a Type V CRISPR/Cas protein as disclosed herein) may be inhibited. This is because the activated programmable nucleases collaterally cleaves any nucleic acids. If total nucleic acids are present in large amounts, they may outcompete reporters for the programmable nucleases. In some instances, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some instances, the sample comprises amplified target nucleic acid. In some instances, the sample comprises an unamplified target nucleic acid. In some instances, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some instances, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

iii. Amplification Reagents Components

In some embodiments, systems comprise a reagent or component for amplifying a nucleic acid. Non-limiting examples of reagents for amplifying a nucleic acid include polymerases, primers, and nucleotides. In some instances, systems comprise reagents for nucleic acid amplification of a target nucleic acid in a sample. Nucleic acid amplification of the target nucleic acid may improve at least one of sensitivity, specificity, or accuracy of the assay in detecting the target nucleic acid. In some instances, nucleic acid amplification is isothermal nucleic acid amplification, providing for the use of the system or system in remote regions or low resource settings without specialized equipment for amplification. In some cases, amplification of the target nucleic acid increases the concentration of the target nucleic acid in the sample relative to the concentration of nucleic acids that do not correspond to the target nucleic acid. In some cases, amplification or amplifying can comprise a process by which a nucleic acid molecule is enzymatically copied to generate a plurality of nucleic acid molecules containing the same sequence as the original nucleic acid molecule or a distinguishable portion thereof.

The reagents for nucleic acid amplification may comprise a recombinase, an oligonucleotide primer, a single-stranded DNA binding (SSB) protein, a polymerase, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some instances, systems comprise a PCR tube, a PCR well or a PCR plate. The wells of the PCR plate may be pre-aliquoted with the reagent for amplifying a nucleic acid, as well as a guide nucleic acid, a programmable nuclease, a multimeric complex, or any combination thereof. The wells of the PCR plate may be pre-aliquoted with a guide nucleic acid targeting a target sequence, a programmable nuclease capable of being activated when complexed with the guide nucleic acid and the target sequence, and at least one population of a single stranded reporter nucleic acid comprising a detection moiety. A user may thus add the biological sample of interest to a well of the pre-aliquoted PCR plate and measure for the detectable signal with a fluorescent light reader or a visible light reader.

In some embodiments, systems comprise a PCR plate; a guide nucleic acid targeting a target sequence; a programmable nuclease capable of being activated when complexed with the guide nucleic acid and the target sequence; and a single stranded reporter nucleic acid comprising a detection moiety, wherein the reporter nucleic acid is capable of being cleaved by the activated nuclease, thereby generating a detectable signal.

In some embodiments, systems comprise a support medium; a guide nucleic acid targeting a target sequence; and a programmable nuclease capable of being activated when complexed with the guide nucleic acid and the target sequence. In some cases, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system for modifying a target nucleic acid comprises a PCR plate; a guide nucleic acid targeting a target sequence; and a programmable nuclease capable of being activated when complexed with the guide nucleic acid and the target sequence. The wells of the PCR plate may be pre-aliquoted with the guide nucleic acid targeting a target sequence, and a programmable nuclease capable of being activated when complexed with the guide nucleic acid and the target sequence. A user may thus add the biological sample of interest to a well of the pre-aliquoted PCR plate.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. Sometimes, the nucleic acid amplification is performed for 1 to 60, 5 to 55, 10 to 50, 15 to 45, 20 to 40, or 25 to 35 minutes. Sometimes, the nucleic acid amplification reaction is performed at a temperature of around 20-45° C. In some cases, the nucleic acid amplification reaction is performed at a temperature no greater than 20° C., 25° C., 30° C., 35° C., 37° C., 40° C., 45° C., or any value 20° C. to 45° C. In some cases, the nucleic acid amplification reaction is performed at a temperature of at least 20° C., 25° C., 30° C., 35° C., 37° C., 40° C., or 45° C., or any value 20° C. to 45° C. In some cases, the nucleic acid amplification reaction is performed at a temperature of 20° C. to 45° C., 25° C. to 40° C., 30° C. to 40° C., or 35° C. to 40° C.

Often, systems comprise primers for amplifying a target nucleic acid to produce an amplification product comprising the target nucleic acid and a PAM. For instance, at least one of the primers may comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid sequence, methods of assaying for a target nucleic acid that lacks a PAM by amplifying the target nucleic acid sequence to introduce a PAM, and compositions used in introducing a PAM via amplification into the target nucleic acid sequence.

iv. Additional System Components

In some instances, systems include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of use.

A system may include labels listing contents and/or instructions for use, or package inserts with instructions for use. A set of instructions will also typically be included. In one embodiment, a label is on or associated with the container. In some instances, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

In some instances, systems comprises a solid support. An RNP or programmable nuclease may be attached to a solid support. The solid support may be an electrode or a bead. The bead may be a magnetic bead. Upon cleavage, the RNP is liberated from the solid support and interacts with other mixtures. For example, upon cleavage of the nucleic acid of the RNP, the programmable nuclease of the RNP flows through a chamber into a mixture comprising a substrate. When the programmable nuclease meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### B. Certain Conditions of Systems

In some instances, systems and methods are employed under certain conditions that enhance an activity of the programmable nuclease, a dimer thereof, or a multimeric complex thereof, relative to alternative conditions, as measured by a detectable signal released from cleavage of a reporter in the presence of the target nucleic acid. The detectable signal may be generated at about the rate of transcollateral cleavage of a reporter nucleic acid. In some instances, the reporter nucleic acid is a homopolymeric reporter nucleic acid comprising 5 to 20 consecutive adenines (SEQ ID NO: 314), 5 to 20 consecutive thymines (SEQ ID NO: 315), 5 to 20 consecutive cytosines (SEQ ID NO: 316), or 5 to 20 consecutive guanines (SEQ ID NO: 317). In some instances, the reporter is an RNA-FQ reporter.

In some instances, programmable nucleases, dimers, multimeric complexes, or combinations thereof recognize, bind, or are activated by, different target nucleic acids having different sequences, but are active toward the same reporter nucleic acid, allowing for facile multiplexing in a single assay having a single ssRNA-FQ reporter.

In some instances, systems are employed under certain conditions that enhance transcollateral cleavage activity of the programmable nuclease. In some instances, under certain conditions, transcolatteral cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some instances, systems and methods are employed under certain conditions that enhance cis-cleavage activity of the programmable nuclease.

Certain conditions that may enhance the activity of a programmable nuclease include a certain salt presence or salt concentration of the solution in which the activity occurs. For example, cis-cleavage activity of a programmable nuclease may be inhibited or halted by a high salt concentration. The salt may be a sodium salt, a potassium salt, or a magnesium salt. In some instances, the salt is NaCl. In some instances, the salt is KNO₃. In some instances, the salt concentration is less than 150 mM, less than 125 mM, less than 100 mM, less than 75 mM, less than 50 mM, or less than 25 mM.

Certain conditions that may enhance the activity of a programmable nuclease includes the pH of a solution in which the activity. For example, increasing pH may enhance transcollateral activity. For example, the rate of transcollateral activity may increase with increase in pH up to pH 9. In some instances, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some instances, the pH is 7 to 7.5, 7.5 to 8, 8 to 8.5, 8.5 to 9, or 7 to 8.5. In some cases, the pH is less than 7. In some cases, the pH is greater than 7.

Certain conditions that may enhance the activity of a programmable nuclease includes the temperature at which the activity is performed. In some instances, the temperature is about 25° C. to about 50° C. In some instances, the temperature is about 20° C. to about 40° C., about 30° C. to about 50° C., or about 40° C. to about 60° C. In some instances the temperature is about 25° C., about 30° C., about 35° C., about 40° C., about 45° C., or about 50° C.

In some instances, a final concentration of a programmable nuclease or multimeric complex thereof in a buffer of a system is 1 pM to 1 nM, 1 pM to 10 pM, 10 pM to 100 pM, 100 pM to 1 nM, 1 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1000 nM. The final concentration of the sgRNA complementary to the target nucleic acid may be 1 pM to 1 nM, 1 pM to 10 pM, 10 pM to 100 pM, 100 pM to 1 nM, 1 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1000 nM. The concentration of the ssDNA-FQ reporter may be 1 pM to 1 nM, 1 pM to 10 pM, 10 pM to 100 pM, 100 pM to 1 nM, 1 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1000 nM.

In some instances, systems comprise an excess volume of solution comprising the guide nucleic acid, the programmable nuclease (e.g., a Type V CRISPR/Cas protein as disclosed herein), and the reporter, which contacts a smaller volume comprising a sample with a target nucleic acid. The smaller volume comprising the sample may be unlysed sample, lysed sample, or lysed sample which has undergone any combination of reverse transcription, amplification, and in vitro transcription. The presence of various reagents, (such as buffer, magnesium sulfate, salts, the pH, a reducing agent, primers, dNTPs, NTPs, cellular lysates, non-target nucleic acids, primers, or other components), in a crude, non-lysed sample, a lysed sample, or a lysed and amplified sample, may inhibit the ability of the programmable nuclease to become activated or to find and cleave the nucleic acid of the reporter. This may be due to nucleic acids that are not the reporter outcompeting the nucleic acid of the reporter, for the programmable nuclease. Alternatively, various reagents in the sample may simply inhibit the activity of the programmable nuclease. Thus, the compositions and methods provided herein for contacting an excess volume comprising the engineered guide nucleic acid, the programmable nuclease, and the reporter to a smaller volume comprising the sample with the target nucleic acid of interest provides for superior detection of the target nucleic acid by ensuring that the programmable nuclease is able to find and cleaves the nucleic acid of the reporter. In some embodiments, the volume comprising the guide nucleic acid, the programmable nuclease, and the reporter (may be referred to as “a second volume”) is 4-fold greater than a volume comprising the sample (may be referred to as “a first volume”). In some embodiments, the volume comprising the guide nucleic acid, the programmable nuclease, and the reporter (may be referred to as “a second volume”) is at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold greater than a volume comprising the sample (may be referred to as “a first volume”). In some embodiments, the volume comprising the sample is at least 0.5 μL, at least 1 μL, at least at least 1 μL, at least 2 μL, at least 3 μL, at least 4 μL, at least 5 μL, at least 6 μL, at least 7 μL, at least 8 μL, at least 9 μL, at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 25 μL, at least 30 μL, at least 35 μL, at least 40 μL, at least 45 μL, at least 50 μL, at least 55 μL, at least 60 μL, at least 65 μL, at least 70 μL, at least 75 μL, at least 80 μL, at least 85 μL, at least 90 μL, at least 95 μL, at least 100 μL, 0.5 μL to 5 μL μL, 5 μL to 10 μL, 10 μL to 15 μL, 15 μL to 20 μL, 20 μL to 25 μL, 25 μL to 30 μL, 30 μL to 35 μL, 35 μL to 40 μL, 40 μL to 45 μL, 45 μL to 50 μL, 10 μL to 20 μL, 5 μL to 20 μL, 1 μL to 40 μL, 2 μL to 10 μL, or 1 μL to 10 μL. In some embodiments, the volume comprising the programmable nuclease, the guide nucleic acid, and the reporter is at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 21 μL, at least 22 μL, at least 23 μL, at least 24 μL, at least 25 μL, at least 26 μL, at least 27 μL, at least 28 μL, at least 29 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 350 μL, at least 400 μL, at least 450 μL, at least 500 μL, 10 μL to 15 μL μL, 15 μL to 20 μL, 20 μL to 25 μL, 25 μL to 30 μL, 30 μL to 35 μL, 35 μL to 40 μL, 40 μL to 45 μL, 45 μL to 50 μL, 50 μL to 55 μL, 55 μL to 60 μL, 60 μL to 65 μL, 65 μL to 70 μL, 70 μL to 75 μL, 75 μL to 80 μL, 80 μL to 85 μL, 85 μL to 90 μL, 90 μL to 95 μL, 95 μL to 100 μL, 100 μL to 150 μL, 150 μL to 200 μL, 200 μL to 250 μL, 250 μL to 300 μL, 300 μL to 350 μL, 350 μL to 400 μL, 400 μL to 450 μL, 450 μL to 500 μL, 10 μL to 20 μL, 10 μL to 30 μL, 25 μL to 35 μL, 10 μL to 40 μL, 20 μL to 50 μL, 18 μL to 28 μL, or 17 μL to 22 μL.

In some instances, systems comprise a programmable nuclease that nicks a target nucleic acid, thereby producing a nicked product. In some instances, systems cleave a target nucleic acid, thereby producing a linearized product. In some cases, systems produce at least 50%, at least 55%, at least 60%, at least 65%, at least 70%, at least 75%, at least 80%, at least 85%, at least 90 or at least 95% of a maximum amount of nicked product within 1 minute, where the maximum amount of nicked product is the maximum amount detected within a 60 minute period from when the target nucleic acid is mixed with the programmable nuclease or the multimeric complex thereof. In some cases, systems produce at least 50%, at least 55%, at least 60%, at least 65%, at least 70%, at least 75%, at least 80%, at least 85%, at least 90 or at least 95% of a maximum amount of linearized product within 1 minute, where the maximum amount of linearized product is the maximum amount detected within a 60 minute period from when the target nucleic acid is mixed with the programmable nuclease. In some cases, at least 80% of the maximum amount of linearized product is produced within 1 minute. In some cases, at least 90% of the maximum amount of linearized product is produced within 1 minute.

### C. Certain Systems

In some cases, systems comprise a DNA Endonuclease Targeted CRISPR TransReporter (DETECTR) assay, and a programmable nuclease disclosed herein, a dimer thereof, or a multimeric complex thereof. The principles of the DETECTR assay are described in Chen et al. (Science 2018 Apr. 27; 360(6387): 436-439) and may be modified to facilitate the use of the programmable nucleases described herein. A DETECTR assay may utilize the trans-cleavage abilities of programmable nucleases to achieve fast and high-fidelity detection of a target nucleic acid in a sample. For example, following target RNA extraction from a biological sample, crRNA comprising a portion that is complementary to the target RNA of interest may bind to the target RNA sequence, initiating indiscriminate ssRNase activity by the programmable nuclease. Upon hybridization with the target RNA, the trans-cleavage activity of the programmable nuclease is activated, which may then cleave an ssDNA fluorescence-quenching (FQ) reporter molecule (e.g., an RNA molecule comprising a fluorophore and a fluorescence quenching moiety that may separate upon cleavage of the RNA molecule). Cleavage of the reporter molecule may provide a fluorescent readout indicating the presence of the target RNA in the sample. In some embodiments, the programmable nucleases disclosed herein may be combined, or multiplexed, with other programmable nucleases in a DETECTR assay.

An example of a system for a DETECTR assay comprises final concentrations of 100 nM Type V CRISPR/Cas protein, 125 nM sgRNA, and 50 nM ssRNA-FQ reporter in a total reaction volume of 20 μL. The Type V CRISPR/Cas protein or variant thereof may form a homodimeric complex configured to bind a single guide nucleic acid and a single target nucleic acid molecule. Reactions are incubated in a fluorescence plate reader (Tecan Infinite Pro 200 M Plex) for 2 hours at 37° C. with fluorescence measurements taken every 30 seconds (e.g., λex: 485 nm; λem: 535 n). The fluorescence wavelength detected may vary depending on the reporter molecule.

In some instances, a DETECTR assay is used to detect an amplified target nucleic acid, wherein the amplified target nucleic acid is present in an amount relative to an amount of a programmable nuclease. In some embodiments, the amplified target nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the programmable nuclease. In some embodiments, the amplified target nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the programmable nuclease. In some embodiments, the amplified target nucleic acid is present at 1-fold to 2-fold, 1-fold to 3-fold, 1-fold to 4-fold, 1-fold to 5-fold, 1-fold to 10-fold, 1-fold to 25-fold, 1-fold to 50-fold, 1-fold to 100-fold, 1-fold to 500-fold, 1-fold to 1000-fold, 1-fold to 10,000-fold, 1-fold to 100,000-fold, 5-fold to 10-fold, 5-fold to 25-fold, 5-fold to 50-fold, 5-fold to 100-fold, 5-fold to 500-fold, 5-fold to 1000-fold, 5-fold to 10,000-fold, 5-fold to 100,000-fold, 10-fold to 25-fold, 10-fold to 50-fold, 10-fold to 100-fold, 10-fold to 500-fold, 10-fold to 1000-fold, 10-fold to 10,000-fold, 10-fold to 100,000-fold, 100-fold to 500-fold, 100-fold to 1000-fold, 100-fold to 10,000-fold, 100-fold to 100,000-fold, 1000-fold to 10,000-fold, 1000-fold to 100,000-fold, or 10,000-fold to 100,000-fold molar excess relative to the amount of the programmable nuclease. In some embodiments, the programmable nuclease is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the programmable nuclease is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the programmable nuclease is present in 1-fold to 2-fold, 1-fold to 3-fold, 1-fold to 4-fold, 1-fold to 5-fold, 1-fold to 10-fold, 1-fold to 25-fold, 1-fold to 50-fold, 1-fold to 100-fold, 1-fold to 500-fold, 1-fold to 1000-fold, 1-fold to 10,000-fold, 1-fold to 100,000-fold, 5-fold to 10-fold, 5-fold to 25-fold, 5-fold to 50-fold, 5-fold to 100-fold, 5-fold to 500-fold, 5-fold to 1000-fold, 5-fold to 10,000-fold, 5-fold to 100,000-fold, 10-fold to 25-fold, 10-fold to 50-fold, 10-fold to 100-fold, 10-fold to 500-fold, 10-fold to 1000-fold, 10-fold to 10,000-fold, 10-fold to 100,000-fold, 100-fold to 500-fold, 100-fold to 1000-fold, 100-fold to 10,000-fold, 100-fold to 100,000-fold, 1000-fold to 10,000-fold, 1000-fold to 100,000-fold, or 10,000-fold to 100,000-fold molar excess relative to the amount of the target nucleic acid. In some embodiments, the target nucleic acid is not present in the sample.

In some instances, a DETECTR assay is used to detect an amplified target nucleic acid, wherein the amplified target nucleic acid is present in an amount relative to an amount of a guide nucleic acid. In some embodiments, the amplified target nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the guide nucleic acid. In some embodiments, the amplified target nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the guide nucleic acid. In some embodiments, the amplified target nucleic acid is present in 1-fold to 2-fold, 1-fold to 3-fold, 1-fold to 4-fold, 1-fold to 5-fold, 1-fold to 10-fold, 1-fold to 25-fold, 1-fold to 50-fold, 1-fold to 100-fold, 1-fold to 500-fold, 1-fold to 1000-fold, 1-fold to 10,000-fold, 1-fold to 100,000-fold, 5-fold to 10-fold, 5-fold to 25-fold, 5-fold to 50-fold, 5-fold to 100-fold, 5-fold to 500-fold, 5-fold to 1000-fold, 5-fold to 10,000-fold, 5-fold to 100,000-fold, 10-fold to 25-fold, 10-fold to 50-fold, 10-fold to 100-fold, 10-fold to 500-fold, 10-fold to 1000-fold, 10-fold to 10,000-fold, 10-fold to 100,000-fold, 100-fold to 500-fold, 100-fold to 1000-fold, 100-fold to 10,000-fold, 100-fold to 100,000-fold, 1000-fold to 10,000-fold, 1000-fold to 100,000-fold, or 10,000-fold to 100,000-fold molar excess relative to the amount of the guide nucleic acid. In some embodiments, the guide nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the guide nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the guide nucleic acid is present in 1-fold to 2-fold, 1-fold to 3-fold, 1-fold to 4-fold, 1-fold to 5-fold, 1-fold to 10-fold, 1-fold to 25-fold, 1-fold to 50-fold, 1-fold to 100-fold, 1-fold to 500-fold, 1-fold to 1000-fold, 1-fold to 10,000-fold, 1-fold to 100,000-fold, 5-fold to 10-fold, 5-fold to 25-fold, 5-fold to 50-fold, 5-fold to 100-fold, 5-fold to 500-fold, 5-fold to 1000-fold, 5-fold to 10,000-fold, 5-fold to 100,000-fold, 10-fold to 25-fold, 10-fold to 50-fold, 10-fold to 100-fold, 10-fold to 500-fold, 10-fold to 1000-fold, 10-fold to 10,000-fold, 10-fold to 100,000-fold, 100-fold to 500-fold, 100-fold to 1000-fold, 100-fold to 10,000-fold, 100-fold to 100,000-fold, 1000-fold to 10,000-fold, 1000-fold to 100,000-fold, or 10,000-fold to 100,000-fold molar excess relative to the amount of the target nucleic acid. In some embodiments, the target nucleic acid is not present in the sample.

In some cases, systems comprise a specific high-sensitivity enzymatic reporter unlocking (SHERLOCK) assay, and a programmable nuclease disclosed herein, a dimer thereof, or a multimeric complex thereof. The SHERLOCK assay is described in Kellner et al. (Nat Protoc. 2019 October; 14(10):2986-3012) and may be modified to facilitate the use of the programmable nucleases described herein.

In some instances, systems for detecting a target nucleic acid comprises a support medium; a guide nucleic acid targeting a target sequence; a programmable nuclease capable of being activated when complexed with the guide nucleic acid and the target sequence; and a reporter nucleic acid comprising a detection moiety, wherein the reporter nucleic acid is capable of being cleaved by the activated nuclease, thereby generating a first detectable signal.

### IV. Methods of Nucleic Acid Detection

Provided herein are methods of detecting target nucleic acids. Methods may comprise detecting target nucleic acids with compositions or systems described herein. Methods may comprise detecting a target nucleic acid with systems described herein that comprise a DETECTR assay. Methods may comprise detecting a target nucleic acid in a sample, e.g., a cell lysate, a biological fluid, or environmental sample. Methods may comprise detecting a target nucleic acid in a cell. In some instances, methods of detecting a target nucleic acid in a sample or cell comprises contacting the sample or cell with a programmable nuclease or a multimeric complex thereof, a guide nucleic acid, wherein at least a portion of the guide nucleic acid is complementary to at least a portion of the target nucleic acid, and a reporter nucleic acid that is cleaved in the presence of the programmable nuclease, the guide nucleic acid, and the target nucleic acid, and detecting a signal produced by cleavage of the reporter nucleic acid, thereby detecting the target nucleic acid in the sample. In some instances, methods result in transcollateral cleavage of the reporter nucleic acid. In some instances, methods result in cis cleavage of the reporter nucleic acid. In some instances, the programmable nuclease comprises an amino acid sequence that is at least is at least 70%, at least 75%, at least 80%, at least 85%, at least 90%, at least 92%, at least 95%, at least 97%, at least 98%, at least 99%, or 100% identical to any one of SEQ ID NOs: 1-5. In some instances, the amino acid sequence of the programmable nuclease is at least is at least 70%, at least 75%, at least 80%, at least 85%, at least 90%, at least 92%, at least 95%, at least 97%, at least 98%, at least 99%, or 100% identical to any one of SEQ ID NOs: 1-5. In some cases, a reporter and/or a reporter nucleic acid comprise a non-target nucleic acid molecule that can provide a detectable signal upon cleavage by a programmable nuclease. Examples of detectable signals and detectable moieties that generate detectable signals are provided herein.

In some embodiments, target nucleic acid comprises a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA). The target nucleic acid may be from any organism, including, but not limited to, a bacterium, a virus, a parasite, a protozoon, a fungus, a mammal, a plant, and an insect. As another non-limiting example, the target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides).

Methods may comprise contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

Methods may comprise contacting the sample comprising the target nucleic acid with a guide nucleic acid targeting a target nucleic acid segment, a programmable nuclease capable of being activated when complexed with the guide nucleic acid and the target nucleic acid segment, a single stranded nucleic acid of a reporter comprising a detection moiety, wherein the nucleic acid of a reporter is capable of being cleaved by the activated programmable nuclease, thereby generating a first detectable signal, cleaving the single stranded nucleic acid of a reporter using the programmable nuclease that cleaves as measured by a change in color, and measuring the first detectable signal on the support medium.

Methods may comprise contacting the sample or cell with a programmable nuclease or a multimeric complex thereof and a guide nucleic acid at a temperature of at least about 25° C., at least about 30° C., at least about 35° C., at least about 40° C., at least about 50° C., or at least about 65° C. In some instances, the temperature is not greater than 80° C. In some instances, the temperature is about 25° C., about 30° C., about 35° C., about 40° C., about 45° C., about 50° C., about 55° C., about 60° C., about 65° C., or about 70° C. In some instances, the temperature is about 25° C. to about 45° C., about 35° C. to about 55° C., or about 55° C. to about 65° C.

Methods may comprise cleaving a strand of a single-stranded target nucleic acid with a Type V CRISPR/Cas protein or a multimeric complex thereof, as assessed with an in vitro cis-cleavage assay. In some embodiments, a cleavage assay comprises an assay designed to visualize, quantitate or identify cleavage of a nucleic acid. In some cases, the cleavage activity may be cis-cleavage activity. In some cases, the cleavage activity may be trans-cleavage activity. An example of such an assay may follow a procedure comprising: (i) providing equimolar (e.g., 500 nM) amounts of a programmable nuclease comprising at least 70% sequence identity to any one of SEQ ID NOs: 1-5 and a guide nucleic acid at 40 to 45° C. for 5 minutes in pH 7.5 Tris-HCl buffer, 40 mM NaCl, 2 mM Ca(NO₃)₂, 1 mM BME, thereby forming a ribonucleoprotein complex comprising a dimer of the programmable nuclease and the guide nucleic acid; (ii) adding linear dsDNA comprising a nucleic acid sequence targeted by the guide nucleic acid and adjacent to a PAM comprising the sequence 5′-TTTA-3′; (iii) incubating the mixture at 45° C. for 20 minutes, thereby enabling cleavage of the plasmid; (iv) quenching the reaction with EDTA and a protease; and (v) analyzing the reaction products (e.g., viewing the cleaved and uncleaved linear dsDNA with gel electrophoresis).

In some embodiments, cleave, cleaving, and cleavage, with reference to a nucleic acid molecule or nuclease activity of a programmable nuclease, comprises the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the programmable nuclease.

In some cases, there is a threshold of detection for methods of detecting target nucleic acids. In some instances, methods are not capable of detecting target nucleic acids that are present in a sample or solution at a concentration less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases the threshold of detection is in a range of from 2 aM to 100 pM, from 20 aM to 50 pM, from 50 aM to 20 pM, from 200 aM to 5 pM, or from 500 aM to 2 pM. In some cases, the minimum concentration at which a target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a target nucleic acid is detected in a sample is in a range of from 2 aM to 100 pM, from 20 aM to 50 pM, from 50 aM to 20 pM, from 200 aM to 5 pM, or from 500 aM to 2 pM. In some cases, the minimum concentration at which a single stranded target nucleic acid can be detected in a sample is in a range of from 1 aM to 100 pM. In some cases, the minimum concentration at which a target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some embodiments, the target nucleic acid is present in a cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in the cleavage reaction at a concentration of from 20 nM to 50 μM, from 50 nM to 20 μM, or from 200 nM to 5 μM.

In some cases, methods detect a target nucleic acid in less than 60 minutes. In some cases, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

In some cases, methods require at least about 120 minutes, at least about 110 minutes, at least about 100 minutes, at least about 90 minutes, at least about 80 minutes, at least about 70 minutes, at least about 60 minutes, at least about 55 minutes, at least about 50 minutes, at least about 45 minutes, at least about 40 minutes, at least about 35 minutes, at least about 30 minutes, at least about 25 minutes, at least about 20 minutes, at least about 15 minutes, at least about 10 minutes, or at least about 5 minutes to detect a target nucleic acid. In some cases, the sample is contacted with the reagents for from 5 minutes to 120 minutes, from 5 minutes to 100 minutes, from 10 minutes to 90 minutes, from 15 minutes to 45 minutes, or from 20 minutes to 35 minutes.

In some cases, methods of detecting are performed in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some cases, methods of detecting are performed in about 5 minutes to about 10 hours, about 10 minutes to about 8 hours, about 15 minutes to about 6 hours, about 20 minutes to about 5 hours, about 30 minutes to about 2 hours, or about 45 minutes to about 1 hour.

Methods may comprise detecting a detectable signal within 5 minutes of contacting the sample and/or the target nucleic acid with the guide nucleic acid and/or the programmable nuclease. In some cases, detecting occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some embodiments, detecting occurs within 1 to 120, 5 to 100, 10 to 90, 15 to 80, 20 to 60, or 30 to 45 minutes of contacting the target nucleic acid.

### A. Amplification of a Target Nucleic Acid

Methods may comprise amplifying a target nucleic acid for detection using any of the compositions or systems described herein. Amplifying may comprise changing the temperature of the amplification reaction, also known as thermal amplification (e.g., PCR). Amplifying may be performed at essentially one temperature, also known as isothermal amplification. Amplifying may improve at least one of sensitivity, specificity, or accuracy of the detection of the target nucleic acid.

Amplifying may comprise subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some embodiments, amplification of the target nucleic acid comprises modifying the sequence of the target nucleic acid. For example, amplification may be used to insert a PAM sequence into a target nucleic acid that lacks a PAM sequence. In some cases, amplification may be used to increase the homogeneity of a target nucleic acid in a sample. For example, amplification may be used to remove a nucleic acid variation that is not of interest in the target nucleic acid sequence.

Amplifying may take 1, 2, 3, 4, 5, 6, 7, 8, 9, 10, 11, 12, 13, 14, 15, 16, 17, 18, 19, 20, 25, 30, 40, 50, or 60 minutes. Amplifying may be performed at a temperature of around 20-45° C. Amplifying may be performed at a temperature of less than about 20° C., less than about 25° C., less than about 30° C., 35° C., less than about 37° C., less than about 40° C., or less than about 45° C. The nucleic acid amplification reaction may be performed at a temperature of at least about 20° C., at least about 25° C., at least about 30° C., at least about 35° C., at least about 37° C., at least about 40° C., or at least about 45° C.

### B. Certain Methods of Detection

An illustrative method for detecting a target nucleic acid molecule in a sample comprises contacting the sample comprising the target nucleic acid molecule with (i) a Type V CRISPR/Cas protein comprising at least 75% sequence identity to a sequence selected from the group consisting of SEQ ID NOs: 1-5; (ii) an engineered guide nucleic acid comprising a region that binds to the Type V CRISPR/Cas protein and an additional region that binds to the target nucleic acid; and (iii) a labeled, single stranded RNA reporter; cleaving the labeled single stranded RNA reporter by the Type V CRISPR/Cas protein to release a detectable label; and detecting the target nucleic acid by measuring a signal from the detectable label.

A further illustrative method for detecting a target nucleic acid molecule in a sample comprises contacting the sample comprising the target nucleic acid molecule with (i) a dimeric protein complex comprising a Type V CRISPR/Cas protein comprising at least 75% sequence identity to a sequence selected from the group consisting of SEQ ID NOs: 1-5; (ii) an engineered guide nucleic acid comprising a first region that binds to the target nucleic acid; (iii) a nucleic acid comprising a first region that binds to the Type V CRISPR/Cas protein and an additional region that hybridizes to second region of the engineered guide nucleic acid; and (iv) a labeled, single stranded RNA reporter; cleaving the labeled single stranded RNA reporter by the Type V CRISPR/Cas protein to release a detectable label; and detecting the target nucleic acid by measuring a signal from the detectable label.

### V. Methods of Nucleic Acid Editing

Provided herein are methods of editing target nucleic acids. In general, editing refers to modifying the nucleobase sequence of a target nucleic acid. However, compositions and systems disclosed herein may also be capable of making epigenetic modifications of target nucleic acids. Programmable nucleases, multimeric complexes thereof and systems described herein may be used for editing or modifying a target nucleic acid. Editing a target nucleic acid may comprise one or more of cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid. Methods of editing may comprise contacting a target nucleic acid with a Type V CRISPR/Cas protein and a guide nucleic acid, wherein the Type V CRISPR/Cas protein comprises an amino acid sequence that is at least 70%, at least 75%, at least 80%, at least 85%, at least 90%, at least 95%, at least 97%, or at least 98%, at least 99%, or 100% identical to any one of SEQ ID NOs: 1-5.

Editing may introduce a mutation (e.g., point mutations, deletions) in a target nucleic acid relative to a corresponding wildtype nucleobase sequence. Editing may remove or correct a disease-causing mutation in a nucleic acid sequence to produce a corresponding wildtype nucleobase sequence. Editing may remove/correct point mutations, deletions, null mutations, or tissue-specific mutations in a target nucleic acid. Editing may be used to generate gene knock-out, gene knock-in, gene editing, gene tagging, or a combination thereof. Methods of the disclosure may be targeted to any locus in a genome of a cell.

Editing may comprise single stranded cleavage, double stranded cleavage, donor nucleic acid insertion, epigenetic modification (e.g., methylation, demethylation, acetylation, or deacetylation), or a combination thereof. In some instances, cleavage (single-stranded or double-stranded) is site-specific, meaning cleavage occurs at a specific site in the target nucleic acid, often within the region of the target nucleic acid that hybridizes with the guide nucleic acid spacer region. In some cases, Type V CRISPR/Cas proteins introduce a single-stranded break in a target nucleic acid to produce a cleaved nucleic acid. In some cases, the Type V CRISPR/Cas protein is capable of introducing a break in a single stranded RNA (ssRNA). The Type V CRISPR/Cas protein may be coupled to a guide nucleic acid that targets a particular region of interest in the ssRNA. In some instances, the target nucleic acid, and the resulting cleaved nucleic acid is contacted with a nucleic acid for homologous recombination (e.g., homology directed repair (HDR)) or non-homologous end joining (NHEJ). In some cases, a double-stranded break in the target nucleic acid may be repaired (e.g., by NHEJ or HDR) without insertion of a donor template, such that the repair results in an indel in the target nucleic acid at or near the site of the double-stranded break.

In some instances, the Type V CRISPR/Cas protein is fused to a chromatin-modifying enzyme. In some cases, the fusion protein chemically modifies the target nucleic acid, for example by methylating, demethylating, or acetylating the target nucleic acid in a sequence specific or non-specific manner.

Methods may comprise use of two or more Type V CRISPR/Cas proteins. An illustrative method for introducing a break in a target nucleic acid comprises contacting the target nucleic acid with: (a) a first engineered guide nucleic acid comprising a region that binds to a first programmable nuclease comprising at least 75% sequence identity to a sequence selected from the group consisting of SEQ ID NOs: 1-5; and (b) a second engineered guide nucleic acid comprising a region that binds to a second programmable nuclease comprising at least 75% sequence identity to a sequence selected from the group consisting of SEQ ID NOs: 1-198, wherein the first engineered guide nucleic acid comprises an additional region that binds to the target nucleic acid and wherein the second engineered guide nucleic acid comprises an additional region that binds to the target nucleic acid.

In some embodiments, editing a target nucleic acid comprises genome editing. Genome editing may comprise modifying a genome, chromosome, plasmid, or other genetic material of a cell or organism. In some embodiments the genome, chromosome, plasmid, or other genetic material of the cell or organism is modified in vivo. In some embodiments the genome, chromosome, plasmid, or other genetic material of the cell or organism is modified in a cell. In some embodiments the genome, chromosome, plasmid, or other genetic material of the cell or organism is modified in vitro. For example, a plasmid may be modified in vitro using a composition described herein and introduced into a cell or organism. In some embodiments, modifying a target nucleic acid may comprise deleting a sequence from a target nucleic acid. For example, a mutated sequence or a sequence associated with a disease may be removed from a target nucleic acid. In some embodiments, modifying a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, modifying a target nucleic acid may comprise introducing a sequence into a target nucleic acid. For example, a beneficial sequence or a sequence that may reduce or eliminate a disease may be inserted into the target nucleic acid.

In some instances, methods comprise inserting a donor nucleic acid into a cleaved target nucleic acid. The donor nucleic acid may be inserted at a specified (e.g., programmable nuclease-targeted) point within the target nucleic acid. In some instances, methods comprise contacting a target nucleic acid with a programmable nuclease comprising an amino acid sequence that is at least 70%, at least 75%, at least 80%, at least 85%, at least 90%, at least 95%, at least 97%, at least 98%, at least 99%, or 100% identical to any one of SEQ ID NOs: 1-5, thereby introducing a single-stranded break in the target nucleic acid; contacting the target nucleic acid with a second programmable nuclease, optionally comprising an amino acid sequence that is at least 70%, at least 75%, at least 80%, at least 85%, at least 90%, at least 95%, at least 97%, at least 98%, or at least 99% identical to any one of SEQ ID NOs: 1-5, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally via HDR or NHEJ, thereby introducing a new sequence into the target nucleic acid (e.g., at a cleavage site or in between two cleavage sites).

In some cases, methods comprise editing a target nucleic acid with two or more programmable nickases. Editing a target nucleic acid may comprise introducing a two or more single-stranded breaks in a target nucleic acid. In some embodiments, a break may be introduced by contacting a target nucleic acid with a programmable nickase and a guide nucleic acid. The guide nucleic acid may bind to the programmable nickase and hybridize to a region of the target nucleic acid, thereby recruiting the programmable nickase to the region of the target nucleic acid. Binding of the programmable nickase to the guide nucleic acid and the region of the target nucleic acid may activate the programmable nickase, and the programmable nickase may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first programmable nickase and hybridizes to a first region of the target nucleic acid and a second guide nucleic acid that binds to a second programmable nickase and hybridizes to a second region of the target nucleic acid. The first programmable nickase may introduce a first break in a first strand at the first region of the target nucleic acid, and the second programmable nickase may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with donor nucleic acid), thereby modifying the target nucleic acid.

In some cases, editing is achieved by fusing a programmable nuclease such as a Type V CRISPR/Cas protein to a heterologous sequence. In some cases, a heterologous sequence comprises a nucleotide or polypeptide sequence that is not found in a native nucleic acid or protein, respectively. In some embodiments, fusion proteins comprise a programmable nuclease and a fusion partner protein, wherein the fusion partner protein is heterologous to a programmable nuclease. The heterologous sequence may be a suitable fusion partner, e.g., a protein that provides recombinase activity by acting on the target nucleic acid sequence. In some embodiments, the fusion protein comprises a programmable nuclease such as a Type V CRISPR/Cas protein fused to a heterologous sequence by a linker. The heterologous sequence or fusion partner may be a base editing domain. The base editing domain may be an ADAR1/2 or any functional variant thereof. The heterologous sequence or fusion partner may be fused to the C-terminus, N-terminus, or an internal portion (e.g., a portion other than the N- or C-terminus) of the programmable nuclease. The heterologous sequence or fusion partner may be fused to the programmable nuclease by a linker. In some cases, a heterologous sequence (e.g., a heterologous moiety) can comprise a protein purification tag. A linker may be a peptide linker or a non-peptide linker. In some embodiments, the linker is an XTEN linker. In some embodiments, the linker comprises one or more repeats a tri-peptide GGS. In some embodiments, the linker is from 1 to 100 amino acids in length. In some embodiments, the linker is more 100 amino acids in length. In some embodiments, the linker is from 10 to 27 amino acids in length. A non-peptide linker may be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

### A. Donor Nucleic Acids

In some embodiments, a donor nucleic acid comprises a nucleic acid that is incorporated into a target nucleic acid or target sequence. In some embodiments, in a viral vector, a nucleic acid comprises a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome. As another example, when used in reference to the activity of a programmable nuclease, the donor nucleic acid comprises a sequence of nucleotides that will be or has been inserted at the site of cleavage by the programmable nuclease (cleaving (hydrolysis of a phosphodiester bond) of a nucleic acid resulting in a nick or double strand break-nuclease activity). As yet another example, when used in reference to homologous recombination, a donor nucleic acid comprises a sequence of DNA that serves as a template in the process of homologous recombination, which may carry the modification that is to be or has been introduced into the target nucleic acid. By using this donor nucleic acid as a template, the genetic information, including the modification, is copied into the target nucleic acid by way of homologous recombination.

Donor nucleic acids of any suitable size may be integrated into a target nucleic acid or genome. In some embodiments, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some instances, donor nucleic acids are more than 500 kilobases (kb) in length.

The donor nucleic acid may comprise a sequence that is derived from a plant, bacteria, virus or an animal. The animal may be human. The animal may be a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). The non-human animal may be a domesticated mammal or an agricultural mammal.

In certain embodiments, a donor nucleic acid comprises a transgene. A transgene can be a nucleic acid, such as DNA. In some embodiments, transgenes described herein can be inserted or integrated into the target nucleic acid or target sequence.

### VI. Methods of Introduction to a Host

Described herein are methods of introducing various components described herein to a host. A host can be any suitable host, such as a host cell. When described herein, a host cell can be an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for methods of introduction described herein, and include the progeny of the original cell which has been transformed by the methods of introduction described herein. It is understood that the progeny of a single cell may not necessarily be completely identical in morphology or in genomic or total DNA complement as the original parent, due to natural, accidental, or deliberate mutation. A host cell can be a recombinant host cell or a genetically modified host cell, if a heterologous nucleic acid, e.g., an expression vector, has been introduced into the cell.

In certain embodiments, molecules of interest, such as nucleic acids of interest, are introduced to a host. In certain embodiments, polypeptides, such as a programmable nuclease are introduced to a host. In certain embodiments, vectors, such as lipid particles and/or viral vectors can be introduced to a host. Introduction can be for contact with a host or for assimilation into the host, for example, introduction into a host cell.

In some instances, described herein are methods of introducing one or more nucleic acids, such as a nucleic acid encoding a programmable nuclease, a nucleic acid encoding an engineered guide nucleic acid, and/or a donor nucleic acid, or combinations thereof, into a host cell. Any suitable method can be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout.

Introducing one or more nucleic acids into a host cell can occur in any culture media and under any culture conditions that promote the survival of the cells. Introducing one or more nucleic acids into a host cell can be carried out in vivo or ex vivo. Introducing one or more nucleic acids into a host cell can be carried out in vitro.

In some embodiments, a programmable nuclease can be provided as RNA. The RNA can be provided by direct chemical synthesis or may be transcribed in vitro from a DNA (e.g., encoding the programmable nuclease). Once synthesized, the RNA may be introduced into a cell by way of any suitable technique for introducing nucleic acids into cells (e.g., microinjection, electroporation, transfection, etc.). In some embodiments, introduction of one or more nucleic acid can be through the use of a vector and/or a vector system, accordingly, in some embodiments, compositions and system described herein comprise a vector and/or a vector system.

Vectors may be introduced directly to a host. In certain embodiments, host cells can be contacted with one or more vectors as described herein, and in certain embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

Components described herein can also be introduced directly to a host. For example, an engineered guide nucleic acid can be introduced to a host, specifically introduced into a host cell. Methods of introducing nucleic acids, such as RNA into cells include, but are not limited to direct injection, transfection, or any other method used for the introduction of nucleic acids.

Polypeptides (e.g., programmable nucleases) described herein can also be introduced directly to a host. In some embodiments, polypeptides described herein can be modified to promote introduction to a host. For example, polypeptides described herein can be modified to increase the solubility of the polypeptide. Such a polypeptide may optionally be fused to a polypeptide domain that increases solubility. The domain may be linked to the polypeptide through a defined protease cleavage site, such as TEV sequence which is cleaved by TEV protease. The linker may also include one or more flexible sequences, e.g. from 1 to 10 glycine residues. In some embodiments, the cleavage of the polypeptide is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. In another example, the polypeptide can be modified to improve stability. For example, the polypeptides may be PEGylated, where the polyethyleneoxy group provides for enhanced lifetime in the blood stream. Polypeptides can also be modified to promote uptake by a host, such as a host cell. For example, a polypeptide described herein can be fused to a polypeptide permeant domain to promote uptake by a host cell. Any suitable permeant domains can be used in the non-integrating polypeptides of the present disclosure, including peptides, peptidomimetics, and non-peptide carriers. Examples include penetratin, a permeant peptide may be derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine, and the like. The site at which the fusion is made may be selected in order to optimize the biological activity, secretion or binding characteristics of the polypeptide. The optimal site can be determined by suitable methods.

### A. Vectors and Multiplexed Expression Vectors

In some instances, compositions and systems provided herein comprise a vector system, wherein the vector system comprises one or more vectors. When a vector is described herein, such a vector can be used as a vehicle to introduce one or more molecules of interest into a host cell. A molecule of interest can comprise a polypeptide (e.g., a programmable nuclease), an engineered guide or a component thereof (e.g., crRNA, tracrRNA, or sgRNA), a donor nucleic acid, a nucleic acid encoding a polypeptide, a nucleic acid encoding an engineered guide or a component thereof. For example, vector systems described herein can comprise one or more vectors comprising a polypeptide (e.g., a programmable nuclease), an engineered guide (e.g., crRNA, tracrRNA, or sgRNA), or encoding for, or a nucleic acid or nucleic acids encoding a polypeptide, engineered guide, a donor nucleic acid, or any combination thereof.

In some instances, compositions and systems provided herein comprise a vector system comprising a polypeptide (e.g., a programmable nuclease) described herein. In some instances, compositions and systems provided herein comprise a vector system comprising a guide nucleic acid (e.g., crRNA, tracrRNA, or sgRNA) described herein. In some instances, compositions and systems provided herein comprise a vector system comprising a donor nucleic acid described herein.

In some instances, compositions and systems provided herein comprise a vector system encoding a polypeptide (e.g., a programmable nuclease) described herein. In some instances, compositions and systems provided herein comprise a vector system encoding a guide nucleic acid (e.g., crRNA, tracrRNA, or sgRNA) described herein. In some instances, compositions and systems provided herein comprise a multi-vector system encoding a programmable nuclease and a guide nucleic acid described herein, wherein the guide nucleic acid and the programmable nuclease are encoded by the same or different vectors. In some instances, the guide nucleic acid and the programmable nuclease are encoded by different vectors of the system. In some embodiments, a nucleic acid encoding a polypeptide (e.g., a programmable nuclease) comprises an expression vector. In some embodiments, a nucleic acid encoding a polypeptide is a messenger RNA. In some embodiments, an expression vector comprises or encodes an engineered guide nucleic acid. In some cases, the expression vector encodes the crRNA or sgRNA.

In some instances, a vector may encode one or more programmable nucleases. In some instances, a vector may encode 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 programmable nucleases. In some instances, a vector can encode one or more programmable nucleases comprising an amino acid sequence of any one of SEQ ID NOs: 1-5. In some instances, a vector can encode one or more programmable nucleases comprising an amino acid sequence of any one of SEQ ID NOs: 6-198. In some instances, a vector can encode one or more programmable nucleases comprising an amino acid sequence with at least 75%, 80%, 85%, 90%, 95% or 98% sequence identity to any one of SEQ ID NOs: 1-198.

In some instances, a vector may encode one or more guide nucleic acids. In some instances, a vector may encode 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 different guide nucleic acids. In some instances, a vector can encode one or more guide nucleic acids comprising a crRNA sequence of any one of SEQ ID NOs: 233-238 or 255-262. In some instances, a vector can encode one or more guide nucleic acids comprising a crRNA sequence with at least 75%, 80%, 85%, 90%, 95% or 98% sequence identity to any one of SEQ ID NOs: 233-238 or 255-262. In some instances, a vector can encode one or more guide nucleic acids comprising a tracrRNA sequence with at least 75%, 80%, 85%, 90%, 95% or 98% sequence identity to any one of SEQ ID NOs: 220-232. In some instances, the tracrRNA and the crRNA may be linked into a single guide RNA.

In some instances, a vector can comprise or encode one or more regulatory elements. Regulatory elements can refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some instances, a vector can comprise or encode for one or more additional elements, such as, for example, replication origins, antibiotic resistance (or a nucleic acid encoding the same), a tag (or a nucleic acid encoding the same), selectable markers, and the like.

Vectors described herein can encode a promoter—a regulatory region on a nucleic acid, such as a DNA sequence, capable of initiating transcription of a downstream (3′ direction) coding or non-coding sequence. As used herein, a promoter can be bound at its 3′ terminus to a nucleic acid the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. A promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter. Promotors can be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human Hl promoter (Hl). By transcriptional activation, it is intended that transcription will be increased above basal levels in the target cell by 10 fold, by 100 fold, or by 1000 fold, or more. In addition, vectors used for providing a nucleic acid encoding an engineered guide nucleic acid and/or a programmable nuclease to a cell may include nucleic acid sequences that encode for selectable markers in the target cells, so as to identify cells that have taken up the engineered guide nucleic acid and/or a programmable nuclease.

In some embodiments, a programmable nuclease (or a nucleic acid encoding same) and/or an engineered guide nucleic acid (or a nucleic acid encoding same) are coadministered with a donor nucleic acid. Coadministration can be contact with a target nucleic acid, administered to a cell, such as a host cell, or administered as method of nucleic acid detection, editing, and/or treatment as described herein, in a single vehicle, such as a single expression vector. In certain embodiments, a programmable nuclease (or a nucleic acid encoding same) and/or an engineered guide nucleic acid (or a nucleic acid encoding same) are not co-administered with donor nucleic acid in a single vehicle. In certain embodiments, a programmable nuclease (or a nucleic acid encoding same), an engineered guide nucleic acid (or a nucleic acid encoding same), and/or donor nucleic acid are administered in one or more or two or more vehicles, such as one or more, or two or more expression vectors.

### B. Lipid Particles and Non-Viral Vectors

In some instances, compositions and systems provided herein comprise a lipid particle. In some embodiments, a lipid particle is a lipid nanoparticle (LNP). In some embodiments, a lipid or a lipid nanoparticle can encapsulate an expression vector. In some embodiments, a lipid or a lipid nanoparticle can encapsulate the programmable nuclease, the sgRNA or crRNA, the nucleic acid encoding the programmable nuclease and/or the DNA molecule encoding the sgRNA or crRNA. LNPs are a non-viral delivery system for gene therapy. LNPs are effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3):146-157). In some cases, a method can comprise contacting a cell with an expression vector. In some cases, contacting can comprise electroporation, lipofection, or lipid nanoparticle (LNP) delivery of an expression vector. In some embodiments, a nucleic acid expression vector is a non-viral vector. In some embodiments, compositions and methods comprise a lipid, polymer, nanoparticle, or a combination thereof, or use thereof, to introduce a Cas protein, guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

### C. Viral Vectors

An expression vector can be a viral vector. In some embodiments, a viral vector comprises a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. In some embodiments, the expression vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the viral vector is a replication-defective viral vector, comprising an insertion of a therapeutic gene inserted in genes essential to the lytic cycle, preventing the virus from replicating and exerting cytotoxic effects. A viral vector provided herein can be derived from or based on any such virus. Often the viral vectors provided herein are an adeno-associated viral vector (AAV vector). Generally, an AAV vector has two inverted terminal repeats (ITRs). According, in some embodiments, the viral vector provided herein comprises two inverted terminal repeats of AAV. The DNA sequence in between the ITRs of an AAV vector provided herein may be referred to herein as the sequence encoding the genome editing tools. These genome editing tools can include, but are not limited to, a programmable nuclease, programmable nuclease modifications (e.g., nuclear localization signal (NLS), polyA tail), guide nucleic acid(s), respective promoter(s), and a donor nucleic acid, or combinations thereof.

In general, viral vectors provided herein comprise at least one promotor or a combination of promoters driving expression or transcription of one or more genome editing tools described herein. In some embodiments, the length of the promoter is less than about 500, less than about 400, or less than about 300 linked nucleotides. In some embodiments, the length of the promoter is at least 100 linked nucleotides. Non-limiting examples of promoters include CMV, EF1a, RPBSA, hPGK, EFS, SV40, PGK1, Ube, human beta actin promoter, CAG, TRE, UAS, Ac5, Polyhedrin, CaMKIIa, GAL1, H1, TEF1, GDS, ADH1, CaMV35S, Ubi, U6, MNDU3, and MSCV. In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44.

In some embodiments, the coding region of the AAV vector forms an intramolecular double-stranded DNA template thereby generating an AAV vector that is a self-complementary AAV (scAAV) vector. In general, the sequence encoding the genome editing tools of an scAAV vector has a length of about 2 kb to about 3 kb. The scAAV vector can comprise nucleotide sequences encoding a programmable nuclease, providing guide nucleic acids described herein, and a donor nucleic acid described herein. In some embodiments, the AAV vector provided herein is a self-inactivating AAV vector.

In some embodiments, an AAV vector provided herein comprises a modification, such as an insertion, deletion, chemical alteration, or synthetic modification, relative to a wild-type AAV vector.

In some embodiments, the viral particle that delivers the viral vector described herein is an AAV. AAVs are characterized by their serotype. Non-limiting examples of AAV serotypes are AAV1, AAV2, AAV3, AAV4, AAV5, AAV6, AAV7, AAV8, AAV9, AAV10, AAV11, AAV12, scAAV, AAV-rh10, chimeric or hybrid AAV, or any combination, derivative, or variant thereof

### D. Producing AAV Particles

The AAV particles described herein can be referred to as recombinant AAV (rAAV). Often, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E40RF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles can be produced in the mammalian cell culture system by providing the rAAV plasmid to the mammalian cell. In some embodiments, producing rAAV virus particles in a mammalian cell can comprise transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, an insect cell for producing rAAV viral particles comprises a Sf9 cell. In some embodiments, production of rAAV virus particles in insect cells can comprise baculovirus. In some embodiments, production of rAAV virus particles in insect cells can comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

### E. Genetically Modified Cells and Organisms

Methods of editing described herein may be employed to generate a genetically modified cell. The cell may be a eukaryotic cell (e.g., a mammalian cell) or a prokaryotic cell (e.g., an archaeal cell). The cell may be derived from a multicellular organism and cultured as a unicellular entity. The cell may comprise a heritable genetic modification, such that progeny cells derived therefrom comprise the heritable genetic mutation. The cell may be progeny of a genetically modified cell comprising a genetic modification of the genetically modified parent cell. A genetically modified cell may comprise a deletion, insertion, mutation, or non-native sequence relative to a wild-type version of the cell or the organism from which the cell was derived.

Methods may comprise contacting a cell with a nucleic acid (e.g., a plasmid or mRNA) comprising a nucleobase sequence encoding a programmable nuclease, wherein the programmable nuclease comprise comprises an amino acid sequence that is at least 70%, at least 75%, at least 80%, at least 85%, at least 90%, at least 95%, at least 97%, at least 98%, or at least 99% identical to any one of SEQ ID NOs: 1-5. Methods may comprise contacting cells with a nucleic acid (e.g., a plasmid or mRNA) comprising a nucleobase sequence encoding a guide nucleic acid, a tracrRNA, a crRNA, or any combination thereof. Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof.

Methods may comprise contacting a cell with a programmable nuclease or a multimeric complex thereof, wherein the programmable nuclease comprises an amino acid sequence that is at least 70%, at least 75%, at least 80%, at least 85%, at least 90%, at least 95%, at least 97%, at least 98%, or at least 99% identical to any one of SEQ ID NOs: 1-5. Methods may comprise contacting a cell with a programmable nuclease, wherein the amino acid sequence of the programmable nuclease is at least 70%, at least 75%, at least 80%, at least 85%, at least 90%, at least 95%, at least 97%, at least 98%, or at least 99% identical to any one of SEQ ID NOs: 1-5.

Methods may comprise cell line engineering (e.g., engineering a cell from a cell line for bioproduction). Cell lines may be used to produce a desired protein. In some embodiments, target nucleic acids comprise a genomic sequence. In some embodiments, the cell line is a Chinese hamster ovary cell line (CHO), human embryonic kidney cell line (HEK), cell lines derived from cancer cells, cell lines derived from lymphocytes, and the like. Non-limiting examples of cell lines includes: C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DUi45, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR.

Non-limiting examples of cells that may be engineered or modified with compositions and methods described herein include immune cells, such as CART, T-cells, B-cells, NK cells, granulocytes, basophils, eosinophils, neutrophils, mast cells, monocytes, macrophages, dendritic cells, antigen-presenting cells (APC), or adaptive cells. Non-limiting examples of cells that may be engineered or modified with compositions and methods described herein include include plant cells, such as parenchyma, sclerenchyma, collenchyma, xylem, phloem, germline (e.g., pollen). Cells from lycophytes, ferns, gymnosperms, angiosperms, bryophytes, charophytes, chloropytes, rhodophytes, or glaucophytes. Non-limiting examples of cells that may be engineered or modified with compositions and methods described herein include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

Methods of the disclosure may be performed in a subject. Compositions of the disclosure may be administered to a subject. A subject may be a human. A subject may be a mammal (e.g., rat, mouse, cow, dog, pig, sheep, horse). A subject may be a vertebrate or an invertebrate. A subject may be a laboratory animal. A subject may be a patient. A subject may be suffering from a disease. A subject may display symptoms of a disease. A subject may not display symptoms of a disease, but still have a disease. A subject may be under medical care of a caregiver (e.g., the subject is hospitalized and is treated by a physician). Methods of the disclosure may be performed in a plant, bacteria, or a fungus.

Methods of the disclosure may be performed in a cell. A cell may be in vitro. A cell may be in vivo. A cell may be ex vivo. A cell may be an isolated cell. A cell may be a cell inside of an organism. A cell may be an organism. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. A cell may be a prokaryotic cell or derived from a prokaryotic cell. A cell may be a bacterial cell or may be derived from a bacterial cell. A cell may be an archaeal cell or derived from an archaeal cell. A cell may be a eukaryotic cell or derived from a eukaryotic cell. A cell may be a pluripotent stem cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell. A cell may be a microbe cell or derived from a microbe cell. A cell may be a fungi cell or derived from a fungi cell. A cell may be from a specific organ or tissue.

Methods of the disclosure may be performed in a eukaryotic cell or cell line. In some embodiments, the eukaryotic cell is a Chinese hamster ovary (CHO) cell. In some embodiments, the eukaryotic cell is a Human embryonic kidney 293 cells (also referred to as HEK or HEK 293) cell. Non-limiting examples of cell lines that may be used with compositions, systems and methods of the present disclosure include C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR. Non-limiting examples of other cells that may be used with the disclosure include immune cells, such as CART, T-cells, B-cells, NK cells, granulocytes, basophils, eosinophils, neutrophils, mast cells, monocytes, macrophages, dendritic cells, antigen-presenting cells (APC), or adaptive cells. Non-limiting examples of cells that may be used with this disclosure also include plant cells, such as Parenchyma, sclerenchyma, collenchyma, xylem, phloem, germline (e.g., pollen). Cells from lycophytes, ferns, gymnosperms, angiosperms, bryophytes, charophytes, chloropytes, rhodophytes, or glaucophytes. Non-limiting examples of cells that may be used with this disclosure also include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

### Agricultural Engineering

Compositions and methods of the disclosure may be used for agricultural engineering. For example, compositions and methods of the disclosure may be used to confer desired traits on a plant. A plant may be engineered for the desired physiological and agronomic characteristic using the present disclosure. In some embodiments, the target nucleic acid sequence comprises a nucleic acid sequence of a plant. In some embodiments, the target nucleic acid sequence comprises a genomic nucleic acid sequence of a plant cell. In some embodiments, the target nucleic acid sequence comprises a nucleic acid sequence of an organelle of a plant cell. In some embodiments, the target nucleic acid sequence comprises a nucleic acid sequence of a chloroplast of a plant cell.

The plant may be a dicotyledonous plant. Non-limiting examples of orders of dicotyledonous plants include Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales.

The plant may be a monocotyledonous plant. Non-limiting examples of orders of monocotyledonous plants include Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales. A plant may belong to the order, for example, Gymnospermae, Pinales, Ginkgoales, Cycadales, Araucariales, Cupressales and Gnetales.

Non-limiting examples of plants include plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses, wheat, maize, rice, millet, barley, tomato, apple, pear, strawberry, orange, acacia, carrot, potato, sugar beets, yam, lettuce, spinach, sunflower, rape seed, Arabidopsis, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. A plant may include algae.

### VII. Guide Nucleic Acids

The compositions, systems, and methods of the present disclosure may comprise a guide nucleic acid, or a nucleic acid molecule (e.g., DNA molecule) encoding the guide nucleic acid, or a use thereof. In general, a guide nucleic acid is a nucleic acid molecule that binds to a programmable nuclease (e.g., a CRISPR/Cas protein), thereby forming a ribonucleoprotein complex (RNP) a target nucleic acid, thereby targeting the RNP to the target nucleic acid. A guide RNA generally comprises a CRISPR RNA (crRNA), at least a portion of which is complementary to a target sequence of a target nucleic acid. In some cases, a target sequence when used in reference to a target nucleic acid, comprises a sequence of nucleotides found within a target nucleic acid. Such a sequence of nucleotides can, for example, hybridize to an equal length portion of a guide nucleic acid. Hybridization of the guide nucleic acid to the target sequence may bring a programmable nuclease into contact with the target nucleic acid. In some instances, the guide RNA comprises a trans-activating CRISPR RNA (tracrRNA) that is bound by the programmable nuclease. In some instances, a crRNA and tracrRNA function as two separate, unlinked molecules. For example, an engineered guide nucleic acid herein can comprise a dual guide system that comprises a crRNA and a tracrRNA that are not connected or linked by a covalent bond. In some cases, the guide RNA is a single guide RNA (sgRNA) (e.g., a crRNA linked to a tracrRNA). The term, “tracrRNA” in the context of a sgRNA, is used for simplicity. However, a tracrRNA sequence linked to a crRNA in a sgRNA is not functioning in trans and may not be considered to be a tracrRNA. For instance, the sgRNA often comprises only a portion of a tracrRNA sequence. In some embodiments, the sgRNA comprises only a portion of a naturally occurring tracrRNA sequence. Guide nucleic acids are often referred to as “guide RNA.” However, a guide nucleic acid may comprise deoxyribonucleotides and/or chemically modified nucleotides.

The term “guide RNA,” as well as crRNA and tracrRNA, includes guide nucleic acids comprising DNA bases and RNA bases. In some cases, an RNA sequence is readily derivable from a DNA sequence. An RNA sequence can be derived from a DNA sequence by converting all “T's (Thymine)” to “U's (Uracil)”. Guide nucleic acids, when complexed with a programmable nuclease, may bring the programmable nuclease into proximity of a target nucleic acid. Sufficient conditions for hybridization of a guide nucleic acid to a target nucleic acid and/or for binding of a guide nucleic acid to a programmable nuclease include in vivo physiological conditions of a desired cell type or in vitro conditions sufficient for assaying catalytic activity of a protein, polypeptide or peptide described herein, such as the nuclease activity of a programmable nuclease. Guide nucleic acids may comprise DNA, RNA, or a combination thereof (e.g., RNA with a thymine base). Guide nucleic acids may include a chemically modified nucleobase or phosphate backbone. For example, a DNA sequence of a tracrRNA sequence comprising the sequence: CTTCACTGATAAAGTGGAGAACCGCTTCACCAAAAGCTGTCCCTTAGGGGATTAGAACTTGA GTGAAGGTGGGCTGCTTGCATCAGCCTAATGTCGAGAAGTGCTTTCTTCGGAAAGTAACCCT CGAAACAAATTCATTT (SEQ ID NO: 312) can be derived to the RNA sequence of CUUCACUGAUAAAGUGGAGAACCGCUUCACCAAAAGCUGUCCCUUAGGGGAUUAGAACUU GAGUGAAGGUGGGCUGCUUGCAUCAGCCUAAUGUCGAGAAGUGCUUUCUUCGGAAAGUA ACCCUCGAAACAAAUUCAUUU (SEQ ID NO: 220). In another example, a crRNA comprising the sequence of: GTTGCAGAACCCGAATAGACGAATGAAGGAATGCAACTATTAAATACTCGTATTGCT (SEQ ID NO: 313) can be derived to the RNA sequence of GUUGCAGAACCCGAAUAGACGAAUGAAGGAAUGCAACUAUUAAAUACUCGUAUUGCU (SEQ ID NO: 233). Guide nucleic acids described herein may bind to a Cas 14 protein or multimeric complex thereof, wherein the amino acid sequence of the Cas14 protein is at least 70%, at least 75%, at least 80%, at least 85%, at least 90%, at least 92%, at least 95%, at least 97%, at least 98%, at least 99%, or 100% identical to any one of SEQ ID NO: 1-5.

In some embodiments, a tracrRNA may include deoxyribonucleosides, ribonucleosides, chemically modified nucleosides, or any combination thereof. A tracrRNA may be separate from, but form a complex with, a guide nucleic acid and a programmable nuclease. The tracrRNA may be attached (e.g., covalently) by an artificial linker to a guide nucleic acid. A tracrRNA may include a nucleotide sequence that hybridizes with a portion of a guide nucleic acid. A tracrRNA may also form a secondary structure (e.g., one or more hairpin loops) that facilitates the binding of a programmable nuclease to a guide nucleic acid and/or modification activity of a programmable nuclease on a target nucleic acid. A tracrRNA may include a repeat hybridization region and a hairpin region. The repeat hybridization region may hybridize to all or part of the repeat sequence of a guide nucleic acid. The repeat hybridization region may be positioned 3′ of the hairpin region. The hairpin region may include a first sequence, a second sequence that is reverse complementary to the first sequence, and a stem-loop linking the first sequence and the second sequence.

In some embodiments, the guide nucleic acid comprises a nucleotide sequence as described herein (e.g., TABLE 5). Such nucleotide sequences described herein (e.g., TABLE 5) may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that produces a guide nucleic acid, such as a nucleotide sequence described herein for a vector. Similarly, disclosure of the nucleotide sequences described herein (e.g., TABLE 5) also discloses the complementary nucleotide sequence, the reverse nucleotide sequence, and the reverse complement nucleotide sequence, any one of which can be a nucleotide sequence for use in a guide nucleic acid as described herein.

In some instances, a guide nucleic acid can comprise a repeat sequence as shown in TABLE 4. In some instances, a crRNA or a sgRNA comprises a repeat sequence as shown in TABLE 4. In some instances, a guide nucleic acid comprises a sequence that is at least 70%, at least 75%, at least 80%, at least 85%, at least 90%, or at least 95% identical to any one of SEQ ID Nos 255-262. In some instances, a crRNA or a sgRNA comprises a sequence that is at least 70%, at least 75%, at least 80%, at least 85%, at least 90%, or at least 95% identical to any one of SEQ ID Nos 255-262. In some instances, guide nucleic acids comprise at least 8, at least 9, at least 10, at least 11, at least 12, at least 13, at least 14, at least 15, at least 16, at least 17, at least 18, at least 19, at least 20 at least 25, at least 30, or at least 35 contiguous nucleotides of a nucleotide sequence in TABLE 4. In some cases, compositions comprise a programmable nuclease that is at least 70%, at least 80%, at least 90%, or 100% identical to SEQ ID NO: 1, and a guide nucleic acid comprising a nucleotide sequence that is at least 70%, at least 80%, at least 90%, or 100% identical to SEQ ID NO: 255. In some cases, compositions comprise a programmable nuclease that is at least 70%, at least 80%, at least 90%, or 100% identical to SEQ ID NO: 2, 4, or 5, and a guide nucleic acid comprising a nucleotide sequence that is at least 70%, at least 80%, at least 90%, or 100% identical to SEQ ID NO: 256. In some cases, compositions comprise a programmable nuclease that is at least 70%, at least 80%, at least 90%, or 100% identical to SEQ ID NO: 3, and a guide nucleic acid comprising a nucleotide sequence that is at least 70%, at least 80%, at least 90%, or 100% identical to SEQ ID NO: 257. In some cases, compositions comprise a programmable nuclease that is at least 70%, at least 80%, at least 90%, or 100% identical to SEQ ID NO: 1, and a guide nucleic acid comprising a nucleotide sequence that is at least 70%, at least 80%, at least 90%, or 100% identical to SEQ ID NO: 258. In some cases, compositions comprise a programmable nuclease that is at least 70%, at least 80%, at least 90%, or 100% identical to SEQ ID NO: 2, and a guide nucleic acid comprising a nucleotide sequence that is at least 70%, at least 80%, at least 90%, or 100% identical to SEQ ID NO: 259. In some cases, compositions comprise a programmable nuclease that is at least 70%, at least 80%, at least 90%, or 100% identical to SEQ ID NO: 3, and a guide nucleic acid comprising a nucleotide sequence that is at least 70%, at least 80%, at least 90%, or 100% identical to SEQ ID NO: 260. In some cases, compositions comprise a programmable nuclease that is at least 70%, at least 80%, at least 90%, or 100% identical to SEQ ID NO: 4, and a guide nucleic acid comprising a nucleotide sequence that is at least 70%, at least 80%, at least 90%, or 100% identical to SEQ ID NO: 261. In some cases, compositions comprise a programmable nuclease that is at least 70%, at least 80%, at least 90%, or 100% identical to SEQ ID NO: 5, and a guide nucleic acid comprising a nucleotide sequence that is at least 70%, at least 80%, at least 90%, or 100% identical to SEQ ID NO: 262. In some cases, SEQ ID NO: 255 is associated with the programable nuclease of SEQ ID NO: 1. In some cases, SEQ ID NO: 256 is associated with the programable nuclease of SEQ ID NO: 2, 4, or 5. In some cases, SEQ ID NO: 257 is associated with the nuclease of SEQ ID NO: 3. In some cases, SEQ ID NO: 258 is associated with the programable nuclease of SEQ ID NO: 1. In some cases, SEQ ID NO: 259 is associated with the programable nuclease of SEQ ID NO: 2. In some cases, SEQ ID NO: 260 is associated with the nuclease of SEQ ID NO: 3. In some cases, SEQ ID NO: 261 is associated with the programable nuclease of SEQ ID NO: 4. In some cases, SEQ ID NO: 262 is associated with the programable nuclease of SEQ ID NO: 5.

TABLE 4
Examples of Repeat Sequences

| Type of | |
|---|---|
| Guide | |
| Nucleic | |
| Acid | Repeat Sequence in Guide Nucleic Acid |
| sgRNA | AGAAGAAGGAUUGGGAC (SEQ ID NO: 255) |
| sgRNA | AAAACAAGGAUUGAAAC (SEQ ID NO: 256) |
| sgRNA | CGUACGUGGAUUGAAAC (SEQ ID NO: 257) |
| crRNA | GUCUCAGAGCAUGUUCUGAUAGAAGAAGGAUUGGGAC |
| | (SEQ ID NO: 258) |
| crRNA | GUCUCAGAGAUACUUCCAAUAAAACAAGGAUUGAAAC |
| | (SEQ ID NO: 259) |
| crRNA | GCCGCAUCUCGCACGCGUACGUGGAUUGAAAC |
| | (SEQ ID NO: 260) |
| crRNA | GUCUGAAAGCUACUUCCAAUAAAACAAGGAUUGAAAC |
| | (SEQ ID NO: 261) |
| crRNA | GUCUGAAUCACAAUUCCAUUAAAACAAGGAUUGAAAC |
| | (SEQ ID NO: 262) |

In some instances, a guide nucleic acid can comprise a nucleotide sequence as shown in TABLE 4. In some instances, a guide nucleic acid comprises a nucleotide sequence that is at least 70%, at least 75%, at least 80%, at least 85%, at least 90%, or at least 95% identical to any one of SEQ ID Nos 255-257. In some instances, a guide nucleic acid comprises a nucleotide sequence that is at least 70%, at least 75%, at least 80%, at least 85%, at least 90%, or at least 95% identical to any one of SEQ ID Nos 258-262. In some instances, guide nucleic acids comprise at least 8, at least 9, at least 10, at least 11, at least 12, at least 13, at least 14, at least 15, at least 16, at least 17, at least 18, at least 19, at least 20 at least 25, at least 30, or at least 35 contiguous nucleotides of any one of SEQ ID Nos 255-262.

In some instances, a sgRNA can comprise a nucleotide sequence as shown in TABLE 10. In some instances, a sgRNA comprises a nucleotide sequence that is at least 70%, at least 75%, at least 80%, at least 85%, at least 90%, or at least 95% identical to any one of SEQ ID Nos 263-272. In some instances, a sgRNA comprises at least 20, at least 30, at least 40, at least 50, at least 60, at least 70, at least 80, at least 90, at least 100, at least 110, at least 120, at least 130, at least 140, at least 150, at least 160, at least 170, at least 180, or at least 190 contiguous nucleotides of any one of SEQ ID Nos 263-272.

In some instances, a crRNA can comprise a nucleotide sequence as shown in TABLE 7. In some instances, a crRNA comprises a nucleotide sequence that is at least 70%, at least 75%, at least 80%, at least 85%, at least 90%, or at least 95% identical to any one of SEQ ID Nos 258-262. In some instances, a crRNA comprises at least 8, at least 9, at least 10, at least 11, at least 12, at least 13, at least 14, at least 15, at least 16, at least 17, at least 18, at least 19, at least 20 at least 25, at least 30, or at least 35 contiguous nucleotides of any one of SEQ ID Nos 258-262.

In some instances, the tracrRNA comprises a stem-loop structure comprising a stem region and a loop region. In some cases, the stem region is 4 to 8 linked nucleosides in length. In some cases, the stem region is 5 to 6 linked nucleosides in length. In some cases, the stem region is 4 to 5 linked nucleosides in length. In some cases, the tracrRNA comprises a pseudoknot (e.g., a secondary structure comprising a stem at least partially hybridized to a second stem or half-stem secondary structure). A programmable nuclease or a multimeric complex thereof may recognize a tracrRNA comprising multiple stem regions. In some instances, the amino acid sequences of the multiple stem regions are identical to one another. In some instances, the amino acid sequences of at least one of the multiple stem regions is not identical to those of the others. In some cases, the tracrRNA comprises at least 2, at least 3, at least 4, or at least 5 stem regions.

A programmable nuclease may form a multimeric complex that binds a guide RNA. The programmable nucleases of the multimeric complex may bind the guide RNA in an asymmetric fashion. In some cases, one programmable nuclease of the multimeric complex interacts more strongly with the guide RNA than another programmable nuclease of the multimeric complex. In some cases, a programmable nuclease or a multimeric complex thereof interacts more strongly with a target nucleic acid when it is complexed with the guide RNA relative to when the programmable nuclease or the multimeric complex is not complexed with the guide RNA.

In some cases, a programmable nuclease or a multimeric complex thereof recognizes a PAM on a target nucleic acid. In some cases, multiple programmable nucleases of the multimeric complex recognize a PAM on a target nucleic acid. In some cases, only one programmable nuclease of the multimeric complex recognizes a PAM on a target nucleic acid. In some cases, the PAM is 3′ to the spacer region of the crRNA. In some cases, the PAM is directly 3′ to the spacer region of the crRNA. In some embodiments, a protospacer adjacent motif (PAM) comprises a nucleotide sequence found in a target nucleic acid that directs a programmable nuclease to modify the target nucleic acid at a specific location. A PAM sequence may be required for a complex having a programmable nuclease and a guide nucleic acid to hybridize to and modify the target nucleic acid. However, a given programmable nuclease may not require a PAM sequence being present in a target nucleic acid for the programmable nuclease to modify the target nucleic acid.

The spacer region of the guide RNA may comprise complementarity with (e.g., hybridize to) a target sequence of a target nucleic acid. In some cases, the spacer region is 15-28 linked nucleosides in length. In some cases, the spacer region is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleosides in length. In some cases, the spacer region is 18-24 linked nucleosides in length. In some cases, the spacer region is at least 15 linked nucleosides in length. In some cases, the spacer region is at least 16, 18, 20, or 22 linked nucleosides in length. In some cases, the spacer region comprises at least 10, 11, 12, 13, 14, 15, 16, 17, 18, 19, 20, 21, 22, 23, 24, 25, 26, 27, 28, 29, or 30 nucleotides. In some cases, the spacer region is at least 17 linked nucleosides in length. In some cases, the spacer region is at least 18 linked nucleosides in length. In some cases, the spacer region is at least 20 linked nucleosides in length. In some cases, the spacer region is at least 80%, at least 85%, at least 90%, at least 95% or 100% complementary to a target sequence of the target nucleic acid. In some cases, the spacer region is 100% complementary to the target sequence of the target nucleic acid. In some cases, the spacer region comprises at least 15 contiguous nucleobases that are complementary to the target nucleic acid.

In some embodiments, the engineered guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a sequence of nucleotides that is present in a host eukaryotic cell. Such a sequence of nucleotides is distinguished from nucleotide sequences present in other host cells, such as prokaryotic cells, or viruses. Said sequences present in a eukaryotic cell can be located a gene, an exon, an intron, a non-coding (e.g., promoter or enhancer) region, a selectable marker, tag, signal, and the like. In some cases, the engineered guide nucleic acid comprises at least 10 contiguous nucleotides that are complementary to a eukaryotic sequence. In some cases, the engineered guide nucleic acid comprises at least 11 contiguous nucleotides that are complementary to a eukaryotic sequence. In some cases, the engineered guide nucleic acid comprises at least 12 contiguous nucleotides that are complementary to a eukaryotic sequence. In some cases, the engineered guide nucleic acid comprises at least 13 contiguous nucleotides that are complementary to a eukaryotic sequence. In some cases, the engineered guide nucleic acid comprises at least 14 contiguous nucleotides that are complementary to a eukaryotic sequence. In some cases, the engineered guide nucleic acid comprises at least 15 contiguous nucleotides that are complementary to a eukaryotic sequence. In some cases, the engineered guide nucleic acid comprises at least 16 contiguous nucleotides that are complementary to a eukaryotic sequence. In some cases, the engineered guide nucleic acid comprises at least 17 contiguous nucleotides that are complementary to a eukaryotic sequence. In some cases, the engineered guide nucleic acid comprises at least 18 contiguous nucleotides that are complementary to a eukaryotic sequence. In some cases, the engineered guide nucleic acid comprises at least 19 contiguous nucleotides that are complementary to a eukaryotic sequence. In some cases, the engineered guide nucleic acid comprises at least 20 contiguous nucleotides that are complementary to a eukaryotic sequence. In some cases, the engineered guide nucleic acid comprises at least 21 contiguous nucleotides that are complementary to a eukaryotic sequence. In some cases, the engineered guide nucleic acid comprises at least 22 contiguous nucleotides that are complementary to a eukaryotic sequence. In some cases, the engineered guide nucleic acid comprises at least 23 contiguous nucleotides that are complementary to a eukaryotic sequence. In some cases, the engineered guide nucleic acid comprises at least 24 contiguous nucleotides that are complementary to a eukaryotic sequence. In some cases, the engineered guide nucleic acid comprises at least 25 contiguous nucleotides that are complementary to a eukaryotic sequence. In some cases, the engineered guide nucleic acid comprises at least 26 contiguous nucleotides that are complementary to a eukaryotic sequence. In some cases, the engineered guide nucleic acid comprises at least 27 contiguous nucleotides that are complementary to a eukaryotic sequence. In some cases, the engineered guide nucleic acid comprises at least 28 contiguous nucleotides that are complementary to a eukaryotic sequence. In some cases, the engineered guide nucleic acid comprises at least 29 contiguous nucleotides that are complementary to a eukaryotic sequence. In some cases, the engineered guide nucleic acid comprises at least 30 or more contiguous nucleotides that are complementary to a eukaryotic sequence.

In some embodiments, complementary and complementarity with reference to a nucleic acid molecule or nucleotide sequence, comprise to the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The guide RNA may be an engineered guide nucleic acid (e.g., chemically or recombinantly produced). The sequence of the engineered guide nucleic acid, or a portion thereof, may be different from the sequence of a naturally occurring nucleic acid. The engineered guide nucleic acid may bind to a Type V CRISPR/Cas protein disclosed herein or a multimeric complex thereof. The engineered guide nucleic acid may bind a Cas14 protein, wherein the amino acid sequence of the Cas14 protein is at least 70%, at least 75%, at least 80%, at least 85%, at least 90%, at least 95%, at least 97%, at least 98%, or at least 99% identical to any one of SEQ ID NOs: 1-5. The engineered guide nucleic acid may bind to two Type V CRISPR/Cas proteins, thereby facilitating the formation of a multimeric complex.

The guide RNA may bind to a target nucleic acid (e.g., a single strand of a target nucleic acid) or a portion thereof. The guide nucleic acid may bind to a target nucleic acid such as a nucleic acid from a bacterium, a virus, a parasite, a protozoa, a fungus or other agents responsible for a disease, or an amplicon thereof. The target nucleic acid may comprise a mutation, such as a single nucleotide polymorphism (SNP). A mutation may confer for example, resistance to a treatment, such as antibiotic treatment. The guide nucleic acid may bind to a target nucleic acid, such as DNA or RNA, from a cancer gene or gene associated with a genetic disorder, or an amplicon thereof, as described herein.

In some cases, a programmable nuclease or a multimeric complex thereof cleaves a precursor RNA (“pre-crRNA”) to produce a guide RNA, also referred to as a “mature guide RNA.” A programmable nuclease that cleaves pre-crRNA to produce a mature guide RNA is said to have pre-crRNA processing activity. In some cases, a repeat region of a guide RNA comprises mutations or truncations relative to respective regions in a corresponding pre-crRNA. Guide nucleic acids and portions thereof may be found in or identified from a CRISPR array present in the genome of a host organism. A crRNA may be the product of processing of a longer precursor CRISPR RNA (pre-crRNA) transcribed from the CRISPR array by cleavage of the pre-crRNA within each direct repeat sequence to afford shorter, mature crRNAs. A crRNA may be generated by a variety of mechanisms, including the use of dedicated endonucleases (e.g., Cas6 or Cas5d in Type I and III systems), coupling of a host endonuclease (e.g., RNase III) with tracrRNA (Type II systems), or a ribonuclease activity endogenous to the programmable nuclease itself (e.g., Cpf1, from Type V systems). A crRNA may also be specifically generated outside of processing of a pre-crRNA and individually contacted to a programmable nuclease in vivo or in vitro.

The guide nucleic acid may comprise a first region complementary to a target nucleic acid (FR1) and a second region that is not complementary to the target nucleic acid (FR2). In some cases, FR1 is located 5′ to FR2 (FR1-FR2). In some cases, FR2 is located 5′ to FR1 (FR2-FR1).

In some cases, the guide comprises 10, 11, 12, 13, 14, 15, 16, 17, 18, 19, 20, 21, 22, 23, 24, 25, 26, 27, 28, 29, or 30 linked nucleosides. In general, a guide nucleic acid comprises at least linked nucleosides. In some instances, a guide nucleic acid comprises at least 25 linked nucleosides. A guide nucleic acid may comprise 10 to 50 linked nucleosides. In some cases, the guide nucleic acid comprises or consists essentially of about 12 to about 80 linked nucleosides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleosides. In some cases, the guide nucleic acid has about 10 to about 60, about 20 to about 50, or about 30 to about 40 linked nucleosides.

It is understood that the sequence of a spacer region need not be 100% complementary to that of a target sequence of a target nucleic acid to hybridize or hybridize specifically to the target sequence. The guide nucleic acid may comprise at least one uracil between nucleic acid residues 5 to 20 of the spacer region that is not complementary to the corresponding nucleoside of the target sequence. The guide nucleic acid may comprise at least one uracil between nucleic acid residues 5 to 9, 10 to 14, or 15 to 20 of the spacer region that is not complementary to the corresponding nucleoside of the target sequence. In some cases, the region of the target nucleic acid that is complementary to the spacer region comprises an epigenetic modification or a post-transcriptional modification. In some cases, the epigenetic modification comprises an acetylation, methylation, or thiol modification.

### A. Pooling Guide Nucleic Acids

In some instances, compositions, systems or methods provided herein comprise a pool of guide nucleic acids. In some instances, the pool of guide nucleic acids were tiled against a target nucleic acid, e.g., the genomic locus of interest or uses thereof. In some instances, a guide nucleic acid is selected from a group of guide nucleic acids that have been tiled against a nucleic acid sequence of a genomic locus of interest. The genomic locus of interest may belong to a viral genome, a bacterial genome, or a mammalian genome. Non-limiting examples of viral genomes are an HPV genome, an HIV genome, an influenza genome, or a coronavirus genome. Often, these guide nucleic acids are pooled for detecting a target nucleic acid in a single assay. Pooling of guide nucleic acids may ensure broad spectrum identification, or broad coverage, of a target species within a single reaction. This may be particularly helpful in diseases or indications, like sepsis, that may be caused by multiple organisms. The pool of guide nucleic acids may enhance the detection of a target nucleic using systems of methods described herein relative to detection with a single guide nucleic acid. The pool of guide nucleic acids may ensure broad coverage of the target nucleic acid within a single reaction using the methods described herein. In some instances, the pool of guide nucleic acids are collectively complementary to at least 50%, at least 55%, at least 60%, at least 65%, at least 70%, at least 75%, at least 80%, at least 85%, at least 90%, at least 95% or 100% of the target nucleic acid. In some instances, at least a portion of the guide nucleic acids of the pool overlap in sequence. In some instances, at least a portion of the guide nucleic acids of the pool do not overlap in sequence. In some cases, the pool of guide nucleic acids comprises at least 2, at least 3, at least 4, at least 5, or at least 6 guide nucleic acids targeting different sequences of a target nucleic acid.

### B. Intermediary Nucleic Acids

A guide nucleic acid may comprise or be coupled to an intermediary nucleic acid. The intermediary nucleic acid may also be referred to as an intermediary RNA, although it may comprise deoxyribonucleosides in addition to ribonucleosides. The intermediary RNA may be separate from, but form a complex with a crRNA to form a discrete gRNA system. The intermediary RNA may be linked to a crRNA to form a composite gRNA. A programmable nuclease may bind a crRNA and an intermediary RNA. In some cases, the crRNA and the intermediary RNA are provided as a single nucleic acid (e.g., covalently linked). In some embodiments, the crRNA and the intermediary RNA are separate polynucleotides (e.g., a discrete gRNA system). An intermediary RNA may comprise a repeat hybridization region and a hairpin region. The repeat hybridization region may hybridize to all or part of the sequence of the repeat of a crRNA. The repeat hybridization region may be positioned 3′ of the hairpin region. The hairpin region may comprise a first sequence, a second sequence that is reverse complementary to the first sequence, and a stem-loop linking the first sequence and the second sequence.

The CRISPR/Cas ribonucleoprotein (RNP) complex may comprise a Cas protein complexed with a guide nucleic acid (e.g., a crRNA) and an intermediary RNA. Sometimes, a guide nucleic acid comprises a crRNA and an intermediary RNA (e.g., the crRNA and intermediary RNA are provided as a single nucleic acid molecule). A composition may comprise a crRNA, an intermediary RNA, a Cas protein, and a detector nucleic acid.

In some instances, the length of intermediary RNAs is not greater than 50, 56, 68, 71, 73, 95, or 105 linked nucleosides. In some embodiments, the length of an intermediary RNA is about 30 to about 120 linked nucleosides. In some embodiments, the length of an intermediary RNA is about 50 to about 105, about 50 to about 95, about 50 to about 73, about 50 to about 71, about 50 to about 68, or about 50 to about 56 linked nucleosides. In some embodiments, the length of an intermediary RNA is 56 to 105 linked nucleosides, from 56 to 105 linked nucleosides, 68 to 105 linked nucleosides, 71 to 105 linked nucleosides, 73 to 105 linked nucleosides, or 95 to 105 linked nucleosides. In some embodiments, the length of an intermediary RNA is 40 to 60 nucleotides. In some embodiments, the length of the intermediary RNA is 50, 56, 68, 71, 73, 95, or 105 linked nucleosides. In some embodiments, the length of the intermediary RNA is 50 nucleotides.

An exemplary intermediary RNA may comprise, from 5′ to 3′, a 5′ region, a hairpin region, a repeat hybridization region, and a 3′ region. In some cases, the 5′ region may hybridize to the 3′ region. In some embodiments, the 5′ region does not hybridize to the 3′ region. In some cases, the 3′ region is covalently linked to the crRNA (e.g., through a phosphodiester bond). In some embodiments, an intermediary RNA may comprise an un-hybridized region at the 3′ end of the intermediary RNA. The un-hybridized region may have a length of about 1, about 2, about 3, about 4, about 5, about 6, about 7, about 8, about 9, about 10, about 12, about 14, about 16, about 18, or about 20 linked nucleosides. In some embodiments, the length of the un-hybridized region is 0 to 20 linked nucleosides.

### VIII. Modifications

Polypeptides (e.g., programmable nucleases) and nucleic acids (e.g., engineered guide nucleic acids) described herein can be further modified as described throughout and as further described herein.

Examples are modifications of interest that do not alter primary sequence, including chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Modifications disclosed herein can also include modification of described polypeptides and/or engineered guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g. D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or engineered guide nucleic acids described herein. For example, groups can be introduced during synthesis or during expression of a polypeptide (e.g., a programmable nuclease), which allow for linking to other molecules or to a surface. Thus, e.g., cysteines can be used to make thioethers, histidines for linking to a metal ion complex, carboxyl groups for forming amides or esters, amino groups for forming amides, and the like.

Modifications can further include modification of nucleic acids described herein (e.g., engineered guide nucleic acids) to provide the nucleic acid with a new or enhanced feature, such as improved stability. Such modifications of a nucleic acid include a base modification, a backbone modification, a sugar modification, or combinations thereof, of one or more nucleotides, nucleosides, or nucleobases in a nucleic acid.

In some embodiments, nucleic acids (e.g., engineered guide nucleic acids) described herein comprise one or more modifications comprising: 2′O-methyl modified nucleotides, 2′ Fluoro modified nucleotides; locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH2-NH—O-CH2-, —CH2-N(CH3)-O-CH2- (known as a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O-CH2-); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH2 component parts; and combinations thereof.

### IX. Target Nucleic Acids and Samples

### A. Certain Target Nucleic Acids

Disclosed herein are compositions, systems and methods for detecting and/or modifying a target nucleic acid. In some instances, the target nucleic acid is a single stranded nucleic acid. Alternatively, or in combination, the target nucleic acid is a double stranded nucleic acid and is prepared into single stranded nucleic acids before or upon contacting the reagents. In some embodiments, the target nucleic acid is a double stranded nucleic acid. In certain embodiments, the target nucleic acid is a double stranded nucleic acid comprising a target strand and a non-target strand, wherein the target strand comprises a target sequence. In some embodiments, where a target strand comprises a target sequence, at least a portion of the engineered guide nucleic acid is complementary to the target sequence on the target strand. In some embodiments, where the target nucleic acid is a double stranded nucleic acid comprising a target strand and a non-target strand, and wherein the target strand comprises a target sequence, at least a portion of the engineered guide nucleic acid is complementary to the target sequence on the target strand. In some embodiments, a target nucleic acid comprises a PAM as described herein that is located on the non-target strand. Such a PAM described herein, in some embodiments, is adjacent (e.g., within 1, 2, 3, 4 or 5 nucleotides) to the 5′ end of the target sequence on the non-target strand of the double stranded DNA molecule. In certain embodiments, such a PAM described herein is directly adjacent to the 5′ end of a target sequence on the non-target strand of the double stranded DNA molecule.

In some embodiments, the double stranded nucleic acid is DNA. The target nucleic acid may be a RNA. The target nucleic acids include but are not limited to mRNA, rRNA, tRNA, non-coding RNA, long non-coding RNA, and microRNA (miRNA). In some instances, the target nucleic acid is complementary DNA (cDNA) synthesized from a single-stranded RNA template in a reaction catalyzed by a reverse transcriptase. In some cases, the target nucleic acid is single-stranded RNA (ssRNA) or mRNA. In some cases, the target nucleic acid is from a virus, a parasite, or a bacterium described herein.

A type V CRISPR/Cas protein of the present disclosure, a dimer thereof, or a multimeric complex thereof may cleave or nick a target nucleic acid within or near a protospacer adjacent motif (PAM) sequence of the target nucleic acid. In some instances, cleavage occurs within 1, 2, 3, 4, 5, 6, 7, 8, 9 or 10 nucleosides of a 5′ or 3′ terminus of a PAM sequence. A target nucleic acid may comprise a PAM sequence adjacent to a sequence that is complementary to a guide nucleic acid spacer region. As used herein for denoting protospacer adjacent motif (PAM) nucleic acid sequences, B is one or more of CG or TA; K is G or T; V is A, C or G; S is C or G, and R is A or G. In some cases, the PAM sequence is 5′-NTTR-3′. In some cases, the PAM sequence is 5′-TTTR-3′. In some cases, the PAM sequence is 5′-TTTG-3′. In some cases, the PAM sequence is 5′-GTTG-3′. In some cases, the PAM sequence is 5′-TTTN-3′. In some cases, the PAM sequence is 5′-TTTA-3′. In some cases, the PAM sequence is 5′-TTAT-3′. In some cases, the PAM sequence is 5′-TBN-3′. In some cases, the PAM sequence is 5′-TTTN-3′. In some cases, the PAM sequence is selected from the group consisting of 5′-TTTV-3′, 5′-CTTV-3′, 5′-TCTV-3′, and 5′-TTCV-3′. In some cases, the type V CRISPR/Cas protein is a Cas12 protein and the PAM sequence is 5′-TTTV-3′ (e.g., 5′-TTTG-3′). In some cases, the type V CRISPR/Cas protein is a Cas12 protein and the PAM sequence is selected from the group consisting of 5′-CTTV-3′, 5′-TCTV-3′, and 5′-TTCV-3′. In some cases, the type V CRISPR/Cas protein is a Casφ protein and the PAM sequence iIn some cases, a Casφ protein comprises a PAM sequence comprising comprises 5′-VTTR-3′, such as GTTA, GTTG, ATTA, ATTG, CTTA, and CTTG. In some cases, the type V CRISPR/Cas protein is a Casφ protein and the PAM sequence is 5′-VTTR-3′. In some embodiments, the type V CRISPR/Cas protein is a Cas 14 protein and the PAM sequence is 5′-NTTR-3′, wherein N is any base, T is thymine, and R is a purine. In some embodiments, the type V CRISPR/Cas protein is a Cas 14 protein and the PAM sequence is 5′-TTTR-3′, wherein T is thymine and R is a purine. In some embodiments, the type V CRISPR/Cas protein is a Cas 14 protein and the PAM sequence is 5′-TTTG-3′, wherein T is thymine, and G is a Guanine. In some embodiments, the type V CRISPR/Cas protein is a Cas 14 protein and the PAM sequence is 5′-GTTG-3′, wherein T is thymine, and G is a Guanine. In some embodiments, the type V CRISPR/Cas protein is a Cas 14 protein and the PAM sequence is 5′-NTTR-3′, wherein N is any base, T is thymine, and R is a purine, and the Cas 14 protein comprises an amino acid sequence that is at least 70%, at least 75%, at least 80%, at least 85%, at least 90%, at least 95%, or 100% identical to SEQ ID NO: 2. In some embodiments, the type V CRISPR/Cas protein is a Cas 14 protein and the PAM sequence is 5′-TTTR-3′, wherein T is thymine and R is a purine, and the Cas 14 protein comprises an amino acid sequence that is at least 70%, at least 75%, at least 80%, at least 85%, at least 90%, at least 95%, or 100% identical to any one of SEQ ID NO: 1 and 3-5.

In some cases, the target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleosides. In some cases, the target nucleic acid comprises 10 to 90, 20 to 80, 30 to 70, or 40 to 60 linked nucleosides. In some cases, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleosides. In some instances, the target nucleic acid comprises at least 10, at least 20, at least 30, at least 40, at least 50, at least 60, at least 70, at least 80, at least 90, or at least 100 linked nucleosides.

A programmable nuclease-guide nucleic acid complex may comprise high selectivity for a target sequence. In some cases, a ribonucleoprotein may comprise a selectivity of at least 200:1, 100:1, 50:1, 20:1, 10:1, or 5:1 for a target nucleic acid over a single nucleotide variant of the target nucleic acid. In some cases, a ribonucleoprotein may comprise a selectivity of at least 5:1 for a target nucleic acid over a single nucleotide variant of the target nucleic acid. Leveraging programmable nuclease selectivity, some methods described herein may detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample comprises 1 to 10,000, 100 to 8000, 400 to 6000, 500 to 5000, 1000 to 4000, or 2000 to 3000 target nucleic acids. In some cases, the method detects target nucleic acid present at least at one copy per 10 non-target nucleic acids, 102 non-target nucleic acids, 103 non-target nucleic acids, 104 non-target nucleic acids, 105 non-target nucleic acids, 106 non-target nucleic acids, 107 non-target nucleic acids, 108 non-target nucleic acids, 109 non-target nucleic acids, or 1010 non-target nucleic acids.

Often, the target nucleic acid may be from 0.05% to 20% of total nucleic acids in the sample. Sometimes, the target nucleic acid is 0.1% to 10% of the total nucleic acids in the sample. The target nucleic acid, in some cases, is 0.1% to 5% of the total nucleic acids in the sample. The target nucleic acid may also be 0.1% to 1% of the total nucleic acids in the sample. The target nucleic acid may be DNA or RNA. The target nucleic acid may be any amount less than 100% of the total nucleic acids in the sample. The target nucleic acid may be 100% of the total nucleic acids in the sample.

The target nucleic acid may be 0.05% to 20% of total nucleic acids in the sample. Sometimes, the target nucleic acid is 0.1% to 10% of the total nucleic acids in the sample. The target nucleic acid, in some cases, is 0.1% to 5% of the total nucleic acids in the sample. Often, a sample comprises the segment of the target nucleic acid and at least one nucleic acid comprising less than 100% sequence identity to the segment of the target nucleic acid but no less than 50% sequence identity to the segment of the target nucleic acid. For example, the segment of the target nucleic acid comprises a mutation as compared to at least one nucleic acid comprising less than 100% sequence identity to the segment of the target nucleic acid but no less than 50% sequence identity to the segment of the target nucleic acid. Often, the segment of the target nucleic acid comprises a single nucleotide mutation as compared to at least one nucleic acid comprising less than 100% sequence identity to the segment of the target nucleic acid but no less than 50% sequence identity to the segment of the target nucleic acid.

A target nucleic acid may be an amplified nucleic acid of interest. The nucleic acid of interest may be any nucleic acid disclosed herein or from any sample as disclosed herein. The nucleic acid of interest may be an RNA that is reverse transcribed before amplification. The nucleic acid of interest may be amplified then the amplicons may be transcribed into RNA.

In some instances, compositions described herein exhibit indiscriminate trans-cleavage of ssRNA, enabling their use for detection of RNA in samples. In some cases, target ssRNA are generated from many nucleic acid templates (RNA) in order to achieve cleavage of the FQ reporter in the DETECTR platform. Certain programmable nucleases may be activated by ssRNA, upon which they may exhibit trans-cleavage of ssRNA and may, thereby, be used to cleave ssRNA FQ reporter molecules in the DETECTR system. These programmable nucleases may target ssRNA present in the sample, or generated and/or amplified from any number of nucleic acid templates (RNA). Described herein are reagents comprising a single stranded reporter nucleic acid comprising a detection moiety, wherein the reporter nucleic acid (e.g., the ssDNA-FQ reporter described above) is capable of being cleaved by the programmable nuclease, upon generation and amplification of ssRNA from a nucleic acid template using the methods disclosed herein, thereby generating a first detectable signal.

In some instances, target nucleic acids comprise at least one nucleic acid comprising at least 50% sequence identity to the target nucleic acid or a portion thereof. Sometimes, the at least one nucleic acid comprises an amino acid sequence that is at least 60%, 70%, 75%, 80%, 85%, 90%, 91%, 92%, 93%, 94%, 95%, 96%, 97%, 98%, or 99% identical to an equal length portion of the target nucleic acid. Sometimes, the at least one nucleic acid comprises an amino acid sequence that is 100% identical to an equal length portion of the target nucleic acid. Sometimes, the amino acid sequence of the at least one nucleic acid is at least 60%, 70%, 75%, 80%, 85%, 90%, 91%, 92%, 93%, 94%, 95%, 96%, 97%, 98%, or 99% identical to the target nucleic acid. Sometimes, the target nucleic acid comprises an amino acid sequence that is less than 60%, 70%, 75%, 80%, 85%, 90%, 91%, 92%, 93%, 94%, 95%, 96%, 97%, 98%, or 99% identical to an equal length portion of the at least one nucleic acid.

In some embodiments, samples comprise a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 pM, less than 7 μM, less than 8 μM, less than 9 μM, less than 10 μM, less than 100 μM, or less than 1 mM. In some embodiments, the sample comprises a target nucleic acid sequence at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM, 3 μM to 4 μM, 4 μM to 5 μM, 5 μM to 6 μM, 6 μM to 7 μM, 7 μM to 8 μM, 8 μM to 9 μM, 9 μM to 10 μM, 10 μM to 100 μM, 100 μM to 1 mM, 1 nM to 10 nM, 1 nM to 100 nM, 1 nM to 1 μM, 1 nM to 10 μM, 1 nM to 100 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 μM, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some embodiments, the sample comprises a target nucleic acid at a concentration of 20 nM to 200 μM, 50 nM to 100 μM, 200 nM to 50 μM, 500 nM to 20 μM, or 2 μM to 10 μM. In some embodiments, the target nucleic acid is not present in the sample.

In some embodiments, samples comprise fewer than 10 copies, fewer than 100 copies, fewer than 1000 copies, fewer than 10,000 copies, fewer than 100,000 copies, or fewer than 1,000,000 copies of a target nucleic acid sequence. In some embodiments, the sample comprises 10 copies to 100 copies, 100 copies to 1000 copies, 1000 copies to 10,000 copies, 10,000 copies to 100,000 copies, 100,000 copies to 1,000,000 copies, 10 copies to 1000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid sequence. In some embodiments, the sample comprises 10 copies to 500,000 copies, 200 copies to 200,000 copies, 500 copies to 100,000 copies, 1000 copies to 50,000 copies, 2000 copies to 20,000 copies, 3000 copies to 10,000 copies, or 4000 copies to 8000 copies. In some embodiments, the target nucleic acid is not present in the sample.

In some embodiments, a sample comprises a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

A number of target nucleic acid populations are consistent with the methods and compositions disclosed herein. Some methods described herein may detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has 3 to 50, 5 to 40, or 10 to 25 target nucleic acid populations. In some cases, the method detects target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations may be present at different concentrations or amounts in the sample.

In some embodiments, target nucleic acids may activate a programmable nuclease to initiate sequence-independent cleavage of a nucleic acid-based reporter (e.g., a reporter comprising an RNA sequence, or a reporter comprising DNA and RNA). For example, a programmable nuclease of the present disclosure is activated by a target nucleic acid to cleave reporters having an RNA (also referred to herein as an “RNA reporter”). Alternatively, a programmable nuclease of the present disclosure is activated by a target nucleic acid to cleave reporters having an RNA. Alternatively, a programmable nuclease of the present disclosure is activated by a target RNA to cleave reporters having an RNA (also referred to herein as a “RNA reporter”). The RNA reporter may comprise a single-stranded RNA labelled with a detection moiety or may be any RNA reporter as disclosed herein.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by a CRISPR/Cas system.

In some embodiments, the target nucleic acid is in a cell. In some embodiments, the cell is a single-cell eukaryotic organism; a plant cell an algal cell; a fungal cell; an animal cell; a cell an invertebrate animal; a cell a vertebrate animal such as fish, amphibian, reptile, bird, and mammal; or a cell a mammal such as a human, a non-human primate, an ungulate, a feline, a bovine, an ovine, and a caprine. In preferred embodiments, the cell is a eukaryotic cell. In preferred embodiments, the cell is a mammalian cell, a human cell, or a plant cell.

In some embodiments, the target nucleic acid comprises a nucleic acid sequence from a pathogen responsible for a disease. Non-limiting examples of pathogens are bacteria, a virus and a fungus. The target nucleic acid, in some cases, is a portion of a nucleic acid from a sexually transmitted infection or a contagious disease. In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to coronavirus (e.g., SARS-CoV-2); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the target nucleic acid sequence comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure may be used to target a viral nucleic acid sequence in a plant. A programmable nuclease of the disclosure (e.g., Cas14) may cleave the viral nucleic acid. In some embodiments, the target nucleic acid sequence comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some cases, is a portion of a nucleic acid from a virus or a bacterium or other agents responsible for a disease in the plant (e.g., a crop). In some cases, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that may be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

In some embodiments, the target nucleic acid comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease.

### B. Mutations

A mutation may be in an open reading frame of a target nucleic acid. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

In some instances, target nucleic acids comprise a mutation. In some instances, a sequence comprising a mutation may be modified to a wildtype sequence with a composition, system or method described herein. In some instances, a sequence comprising a mutation may be detected with a composition, system or method described herein. The mutation may be a mutation of 1, 2, 3, 4, 5, 6, 7, 8, 9, 10, 11, 12, 13, 14, 15, 16, 17, 18, 19, 20 or more nucleotides. The mutation may comprise a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may comprise a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. Non-limiting examples of mutations are insertion-deletion (indel), single nucleotide polymorphism (SNP), and frameshift mutations. In some instances, guide nucleic acids described herein hybridize to a region of the target nucleic acid comprising the mutation. The mutation may be located in a non-coding region or a coding region of a gene.

In some instances, target nucleic acids comprise a mutation, wherein the mutation is a SNP. The single nucleotide mutation or SNP may be associated with a phenotype of the sample or a phenotype of the organism from which the sample was taken. The SNP, in some cases, is associated with altered phenotype from wild type phenotype. The SNP may be a synonymous substitution or a nonsynonymous substitution. The nonsynonymous substitution may be a missense substitution or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a cancer cell.

In some embodiments, mutations comprise a point mutation, a chromosomal mutation, a copy number mutation, or any combination thereof. A point mutation may be a substitution, insertion, or deletion of a single nucleotide. In some embodiments, mutations comprise a chromosomal mutation. A chromosomal mutation may comprise an inversion, a deletion, a duplication, or a translocation of one or more nucleotides. In some embodiments, mutations comprise a copy number variation. A copy number variation may comprise a gene amplification or an expanding trinucleotide repeat. In some embodiments, guide nucleic acids described herein hybridize to a target sequence of a target nucleic acid comprising the mutation. In some embodiments, mutations are located in a non-coding region of a gene.

In some instances, target nucleic acids comprise a mutation, wherein the mutation is a deletion of 1, 2, 3, 4, 5, 6, 7, 8, 9, 10, 11, 12, 13, 14, 15, 16, 17, 18, 19, 20 or more nucleotides. The mutation may be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

### C. Certain Samples

Various sample types comprising a target nucleic acid of interest are consistent with the present disclosure. These samples may comprise a target nucleic acid sequence for detection. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample may be obtained to test for presence of a disease, cancer, genetic disorder, or any mutation of interest.

In some instances, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A tissue sample from a subject may be dissociated or liquified prior to application to detection system of the present disclosure. Non-limiting examples of environmental samples are soil, air, or water. In some instances, an environmental sample is taken as a swab from a surface of interest or taken directly from the surface of interest.

In some instances, the sample is a raw (unprocessed, unmodified) sample. Raw samples may be applied to a system for detecting or modifying a target nucleic acid, such as those described herein. In some instances, the sample is diluted with a buffer or a fluid or concentrated prior to its application to the system or be applied neat to the detection system. Sometimes, the sample contains no more 20 μl of buffer or fluid. The sample, in some cases, is contained in no more than 1, 5, 10, 15, 20, 25, 30, 35 40, 45, 50, 55, 60, 65, 70, 75, 80, 90, 100, 200, 300, 400, 500 μl, or any of value 1 μl to 500 μl, preferably 10 μL to 200 μL, or more preferably 50 μL to 100 μL of buffer or fluid. Sometimes, the sample is contained in more than 500 μl.

In some instances, the sample is taken from a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

In some instances, samples are used for diagnosing a disease. In some instances the disease is cancer. The sample used for cancer testing may comprise at least one target nucleic acid that may bind to a guide nucleic acid of the reagents described herein. The target nucleic acid, in some cases, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some cases, the assay may be used to detect “hotspots” in target nucleic acids that may be predictive of lung cancer. In some cases, the target nucleic acid comprises a portion of a nucleic acid that is associated with a blood fever. In some cases, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, system, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1. Any region of the aforementioned gene loci may be probed for a mutation or deletion using the compositions and methods disclosed herein. For example, in the EGFR gene locus, the compositions and methods for detection disclosed herein may be used to detect a single nucleotide polymorphism or a deletion.

In some embodiments, non-limiting examples of a cancer can comprise: acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytoma; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; Hodgkin's lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenström; male breast cancer; malignant fibrous histiocytoma of bone/osteosarcoma; medulloblastoma; medulloepithelioma; melanoma; melanoma, intraocular (eye); Merkel cell cancer; Merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-Hodgkin's lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; NUT midline carcinoma; retinoblastoma; rhabdomyosarcoma, childhood; salivary gland cancer; sarcoma, Ewing family of tumors; Sézary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sézary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In some instances, samples are used to diagnose a genetic disorder, also referred to as genetic disorder testing. The sample used for genetic disorder testing may comprise at least one target nucleic acid that may bind to a guide nucleic acid of the reagents described herein. In some embodiments, the genetic disorder is hemophilia, sickle cell anemia, β-thalassemia, Duchene muscular dystrophy, severe combined immunodeficiency, Huntington's disease, or cystic fibrosis. The target nucleic acid, in some cases, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid is a nucleic acid from a genomic locus, a transcribed mRNA, or a reverse transcribed mRNA, a DNA amplicon of or a cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing may comprise at least one target nucleic acid that may bind to a guide nucleic acid of the reagents described herein. The target nucleic acid, in some cases, is a nucleic acid encoding a sequence associated with a phenotypic trait.

The sample used for genotyping testing may comprise at least one target nucleic acid that may bind to a guide nucleic acid of the reagents described herein. The target nucleic acid, in some cases, is a nucleic acid encoding a sequence associated with a genotype of interest.

The sample used for ancestral testing may comprise at least one target nucleic acid that may bind to a guide nucleic acid of the reagents described herein. The target nucleic acid, in some cases, is a nucleic acid encoding a sequence associated with a geographic region of origin or ethnic group.

The sample may be used for identifying a disease status. For example, a sample is any sample described herein, and is obtained from a subject for use in identifying a disease status of a subject. The disease may be a cancer or genetic disorder. Sometimes, a method comprises obtaining a serum sample from a subject; and identifying a disease status of the subject. Often, the disease status is prostate disease status, but the status of any disease may be assessed.

Any of the above disclosed samples are consistent with the methods, compositions, reagents, enzymes, and systems disclosed herein.

### X. Diseases and Indications

Described herein are methods of treating a disease comprising administering a composition herein to a subject. In some cases, a composition can be in unit dose form. Treating a disease using a method described herein can include editing a target nucleic acid. Treating a disease using a method described herein can include detecting a target nucleic acid. Such a disease can be a genetic disease. In some cases, a genetic disease comprises a disease caused by one or more mutations in the DNA of an organism. In some instances, a disease can comprise a disorder. Mutations may be due to several different cellular mechanisms, including, but not limited to, an error in DNA replication, recombination, or repair, or due to environmental factors. Mutations may be encoded in the sequence of a target nucleic acid from the germline of an organism. A genetic disease may comprise a single mutation, multiple mutations, or a chromosomal aberration. In some cases, a syndrome can comprise a group of symptoms which, taken together, characterize a condition.

Described herein are compositions and methods for editing or detecting a target nucleic acid, wherein the target nucleic acid is a gene, a portion thereof, a transcript thereof. In some embodiments, the target nucleic acid is a reverse transcript (e.g. a cDNA) of an mRNA transcribed from the gene, or an amplicon thereof. In some embodiments, the target nucleic acid is an amplicon of at least a portion of a gene. Non-limiting examples of genes are: AAVS1, ABCA4, ABCB11, ABCC8, ABCD1, ABCG5, ABCG8, ACAD9, ACADM, ACADVL, ACAT1, ACOX1, ACSF3, ADA, ADAMTS2, ADGRG1, AGA, AGL, AGPS, AGXT, AHI1, AIRE, ALDH3A2, ALDOB, ALG6, ALK, ALKBH5, ALMS1, ALPL, AMRC9, AMT, ANAPC10, ANAPC11, ANGPTL3, APC, Apo(a), APOCHI, APOEe4, APOL1, APP, AQP2, AR, ARFRP1, ARG1, ARH, ARL13B, ARL6, ARSA, ARSB, ASL, ASNS, ASPA, ASS1, ATM ATP6V1B1, ATP7A, ATP7B, ATRX, ATXN1, ATXN10, ATXN2, ATXN3, ATXN7, ATXN8OS, AXIN1, AXIN2, B2M BACE-1, BAK1, BAP1, BARD1, BAX2, BBS1, BBS10, BBS12, BBS2, BCKDHA, BCKDHB, BCL2L2, BCS1L, BEST1, Betaglobin gene, BLM, BMPR1A, BRAF, BRAFV600E, BRCA1, BRCA2, BRIP1, BSND, C9orf72, CA4, CACNA1A, CAPN3, CASR, CBS, CCNB1 CC2D2A, CCR5, CD1, CD2, CD3, CD3D, CD3Z, CD4, CD5, CD6, CD7, CD8A, CD8B, CD9, CD14, CD18, CD19, CD21, CD22, CD23, CD27, CD28, CD30, CD33, CD34, CD36, CD38, CD40, CD40L, CD44, CD46, CD47, CD48, CD52, CD55, CD57, CD58, CD59, CD68, CD69, CD72, CD73, CD74, CD79A, CD80, CD81, CD83, CD84, CD86, CD90, CD93, CD96, CD99, CD100, CD123, CD160, CD163, CD164, CD164L2, CD166, CD200, CD204, CD207, CD209, CD226, CD244, CD247, CD274, CD276, CD300, CD320, CDC73, CDH1, CDH23, CDK11, CDK4, CDKN1A, CDKN1B, CDKN1C, CDKN2A, CDKN2B, CEBPA, CELA3B, CEP290, CERKL, CFB, CFTR, CHCHD10, CHEK2, CHM, CHRNE, CIITA, CLN3, CLN5, CLN6, CLN8, CLRN1, CLTA, CNBP, CNGB1, CNGB3, COL1A1, COL1A2, COL27A1, COL4A3, COL4A4, COL4A5, COL7A1, CPS1, CPT1A, CPT2, CRB1, CREBBP, CRX, CRYAA, CTNNA1, CTNNB1, CTNND2, CTNS, CTSK, CXCL12, CYBA, CYBB, CYP11B1, CYP11B2, CYP17A1, CYP19A1, CYP27A1, DBT, DCC, DCLRE1C, DERL2, DFNA36, DFNB31, DGAT2, DHCR7, DHDDS, DICER1, DIS3L2, DLD, DMD, DMPK, DNAH5, DNAI1, DNAI2, DNM2, DNMT1, DPC4, DYSF, EDA, EDN3, EDNRB, EGFR, EIF2B5, EMC2, EMC3, EMD, EMX1, EN1, EPCAM, ERCC6, ERCC8, ESCO2, ETFA, ETFDH, ETHE1, EVC, EVC2, EYS, F5, F9, FXT, FAH, FAM61A, FANCA, FANCB, FANCC, FANCD1, FANCD2, FANCE, FANCF, FANCG, FANCI, FANCJ, FANCL, FANCM, FANCN, FANCP, FANCS, FBN1, FGF14, FGFR2, FGFR3, FGA, FGB, FGG, FH, FHL1, FIX, FKRP, FKTN, FLCN, FMR1, FOXP3, FSCN2, FUS, FUT8, FVIII, FXII, FXN, G6PC, GAA, GALC, GALK1, GALT, GAMT, GATA2, GATA-4, GBA, GBE1, GCDH, GCGR, GDNF, GFAP, GFM1, GHR, GJB1, GJB2, GLA, GLB1, GLDC, GLE1, GNE, GNPTAB, GNPTG, GNS, GPC3, GPR98, GREM1, GRHPR, GRIN2B, H2AFX, H2AX, HADHA, HAX1, HBA1, HBA2, HBB, HER2, HEXA, HEXB, HFE, HGSNAT, HLCS, HMGCL, HOGA1, HOXB13, HPRPF3, HPRT1, HPS1, HPS3, HRAS, HRD1, HSD17B4, HSD3B2, HTT, HUS1, HYAL1, HYLS1, IDS, IDUA, IFITM5, IKBKAP, IL2RG, IL7R, IMPDH1, INPP5E, IRF4, ITGB2, ITPR1, IVD, JAG1, JAK1, JAK3, KCNC3, KCND3, KCNJ11, KLHL7, KRAS, LAMA2, LAMA3, LAMB3, LAMC2, LCA5, LDLR, LDLRAP1, LHX3, LIFR, LIPA, LMNA, LOR, LOXHD1, LPL, LRAT, LRP6, LRPPRC, LRRK2, MADR2, MAN2B1, MAPT, MAX, MCM6, MCOLN1, MECP2, MED17, MEFV, MEN1, MERTK, MESP2, MET, METex14, MFN2, MFSD8, MJA3, MITTF, MKL2, MKSV, MLC1, MLH1, MLH3, MAA, MMAB, MACHC, MIADHC, MMD, MPI, MPL, MPV17, MSH2, MSH3, MSH6, MTHFD1L, MTHFR, MTM1, MTRR, MTTP, MUT, MUTYH, MYC, MYH7, MYO7A, NAGLU, NAGS, NBN, NDRG1, NDUFAF5, NDUFS6, NEB, NF1, NF2, NKX2-5, NOG, NOTCH1, NOTCH2, NPC1, NPC2, NPHP1, NPHS1, NPHS2, NRAS, NR2E3, NTHL1, NTRK, NTRK1, OAT, OCT4, OFD1, OPA3, OTC, PAH, PALB2, PAQR8, PAX3, PC, PCCA, PCCB, PCDH15, PCSK9, PD1, PDCD1, PDE6B, PDGFRA, PDHA1, PDHB, PEX1, PEX10, PEX12, PEX13, PEX14, PEX16, PEX19, PEX2, PEX26, PEX3, PEX5, PEX6, PEX7, PFKM, PHGDH, PHOX2B, PKD1, PKD2, PKHD1, PKK, PLEKHG4, PMM2, PMP22, PMS1, PMS2, PNPLA3, POLD1, POLE, POMGNT1, POT1, POU5F1, PPM1A, PPP2R2B, PPT1, PRCD, PRKAG2, PRKAR1A, PRKCG, PRNP, PROM1, PROP1, PRPF31, PRPF8, PRPH2, PRPS1, PSAP, PSD95, PSEN1, PSEN2, PSRC1, PTCH1, PTEN, PTS, PUS1, PYGM RAB23, RAD50, RAD51C, RAD51D, RAG1, RAG2, RAPSN, RARS2, RB1, RDH12, RECQL4, RET, RHO, RICTOR, RMRP, ROS1, RP1, RP2, RPE65, RPGR, RPGRIP1L, RPL32P3, RS1, RTCA, RTEL1, RUNX1, SACS, SAMHD1, SCN1A, SCN2A, SDHA, SDHAF2, SDHB, SDHC, SDHD, SEL1L, SEPSECS, SERPINA1, SERPING1, SGCA, SGCB, SGCG, SGSH, SIRT1, SLC12A3, SLC12A6, SLC17A5, SLC22A5, SLC25A13, SLC25A15, SLC26A2, SLC26A4, SLC35A3, SLC35B4 SLC37A4, SLC39A4, SLC4A11, SLC6A8, SLC7A7, SMAD3, SMAD4, SMARCA4, SMARCAL1, SMARCB1, SMARCE1, SMN1, SMPD1, SNAI2, SNCA, SNRNP200, SOD1, SOX10, SPARA7, SPTBN2, STAR, STAT3, STK11, SUFU, SUMF1, SYNE1, SYNE2, SYS1, TARDBP, TAT, TBK1, TBP, TCIRG1, TCTN3, TECPR2, TERC, TERT, TFR2, TGFBR2, TGM1, TH, TLE3, TMEM127, TMEM138, TMEM216, TMEM43, TMEM67, TMPRSS6, TOP1, TOPORS, TP53, TPP1, TRAC, TRMU, TSC1, TSC2, TSFM, TSPAN14, TTBK2, TTC8, TTPA, TTR, TULP1, TYMP, UBE2G2, UBE2J1, UBE3A, USH1C, USH1G, USH2A, VEGF, VHL, VPS13A, VPS13B, VPS35, VPS45, VRK1, VSX2, VWF, WAS, WDR19, WDR48, WNT10A, WRN, WS2B, WS2C, WT1, XPA, XPC, XPF, XRCC3, YAP1, ZAC1, ZEB1, ZFYVE26, and ZNF423.

Described herein are compositions and methods for treating a disease in a subject by modifying a target nucleic acid associated with a gene or expression of a gene related to the disease. In some embodiments, methods comprise administering a composition or cell described herein to a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include, but are not limited to: 11-hydroxylase deficiency; 17,20-desmolase deficiency; 17-hydroxylase deficiency; 3-hydroxyisobutyrate aciduria; 3-hydroxysteroid dehydrogenase deficiency; 46, XY gonadal dysgenesis; AAA syndrome; ABCA3 deficiency; ABCC8-associated hyperinsulinism; aceruloplasminemia; acromegaly; achondrogenesis type 2; acral peeling skin syndrome; acrodermatitis enteropathica; adrenocortical micronodular hyperplasia; adrenoleukodystrophies; adrenomyeloneuropathies; Aicardi-Goutieres syndrome; Alagille disease (also called Alagille Syndrome); Alexander Disease, Alpers syndrome; alpha-1 antitrypsin deficiency (AATD); alpha-mannosidosis; Alstrom syndrome; Alzheimer's disease; amebic dysentery; amelogenesis imperfecta; amish type microcephaly; amyotrophic lateral sclerosis (ALS); anaplastic large cell lymphoma; anauxetic dysplasia; androgen insensitivity syndrome; angiopathic thrombosis; antiphospholipid syndrome; Antley-Bixler syndrome; APECED, Apert syndrome, aplasia of lacrimal and salivary glands, argininemia, arrhythmogenic right ventricular dysplasia, Arts syndrome, ARVD2, arylsulfatase deficiency type metachromatic leokodystrophy, ataxia telangiectasia, autoimmune lymphoproliferative syndrome; autoimmune polyglandular syndrome type 1; autosomal dominant anhidrotic ectodermal dysplasia; autosomal dominant deafness; autosomal dominant polycystic kidney disease; autosomal recessive microtia; autosomal recessive renal glucosuria; autosomal visceral heterotaxy; babesiosis; balantidial dysentery; Bardet-Biedl syndrome; Bartter syndrome; basal cell nevus syndrome; Batten disease; benign recurrent intrahepatic cholestasis; beta-mannosidosis; β-thalassemia; Bethlem myopathy; Blackfan-Diamond anemia; bleeding disorder (coagulation); blepharophimosis; Byler disease; C syndrome; CADASIL; calcific aortic stenosis; calcification of joints and arteries; carbamyl phosphate synthetase deficiency; cardiofaciocutaneous syndrome; Carney triad; carnitine palmitoyltransferase deficiencies; cartilage-hair hypoplasia; cblC type of combined methylmalonic aciduria; CD18 deficiency; CD3Z-associated primary T-cell immunodeficiency; CD40L deficiency; CDAGS syndrome; CDG1A; CDG1B; CDG1M; CDG2C; CEDNIK syndrome; central core disease; centronuclear myopathy; cerebral capillary malformation; cerebrooculofacioskeletal syndrome type 4; cerebrooculogacioskeletal syndrome; cerebrotendinous xanthomatosis; Chaga's Disease; Charcot Marie Tooth Disesase; cherubism; CHILD syndrome; chronic granulomatous disease; chronic recurrent multifocal osteomyelitis; citrin deficiency; classic hemochromatosis; CNPPB syndrome; cobalamin C disease; Cockayne syndrome; coenzyme Q10 deficiency; Coffin-Lowry syndrome; Cohen syndrome; combined deficiency of coagulation factors V; common variable immune deficiency 3; complement hyperactivation; complete androgen insentivity; cone rod dystrophies; conformational diseases; congenital bile adid synthesis defect type 1; congenital bile adid synthesis defect type 2; congenital defect in bile acid synthesis type; congenital erythropoietic porphyria; congenital generalized osteosclerosis; Cornelia de Lange syndrome; coronary heart disease; Cousin syndrome; Cowden disease; COX deficiency; Cri du chat syndrome; Crigler-Najjar disease; Crigler-Najjar syndrome type 1; Crisponi syndrome; Crouzon syndrome; Currarino syndrome; Curth-Macklin type ichthyosis hystrix; cutis laxa; cystic fibrosis; cystinosis; d-2-hydroxyglutaric aciduria; DDP syndrome; Dejerine-Sottas disease; Denys-Drash syndrome; Dercum disease; desmin cardiomyopathy; desmin myopathy; DGUOK-associated mitochondrial DNA depletion; diabetes Type I; diabetes Type II; disorders of glutamate metabolism; distal spinal muscular atrophy type 5; DNA repair diseases; dominant optic atrophy; Doyne honeycomb retinal dystrophy; Dravet Syndrome; Duchenne muscular dystrophy; dyskeratosis congenita; Ehlers-Danlos syndrome type 4; Ehlers-Danlos syndromes; Elejalde disease; Ellis-van Creveld disease; Emery-Dreifuss muscular dystrophies; encephalomyopathic mtDNA depletion syndrome; encephalitis; enzymatic diseases; EPCAM-associated congenital tufting enteropathy; epidermolysis bullosa with pyloric atresia; epilepsy; facioscapulohumeral muscular dystrophy; Factor V Leiden thrombophilia; Faisalabad histiocytosis; familial atypical mycobacteriosis; familial capillary malformation-arteriovenous; Familial Creutzfeld-Jakob disease; familial esophageal achalasia; familial glomuvenous malformation; familial hemophagocytic lymphohistiocytosis; familial mediterranean fever; familial megacalyces; familial schwannomatosis; familial spina bifida; familial splenic asplenia/hypoplasia; familial thrombotic thrombocytopenic purpura; Fanconi disease (Fanconi anemia); Feingold syndrome; FENIB; fibrodysplasia ossificans progressiva; FKTN; Fragile X syndrome; Francois-Neetens fleck corneal dystrophy; Frasier syndrome; Friedreich's ataxia; FTDP-17; Fuchs corneal dystrophy; fucosidosis; G6PD deficiency; galactosialidosis; Galloway syndrome; Gardner syndrome; Gaucher disease; Gitelman syndrome; GLUT1 deficiency; GM2—Gangliosidoses (e.g., Tay Sachs Disease, Sandhoff Disease) glycogen storage disease type 1b; glycogen storage disease type 2; glycogen storage disease type 3; glycogen storage disease type 4; glycogen storage disease type 9a; glycogen storage diseases; GM1-gangliosidosis; Greenberg syndrome; Greig cephalopolysyndactyly syndrome; hair genetic diseases; hairy cell leukemia; HANAC syndrome; harlequin type ichtyosis congenita; HDR syndrome; hearing loss; hemochromatosis type 3; hemochromatosis type 4; hemolytic anemia; hemolytic uremic syndrome; hemophilia A; hemophilia B; hereditary angioedema type 3; hereditary angioedemas; hereditary hemorrhagic telangiectasia; hereditary hypofibrinogenemia; hereditary intraosseous vascular malformation; hereditary leiomyomatosis and renal cell cancer; hereditary neuralgic amyotrophy; hereditary sensory and autonomic neuropathy type; Hermansky-Pudlak disease; HHH syndrome; HHT2; hidrotic ectodermal dysplasia type 1; hidrotic ectodermal dysplasias; histiocytic sarcoma; HNF4A-associated hyperinsulinism; HNPCC; homozygous familial hypercholesterolemia; human immunodeficiency with microcephaly; human papilloma virus (HPV) infection; Huntington's disease; hyper-IgD syndrome; hyperinsulinism-hyperammonemia syndrome; hypercholesterolemia; hypertrophy of the retinal pigment epithelium; hypochondrogenesis; hypohidrotic ectodermal dysplasia; ICF syndrome; idiopathic congenital intestinal pseudo-obstruction; immunodeficiency 13; immunodeficiency 17; immunodeficiency 25; immunodeficiency with hyper-IgM type 1; immunodeficiency with hyper-IgM type 3; immunodeficiency with hyper-IgM type 4; immunodeficiency with hyper-IgM type 5; immunoglobulin alpha deficiency; inborn errors of thyroid metabolism; infantile myofibromatosis; infantile visceral myopathy; infantile X-linked spinal muscular atrophy; intrahepatic cholestasis of pregnancy; IPEX syndrome; IRAK4 deficiency; isolated congenital asplenia; Jeune syndrome; Johanson-Blizzard syndrome; Joubert syndrome; JP-HHT syndrome; juvenile hemochromatosis; juvenile hyalin fibromatosis; juvenile nephronophthisis; Kabuki mask syndrome; Kallmann syndromes; Kartagener syndrome; KCNJ11-associated hyperinsulinism; Kearns-Sayre syndrome; Kostmann disease; Kozlowski type of spondylometaphyseal dysplasia; Krabbe disease; LADD syndrome; late infantile-onset neuronal ceroid lipofuscinosis; LCK deficiency; LDHCP syndrome; Leber Congenital Amaurosis Teyp 10; Legius syndrome; Leigh syndrome; lethal congenital contracture syndrome 2; lethal congenital contracture syndromes; lethal contractural syndrome type 3; lethal neonatal CPT deficiency type 2; lethal osteosclerotic bone dysplasia; leukocyte adhesion deficiency; Li Fraumeni syndrome; LIG4 syndrome; lipodystrophy; lissencephaly type 1; lissencephaly type 3; Loeys-Dietz syndrome; low phospholipid-associated cholelithiasis; Lynch Syndrome; lysinuric protein intolerance; a lysosomal storage disease (e.g., Hunter syndrome, Hurler syndrome); macular dystrophy; Maffucci syndrome; Majeed syndrome; mannose-binding protein deficiency; mantle cell lymphoma; Marfan disease; Marshall syndrome; MASA syndrome; mastocytosis; MCAD deficiency; McCune-Albright syndrome; MCKD2; Meckel syndrome; MECP2 Duplication Syndrome; Meesmann corneal dystrophy; megacystis-microcolon-intestinal hypoperistalsis; megaloblastic anemia type 1; MEHMO; MELAS; Melnick-Needles syndrome; MEN2s; meningitis; Menkes disease; metachromatic leukodystrophies; methymalonic acidemia due to transcobalamin receptor defect; methylmalonic acidurias; methylvalonic aciduria; microcoria-congenital nephrosis syndrome; microvillous atrophy; migraine; mitochondrial neurogastrointestinal encephalomyopathy; monilethrix; monosomy X; mosaic trisomy 9 syndrome; Mowat-Wilson syndrome; mucolipidosis type 2; mucolipidosis type Ma; mucolipidosis type IV; mucopolysaccharidoses; mucopolysaccharidosis type 3A; mucopolysaccharidosis type 3C; mucopolysaccharidosis type 4B; multiminicore disease; multiple acyl-CoA dehydrogenation deficiency; multiple cutaneous and mucosal venous malformations; multiple endocrine neoplasia type 1; multiple sulfatase deficiency; mycosis fungoides; myotonic dystrophy; NAIC; nail-patella syndrome; nemaline myopathies; neonatal diabetes mellitus; neonatal surfactant deficiency; nephronophtisis; Netherton disease; neurofibromatoses; neurofibromatosis type 1; Niemann-Pick disease type A; Niemann-Pick disease type B; Niemann-Pick disease type C; NKX2E; non-alcoholic fatty liver disease (NAFLD); non-alcoholic steatohepatitis (NASH); Noonan syndrome; North American Indian childhood cirrhosis; NROB1 duplication-associated DSD; ocular genetic diseases; oculo-auricular syndrome; OLEDAID; oligomeganephronia; oligomeganephronic renal hypolasia; Ollier disease; Opitz-Kaveggia syndrome; orofaciodigital syndrome type 1; orofaciodigital syndrome type 2; osseous Paget disease; osteogenesis imperfecta; otopalatodigital syndrome type 2; OXPHOS diseases; palmoplantar hyperkeratosis; panlobar nephroblastomatosis; Parkes-Weber syndrome; Parkinson's disease; partial deletion of 21q22.2-q22.3; Pearson syndrome; Pelizaeus-Merzbacher disease; Pendred syndrome; pentalogy of Cantrell; peroxisomal acyl-CoA-oxidase deficiency; Peutz-Jeghers syndrome; Pfeiffer syndrome; Pierson syndrome; pigmented nodular adrenocortical disease; pipecolic acidemia; Pitt-Hopkins syndrome; plasmalogens deficiency; platelet glycoprotein IV deficiency; pleuropulmonary blastoma and cystic nephroma; polycystic kidney disease; polycystic ovarian disease; polycystic lipomembranous osteodysplasia; Pompe disease, including infantile onset Pompe disease (IOPD) and late onset Pompe disease (LOPD); porphyrias; PRKAG2 cardiac syndrome, premature ovarian failure; primary erythermalgia; primary hemochromatoses; primary hyperoxaluria; progressive familial intrahepatic cholestasis; propionic acidemia; protein-losing enteropathy; pyruvate decarboxylase deficiency; RAPADILINO syndrome; renal cystinosis; retinitis pigmentosa; Rett Syndrome; rhabdoid tumor predisposition syndrome; Rieger syndrome; ring chromosome 4; Roberts syndrome; Robinow-Sorauf syndrome; Rothmund-Thomson syndrome; severe combined immunodeficiency disorder (SCID); Saethre-Chotzen syndrome; Sandhoff disease; SC phocomelia syndrome; SCAS; Schinzel phocomelia syndrome; short rib-polydactyly syndrome type 1; short rib-polydactyly syndrome type 4; short-rib polydactyly syndrome type 2; short-rib polydactyly syndrome type 3; Shwachman disease; Shwachman-Diamond disease; sickle cell anemia; Silver-Russell syndrome; Simpson-Golabi-Behmel syndrome; Smith-Lemli-Opitz syndrome; SPG7-associated hereditary spastic paraplegia; spherocytosis; spinocerebellar ataxia; split-hand/foot malformation with long bone deficiencies; spondylocostal dysostosis; sporadic visceral myopathy with inclusion bodies; storage diseases; Stargardt macular dystrophy; STRA6-associated syndrome; stroke; Tay-Sachs disease; thanatophoric dysplasia; thyroid metabolism diseases; Tourette syndrome; transthyretin-associated amyloidosis; trisomy 13; trisomy 22; trisomy 2p syndrome; tuberous sclerosis; tufting enteropathy; urea cycle diseases; Usher Syndrome; Van Den Ende-Gupta syndrome; Van der Woude syndrome; variegated mosaic aneuploidy syndrome; VLCAD deficiency; von Hippel-Lindau disease; von Willebrand disease; Waardenburg syndrome; WAGR syndrome; Walker-Warburg syndrome; Werner syndrome; Wilson disease; Wiskott-Aldrich Syndrome; Wolcott-Rallison syndrome; Wolfram syndrome; X-linked agammaglobulinemia; X-linked chronic idiopathic intestinal pseudo-obstruction; X-linked cleft palate with ankyloglossia; X-linked dominant chondrodysplasia punctata; X-linked ectodermal dysplasia; X-linked Emery-Dreifuss muscular dystrophy; X-linked lissencephaly; X-linked lymphoproliferative disease; X-linked visceral heterotaxy; xanthinuria type 1; xanthinuria type 2; xeroderma pigmentosum; XPV; and Zellweger disease.

In some embodiments, compositions and methods modify at least one gene associated with the disease or the expression thereof. In some embodiments, the disease is Alzheimer's disease and the gene is selected from APP, BACE-1, PSD95, MAPT, PSEN1, PSEN2, and APOEe4. In some embodiments, the disease is Parkinson's disease and the gene is selected from SNCA, GDNF, and LRRK2. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINA1. In some embodiments, the disease is amyotrophic lateral sclerosis (ALS) and the gene is selected from SOD1, FUS, C90RF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises calcific aortic stenosis and the gene is Apo(a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMR1. In some embodiments, the disease comprises Fuchs corneal dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe disease (IOPD) and late onset Pompe disease (LOPD) and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCIII, APOA1, APOL1, ARH, CDKN2B, CFB, CXCL12, FM, FXII, GATA-4, MKA3, MKL2, MTHFD1L, MYH7, NKX2-5, NOTCH1, PKK, PCSK9, PSRC1, SMAD3, and TTR. In some embodiments, the disease comprises acromegaly and the gene is GHR. In some embodiments, the disease comprises acute myeloid leukemia and the gene is CD22. In some embodiments, the disease is diabetes and the gene is GCGR. In some embodiments, the disease is NAFLD/NASH and the gene is selected from DGAT2 and PNPLA3. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne muscular dystrophy and the gene is DMD. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot Marie Tooth disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMIEM43. In some embodiments, the disease comprises Factor V Leiden thrombophilia and the gene is F5. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM FANCN, FANCP, FANCS, RAD51C, and XPF. In some embodiments, the disease comprises Familial Creutzfeld-Jakob disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVIII. In some embodiments, the disease comprises histiocytosis and the gene is CD1. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHI1, NPHP1, CEP290, TMEM67, RPGRIP1L, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TP53. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease is mycosis fungoides and the gene is CD7. In some embodiments, the disease is myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COL1A1, COL1A2, and IFITM5. In some embodiments, the disease is non-small cell lung cancer and the gene is selected from KRAS, EGFR, ALK, METex14, BRAF V600E, ROS1, RET, and NTRK. In some embodiments, the disease comprises Peutz-Jeghers syndrome and the gene is STK11. In some embodiments, the disease comprises polycystic kidney disease and the gene is selected from PKD1 and PKD2. In some embodiments, the disease comprises Severe Combined Immune Deficiency and the gene is selected from IL7R, RAG1, JAK3. In some embodiments, the disease comprises PRKAG2 cardiac syndrome and the gene is PRKAG2. In some embodiments, the disease comprises spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease comprises Usher Syndrome and the gene is selected from MYO7A, USH1C, CDH23, PCDH15, USH1G, USH2A, GPR98, DFNB31, and CLRN1. In some embodiments, the disease comprises von Willebrand disease and the gene is VWT. In some embodiments, the disease comprises Waardenburg syndrome and the gene is selected from PAX3, MITTF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46. In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

### D. Cancer

In some embodiments, the disease is cancer. In some embodiments, the cancer is a solid cancer (i.e., a tumor). In some embodiments, the cancer is selected from a blood cell cancer, a leukemia, and a lymphoma. The cancer can be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL). In some embodiments, the cancer is any one of colon cancer, rectal cancer, renal-cell carcinoma, liver cancer, bladder cancer, cancer of the kidney or ureter, lung cancer, non-small cell lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, and thyroid cancer.

In some embodiments, mutations are associated with cancer or are causative of cancer. The target nucleic acid, in some embodiments, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or a combination thereof. Non-limiting examples of genes comprising a mutation associated with cancer are ABL, ACE, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK CAN, DICER1, DIS3L2, E2A/PBX1, EGFR, ENL/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Cα1, MAS, MAX, MDM-2, MEN1, MET, MITTF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDC1, PDGFRA, PHOX2B, PIM-1, PMS2, POLD1, POLE, POT1, PPARG, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP3, TMEM127, TNF, TP53, TRAC, TSC1, TSC2, TRK, VHL, WRN, and WT1. Non-limiting examples of oncogenes are KRAS, NRAS, BRAF, MYC, CTNNB1, and EGFR. In some instances, the oncogene is a gene that encodes a cyclin dependent kinase (CDK). Non-limiting examples of CDKs are Cdk1, Cdk4, Cdk5, Cdk7, Cdk8, Cdk9, Cdk11 and CDK20. Non-limiting examples of tumor suppressor genes are TP53, RB1, and PTEN.

### E. Infections

Described herein are compositions and methods for treating an infection in a subject. Infections may be caused by a pathogen, e.g., bacteria, viruses, fungi, and parasites. Compositions and methods may modify a target nucleic acid associated with the pathogen or parasite causing the infection. In some embodiments, the target nucleic acid may be in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, methods described herein include treating an infection caused by one or more viral pathogens. Non-limiting examples of viral pathogens include adenovirus, blue tongue virus, chikungunya, coronavirus (e.g., SARS-CoV-2), cytomegalovirus, Dengue virus, Ebola, Epstein-Barr virus, feline leukemia virus, Hemophilus influenzae B, Hepatitis virus A, Hepatitis virus B, Hepatitis virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, methods described herein include treating an infection caused by one or more parasites. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof

### EXAMPLES

The following examples are included for illustrative purposes only and are not intended to limit the scope of the disclosure. It will be understood by those of skill in the art that numerous and various modifications can be made to yield essentially similar results without departing from the spirit of the present disclosure.

### Example 1—Cas14 Proteins Provide Cis Cleavage Activity

Cas nucleases were tested for cis cleavage activity at varying temperatures, salt concentrations and tracrRNA lengths. Briefly, Cas nucleases were incubated at 100 nM with crRNA and tracrRNA in Cutsmart buffer (CS) or low salt buffer (LS) at room temperature for 20 minutes, followed by addition of target nucleic acid (1095 nucleobases (nb) in length) at a final concentration of 10 nM. Cutsmart buffer is 50 mM potassium acetate, 20 mM Tris-acetate, 10 mM magnesium acetate, 100 ug/ml BSA, pH 7.9 at 25 C. Low salt buffer is 20 mM Tricine, 15 mM MgCl₂, 0.2 mg/ml BSA, 1 mM TCEP, pH 9 at 37 C. TracrRNA and crRNA sequences are provided in TABLE 5 and results are provided in TABLE 6. In some cases, the PAM sequence comprises 5′-TTTR-3′ or 5′-NTTR-3′. In some instances, the target nucleic acid PAM was 5′-TTTG-3′, unless noted with an asterisk in TABLE 6, where the PAM was 5′-GTTG-3′. In some cases, the PAM sequence 5′-TTTR-3′ was associated with Enzyme SEQ ID NOs: 1, 3, 4, and 5. In some cases, the PAM sequence 5′-NTTR-3′ was associated with Enzyme SEQ ID NO: 2. The PAM sequences are designated as a DNA sequence where T is Thymine, G is Guanine, R is Adenine or Guanine, and N is any base. Additionally, TABLE 7 denotes Cas nucleases and corresponding repeat sequence of crRNA from this experiment. Cleavage of the target nucleic acid was detected by PCR and gel electrophoresis, wherein cleavage was indicated by the presence of a 770 bp fragment and a 325 bp. % cut values were obtained by densitometry and then corrected for partial mass.

TABLE 5
tracrRNA and crRNA sequences

| | | |
|---|---|---|
| ID | | SEQ ID |
| (length) | Sequence | NO. |

tracrRNA

| | | |
|---|---|---|
| R1518 | CUUCACUGAUAAAGUGGAGAACCGCUUCACCAAAAGCUGUCCCUU | 220 |
| (140 nt) | AGGGGAUUAGAACUUGAGUGAAGGUGGGCUGCUUGCAUCAGCCUA | |
| | AUGUCGAGAAGUGCUUUCUUCGGAAAGUAACCCUCGAAACAAAUU | |
| | CAUUU | |
| R1806 | CGAUUCCUCCCUACAGUAGUUAGGUAUAGCCGAAAGGUAGAGACU | 221 |
| (189 nt) | AAAUCUGUAGUUGGAGUGGGCCGCUUGCAUCGGCCUAAAGUUGAG | |
| | AAGUGUCAGACUCUGAUAACCCUCAACGACGAUAUUCUUUAUUUC | |
| | GGUUCAAAGUUCUGCACAAAACAGGUGAGUCCUUAUAAACCGGUG | |
| | UGCAGAACG | |
| R3249 | GUGAAGGUGGGCUGCUUGCAUCAGCCUAAUGUCGAGAAGUGCUUU | 222 |
| (103 nt) | CUUCGGAAAGUAACCCUCGAAACAAAUUCAUUUUUCCUCUCCAAU | |
| | UCUGCACAAAAAA | |
| R3280 | UUUCUCAGAGAUGGCUGAUCCCAACAGCCAAAUAUCGAGGUGUCU | 223 |
| (103 nt) | GCACCUAUAUGGACGCAGUAAGUAGAACCACCCUCGAAACAGUUC | |
| | UAUCGGAUUUUUU | |
| R3281 | UUUCUCAGAGAUGGCUGAUCCCAACAGCCAAAUAUCGAGGUGUCU | 224 |
| (103 nt) | GCACCUAUAUGGACGCAGUAAGUAGAACCACCCUCGAAACAGUUC | |
| | UAUCGGAUUUUUU | |
| R3283 | GGCUGAUUUCAACAGCCGAAUAUCGAGGUGCCAGAAUCAUUUUUU | 225 |
| (103 nt) | GAUCUUUGGAUAACACCCUCGAAACAACUAACUAUUGGAAGUUAA | |
| | UGAUGUAAAUUUA | |
| R3284 | CAAAGGGUUGGCUAUAGCAGGCUGUUGCUUGCAGAGGAUAAGUUG | 226 |
| (163 nt) | ACAUGAAUUUGUCAUCCAGCCUGAUGGAAUAAGGUGCGGCUGAUU | |
| | UCAACAGCCGAAUAUCGAGGUGCCAGAAUCAUUUUUUGAUCUUUG | |
| | GAUAACACCCUCGAAACAACUAACUAUU | |
| R3285 | GUCCCAAAAUCGCGGAUAAGACGCGGUUGCUUUCAACAGCCGAAA | 227 |
| (103 nt) | AUCGAGCUACCGGCGAACUUGUUUCCGCCGGAUCAUAGCCUCGAA | |
| | ACAACGCGUGCUU | |
| R3287 | CGCGGUGAUGGGUAUAGCCUUUGGGCAGAGUCCCAAAAUCGCGGA | 228 |
| (132 nt) | UAAGACGCGGUUGCUUUCAACAGCCGAAAAUCGAGCUACCGGCGA | |
| | ACUUGUUUCCGCCGGAUCAUAGCCUCGAAACAACGCGUGCUU | |
| R3288 | CGCGGUGAUGGGUAUAGCCUUUGGGCAGAGUCCCAAAAUCGCGGA | 229 |
| (132 nt) | UAAGACGCGGUUGCUUUCAACAGCCGAAAAUCGAGCUACCGGCGA | |
| | ACUUGUUUCCGCCGGAUCAUAGCCUCGAAACAACGCGUGCUU | |
| R3290 | CAAAAAGGUUGGCUAUAGCAGGCUAUUGCUUGCAGAGGAUAAGUU | 230 |
| (160 nt) | GACAAUAAAUUUGUCAUCCAGCCUGAUGGAAUAAGGCGCGGCUGA | |
| | UUUCAACAGCCUAAUAUCGAGGUGCCAGAAUCAUGCAUGAUCUCU | |
| | GGAGGACACCCUCGAAACAAUUAUU | |
| R3295 | GGCUGAUUUCAACAGCCAAAUAUCGAGGUGCCGGAAUUGUUAAAU | 231 |
| (103 nt) | AAUCUCCGGAGUACACCCUCGAAACAAUUUAAUGGAGACUGUUGA | |
| | GGCAAAUUUAUUG | |
| R3296 | AUAUUGCCCCGAAAUGUUGGCUAUAGCAGAUUUUCUAAAUCUGCA | 232 |
| (170 nt) | GAGGAUAAGUUGGCGUAGAUGCCAUUCAACAAAUCGGAAAGAGGC | |
| | AUGGCUGAUUUCAACAGCCAAAUAUCGAGGUGCCGGAAUUGUUAA | |
| | AUAAUCUCCGGAGUACACCCUCGAAACAAUUUAAU | |

crRNA

| | | |
|---|---|---|
| R2424 | GUUGCAGAACCCGAAUAGACGAAUGAAGGAAUGCAACUAUUAAAU | 233 |
| | ACUCGUAUUGCU | |
| R3671 | GUCUCAGAGCAUGUUCUGAUAGAAGAAGGAUUGGGACUAUUAAAU | 234 |
| | ACUCGUAUUGCU | |
| R3676 | GUCUCAGAGAUACUUCCAAUAAAACAAGGAUUGAAACUAUUAAAU | 235 |
| | ACUCGUAUUGCU | |
| R3681 | GUCUGAAAGCUACUUCCAAUAAAACAAGGAUUGAAACUAUUAAAU | 236 |
| | ACUCGUAUUGCU | |
| R3686 | GCCGCAUCUCGCACGCGUACGUGGAUUGAAACUAUUAAAUACUCG | 237 |
| | UAUUGCU | |
| R3691 | GUCUGAAUCACAAUUCCAUUAAAACAAGGAUUGAAACUAUUAAAU | 238 |
| | ACUCGUAUUGCU | |

TABLE 6
Cis cleavage activity provided by Cas nucleases

| Cas nuclease | | | % cut @ | % cut @ | % cut @ | % cut @ |
|---|---|---|---|---|---|---|
| SEQ ID NO. | tracrRNA | crRNA | 37° C., LS | 37º C., CS | 45º C., LS | 45° C., CS |
| SEQ ID NO: 8 | R1806 | R2424 | 39.64 | 42.02 | 65.08 | 69.18 |
| (Cas14a.1) | | | | | | |
| SEQ ID NO: 8 | R1518 | R2424 | 36.7 | 39.74 | 53.08 | 48.95 |
| SEQ ID NO: 8 | R3249 | R2424 | 40.61 | 39.48 | 56.33 | 64.04 |
| SEQ ID NO: 6 | R1806 | R2424 | 3.29 | 0 | 8.92 | 0 |
| (Cas14a.3) | | | | | | |
| SEQ ID NO: 6 | R3249 | R2424 | 0 | 0 | 0 | 0 |
| SEQ ID NO: 1 | R1806 | R2424 | 42.07 | 63.09 | 34.17 | 34.79 |
| SEQ ID NO: 1 | R3281 | R3671 | 56 | 76.66 | 43.63 | 49.22 |
| SEQ ID NO: 1 | R3280 | R3671 | 65.79 | 92.27 | 56.91 | 89.77 |
| SEQ ID NO: 2 | R1806 | R2424 | 37.31 | 16.54 | 74.66 | 70.19 |
| SEQ ID NO: 2 | R3284 | R3676 | 75.9 | 61.3 | 80.06 | 99.01 |
| SEQ ID NO: 2 | R3283 | R3676 | 46.92 | 42.52 | 89.88 | 124.62 |
| SEQ ID NO: 3 | R1806 | R2424 | 13.62 | 19.66 | 49.37 | 72.9 |
| SEQ ID NO: 3 | R3287 | R3686 | 21.43 | 28.46 | 61.35 | 93.51 |
| SEQ ID NO: 3 | R3285 | R3686 | 8.56 | 14.91 | 39.35 | 58.14 |
| SEQ ID NO: 4 | R1806 | R2424 | 41.43 | 38.23 | 69.85 | 97.14 |
| SEQ ID NO: 4 | R3290 | R3681 | 13.95 | 14.08 | 34.05 | 39.93 |
| SEQ ID NO: 4 | R3288 | R3681 | 25.38 | 24.56 | 75.02 | 92.68 |
| SEQ ID NO: 5 | R1806 | R2424 | 10.9 | 17.09 | 69.79 | 66.07 |
| SEQ ID NO: 5 | R3296 | R3691 | 56.46 | 58.99 | 78.4 | 77.98 |
| SEQ ID NO: 5 | R3295 | R3691 | 0 | 0 | 0 | 0 |
| SEQ ID NO: | R1806 | R2424 | 6.35 | 3.25 | 43.43 | 13.41 |
| 2* | | | | | | |
| SEQ ID NO: | R3284 | R3676 | 43.82 | 18.03 | 56 | 60.72 |
| 2* | | | | | | |
| SEQ ID NO: | R3283 | R3676 | 29.61 | 21.99 | 73.21 | 110.37 |
| 2* | | | | | | |

TABLE 7
Additional crRNA sequences

| Cas nuclease | |
|---|---|
| SEQ ID NO: | Repeat Sequence for crRNA |
| SEQ ID NO: 1 | GUCUCAGAGCAUGUUCUGAUAGAAGAAGGAUUGGGAC |
| | (SEQ ID NO: 258) |
| SEQ ID NO: 2 | GUCUCAGAGAUACUUCCAAUAAAACAAGGAUUGAAAC |
| | (SEQ ID NO: 259) |
| SEQ ID NO: 3 | GCCGCAUCUCGCACGCGUACGUGGAUUGAAAC (SEQ |
| | ID NO: 260) |
| SEQ ID NO: 4 | GUCUGAAAGCUACUUCCAAUAAAACAAGGAUUGAAAC |
| | (SEQ ID NO: 261) |
| SEQ ID NO: 5 | GUCUGAAUCACAAUUCCAUUAAAACAAGGAUUGAAAC |
| | (SEQ ID NO: 262) |

### Example 2—Cas14 Proteins Provide Trans Cleavage Activity

Cas nucleases were tested for trans cleavage activity with a variety of tracrRNAs, using a DETECTR assay. Briefly, Cas nucleases were incubated for 20 minutes at room temperature with 50 nM crRNA and 50 nM tracrRNA to produce RNP complexes. RNP complexes were incubated at 40 nM for 1 hour at 45° C. with 0.5 nM target nucleic acid in DOE buffer 5/18 (20 mM Tricine, pH 9, 15 mM Mg++). Upon hybridization of the crRNA with the target nucleic acid, the trans-cleavage activity of the Cas nuclease was activated, cleaving an ssDNA fluorescence-quenching (FQ) reporter molecule to separate a fluorophore and a fluorescence quenching moiety, thereby providing a fluorescent readout indicating the presence of the target nucleic acid in the sample. The target nucleic acid had a PAM of TTTG. Each Cas nuclease was incubated with a crRNA as indicated in TABLE 8, and each of the tracrRNAs shown in TABLE 8. Results are provided in TABLE 9. Cas nucleases represented by SEQ ID NOs: 1-4 provided trans activity under these conditions.

TABLE 8
tracrRNA Sequences Tested

| | | |
|---|---|---|
| ID | | SEQ ID |
| (length) | Sequence | NO. |

tracrRNA

| | | |
|---|---|---|
| R1518 | CUUCACUGAUAAAGUGGAGAACCGCUUCACCAAAAGCUGUCCCUU | 220 |
| (140 nt) | AGGGGAUUAGAACUUGAGUGAAGGUGGGCUGCUUGCAUCAGCCUA | |
| | AUGUCGAGAAGUGCUUUCUUCGGAAAGUAACCCUCGAAACAAAUU | |
| | CAUUU | |
| R1806 | CGAUUCCUCCCUACAGUAGUUAGGUAUAGCCGAAAGGUAGAGACU | 221 |
| (189 nt) | AAAUCUGUAGUUGGAGUGGGCCGCUUGCAUCGGCCUAAAGUUGAG | |
| | AAGUGUCAGACUCUGAUAACCCUCAACGACGAUAUUCUUUAUUUC | |
| | GGUUCAAAGUUCUGCACAAAACAGGUGAGUCCUUAUAAACCGGUG | |
| | UGCAGAACG | |
| R3248 | GUGAAGGUGGGCUGCUUGCAUCAGCCUAAUGUCGAGAAGUGCUUU | 239 |
| (81 nt) | CUUCGGAAAGUAACCCUCGAAACAAAUUCAUUUUUC | |
| R3249 | GUGAAGGUGGGCUGCUUGCAUCAGCCUAAUGUCGAGAAGUGCUUU | 222 |
| (103 nt) | CUUCGGAAAGUAACCCUCGAAACAAAUUCAUUUUUCCUCUCCAAU | |
| | UCUGCACAAAAAA | |
| R3279 | GGCUGAUCCCAACAGCCAAAUAUCGAGGUGUCUGCACCUAUAUGG | 240 |
| (103 nt) | ACGCAGUAAGUAGAACCACCCUCGAAACAGUUCUAUCGGAUUUUU | |
| | UUGGAAAUGAUUC | |
| R3280 | UUUCUCAGAGAUGGCUGAUCCCAACAGCCAAAUAUCGAGGUGUCU | 223 |
| (103 nt) | GCACCUAUAUGGACGCAGUAAGUAGAACCACCCUCGAAACAGUUC | |
| | UAUCGGAUUUUUU | |
| R3282 | CCAGCCUGAUGGAAUAAGGUGCGGCUGAUUUCAACAGCCGAAUAU | 241 |
| (103 nt) | CGAGGUGCCAGAAUCAUUUUUUGAUCUUUGGAUAACACCCUCGAA | |
| | ACAACUAACUAUU | |
| R3283 | GGCUGAUUUCAACAGCCGAAUAUCGAGGUGCCAGAAUCAUUUUUU | 225 |
| (103 nt) | GAUCUUUGGAUAACACCCUCGAAACAACUAACUAUUGGAAGUUAA | |
| | UGAUGUAAAUUUA | |
| R3285 | GUCCCAAAAUCGCGGAUAAGACGCGGUUGCUUUCAACAGCCGAAA | 227 |
| (103 nt) | AUCGAGCUACCGGCGAACUUGUUUCCGCCGGAUCAUAGCCUCGAAA | |
| | CAACGCGUGCUU | |
| R3286 | GGUUGCUUUCAACAGCCGAAAAUCGAGCUACCGGCGAACUUGUUU | 242 |
| (103 nt) | CCGCCGGAUCAUAGCCUCGAAACAACGCGUGCUUGUGUGCCUGUUG | |
| | AUGCGCCGUUUC | |
| R3288 | CGCGGUGAUGGGUAUAGCCUUUGGGCAGAGUCCCAAAAUCGCGGA | 229 |
| (132 nt) | UAAGACGCGGUUGCUUUCAACAGCCGAAAAUCGAGCUACCGGCGA | |
| | ACUUGUUUCCGCCGGAUCAUAGCCUCGAAACAACGCGUGCUU | |
| R3289 | GUCAUCCAGCCUGAUGGAAUAAGGCGCGGCUGAUUUCAACAGCCU | 243 |
| (103 nt) | AAUAUCGAGGUGCCAGAAUCAUGCAUGAUCUCUGGAGGACACCCU | |
| | CGAAACAAUUAUU | |
| R3291 | GGCUGAUUUCAACAGCCCAAAAUCGAGUAGUACCGAUUCGUUAUG | 244 |
| (102 nt) | AUUCGGCAACCCUCGAAACUAUUAUAACUUUUUUCAUGUCUUUAU | |
| | AAAAGAUUGAAA | |
| R3292 | GCGUACUCCGCAGGCGAGGUAUGUACAACUGCACGGCUGAUUUCA | 245 |
| (103 nt) | ACAGCCCAAAAUCGAGUAGUACCGAUUCGUUAUGAUUCGGCAACC | |
| | CUCGAAACUUAUU | |
| R3294 | CAUUCAACAAAUCGGAAAGAGGCAUGGCUGAUUUCAACAGCCAAA | 246 |
| (103 nt) | UAUCGAGGUGCCGGAAUUGUUAAAUAAUCUCCGGAGUACACCCUC | |
| | GAAACAAUUUAAU | |
| R3295 | GGCUGAUUUCAACAGCCAAAUAUCGAGGUGCCGGAAUUGUUAAAU | 231 |
| (103 nt) | AAUCUCCGGAGUACACCCUCGAAACAAUUUAAUGGAGACUGUUGA | |
| | GGCAAAUUUAUUG | |

TABLE 9
Trans cleavage activity provided by Cas nucleases

| Cas | | |
|---|---|---|
| nuclease | | |
| SEQ ID | | tracrRNAs providing |
| NO. | crRNA Sequence | trans cleavage activity |
| SEQ ID | GUCUCAGAGCAUGUUCUGAUAGAAGAAGGAUUGG | R1806, R3279, R3248, |
| NO: 1 | GACGCCGAUAAUGAUGUAGGGAU (SEQ ID NO: 247) | R3280, R3283, R3288, |
| | | R3294, R3295 |
| SEQ ID | GUCUCAGAGAUACUUCCAAUAAAACAAGGAUUGA | R1518, R3283, R3295 |
| NO: 2 | AACGCCGAUAAUGAUGUAGGGAU (SEQ ID NO: 248) | |
| SEQ ID | GCCGCAUCUCGCACGCGUACGUGGAUUGAAACGCC | R1518, R1806, R3283, |
| NO: 3 | GAUAAUGAUGUAGGGAU (SEQ ID NO: 249) | R3285, R3286, R3295 |
| SEQ ID | GUCUGAAAGCUACUUCCAAUAAAACAAGGAUUGA | R1806, R3283, R3295 |
| NO: 4 | AACGCCGAUAAUGAUGUAGGGAU (SEQ ID NO: 250) | |
| SEQ ID | GUCUGAAUCACAAUUCCAUUAAAACAAGGAUUGA | None |
| NO: 5 | AACGCCGAUAAUGAUGUAGGGAU (SEQ ID NO: 251) | |
| SEQ ID | GUUGCAGAACCCGAAUAGACGAAUGAAGGAAUGC | R1806 |
| NO: 6 | AACGCCGAUAAUGAUGUAGGGAU (SEQ ID NO: 252) | |
| SEQ ID | GUUGCAGAACCCGAAUAGACGAAUGAAGGAAUGC | All |
| NO: 8 | AACGCCGAUAAUGAUGUAGGGAU (SEQ ID NO: 253) | |

### Example 3—PAM Screening for Cas14 Proteins

Cas proteins represented by SEQ ID NOs: 1-5 were screened by in vitro enrichment (IVE) for cis cleavage to determine recognized PAMs, using tracrRNA R1806 (SEQ ID NO: 221), and crRNA R3205 having the sequence: GUUGCAGAACCCGAAUAGACGAAUGAAGGAAUGCAACUAUUAAAUACUCGUAUUGCUGU (SEQ ID NO: 254). RNA protein (RNP) complexes were formed at 10×RNP (500 nM): 1.5 μl of 5 μM protein, 1.5 μl of 5 μM RNA (both tracrRNA and crRNA), and 1× CutSmart buffer to 15 μl. RNPs were diluted to 50 nM after complexing. The final RNP concentrations were 50 nM and 5 nM. PAM screening reactions used 10 μl of RNP in 100 μl reactions with a 5′ PAM library and were carried out for 1 hour at 37 C. Reactions were terminated with 1 μl proteinase K and 5 μl EDTA for 30 minutes at 37 C. Next generation sequencing was performed on cut sequences to identify enriched PAMs. Enriched 5′ PAM consensus sequences were TTTR and NTTR, where R is a purine and N is any nucleotide.

### Example 4—Nucleic Acid Detection with a Programmable Nuclease

This example covers the detection of a target nucleic acid sequence with a programmable nuclease. Upon complexation to a target nucleic acid sequence, the programmable nuclease performs transcollateral cleavage of single stranded nucleic acids. Such activity may be utilized for detection of the target nucleic acid by further providing reporter nucleic acids which generate a detectable signal upon cleavage. The programmable nuclease may be part of a multimeric complex of programmable nucleases, including a dimer.

A sample from a subject (e.g., a buccal swab) suspected of having an influenza infection is incubated in viral lysis buffer, resulting in flu virion lysis and solution accessible flu RNA. The sample is then subjected to reverse-transcription loop-mediated amplification (RT-LAMP), such that flu RNA present in the sample is reverse-transcribed and amplified to generate a population of flu DNA amplicons.

Separately, the programmable nuclease comprising an amino acid sequence selected from SEQ ID NOs: 1-5 is combined with a guide nucleic acid targeting a sequence of the flu DNA amplicons and configured to bind to the programmable nuclease under conditions permissive for the programmable nuclease. A complex rapidly forms upon mixing at room temperature the programmable nuclease and a copy of the guide nucleic acid. Alternatively, a multimeric complex forms comprising multiple programmable nucleases and multiple copies of the guide nucleic acid.

1 μM of the programmable nuclease is combined with 5 μM of a reporter molecule comprising SEQ ID NO: 205 and the flu DNA amplicons. Reporter molecule cleavage by the programmable nuclease is monitored by fluorescence. An increase in fluorescence indicates that the programmable nuclease bound to flu DNA amplicons, thereby activating the programmable nuclease for transcollateral reporter molecule cleavage. Negligible change in fluorescence indicates a lack of flu DNA amplicons in the sample, suggesting that the subject is not infected with the influenza virus.

### Example 5—Genetic Modification with a Homodimeric Programmable Nuclease Complex

This example covers genetic engineering with programmable nucleases. A programmable nuclease may cleave a target region of a genome, enabling donor nucleic acid incorporation at the cleavage site, and formation of a stably transfected cell line. The programmable nuclease may be part of a multimeric complex of programmable nucleases, including a dimer.

A population of E. coli is transfected with a plasmid comprising a nucleobase sequence encoding a programmable nuclease comprising an amino acid sequence selected from SEQ ID NOs: 1-5, three separate sequences encoding gRNA under the control of separate U6 promoters, and a donor DNA sequence encoding a selection marker under the control of a P_{BAD} inducible promoter. The first gRNA targets a sequence of the E. coli genome, while the second and third gRNA target sequences immediately flanking the donor DNA sequence of the plasmid, such that expression of the programmable nuclease results in cleavage at a specific site in the E. coli DNA, excision of the donor DNA sequence from the plasmid, and incorporation of the donor DNA selection marker sequence into the E. coli genome. Stable transfection is confirmed by selection for the donor DNA. Transfected cells are cultured, thereby generating a population of cells from the transfected cells.

### Example 6—PAM Recognition by Programmable Nucleases and Single Guide RNA Combinations

Programmable nucleases and single guide RNA combinations were screened by in vitro enrichment (IVE) to detect cis cleavage activity. Programmable nucleases and single guide RNAs were expressed and purified from E. coli. Briefly, programmable nucleases were complexed with corresponding guide RNAs for 15 minutes at 37° C. The complexes were added to an IVE reaction mix. Reactions used 10 μl of RNP in 100 μl reactions with 1,000 ng of a 5′ PAM library in Ix Cutsmart buffer and were carried out for 15 minutes at 25° C., 45 minutes at 37° C. and 15 minutes at 45° C. Reactions were terminated with 1 μl of proteinase K and 5 μl of 500 mM EDTA for 30 minutes at 37° C. Cis cleavage by each complex was confirmed by the presence of band by gel electrophoresis. TABLE 10 shows the sgRNAs that provided cis cleavage for each corresponding nuclease as assayed by gel electrophoresis.

TABLE 10
Cis cleavage of sgRNAs by Cas nucleases

| Cas | | | |
|---|---|---|---|
| nuclease | | | SEQ |
| SEQ ID | | | ID |
| NO: | ID | sgRNA sequence | NO: |
| 1 | R5966 | UUUCUCAGAGAUGGCUGAUCCCAACAGCCAAAU | 263 |
| | | AUCGAGGUGUCUGCACCUAUAUGGACGCAGUAA | |
| | | GUAGAACCACCCUCGAAACAGUUCUGAAAAGAA | |
| | | GAAGGAUUGGGACUAUUAAAUACUCGUAUUGCU | |
| 1 | R5967 | CUAAAGGCUGACCAUAGCAGGUUUCCUGCAGGC | 264 |
| | | GGUUAGUUUUCUCAGAGAUGGCUGAUCCCAACA | |
| | | GCCAAAUAUCGAGGUGUCUGCACCUAUAUGGAC | |
| | | GCAGUAAGUAGAACCACCCUCGAAACAGUUCUG | |
| | | AAAAGAAGAAGGAUUGGGACUAUUAAAUACUCG | |
| | | UAUUGCU | |
| 2 | R5968 | GGCUGAUUUCAACAGCCGAAUAUCGAGGUGCCA | 265 |
| | | GAAUCAUUUUUUGAUCUUUGGAUAACACCCUCG | |
| | | AAACAACUAAGAAAAAAACAAGGAUUGAAACUA | |
| | | UUAAAUACUCGUAUUGCU | |
| 2 | R5969 | GGUUGGCUAUAGCAGGCUGUUGCUUGCAGAGGA | 266 |
| | | UAAGUUGACAUGAAUUUGUCAUCCAGCCUGAUG | |
| | | GAAUAAGGUGCGGCUGAUUUCAACAGCCGAAUA | |
| | | UCGAGGUGCCAGAAUCAUUUUUUGAUCUUUGGA | |
| | | UAACACCCUCGAAACAACUAAGAAAAAAACAAG | |
| | | GAUUGAAACUAUUAAAUACUCGUAUUGCU | |
| 3 | R5972 | GUCCCAAAAUCGCGGAUAAGACGCGGUUGCUUU | 267 |
| | | CAACAGCCGAAAAUCGAGCUACCGGCGAACUUG | |
| | | UUUCCGCCGGAUCAUAGCCUCGAAACAACGCGG | |
| | | AAACGUACGUGGAUUGAAACUAUUAAAUACUCG | |
| | | UAUUGCU | |
| 3 | R5973 | CGCGGUGAUGGGUAUAGCCUUUGGGCAGAGUCC | 268 |
| | | CAAAAUCGCGGAUAAGACGCGGUUGCUUUCAAC | |
| | | AGCCGAAAAUCGAGCUACCGGCGAACUUGUUUC | |
| | | CGCCGGAUCAUAGCCUCGAAACAACGCGGAAAC | |
| | | GUACGUGGAUUGAAACUAUUAAAUACUCGUAUU | |
| | | GCU | |
| 4 | R5970 | CGGCUGAUUUCAACAGCCUAAUAUCGAGGUGCC | 269 |
| | | AGAAUCAUGCAUGAUCUCUGGAGGACACCCUCG | |
| | | AAACAAUUAUUGGAAGCUAAUGAUGCAAAUUUA | |
| | | UGAAAAAAACAAGGAUUGAAACUAUUAAAUACU | |
| | | CGUAUUGCU | |
| 4 | R5971 | GGUUGGCUAUAGCAGGCUAUUGCUUGCAGAGGA | 270 |
| | | UAAGUUGACAAUAAAUUUGUCAUCCAGCCUGAU | |
| | | GGAAUAAGGCGCGGCUGAUUUCAACAGCCUAAU | |
| | | AUCGAGGUGCCAGAAUCAUGCAUGAUCUCUGGA | |
| | | GGACACCCUCGAAACAAUUAUGAAAAAAACAAG | |
| | | GAUUGAAACUAUUAAAUACUCGUAUUGCU | |
| 5 | R5974 | GGCUGAUUUCAACAGCCAAAUAUCGAGGUGCCG | 271 |
| | | GAAUUGUUAAAUAAUCUCCGGAGUACACCCUCG | |
| | | AAACAAUUUAGAAAAAAACAAGGAUUGAAACUA | |
| | | UUAAAUACUCGUAUUGCU | |
| 5 | R5975 | UGCAGAGGAUAAGUUGGCGUAGAUGCCAUUCAA | 272 |
| | | CAAAUCGGAAAGAGGCAUGGCUGAUUUCAACAG | |
| | | CCAAAUAUCGAGGUGCCGGAAUUGUUAAAUAAU | |
| | | CUCCGGAGUACACCCUCGAAACAAUUUAGAAAA | |
| | | AAACAAGGAUUGAAACUAUUAAAUACUCGUAUU | |
| | | GCU | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising a programmable nuclease comprising an amino acid sequence, or a nucleic acid encoding the programmable nuclease, wherein the programmable nuclease is at least 70% identical to any one of SEQ ID NOs: 1-5; and an engineered guide nucleic acid, or a DNA molecule encoding the engineered guide nucleic acid, wherein the programmable nuclease binds to at least a portion of the engineered guide nucleic acid.

2. The composition of claim 1, wherein the engineered guide nucleic acid comprises a crRNA, a tracrRNA, or a combination thereof.

3. The composition of claim 1, wherein the engineered guide nucleic acid comprises a single guide RNA (sgRNA).

4. The composition of claim 1, wherein the engineered guide nucleic acid comprises a nucleotide sequence that is at least 80%, at least 85%, at least 90% or at least 95% identical to any one of SEQ ID NOs: 255-262.

5-7. (canceled)

8. The composition of claim 1, wherein the programmable nuclease comprises no greater than 540 amino acids.

9-13. (canceled)

14. The composition of claim 1, wherein the programmable nuclease recognizes a protospacer adjacent motif (PAM) sequence directly adjacent to a target sequence of a target nucleic acid.

15. The composition of claim 14, wherein the target nucleic acid is a double stranded DNA molecule comprising a target strand and a non-target strand, wherein at least a portion of the engineered guide nucleic acid is complementary to the target sequence on the target strand, and wherein the PAM is directly adjacent to the 5′ end of the target sequence on the non-target strand of the double stranded DNA molecule.

16. The composition of claim 14, wherein the PAM sequence comprises: (i) 5′-NTTR-3′, wherein N is any base, T is thymine, and R is a purine, (ii) 5′-TTTR-3′, wherein T is thymine, and R is a purine, (iii) 5′-TTTG-3′, wherein T is thymine, and G is guanine, or (iv) 5′-GTTG-3′, wherein T is thymine, and G is guanine.

17-19. (canceled)

20. A composition comprising a programmable nuclease, or a nucleic acid encoding the programmable nuclease, and an engineered guide nucleic acid, or a DNA molecule encoding the engineered guide nucleic acid, wherein the programmable nuclease comprises:
(i) an amino acid sequence that is at least 80% identical to SEQ ID NO: 1, and wherein the engineered guide nucleic acid comprises a nucleotide sequence that is at least 80% identical to SEQ ID NO: 255;
(ii) an amino acid sequence that is at least 80% identical to any one of SEQ ID NOs: 2, 4, and 5, and wherein the engineered guide nucleic acid comprises a nucleotide sequence that is at least 80% identical to SEQ ID NO: 256;
(iii) an amino acid sequence that is at least 80% identical to SEQ ID NO: 3, and wherein the engineered guide nucleic acid comprises a nucleotide sequence that is at least 80% identical to SEQ ID NO: 257;
(iv) an amino acid sequence that is at least 80% identical to SEQ ID NO: 1, and wherein the engineered guide nucleic acid comprises a nucleotide sequence that is at least 80% identical to SEQ ID NO: 258;
(v) an amino acid sequence that is at least 80% identical to SEQ ID NO: 2, and wherein the engineered guide nucleic acid comprises a nucleotide sequence that is at least 80% identical to SEQ ID NO: 259;
(vi) an amino acid sequence that is at least 80% identical to SEQ ID NO: 3, and wherein the engineered guide nucleic acid comprises a nucleotide sequence that is at least 80% identical to SEQ ID NO: 260;
(vii) an amino acid sequence that is at least 80% identical to SEQ ID NO: 4, and wherein the engineered guide nucleic acid comprises a nucleotide sequence that is at least 80% identical to SEQ ID NO: 261; or
(viii) an amino acid sequence that is at least 80% identical to SEQ ID NO: 5, and wherein the engineered guide nucleic acid comprises a nucleotide sequence that is at least 80% identical to SEQ ID NO: 262.

21. The composition of claim 20, wherein the engineered guide nucleic acid is a sgRNA.

22-35. (canceled)

36. The composition of claim 1, wherein the programmable nuclease comprises at least one amino acid change relative to a wildtype counterpart that reduces a nucleic acid-cleaving activity of the programmable nuclease relative to that of a wildtype counterpart.

37. The composition of claim 1, wherein the composition comprises a fusion partner fused to the programmable nuclease, or a nucleic acid encoding the fusion partner.

38. The composition of claim 37, wherein the fusion partner comprises a base editing enzyme, a transcription activator domain, a transcription repressor domain, a nuclear localization signal, or a heterologous moiety.

39. The composition of claim 37, wherein the fusion partner provides nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, or glycosylase activity.

40-42. (canceled)

43. The composition of claim 1, wherein the nucleic acid encoding the programmable nuclease is an expression vector.

44. The composition of claim 43, wherein the expression vector is an adeno associated viral vector.

45. The composition of claim 43, wherein the expression vector encodes the engineered guide nucleic acid.

46. (canceled)

47. The composition of claim 1, wherein the composition comprises a lipid or a lipid nanoparticle.

48. (canceled)

49. A pharmaceutical composition comprising the composition of claim 1.

50-51. (canceled)

52. A system for detecting a target nucleic acid comprising the composition of claim 1 in a solution, wherein the solution comprises at least one of a buffering agent, a salt, a crowding agent, a detergent, a reducing agent, a competitor, and a reporter nucleic acid.

53-57. (canceled)

58. A method of modifying a target nucleic acid, comprising contacting the target nucleic acid with the composition of claim 1, thereby modifying the target nucleic acid.

59. The method of claim 58, wherein the target nucleic acid is within a facioscapulohumeral muscular dystrophy gene.


SEQUENCE LISTING (317 sequences)

SEQ ID NO: 1 - protein, 492 aa, synthetic construct
  REGION 1..492: note = Description of Artificial Sequence: Synthetic polypeptide
MAGKKKDKDVINKTLSVRIIRPRYSDDIEKEISDEKAKRKQDGKTGELDRAFFSELKSRNPDIITNDELFPLFTEIQKNLTEIYNKSISLLYMKLIVEEEGGSTASALSAGPYKECKARFNSYISLGLRQKIQSNFRRKELKGFQVSLPTAKSDRFPIPFCHQVENGKGGFKVYETGDDFIFEVPLIKYTATNKKSTSGKNYTKVQLNNPPVPMNVPLLLSTMRRRQTKKGMQWNKDEGTNAELRRVMSGEYKVSYAEIIRRTRFGKHDDWFVNFSIKFKNKTDELNQNVRGGIDIGVSNPLVCAVTNGLDRYIVANNDIMAFNERAMARRRTLLRKNRFKRSGHGAKNKLEPITVLTEKNERFRKSILQRWAREVAEFFKRTSASVVNMEDLSGITEREDFFSTKLRTTWNYRLMQTTIENKLKEYGIAVNYISPKYTSQTCHSCGKRNDYFTFSYRSENNYPPFECKECNKVKCNADFNAAKNIALKVVL

SEQ ID NO: 2 - protein, 477 aa, synthetic construct
  REGION 1..477: note = Description of Artificial Sequence: Synthetic polypeptide
MRISKTLSLRIVRPFYTPEVEAGIKAEKDKREAQGQTRSLDAKFFNELKKKHSEIILSSEFYSLLSEVQRQLTSIYNHAMSNLYHKIIVEGEKTSTSKALSNIGYDECKAIFPSYMALGLRQKIQSNFRRRDLKNFRMAVPTAKSDKFPIPIYRQVDGSKGGFKISENDGKDFIVELPLVDYVAEEVKTAKGRFTKINISKPPKIKNIPVILSTLRRRQSGQWFSDDGTNAEIRRVISGEYKVSWIEIVRRTRFGKHDDWFVNMVIKYDKPEEGLDSKVVGGIDVGVSSPLVCALNNSLDRYFVKSSDIIAFNKRAMARRRTLLRQNKYKRSGHGSKNKLEPITVLTEKNERFKKSIMQRWAKEVAEFFRGKGASVVRMEELSGLKEKDNFFSSYLRMYWNYGQLQQIIENKLKEYGIKVNYVSPKDTSKKCHSCTHINEFFTFEYRQKNNFPLFKCEKCGVECSADYNAAKNMAIA

SEQ ID NO: 3 - protein, 478 aa, synthetic construct
  REGION 1..478: note = Description of Artificial Sequence: Synthetic polypeptide
MKDYIRKTLSLRILRPYYGEEIEKEIAAAKKKSQAEGGDGALDNKFWDRLKAEHPEIISSREFYDLLDAIQRETTLYYNRAISKLYHSLIVEREQVSTAKALSAGPYHEFREKFNAYISLGLREKIQSNFRRKELARYQVALPTAKSDTFPIPIYKGFDKNGKGGFKVREIENGDFVIDLPLMAYHRVGGKAGREYIELDRPPAVLNVPVILSTSRRRANKTWFRDEGTDAEIRRVMAGEYKVSWVEILQRKRFGKPYGGWYVNFTIKYQPRDYGLDPKVKGGIDIGLSSPLVCAVTNSLARLTIRDNDLVAFNRKAMARRRTLLRQNRYKRSGHGSANKLKPIEALTEKNELYRKAIMRRWAREAADFFRQHRAATVNMEDLTGIKDREDYFSQMLRCYWNYSQLQTMLENKLKEYGIAVKYIEPKDTSKTCHSCGHVNEYFDFNYRSAHKFPMFKCEKCGVECGADYNAARNIAQA

SEQ ID NO: 4 - protein, 477 aa, synthetic construct
  REGION 1..477: note = Description of Artificial Sequence: Synthetic polypeptide
VKISKTLSLRIIRPYYTPEVESAIKAEKDKREAQGQTRNLDAKFFNELKKKHPQIILSGEFYSLLFEMQRQLTSIYNRAMSSLYHKIIVEGEKTSTSKALSDIGYDECKSVFPSYIALGLRQKIQSNFRRKELKGFRMAVPTAKSDKFPIPIYKQVDDGKGGFKISENKEGDFIVELPLVEYTAEDVKTAKGKFTKINISKPPKIKNIPVILSTLRRKQSGQWFSDEGTNAEIRRVISGEYKVSWIEVVRRTRFGKHDDWFLNIVIKYDKTEDGLDPEVVGGIDVGVSTPLVCAVNNSLDRYFVKSSDIIAFKKRAMARRRTLLRQNRFKRSGHGSKSKLEPITILTEKNERFKKSIMQRWAKEVAEFFKGERASVVQMEELSGLKEKDNFFGSYLRMYWNYGQLQQIIENKLKEYGIKVNYVSPKDTSKKCHSCGYINEFFTFEFRQKNNFPLFKCKKCGVECNADYNAAKNIAIA

SEQ ID NO: 5 - protein, 472 aa, synthetic construct
  REGION 1..472: note = Description of Artificial Sequence: Synthetic polypeptide
VPITKTISLRILRPYYPPEIEAKIKAEKEKRKENGDTGSLNSSYYRELKKEYPSIIINDEFFPLLSEMQRNITSIYNRTISHLYHRLIIKKESISTAKALSEGPYRDFKSTFNSYIALGLRQKVQSNFRKKDLMAFKIALPTAKSDKFPIPIYMQTNFKIKESPDSDFIIELPLVEYIAKETKGKNKMFTKVEILSPPKVKNIPVILSTRRRKESGQWFSDEGTNAEIRRIISGEYKVSWIEIVKRTRFGKHDWFVNMVISFEESQEGLDPDVIGGIDIGVSKPLICAINNSLDRYIVKGDDIIAFNRRALSRRRSLLRRNRLKRSGHGSRNKLEPITVLTEKNERFKKSIMQRWAKEVAEFFKSKRASIVQMEELTGIKEREDFFSKTLRMYWNYGQLQKTVENKLREYGIEVRYASPKDTSRRCHSCGHINDYFTFEFRQQNNFPLFKCMNCGIECSADYNAARNIAIAR

SEQ ID NO: 6 - protein, 500 aa, synthetic construct
  REGION 1..500: note = Description of Artificial Sequence: Synthetic polypeptide
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 7 - protein, 507 aa, synthetic construct
  REGION 1..507: note = Description of Artificial Sequence: Synthetic polypeptide
MEEAKTVSKTLSLRILRPLYSAEIEKEIKEEKERRKQGGKSGELDSGFYKKLEKKHTQMFGWDKLNLMLSQLQRQIARVFNQSISELYIETVIQGKKSNKHYTSKIVYNRAYSVFYNAYLALGITSKVEANFRSTELLMQKSSLPTAKSDNFPILLHKQKGVEGEEGGFKISADGNDLIFEIPIPFYEYDSANKKEPFKWIKKGGQKPTIKLILSTFRRQRNKGWAKDEGTDAEIRKVIEGKYQVSHIEINRGKKLGDHQKWFVNFTIEQPIYERKLDKNIIGGIDVGIKSPLVCAVNNSFARYSVDSNDVLKFSKQAFAFRRRLLSKNSLKRSGHGSKNKLDPITRMTEKNDRFRKKIIERWAKEVTNFFIKNQVGTVQIEDLSTMKDRQDNFFNQYLRGFWPYYQMQNLIENKLKEYGIETKRIKARYTSQLCSNPSCRHWNSYFSFDHRKTNNFPKFKCEKCALEISADYNAARNISTPDIEKFVAKATKGINLPDKNENVILE

SEQ ID NO: 8 - protein, 529 aa, synthetic construct
  REGION 1..529: note = Description of Artificial Sequence: Synthetic polypeptide
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 9 - protein, 726 aa, synthetic construct
  REGION 1..726: note = Description of Artificial Sequence: Synthetic polypeptide
MERQKVPQIRKIVRVVPLRILRPKYSDVIENALKKFKEKGDDTNTNDFWRAIRDRDTEFFRKELNFSEDEINQLERDTLFRVGLDNRVLFSYFDFLQEKLMKDYNKIISKLFINRQSKSSFENDLTDEEVEELIEKDVTPFYGAYIGKGIKSVIKSNLGGKFIKSVKIDRETKKVTKLTAINIGLMGLPVAKSDTFPIKIIKTNPDYITFQKSTKENLQKIEDYETGIEYGDLLVQITIPWFKNENKDFSLIKTKEAIEYYKLNGVGKKDLLNINLVLTTYHIRKKKSWQIDGSSQSLVREMANGELEEKWKSFFDTFIKKYGDEGKSALVKRRVNKKSRAKGEKGRELNLDERIKRLYDSIKAKSFPSEINLIPENYKWKLHFSIEIPPMVNDIDSNLYGGIDFGEQNIATLCVKNIEKDDYDFLTIYGNDLLKHAQASYARRRIMRVQDEYKARGHGKSRKTKAQEDYSERMQKLRQKITERLVKQISDFFLWRNKFHMAVCSLRYEDLNTLYKGESVKAKRMRQFINKQQLFNGIERKLKDYNSEIYVNSRYPHYTSRLCSKCGKLNLYFDFLKFRTKNIIIRKNPDGSEIKYMPFFICEFCGWKQAGDKNASANIADKDYQDKLNKEKEFCNIRKPKSKKEDIGEENEEERDYSRRFNRNSFIYNSLKKDNKLNQEKLFDEWKNQLKRKIDGRNKFEPKEYKDRFSYLFAYYQEIIKNESES

SEQ ID NO: 10 - protein, 517 aa, synthetic construct
  REGION 1..517: note = Description of Artificial Sequence: Synthetic polypeptide
MVPTELITKTLQLRVIRPLYFEEIEKELAELKEQKEKEFEETNSLLLESKKIDAKSLKKLKRKARSSAAVEFWKIAKEKYPDILTKPEMEFIFSEMQKMMARFYNKSMTNIFIEMNNDEKVNPLSLISKASTEANQVIKCSSISSGLNRKIAGSINKTKFKQVRDGLISLPTARTETFPISFYKSTANKDEIPISKINLPSEEEADLTITLPFPFFEIKKEKKGQKAYSYFNIIEKSGRSNNKIDLLLSTHRRQRRKGWKEEGGTSAEIRRLMEGEFDKEWEIYLGEAEKSEKAKNDLIKNMTRGKLSKDIKEQLEDIQVKYFSDNNVESWNDLSKEQKQELSKLRKKKVEELKDWKHVKEILKTRAKIGWVELKRGKRQRDRNKWFVNITITRPPFINKELDDTKFGGIDLGVKVPFVCAVHGSPARLIIKENEILQFNKMVSARNRQITKDSEQRKGRGKKNKFIKKEIFNERNELFRKKIIERWANQIVKFFEDQKCATVQIENLESFDRTSYK

SEQ ID NO: 11 - protein, 481 aa, synthetic construct
  REGION 1..481: note = Description of Artificial Sequence: Synthetic polypeptide
MKSDTKDKKIIIHQTKTLSLRIVKPQSIPMEEFTDLVRYHQMIIFPVYNNGAIDLYKKLFKAKIQKGNEARAIKYFMNKIVYAPIANTVKNSYIALGYSTKMQSSFSGKRLWDLRFGEATPPTIKADFPLPFYNQSGFKVSSENGEFIIGIPFGQYTKKTVSDIEKKTSFAWDKFTLEDTTKKTLIELLLSTKTRKMNEGWKNNEGTEAEIKRVMDGTYQVTSLEILQRDDSWFVNFNIAYDSLKKQPDRDKIAGIHMGITRPLTAVIYNNKYRALSIYPNTVMHLTQKQLARIKEQRTNSKYATGGHGRNAKVTGTDTLSEAYRQRRKKIIEDWIASIVKFAINNEIGTIYLEDISNTNSFFAAREQKLIYLEDISNTNSFLSTYKYPISAISDTLQHKLEEKAIQVIRKKAYYVNQICSLCGHYNKGFTYQFRRKNKFPKMKCQGCLEATSTEFNAAANVANPDYEKLLIKHGLLQLKK

SEQ ID NO: 12 - protein, 358 aa, synthetic construct
  REGION 1..358: note = Description of Artificial Sequence: Synthetic polypeptide
MSTITRQVRLSPTPEQSRLLMAHCQQYISTVNVLVAAFDSEVLTGKVSTKDFRAALPSAVKNQALRDAQSVFKRSVELGCLPVLKKPHCQWNNQNWRVEGDQLILPICKDGKTQQERFRCAAVALEGKAGILRIKKKRGKWIADLTVTQEDAPESSGSAIMGVDLGIKVPAVAHIGGKGTRFFGNGRSQRSMRRRFYARRKTLQKAKKLRAVRKSKGKEARWMKTINHQLSRQIVNHAHALGVGTIKIEALQGIRKGTTRKSRGAAARKNNRMTNTWSFSQLTLFITYKAQRQGITVEQVDPAYTSQDCPACRARNGAQDRTYVCSECGWRGHRDTVGAINISRRAGLSGHRRGATGA

SEQ ID NO: 13 - protein, 507 aa, synthetic construct
  REGION 1..507: note = Description of Artificial Sequence: Synthetic polypeptide
MIAQKTIKIKLNPTKEQIIKLNSIIEEYIKVSNFTAKKIAEIQESFTDSGLTQGTCSECGKEKTYRKYHLLKKDNKLFCITCYKRKYSQFTLQKVEFQNKTGLRNVAKLPKTYYTNAIRFASDTFSGFDEIIKKKQNRLNSIQNRLNFWKELLYNPSNRNEIKIKVVKYAPKTDTREHPHYYSEAEIKGRIKRLEKQLKKFKMPKYPEFTSETISLQRELYSWKNPDELKISSITDKNESMNYYGKEYLKRYIDLINSQTPQILLEKENNSFYLCFPITKNIEMPKIDDTFEPVGIDWGITRNIAVVSILDSKTKKPKFVKFYSAGYILGKRKHYKSLRKHFGQKKRQDKINKLGTKEDRFIDSNIHKLAFLIVKEIRNHSNKPIILMENITDNREEAEKSMRQNILLHSVKSRLQNYIAYKALWNNIPTNLVKPEHTSQICNRCGHQDRENRPKGSKLFKCVKCNYMSNADFNASINIARKFYIGEYEPFYKDNEKMKSGVNSISM

SEQ ID NO: 14 - protein, 534 aa, synthetic construct
  REGION 1..534: note = Description of Artificial Sequence: Synthetic polypeptide
LKLSEQENITTGVKFKLKLDKETSEGLNDYFDEYGKAINFAIKVIQKELAEDRFAGKVRLDENKKPLLNEDGKKIWDFPNEFCSCGKQVNRYVNGKSLCQECYKNKFTEYGIRKRMYSAKGRKAEQDINIKNSTNKISKTHFNYAIREAFILDKSIKKQRKERFRRLREMKKKLQEFIEIRDGNKILCPKIEKQRVERYIHPSWINKEKKLEDFRGYSMSNVLGKIKILDRNIKREEKSLKEKGQINFKARRLMLDKSVKFLNDNKISFTISKNLPKEYELDLPEKEKRLNWLKEKIKIIKNQKPKYAYLLRKDDNFYLQYTLETEFNLKEDYSGIVGIDRGVSHIAVYTFVHNNGKNERPLFLNSSEILRLKNLQKERDRFLRRKHNKKRKKSNMRNIEKKIQLILHNYSKQIVDFAKNKNAFIVFEKLEKPKKNRSKMSKKSQYKLSQFTFKKLSDLVDYKAKREGIKVLYISPEYTSKECSHCGEKVNTQRPFNGNSSLFKCNKCGVELNADYNASINIAKKGLNILNSTN

SEQ ID NO: 15 - protein, 537 aa, synthetic construct
  REGION 1..537: note = Description of Artificial Sequence: Synthetic polypeptide
MEESIITGVKFKLRIDKETTKKLNEYFDEYGKAINFAVKIIQKELADDRFAGKAKLDQNKNPILDENGKKIYEFPDEFCSCGKQVNKYVNNKPFCQECYKIRFTENGIRKRMYSAKGRKAEHKINILNSTNKISKTHFNYAIREAFILDKSIKKQRKKRNERLRESKKRLQQFIDMRDGKREICPTIKGQKVDRFIHPSWITKDKKLEDFRGYTLSIINSKIKILDRNIKREEKSLKEKGQIIFKAKRLMLDKSIRFVGDRKVLFTISKTLPKEYELDLPSKEKRLNWLKEKIEIIKNQKPKYAYLLRKNIESEKKPNYEYYLQYTLEIKPELKDFYDGAIGIDRGINHIAVCTFISNDGKVTPPKFFSSGEILRLKNLQKERDRFLLRKHNKNRKKGNMRVIENKINLILHRYSKQIVDMAKKLNASIVFEELGRIGKSRTKMKKSQRYKLSLFIFKKLSDLVDYKSRREGIRVTYVPPEYTSKECSHCGEKVNTQRPFNGNYSLFKCNKCGIQLNSDYNASINIAKKGLKIPNST

SEQ ID NO: 16 - protein, 540 aa, synthetic construct
  REGION 1..540: note = Description of Artificial Sequence: Synthetic polypeptide
LWTIVIGDFIEMPKQDLVTTGIKFKLDVDKETRKKLDDYFDEYGKAINFAVKIIQKNLKEDRFAGKIALGEDKKPLLDKDGKKIYNYPNESCSCGNQVRRYVNAKPFCVDCYKLKFTENGIRKRMYSARGRKADSDINIKNSTNKISKTHFNYAIREGFILDKSLKKQRSKRIKKLLELKRKLQEFIDIRQGQMVLCPKIKNQRVDKFIHPSWLKRDKKLEEFRGYSLSVVEGKIKIFNRNILREEDSLRQRGHVNFKANRIMLDKSVRFLDGGKVNFNLNKGLPKEYLLDLPKKENKLSWLNEKISLIKLQKPKYAYLLRREGSFFIQYTIENVPKTFSDYLGAIGIDRGISHIAVCTFVSKNGVNKAPVFFSSGEILKLKSLQKQRDLFLRGKHNKIRKKSNMRNIDNKINLILHKYSRNIVNLAKSEKAFIVFEKLEKIKKSRFKMSKSLQYKLSQFTFKKLSDLVEYKAKIEGIKVDYVPPEYTSKECSHCGEKVDTQRPFNGNSSLFKCNKCRVQLNADYNASINIAKKSLNISN

SEQ ID NO: 17 - protein, 542 aa, synthetic construct
  REGION 1..542: note = Description of Artificial Sequence: Synthetic polypeptide
MSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 18 - protein, 564 aa, synthetic construct
  REGION 1..564: note = Description of Artificial Sequence: Synthetic polypeptide
MARAKNQPYQKLTTTTGIKFKLDLSEEEGKRFDEYFSEYAKAVNFCAKVIYQLRKNLKFAGKKELAAKEWKFEISNCDFCNKQKEIYYKNIANGQKVCKGCHRTNFSDNAIRKKMIPVKGRKVESKFNIHNTTKKISGTHRHWAFEDAADIIESMDKQRKEKQKRLRREKRKLSYFFELFGDPAKRYELPKVGKQRVPRYLHKIIDKDSLTKKRGYSLSYIKNKIKISERNIERDEKSLRKASPIAFGARKIKMSKLDPKRAFDLENNVFKIPGKVIKGQYKFFGTNVANEHGKKFYKDRISKILAGKPKYFYLLRKKVAESDGNPIFEYYVQWSIDTETPAITSYDNILGIDAGITNLATTVLIPKNLSAEHCSHCGNNHVKPIFTKFFSGKELKAIKIKSRKQKYFLRGKHNKLVKIKRIRPIEQKVDGYCHVVSKQIVEMAKERNSCIALEKLEKPKKSKFRQRRREKYAVSMFVFKKLATFIKYKAAREGIEIIPVEPEGTSYTCSHCKNAQNNQRPYFKPNSKKSWTSMFKCGKCGIELNSDYNAAFNIAQKALNMTSA

SEQ ID NO: 19 - protein, 610 aa, synthetic construct
  REGION 1..610: note = Description of Artificial Sequence: Synthetic polypeptide
MDEKHFFCSYCNKELKISKNLINKISKGSIREDEAVSKAISIHNKKEHSLILGIKFKLFIENKLDKKKLNEYFDNYSKAVTFAARIFDKIRSPYKFIGLKDKNTKKWTFPKAKCVFCLEEKEVAYANEKDNSKICTECYLKEFGENGIRKKIYSTRGRKVEPKYNIFNSTKELSSTHYNYAIRDAFQLLDALKKQRQKKLKSIFNQKLRLKEFEDIFSDPQKRIELSLKPHQREKRYIHLSKSGQESINRGYTLRFVRGKIKSLTRNIEREEKSLRKKTPIHFKGNRLMIFPAGIKFDFASNKVKISISKNLPNEFNFSGTNVKNEHGKSFFKSRIELIKTQKPKYAYVLRKIKREYSKLRNYEIEKIRLENPNADLCDFYLQYTIETESRNNEEINGIIGIDRGITNLACLVLLKKGDKKPSGVKFYKGNKILGMKIAYRKHLYLLKGKRNKLRKQRQIRAIEPKINLILHQISKDIVKIAKEKNFAIALEQLEKPKKARFAQRKKEKYKLALFTFKNLSTLIEYKSKREGIPVIYVPPEKTSQMCSHCAINGDEHVDTQRPYKKPNAQKPSYSLFKCNKCGIELNADYNAAFNIAQKGLKTLMLNHSH

SEQ ID NO: 20 - protein, 369 aa, synthetic construct
  REGION 1..369: note = Description of Artificial Sequence: Synthetic polypeptide
MLQTLLVKLDPSKEQYKMLYETMERFNEACNQIAETVFAIHSANKIEVQKTVYYPIREKFGLSAQLTILAIRKVCEAYKRDKSIKPEFRLDGALVYDQRVLSWKGLDKVSLVTLQGRQIIPIKFGDYQKARMDRIRGQADLILVKGVFYLCVVVEVSEESPYDPKGVLGVDLGIKNLAVDSDGEVHSGEQTTNTRERLDSLKARLQSKGTKSAKRHLKKLSGRMAKFSKDVNHCISKKLVAKAKGTLMSIALEDLQGIRDRVTVRKAQRRNLHTWNFGLLRMFVDYKAKIAGVPLVFVDPRNTSRTCPSCGHVAKANRPTRDEFRCVSCGFAGAADHIAAMNIAFRAEVSQPIVTRFFVQSQAPSFRVG

SEQ ID NO: 21 - protein, 552 aa, synthetic construct
  REGION 1..552: note = Description of Artificial Sequence: Synthetic polypeptide
MDEEPDSAEPNLAPISVKLKLVKLDGEKLAALNDYFNEYAKAVNFCELKMQKIRKNLVNIRGTYLKEKKAWINQTGECCICKKIDELRCEDKNPDINGKICKKCYNGRYGNQMIRKLFVSTNKRAVPKSLDIRKVARLHNTHYHRIPPEAADIIKAIETAERKRRNRILFDERRYNELKDALENEEKRVARPKKPKEREVRYVPISKKDTPSKGYTMNALVRKVSGMAKKIERAKRNLNKRKKIEYLGRRILLDKNWVRFDFDKSEISIPTMKEFFGEMRFEITGPSNVMSPNGREYFTKWFDRIKAQPDNYCYLLRKESEDETDFYLQYTWRPDAHPKKDYTGCLGIDIGGSKLASAVYFDADKNRAKQPIQIFSNPIGKWKTKRQKVIKVLSKAAVRHKTKKLESLRNIEPRIDVHCHRIARKIVGMALAANAFISMENLEGGIREKQKAKETKKQKFSRNMFVFRKLSKLIEYKALMEGVKVVYIVPDYTSQLCSSCGTNNTKRPKQAIFMCQNTECRYFGKNINADFNAAINIAKKALNRKDIVRELS

SEQ ID NO: 22 - protein, 534 aa, synthetic construct
  REGION 1..534: note = Description of Artificial Sequence: Synthetic polypeptide
MEKNNSEQTSITTGIKFKLKLDKETKEKLNNYFDEYGKAINFAVRIIQMQLNDDRLAGKYKRDEKGKPILGEDGKKILEIPNDFCSCGNQVNHYVNGVSFCQECYKKRFSENGIRKRMYSAKGRKAEQDINIKNSTNKISKTHFNYAIREAFNLDKSIKKQREKRFKKLKDMKRKLQEFLEIRDGKRVICPKIEKQKVERYIHPSWINKEKKLEEFRGYSLSIVNSKIKSFDRNIQREEKSLKEKGQINFKAQRLMLDKSVKFLKDNKVSFTISKELPKTFELDLPKKEKKLNWLNEKLEIIKNQKPKYAYLLRKENNIFLQYTLDSIPEIHSEYSGAVGIDRGVSHIAVYTFLDKDGKNERPFFLSSSGILRLKNLQKERDKFLRKKHNKIRKKGNMRNIEQKINLILHEYSKQIVNFAKDKNAFIVFELLEKPKKSRERMSKKIQYKLSQFTFKKLSDLVDYKAKREGIKVIYVEPAYTSKDCSHCGERVNTQRPFNGNFSLFKCNKCGIVLNSDYNASLNIARKGLNISAN

SEQ ID NO: 23 - protein, 577 aa, synthetic construct
  REGION 1..577: note = Description of Artificial Sequence: Synthetic polypeptide
MAEEKFFFCEKCNKDIKIPKNYINKQGAEEKARAKHEHRVHALILGIKFKIYPKKEDISKLNDYFDEYAKAVTFTAKIVDKLKAPFLFAGKRDKDTSKKKWVFPVDKCSFCKEKTEINYRTKQGKNICNSCYLTEFGEQGLLEKIYATKGRKVSSSFNLFNSTKKLTGTHNNYVVKESLQLLDALKKQRSKRLKKLSNTRRKLKQFEEMFEKEDKRFQLPLKEKQRELRFIHVSQKDRATEFKGYTMNKIKSKIKVLRRNIEREQRSLNRKSPVFFRGTRIRLSPSVQFDDKDNKIKLTLSKELPKEYSFSGLNVANEHGRKFFAEKLKLIKENKSKYAYLLRRQVNKNNKKPIYDYYLQYTVEFLPNIITNYNGILGIDRGINTLACIVLLENKKEKPSFVKFFSGKGILNLKNKRRKQLYFLKGVHNKYRKQQKIRPIEPRIDQILHDISKQIIDLAKEKRVAISLEQLEKPQKPKFRQSRKAKYKLSQFNFKTLSNYIDYKAKKEGIRVIYIAPEMTSQNCSRCAMKNDLHVNTQRPYKNTSSLFKCNKCGVELNADYNAAFNIAQKGLKILNS

SEQ ID NO: 24 - protein, 613 aa, synthetic construct
  REGION 1..613: note = Description of Artificial Sequence: Synthetic polypeptide
MISLKLKLLPDEEQKKLLDEMFWKWASICTRVGFGRADKEDLKPPKDAEGVWFSLTQLNQANTDINDLREAMKHQKHRLEYEKNRLEAQRDDTQDALKNPDRREISTKRKDLFRPKASVEKGFLKLKYHQERYWVRRLKEINKLIERKTKTLIKIEKGRIKFKATRITLHQGSFKIRFGDKPAFLIKALSGKNQIDAPFVVVPEQPICGSVVNSKKYLDEITTNFLAYSVNAMLFGLSRSEEMLLKAKRPEKIKKKEEKLAKKQSAFENKKKELQKLLGRELTQQEEAIIEETRNQFFQDFEVKITKQYSELLSKIANELKQKNDFLKVNKYPILLRKPLKKAKSKKINNLSPSEWKYYLQFGVKPLLKQKSRRKSRNVLGIDRGLKHLLAVTVLEPDKKTFVWNKLYPNPITGWKWRRRKLLRSLKRLKRRIKSQKHETIHENQTRKKLKSLQGRIDDLLHNISRKIVETAKEYDAVIVVEDLQSMRQHGRSKGNRLKTLNYALSLFDYANVMQLIKYKAGIEGIQIYDVKPAGTSQNCAYCLLAQRDSHEYKRSQENSKIGVCLNPNCQNHKKQIDADLNAARVIASCYALKINDSQPFGTRKRFKKRTTN

SEQ ID NO: 25 - protein, 615 aa, synthetic construct
  REGION 1..615: note = Description of Artificial Sequence: Synthetic polypeptide
METLSLKLKLNPSKEQLLVLDKMFWKWASICTRLGLKKAEMSDLEPPKDAEGVWFSKTQLNQANTDVNDLRKAMQHQGKRIEYELDKVENRRNEIQEMLEKPDRRDISPNRKDLFRPKAAVEKGYLKLKYHKLGYWSKELKTANKLIERKRKTLAKIDAGKMKFKPTRISLHTNSFRIKFGEEPKIALSTTSKHEKIELPLITSLQRPLKTSCAKKSKTYLDAAILNFLAYSTNAALFGLSRSEEMLLKAKKPEKIEKRDRKLATKRESFDKKLKTLEKLLERKLSEKEKSVFKRKQTEFFDKFCITLDETYVEALHRIAEELVSKNKYLEIKKYPVLLRKPESRLRSKKLKNLKPEDWTYYIQFGFQPLLDTPKPIKTKTVLGIDRGVRHLLAVSIFDPRTKTFTFNRLYSNPIVDWKWRRRKLLRSIKRLKRRLKSEKHVHLHENQFKAKLRSLEGRIEDHFHNLSKEIVDLAKENNSVIVVENLGGMRQHGRGRGKWLKALNYALSHFDYAKVMQLIKYKAELAGVFVYDVAPAGTSINCAYCLLNDKDASNYTRGKVINGKKNTKIGECKTCKKEFDADLNAARVIALCYEKRLNDPQPFGTRKQFKPKKP

SEQ ID NO: 26 - protein, 775 aa, synthetic construct
  REGION 1..775: note = Description of Artificial Sequence: Synthetic polypeptide
MKALKLQLIPTRKQYKILDEMFWKWASLANRVSQKGESKETLAPKKDIQKIQFNATQLNQIEKDIKDLRGAMKEQQKQKERLLLQIQERRSTISEMLNDDNNKERDPHRPLNFRPKGWRKFHTSKHWVGELSKILRQEDRVKKTIERIVAGKISFKPKRIGIWSSNYKINFFKRKISINPLNSKGFELTLMTEPTQDLIGKNGGKSVLNNKRYLDDSIKSLLMFALHSRFFGLNNTDTYLLGGKINPSLVKYYKKNQDMGEFGREIVEKFERKLKQEINEQQKKIIMSQIKEQYSNRDSAFNKDYLGLINEFSEVFNQRKSERAEYLLDSFEDKIKQIKQEIGESLNISDWDFLIDEAKKAYGYEEGFTEYVYSKRYLEILNKIVKAVLITDIYFDLRKYPILLRKPLDKIKKISNLKPDEWSYYIQFGYDSINPVQLMSTDKFLGIDRGLTHLLAYSVFDKEKKEFIINQLEPNPIMGWKWKLRKVKRSLQHLERRIRAQKMVKLPENQMKKKLKSIEPKIEVHYHNISRKIVNLAKDYNASIVVESLEGGGLKQHGRKKNARNRSLNYALSLFDYGKIASLIKYKADLEGVPMYEVLPAYTSQQCAKCVLEKGSFVDPEIIGYVEDIGIKGSLLDSLFEGTELSSIQVLKKIKNKIELSARDNHNKEINLILKYNFKGLVIVRGQDKEEIAEHPIKEINGKFAILDFVYKRGKEKVGKKGNQKVRYTGNKKVGYCSKHGQVDADLNASRVIALCKYLDINDPILFGEQRKSFK

SEQ ID NO: 27 - protein, 777 aa, synthetic construct
  REGION 1..777: note = Description of Artificial Sequence: Synthetic polypeptide
MVTRAIKLKLDPTKNQYKLLNEMFWKWASLANRFSQKGASKETLAPKDGTQKIQFNATQLNQIKKDVDDLRGAMEKQGKQKERLLIQIQERLLTISEILRDDSKKEKDPHRPQNFRPFGWRRFHTSAYWSSEASKLTRQVDRVRRTIERIKAGKINFKPKRIGLWSSTYKINFLKKKINISPLKSKSFELDLITEPQQKIIGKEGGKSVANSKKYLDDSIKSLLIFAIKSRLFGLNNKDKPLFENIITPNLVRYHKKGQEQENFKKEVIKKFENKLKKEISQKQKEIIFSQIERQYENRDATFSEDYLRAISEFSEIFNQRKKERAKELLNSFNEKIRQLKKEVNGNISEEDLKILEVEAEKAYNYENGFIEWEYSEQFLGVLEKIARAVLISDNYFDLKKYPILIRKPTNKSKKITNLKPEEWDYYIQFGYGLINSPMKIETKNFMGIDRGLTHLLAYSIFDRDSEKFTINQLELNPIKGWKWKLRKVKRSLQHLERRMRAQKGVKLPENQMKKRLKSIEPKIESYYHNLSRKIVNLAKANNASIVVESLEGGGLKQHGRKKNSRHRALNYALSLFDYGKIASLIKYKSDLEGVPMYEVLPAYTSQQCAKCVLKKGSFVEPEIIGYIEEIGFKENLLTLLFEDTGLSSVQVLKKSKNKMTLSARDKEGKMVDLVLKYNFKGLVISQEKKKEEIVEFPIKEIDGKFAVLDSAYKRGKERISKKGNQKLVYTGNKKVGYCSVHGQVDADLNASRVIALCKYLGINEPIVFGEQRKSFK

SEQ ID NO: 28 - protein, 610 aa, synthetic construct
  REGION 1..610: note = Description of Artificial Sequence: Synthetic polypeptide
LDLITEPIQPHKSSSLRSKEFLEYQISDFLNFSLHSLFFGLASNEGPLVDFKIYDKIVIPKPEERFPKKESEEGKKLDSFDKRVEEYYSDKLEKKIERKLNTEEKNVIDREKTRIWGEVNKLEEIRSIIDEINEIKKQKHISEKSKLLGEKWKKVNNIQETLLSQEYVSLISNLSDELTNKKKELLAKKYSKFDDKIKKIKEDYGLEFDENTIKKEGEKAFLNPDKFSKYQFSSSYLKLIGEIARSLITYKGFLDLNKYPIIFRKPINKVKKIHNLEPDEWKYYIQFGYEQINNPKLETENILGIDRGLTHILAYSVFEPRSSKFILNKLEPNPIEGWKWKLRKLRRSIQNLERRWRAQDNVKLPENQMKKNLRSIEDKVENLYHNLSRKIVDLAKEKNACIVFEKLEGQGMKQHGRKKSDRLRGLNYKLSLFDYGKIAKLIKYKAEIEGIPIYRIDSAYTSQNCAKCVLESRRFAQPEEISCLDDFKEGDNLDKRILEGTGLVEAKIYKKLLKEKKEDFEIEEDIAMFDTKKVIKENKEKTVILDYVYTRRKEIIGTNHKKNIKGIAKYTGNTKIGYCMKHGQVDADLNASRTIALCKNFDINNPEIWK

SEQ ID NO: 29 - protein, 632 aa, synthetic construct
  REGION 1..632: note = Description of Artificial Sequence: Synthetic polypeptide
MSDESLVSSEDKLAIKIKIVPNAEQAKMLDEMFKKWSSICNRISRGKEDIETLRPDEGKELQFNSTQLNSATMDVSDLKKAMARQGERLEAEVSKLRGRYETIDASLRDPSRRHTNPQKPSSFYPSDWDISGRLTPRFHTARHYSTELRKLKAKEDKMLKTINKIKNGKIVFKPKRITLWPSSVNMAFKGSRLLLKPFANGFEMELPIVISPQKTADGKSQKASAEYMRNALLGLAGYSINQLLFGMNRSQKMLANAKKPEKVEKFLEQMKNKDANFDKKIKALEGKWLLDRKLKESEKSSIAVVRTKFFKSGKVELNEDYLKLLKHMANEILERDGFVNLNKYPILSRKPMKRYKQKNIDNLKPNMWKYYIQFGYEPIFERKASGKPKNIMGIDRGLTHLLAVAVFSPDQQKFLFNHLESNPIMHWKWKLRKIRRSIQHMERRIRAEKNKHIHEAQLKKRLGSIEEKTEQHYHIVSSKIINWAIEYEAAIVLESLSHMKQRGGKKSVRTRALNYALSLFDYEKVARLITYKARIRGIPVYDVLPGMTSKTCATCLLNGSQGAYVRGLETTKAAGKATKRKNMKIGKCMVCNSSENSMIDADLNAARVIAICKYKNLNDPQPAGSRKVFKRF

SEQ ID NO: 30 - protein, 625 aa, synthetic construct
  REGION 1..625: note = Description of Artificial Sequence: Synthetic polypeptide
MLALKLKIMPTEKQAEILDAMFWKWASICSRIAKMKKKVSVKENKKELSKKIPSNSDIWFSKTQLCQAEVDVGDHKKALKNFEKRQESLLDELKYKVKAINEVINDESKREIDPNNPSKFRIKDSTKKGNLNSPKFFTLKKWQKILQENEKRIKKKESTIEKLKRGNIFFNPTKISLHEEEYSINFGSSKLLLNCFYKYNKKSGINSDQLENKFNEFQNGLNIICSPLQPIRGSSKRSFEFIRNSIINFLMYSLYAKLFGIPRSVKALMKSNKDENKLKLEEKLKKKKSSFNKTVKEFEKMIGRKLSDNESKILNDESKKFFEIIKSNNKYIPSEEYLKLLKDISEEIYNSNIDFKPYKYSILIRKPLSKFKSKKLYNLKPTDYKYYLQLSYEPFSKQLIATKTILGIDRGLKHLLAVSVFDPSQNKFVYNKLIKNPVFKWKKRYHDLKRSIRNRERRIRALTGVHIHENQLIKKLKSMKNKINVLYHNVSKNIVDLAKKYESTIVLERLENLKQHGRSKGKRYKKLNYVLSNFDYKKIESLISYKAKKEGVPVSNINPKYTSKTCAKCLLEVNQLSELKNEYNRDSKNSKIGICNIHGQIDADLNAARVIALCYSKNLNEPHFK

SEQ ID NO: 31 - protein, 517 aa, synthetic construct
  REGION 1..517: note = Description of Artificial Sequence: Synthetic polypeptide
VINLFGYKFALYPNKTQEELLNKHLGECGWLYNKAIEQNEYYKADSNIEEAQKKFELLPDKNSDEAKVLRGNISKDNYVYRTLVKKKKSEINVQIRKAVVLRPAETIRNLAKVKKKGLSVGRLKFIPIREWDVLPFKQSDQIRLEENYLILEPYGRLKFKMHRPLLGKPKTFCIKRTATDRWTISFSTEYDDSNMRKNDGGQVGIDVGLKTHLRLSNENPDEDPRYPNPKIWKRYDRRLTILQRRISKSKKLGKNRTRLRLRLSRLWEKIRNSRADLIQNETYEILSENKLIAIEDLNVKGMQEKKDKKGRKGRTRAQEKGLHRSISDAAFSEFRRVLEYKAKRFGSEVKPVSAIDSSKECHNCGNKKGMPLESRIYECPKCGLKIDRDLNSAKVILARATGVRPGSNARADTKISATAGASVQTEGTVSEDFRQQMETSDQKPMQGEGSKEPPMNPEHKSSGRGSKHVNIGCKNKVGLYNEDENSRSTEKQIMDENRSTTEDMVEIGALHSPVLTT

SEQ ID NO: 32 - protein, 410 aa, synthetic construct
  REGION 1..410: note = Description of Artificial Sequence: Synthetic polypeptide
MIASIDYEAVSQALIVFEFKAKGKDSQYQAIDEAIRSYRFIRNSCLRYWMDNKKVGKYDLNKYCKVLAKQYPFANKLNSQARQSAAECSWSAISRFYDNCKRKVSGKKGFPKFKKHARSVEYKTSGWKLSENRKAITFTDKNGIGKLKLKGTYDLHFSQLEDMKRVRLVRRADGYYVQFCISVDVKVETEPTGKAIGLDVGIKYFLADSSGNTIENPQFYRKAEKKLNRANRRKSKKYIRGVKPQSKNYHKARCRYARKHLRVSRQRKEYCKRVAYCVIHSNDVVAYEDLNVKGMVKNRHLAKSISDVAWSTFRHWLEYFAIKYGKLTIPVAPHNTSQNCSNCDKKVPKSLSTRTHICHHCGYSEDRDVNAAKNILKKALSTVGQTGSLKLGEIEPLLVLEQSCTRKFDL

SEQ ID NO: 33 - protein, 486 aa, synthetic construct
  REGION 1..486: note = Description of Artificial Sequence: Synthetic polypeptide
LAEENTLHLTLAMSLPLNDLPENRTRSELWRRQWLPQKKLSLLLGVNQSVRKAAADCLRWFEPYQELLWWEPTDPDGKKLLDKEGRPIKRTAGHMRVLRKLEEIAPFRGYQLGSAVKNGLRHKVADLLLSYAKRKLDPQFTDKTSYPSIGDQFPIVWTGAFVCYEQSITGQLYLYLPLFPRGSHQEDITNNYDPDRGPALQVFGEKEIARLSRSTSGLLLPLQFDKWGEATFIRGENNPPTWKATHRRSDKKWLSEVLLREKDFQPKRVELLVRNGRIFVNVACEIPTKPLLEVENFMGVSFGLEHLVTVVVINRDGNVVHQRQEPARRYEKTYFARLERLRRRGGPFSQELETFHYRQVAQIVEEALRFKSVPAVEQVGNIPKGRYNPRLNLRLSYWPFGKLADLTSYKAVKEGLPKPYSVYSATAKMLCSTCGAANKEGDQPISLKGPTVYCGNCGTRHNTGFNTALNLARRAQELFVKGVVAR

SEQ ID NO: 34 - protein, 602 aa, synthetic construct
  REGION 1..602: note = Description of Artificial Sequence: Synthetic polypeptide
MSQSLLKWHDMAGRDKDASRSLQKSAVEGVLLHLTASHRVALEMLEKSVSQTVAVTMEAAQQRLVIVLEDDPTKATSRKRVISADLQFTREEFGSLPNWAQKLASTCPEIATKYADKHINSIRIAWGVAKESTNGDAVEQKLQWQIRLLDVTMFLQQLVLQLADKALLEQIPSSIRGGIGQEVAQQVTSHIQLLDSGTVLKAELPTISDRNSELARKQWEDAIQTVCTYALPFSRERARILDPGKYAAEDPRGDRLINIDPMWARVLKGPTVKSLPLLFVSGSSIRIVKLTLPRKHAAGHKHTFTATYLVLPVSREWINSLPGTVQEKVQWWKKPDVLATQELLVGKGALKKSANTLVIPISAGKKRFFNHILPALQRGFPLQWQRIVGRSYRRPATHRKWFAQLTIGYTNPSSLPEMALGIHFGMKDILWWALADKQGNILKDGSIPGNSILDFSLQEKGKIERQQKAGKNVAGKKYGKSLLNATYRVVNGVLEFSKGISAEHASQPIGLGLETIRFVDKASGSSPVNARHSNWNYGQLSGIFANKAGPAGFSVTEITLKKAQRDLSDAEQARVLAIEATKRFASRIKRLATKRKDDTLFV

SEQ ID NO: 35 - protein, 494 aa, synthetic construct
  REGION 1..494: note = Description of Artificial Sequence: Synthetic polypeptide
VEPVEKERFYYRTYTFRLDGQPRTQNLTTQSGWGLLTKAVLDNTKHYWEIVHHARIANQPIVFENPVIDEQGNPKLNKLGQPRFWKRPISDIVNQLRALFENQNPYQLGSSLIQGTYWDVAENLASWYALNKEYLAGTATWGEPSFPEPHPLTEINQWMPLTFSSGKVVRLLKNASGRYFIGLPILGENNPCYRMRTIEKLIPCDGKGRVTSGSLILFPLVGIYAQQHRRMTDICESIRTEKGKLAWAQVSIDYVREVDKRRRMRRTRKSQGWIQGPWQEVFILRLVLAHKAPKLYKPRCFAGISLGPKTLASCVILDQDERVVEKQQWSGSELLSLIHQGEERLRSLREQSKPTWNAAYRKQLKSLINTQVFTIVTFLRERGAAVRLESIARVRKSTPAPPVNFLLSHWAYRQITERLKDLAIRNGMPLTHSNGSYGVRFTCSQCGATNQGIKDPTKYKVDIESETFLCSICSHREIAAVNTATNLAKQLLDE

SEQ ID NO: 36 - protein, 526 aa, synthetic construct
  REGION 1..526: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANHEIVEQPHHSLTR

SEQ ID NO: 37 - protein, 649 aa, synthetic construct
  REGION 1..649: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGRHGHTRTDRLPAGNTLWRADFATSAEVAAPKWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANRRFSMWNYSRLATLIEWLGTDIATRDCGTAAPLAHKVSDYLTHFTCPECGACRKAGQKKEIADTVRAGDILTCRKCGFSGPIPDNFIAEFVAKKALERMLKKKPV

SEQ ID NO: 38 - protein, 414 aa, synthetic construct
  REGION 1..414: note = Description of Artificial Sequence: Synthetic polypeptide
MAKRNFGEKSEALYRAVRFEVRPSKEELSILLAVSEVLRMLFNSALAERQQVFTEFIASLYAELKSASVPEEISEIRKKLREAYKEHSISLFDQINALTARRVEDEAFASVTRNWQEETLDALDGAYKSFLSLRRKGDYDAHSPRSRDSGFFQKIPGRSGFKIGEGRIALSCGAGRKLSFPIPDYQQGRLAETTKLKKFELYRDQPNLAKSGRFWISVVYELPKPEATTCQSEQVAFVALGASSIGVVSQRGEEVIALWRSDKHWVPKIEAVEERMKRRVKGSRGWLRLLNSGKRRMHMISSRQHVQDEREIVDYLVRNHGSHFVVTELVVRSKEGKLADSSKPERGGSLGLNWAAQNTGSLSRLVRQLEEKVKEHGGSVRKHKLTLTEAPPARGAENKLWMARKLRESFLKEV

SEQ ID NO: 39 - protein, 413 aa, synthetic construct
  REGION 1..413: note = Description of Artificial Sequence: Synthetic polypeptide
LAKNDEKELLYQSVKFEIYPDESKIRVLTRVSNILVLVWNSALGERRARFELYIAPLYEELKKFPRKSAESNALRQKIREGYKEHIPTFFDQLKKLLTPMRKEDPALLGSVPRAYQEETLNTLNGSFVSFMTLRRNNDMDAKPPKGRAEDRFHEISGRSGFKIDGSEFVLSTKEQKLRFPIPNYQLEKLKEAKQIKKFTLYQSRDRRFWISIAYEIELPDQRPFNPEEVIYIAFGASSIGVISPEGEKVIDFWRPDKHWKPKIKEVENRMRSCKKGSRAWKKRAAARRKMYAMTQRQQKLNHREIVASLLRLGFHFVVTEYTVRSKPGKLADGSNPKRGGAPQGFNWSAQNTGSFGEFILWLKQKVKEQGGTVQTFRLVLGQSERPEKRGRDNKIEMVRLLREKYLESQTIVV

SEQ ID NO: 40 - protein, 449 aa, synthetic construct
  REGION 1..449: note = Description of Artificial Sequence: Synthetic polypeptide
MAKGKKKEGKPLYRAVRFEIFPTSDQITLFLRVSKNLQQVWNEAWQERQSCYEQFFGSIYERIGQAKKRAQEAGFSEVWENEAKKGLNKKLRQQEISMQLVSEKESLLQELSIAFQEHGVTLYDQINGLTARRIIGEFALIPRNWQEETLDSLDGSFKSFLALRKNGDPDAKPPRQRVSENSFYKIPGRSGFKVSNGQIYLSFGKIGQTLTSVIPEFQLKRLETAIKLKKFELCRDERDMAKPGRFWISVAYEIPKPEKVPVVSKQITYLAIGASRLGVVSPKGEFCLNLPRSDYHWKPQINALQERLEGVVKGSRKWKKRMAACTRMFAKLGHQQKQHGQYEVVKKLLRHGVHFVVTELKVRSKPGALADASKSDRKGSPTGPNWSAQNTGNIARLIQKLTDKASEHGGTVIKRNPPLLSLEERQLPDAQRKIFIAKKLREEFLADQK

SEQ ID NO: 41 - protein, 711 aa, synthetic construct
  REGION 1..711: note = Description of Artificial Sequence: Synthetic polypeptide
MAKREKKDDVVLRGTKMRIYPTDRQVTLMDMWRRRCISLWNLLLNLETAAYGAKNTRSKLGWRSIWARVVEENHAKALIVYQHGKCKKDGSFVLKRDGTVKHPPRERFPGDRKILLGLFDALRHTLDKGAKCKCNVNQPYALTRAWLDETGHGARTADIIAWLKDFKGECDCTAISTAAKYCPAPPTAELLTKIKRAAPADDLPVDQAILLDLFGALRGGLKQKECDHTHARTVAYFEKHELAGRAEDILAWLIAHGGTCDCKIVEEAANHCPGPRLFIWEHELAMIMARLKAEPRTEWIGDLPSHAAQTVVKDLVKALQTMLKERAKAAAGDESARKTGFPKFKKQAYAAGSVYFPNTTMFFDVAAGRVQLPNGCGSMRCEIPRQLVAELLERNLKPGLVIGAQLGLLGGRIWRQGDRWYLSCQWERPQPTLLPKTGRTAGVKIAASIVFTTYDNRGQTKEYPMPPADKKLTAVHLVAGKQNSRALEAQKEKEKKLKARKERLRLGKLEKGHDPNALKPLKRPRVRRSKLFYKSAARLAACEAIERDRRDGFLHRVTNEIVHKFDAVSVQKMSVAPMMRRQKQKEKQIESKKNEAKKEDNGAAKKPRNLKPVRKLLRHVAMARGRQFLEYKYNDLRGPGSVLIADRLEPEVQECSRCGTKNPQMKDGRRLLRCIGVLPDGTDCDAVLPRNRNAARNAEKRLRKHREAHNA

SEQ ID NO: 42 - protein, 574 aa, synthetic construct
  REGION 1..574: note = Description of Artificial Sequence: Synthetic polypeptide
MNEVLPIPAVGEDAADTIMRGSKMRIYPSVRQAATMDLWRRRCIQLWNLLLELEQAAYSGENRRTQIGWRSIWATVVEDSHAEAVRVAREGKKRKDGTFRKAPSGKEIPPLDPAMLAKIQRQMNGAVDVDPKTGEVTPAQPRLFMWEHELQKIMARLKQAPRTHWIDDLPSHAAQSVVKDLIKALQAMLRERKKRASGIGGRDTGFPKFKKNRYAAGSVYFANTQLRFEAKRGKAGDPDAVRGEFARVKLPNGVGWMECRMPRHINAAHAYAQATLMGGRIWRQGENWYLSCQWKMPKPAPLPRAGRTAAIKIAAAIPITTVDNRGQTREYAMPPIDRERIAAHAAAGRAQSRALEARKRRAKKREAYAKKRHAKKLERGIAAKPPGRARIKLSPGFYAAAAKLAKLEAEDANAREAWLHEITTQIVRNFDVIAVPRMEVAKLMKKPEPPEEKEEQVKAPWQGKRRSLKAARVMMRRTAMALIQTTLKYKAVDLRGPQAYEEIAPLDVTAAACSGCGVLKPEWKMARAKGREIMRCQEPLPGGKTCNTVLTYTRNSARVIGRELAVRLAERQKA

SEQ ID NO: 43 - protein, 400 aa, synthetic construct
  REGION 1..400: note = Description of Artificial Sequence: Synthetic polypeptide
MTTQKTYNFCFYDQRFFELSKEAGEVYSRSLEEFWKIYDETGVWLSKFDLQKHMRNKLERKLLHSDSFLGAMQQVHANLASWKQAKKVVPDACPPRKPKFLQAILFKKSQIKYKNGFLRLTLGTEKEFLYLKWDINIPLPIYGSVTYSKTRGWKINLCLETEVEQKNLSENKYLSIDLGVKRVATIFDGENTITLSGKKFMGLMHYRNKLNGKTQSRLSHKKKGSNNYKKIQRAKRKTTDRLLNIQKEMLHKYSSFIVNYAIRNDIGNIIIGDNSSTHDSPNMRGKTNQKISQNPEQKLKNYIKYKFESISGRVDIVPEPYTSRKCPHCKNIKKSSPKGRTYKCKKCGFIFDRDGVGAINIYNENVSFGQIISPGRIRSLTEPIGMKFHNEIYFKSYVAA

SEQ ID NO: 44 - protein, 743 aa, synthetic construct
  REGION 1..743: note = Description of Artificial Sequence: Synthetic polypeptide
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 45 - protein, 769 aa, synthetic construct
  REGION 1..769: note = Description of Artificial Sequence: Synthetic polypeptide
MHLWRTHCVFNQRLPALLKRLFAMRRGEVGGNEAQRQVYQRVAQFVLARDAKDSVDLLNAVSLRKRSANSAFKKKATISCNGQAREVTGEEVFAEAVALASKGVFAYDKDDMRAGLPDSLFQPLTRDAVACMRSHEELVATWKKEYREWRDRKSEWEAEPEHALYLNLRPKFEEGEAARGGRFRKRAERDHAYLDWLEANPQLAAWRRKAPPAVVPIDEAGKRRIARAKAWKQASVRAEEFWKRNPELHALHKIHVQYLREFVRPRRTRRNKRREGFKQRPTFTMPDPVRHPRWCLFNAPQTSPQGYRLLRLPQSRRTVGSVELRLLTGPSDGAGFPDAWVNVRFKADPRLAQLRPVKVPRTVTRGKNKGAKVEADGFRYYDDQLLIERDAQVSGVKLLFRDIRMAPFADKPIEDRLLSATPYLVFAVEIKDEARTERAKAIRFDETSELTKSGKKRKTLPAGLVSVAVDLDTRGVGFLTRAVIGVPEIQQTHHGVRLLQSRYVAVGQVEARASGEAEWSPGPDLAHIARHKREIRRLRQLRGKPVKGERSHVRLQAHIDRMGEDRFKKAARKIVNEALRGSNPAAGDPYTRADVLLYESLETLLPDAERERGINRALLRWNRAKLIEHLKRMCDDAGIRHFPVSPFGTSQVCSKCGALGRRYSLARENGRAVIRFGWVERLFACPNPECPGRRPDRPDRPFTCNSDHNASVNLHRVFALGDQAVAAFRALAPRDSPARTLAVKRVEDTLRPQLMRVHKLADAGVDSPF

SEQ ID NO: 46 - protein, 666 aa, synthetic construct
  REGION 1..666: note = Description of Artificial Sequence: Synthetic polypeptide
MATLVYRYGVRAHGSARQQDAVVSDPAMLEQLRLGHELRNALVGVQHRYEDGKRAVWSGFASVAAADHRVTTGETAVAELEKQARAEHSADRTAATRQGTAESLKAARAAVKQARADRKAAMAAVAEQAKPKIQALGDDRDAEIKDLYRRFCQDGVLLPRCGRCAGDLRSDGDCTDCGAAHEPRKLYWATYNAIREDHQTAVKLVEAKRKAGQPARLRFRRWTGDGTLTVQLQRMHGPACRCVTCAEKLTRRARKTDPQAPAVAADPAYPPTDPPRDPALLASGQGKWRNVLQLGTWIPPGEWSAMSRAERRRVGRSHIGWQLGGGRQLTLPVQLHRQMPADADVAMAQLTRVRVGGRHRMSVALTAKLPDPPQVQGLPPVALHLGWRQRPDGSLRVATWACPQPLDLPPAVADVVVSHGGRWGEVIMPARWLADAEVPPRLLGRRDKAMEPVLEALADWLEAHTEACTARMTPALVRRWRSQGRLAGLTNRWRGQPPTGSAEILTYLEAWRIQDKLLWERESHLRRRLAARRDDAWRRVASWLARHAGVLVVDDADIAELRRRDDPADTDPTMPASAAQAARARAALAAPGRLRHLATITATRDGLGVHTVASAGLTRLHRKCGHQAQPDPRYAASAVVTCPGCGNGYDQDYNAAMLMLDRQQQP

SEQ ID NO: 47 - protein, 564 aa, synthetic construct
  REGION 1..564: note = Description of Artificial Sequence: Synthetic polypeptide
MSRVELHRAYKFRLYPTPAQVAELAEWERQLRRLYNLAHSQRLAAMQRHVRPKSPGVLKSECLSCGAVAVAEIGTDGKAKKTVKHAVGCSVLECRSCGGSPDAEGRTAHTAACSFVDYYRQGREMTQLLEEDDQLARVVCSARQETLRDLEKAWQRWHKMPGFGKPHFKKRIDSCRIYFSTPKSWAVDLGYLSFTGVASSVGRIKIRQDRVWPGDAKFSSCHVVRDVDEWYAVFPLTFTKEIEKPKGGAVGINRGAVHAIADSTGRVVDSPKFYARSLGVIRHRARLLDRKVPFGRAVKPSPTKYHGLPKADIDAAAARVNASPGRLVYEARARGSIAAAEAHLAALVLPAPRQTSQLPSEGRNRERARRFLALAHQRVRRQREWFLHNESAHYAQSYTKIAIEDWSTKEMTSSEPRDAEEMKRVTRARNRSILDVGWYELGRQIAYKSEATGAEFAKVDPGLRETETHVPEAIVRERDVDVSGMLRGEAGISGTCSRCGGLLRASASGHADAECEVCLHVEVGDVNAAVNVLKRAMFPGAAPPSKEKAKVTIGIKGRKKKRAA

SEQ ID NO: 48 - protein, 565 aa, synthetic construct
  REGION 1..565: note = Description of Artificial Sequence: Synthetic polypeptide
MSRVELHRAYKFRLYPTPVQVAELSEWERQLRRLYNLGHEQRLLTLTRHLRPKSPGVLKGECLSCDSTQVQEVGADGRPKTTVRHAEQCPTLACRSCGALRDAEGRTAHTVACAFVDYYRQGREMTELLAADDQLARVVCSARQEVLRDLDKAWQRWRKMPGFGKPRFKRRTDSCRIYFSTPKAWKLEGGHLSFTGAATTVGAIKMRQDRNWPASVQFSSCHVVRDVDEWYAVFPLTFVAEVARPKGGAVGINRGAVHAIADSTGRVVDSPRYYARALGVIRHRARLFDRKVPSGHAVKPSPTKYRGLSAIEVDRVARATGFTPGRVVTEALNRGGVAYAECALAAIAVLGHGPERPLTSDGRNREKARKFLALAHQRVRRQREWFLHNESAHYARTYSKIAIEDWSTKEMTASEPQGEETRRVTRSRNRSILDVGWYELGRQLAYKTEATGAEFAQVDPGLKETETNVPKAIADARDVDVSGMLRGEAGISGTCSKCGGLLRAPASGHADAECEICLNVEVGDVNAAVNVLKRAMFPGDAPPASGEKPKVSIGIKGRQKKKKAA

SEQ ID NO: 49 - protein, 499 aa, synthetic construct
  REGION 1..499: note = Description of Artificial Sequence: Synthetic polypeptide
MEAIATGMSPERRVELGILPGSVELKRAYKFRLYPMKVQQAELSEWERQLRRLYNLAHEQRLAALLRYRDWDFQKGACPSCRVAVPGVHTAACDHVDYFRQAREMTQLLEVDAQLSRVICCARQEVLRDLDKAWQRWRKKLGGRPRFKRRTDSCRIYLSTPKHWEIAGRYLRLSGLASSVGEIRIEQDRAFPEGALLSSCSIVRDVDEWYACLPLTFTQPIERAPHRSVGLNRGVVHALADSDGRVVDSPKFFERALATVQKRSRDLARKVSGSRNAHKARIKLAKAHQRVRRQRAAFLHQESAYYSKGFDLVALEDMSVRKMTATAGEAPEMGRGAQRDLNRGILDVGWYELARQIDYKRLAHGGELLRVDPGQTTPLACVTEEQPARGISSACAVCGIPLARPASGNARMRCTACGSSQVGDVNAAENVLTRALSSAPSGPKSPKASIKIKGRQKRLGTPANRAGEASGGDPPVRGPVEGGTLAYVVEPVSESQSDT

SEQ ID NO: 50 - protein, 560 aa, synthetic construct
  REGION 1..560: note = Description of Artificial Sequence: Synthetic polypeptide
MTVRTYKYRAYPTPEQAEALTSWLRFASQLYNAALEHRKNAWGRHDAHGRGFRFWDGDAAPRKKSDPPGRWVYRGGGGAHISKNDQGKLLTEFRREHAELLPPGMPALVQHEVLARLERSMAAFFQRATKGQKAGYPRWRSEHRYDSLTFGLTSPSKERFDPETGESLGRGKTVGAGTYHNGDLRLTGLGELRILEHRRIPMGAIPKSVIVRRSGKRWFVSIAMEMPSVEPAASGRPAVGLDMGVVTWGTAFTADTSAAAALVADLRRMATDPSDCRRLEELEREAAQLSEVLAHCRARGLDPARPRRCPKELTKLYRRSLHRLGELDRACARIRRRLQAAHDIAEPVPDEAGSAVLIEGSNAGMRHARRVARTQRRVARRTRAGHAHSNRRKKAVQAYARAKERERSARGDHRHKVSRALVRQFEEISVEALDIKQLTVAPEHNPDPQPDLPAHVQRRRNRGELDAAWGAFFAALDYKAADAGGRVARKPAPHTTQECARCGTLVPKPISLRVHRCPACGYTAPRTVNSARNVLQRPLEEPGRAGPSGANGRGVPHAVA

SEQ ID NO: 51 - protein, 404 aa, synthetic construct
  REGION 1..404: note = Description of Artificial Sequence: Synthetic polypeptide
MNCRYRYRIYPTPGQRQSLARLFGCVRVVWNDALFLCRQSEKLPKNSELQKLCITQAKKTEARGWLGQVSAIPLQQSVADLGVAFKNFFQSRSGKRKGKKVNPPRVKRRNNRQGARFTRGGFKVKTSKVYLARIGDIKIKWSRPLPSEPSSVTVIKDCAGQYFLSFVVEVKPEIKPPKNPSIGIDLGLKTFASCSNGEKIDSPDYSRLYRKLKRCQRRLAKRQRGSKRRERMRVKVAKLNAQIRDKRKDFLHKLSTKVVNENQVIALEDLNVGGMLKNRKLSRAISQAGWYEFRSLCEGKAEKHNRDFRVISRWEPTSQVCSECGYRWGKIDLSVRSIVCINCGVEHDRDDNASVNIEQAGLKVGVGHTHDSKRTGSACKTSNGAVCVEPSTHREYVQLTLFDW

SEQ ID NO: 52 - protein, 392 aa, synthetic construct
  REGION 1..392: note = Description of Artificial Sequence: Synthetic polypeptide
MKSRWTFRCYPTPEQEQHLARTFGCVRFVWNWALRARTDAFRAGERIGYPATDKALTLLKQQPETVWLNEVSSVCLQQALRDLQVAFSNFFDKRAAHPSFKRKEARQSANYTERGFSFDHERRILKLAKIGAIKVKWSRKAIPHPSSIRLIRTASGKYFVSLVVETQPAPMPETGESVGVDFGVARLATLSNGERISNPKHGAKWQRRLAFYQKRLARATKGSKRRMRIKRHVARIHEKIGNSRSDTLHKLSTDLVTRFDLICVEDLNLRGMVKNHSLARSLHDASIGSAIRMIEEKAERYGKNVVKIDRWFPSSKTCSDCGHIVEQLPLNVREWTCPECGTTHDRDANAAANILAVGQTVSAHGGTVRRSRAKASERKSQRSANRQGVNRA

SEQ ID NO: 53 - protein, 500 aa, synthetic construct
  REGION 1..500: note = Description of Artificial Sequence: Synthetic polypeptide
KEPLNIGKTAKAVFKEIDPTSLNRAANYDASIELNCKECKFKPFKNVKRYEFNFYNNWYRCNPNSCLQSTYKAQVRKVEIGYEKLKNEILTQMQYYPWFGRLYQNFFHDERDKMTSLDEIQVIGVQNKVFFNTVEKAWREIIKKRFKDNKETMETIPELKHAAGHGKRKLSNKSLLRRRFAFVQKSFKFVDNSDVSYRSFSNNIACVLPSRIGVDLGGVISRNPKREYIPQEISFNAFWKQHEGLKKGRNIEIQSVQYKGETVKRIEADTGEDKAWGKNRQRRFTSLILKLVPKQGGKKVWKYPEKRNEGNYEYFPIPIEFILDSGETSIRFGGDEGEAGKQKHLVIPFNDSKATPLASQQTLLENSRFNAEVKSCIGLAIYANYFYGYARNYVISSIYHKNSKNGQAITAIYLESIAHNYVKAIERQLQNLLLNLRDFSFMESHKKELKKYFGGDLEGTGGAQKRREKEEKIEKEIEQSYLPRLIRLSLTKMVTKQVEM

SEQ ID NO: 54 - protein, 507 aa, synthetic construct
  REGION 1..507: note = Description of Artificial Sequence: Synthetic polypeptide
ELIVNENKDPLNIGKTAKAVFKEIDPTSINRAANYDASIELACKECKFKPFNNTKRHDFSFYSNWHRCSPNSCLQSTYRAKIRKTEIGYEKLKNEILNQMQYYPWFGRLYQNFFNDQRDKMTSLDEIQVTGVQNKIFFNTVEKAWREIIKKRFRDNKETMRTIPDLKNKSGHGSRKLSNKSLLRRRFAFAQKSFKLVDNSDVSYRAFSNNVACVLPSKIGVDIGGIINKDLKREYIPQEITFNVFWKQHDGLKKGRNIEIHSVQYKGEIVKRIEADTGEDKAWGKNRQRRFTSLILKITPKQGGKKIWKFPEKKNASDYEYFPIPIEFILDNGDASIKFGGEEGEVGKQKHLLIPFNDSKATPLSSKQMLLETSRFNAEVKSTIGLALYANYFVSYARNYVIKSTYHKNSKKGQIVTEIYLESISQNFVRAIQRQLQSLMLNLKDWGFMQTHKKELKKYFGSDLEGSKGGQKRREKEEKIEKEIEASYLPRLIRLSLTKSVTKAEEM

SEQ ID NO: 55 - protein, 529 aa, synthetic construct
  REGION 1..529: note = Description of Artificial Sequence: Synthetic polypeptide
PEEKTSKLKPNSINLAANYDANEKFNCKECKFHPFKNKKRYEFNFYNNLHGCKSCTKSTNNPAVKRIEIGYQKLKFEIKNQMEAYPWFGRLRINFYSDEKRKMSELNEMQVTGVKNKIFFDAIECAWREILKKRFRESKETLITIPKLKNKAGHGARKHRNKKLLIRRRAFMKKNFHFLDNDSISYRSFANNIACVLPSKVGVDIGGIISPDVGKDIKPVDISLNLMWASKEGIKSGRKVEIYSTQYDGNMVKKIEAETGEDKSWGKNRKRRQTSLLLSIPKPSKQVQEFDFKEWPRYKDIEKKVQWRGFPIKIIFDSNHNSIEFGTYQGGKQKVLPIPFNDSKTTPLGSKMNKLEKLRFNSKIKSRLGSAIAANKFLEAARTYCVDSLYHEVSSANAIGKGKIFIEYYLEILSQNYIEAAQKQLQRFIESIEQWFVADPFQGRLKQYFKDDLKRAKCFLCANREVQTTCYAAVKLHKSCAEKVKDKNKELAIKERNNKEDAVIKEVEASNYPRVIRLKLTKTITNKAM

SEQ ID NO: 56 - protein, 726 aa, synthetic construct
  REGION 1..726: note = Description of Artificial Sequence: Synthetic polypeptide
SESENKIIEQYYAFLYSFRDKYEKPEFKNRGDIKRKLQNKWEDFLKEQNLKNDKKLSNYIFSNRNFRRSYDREEENEEGIDEKKSKPKRINCFEKEKNLKDQYDKDAINASANKDGAQKWGCFECIFFPMYKIESGDPNKRIIINKTRFKLFDFYLNLKGCKSCLRSTYHPYRSNVYIESNYDKLKREIGNFLQQKNIFQRMRKAKVSEGKYLTNLDEYRLSCVAMHFKNRWLFFDSIQKVLRETIKQRLKQMRESYDEQAKTKRSKGHGRAKYEDQVRMIRRRAYSAQAHKLLDNGYITLFDYDDKEINKVCLTAINQEGFDIGGYLNSDIDNVMPPIEISFHLKWKYNEPILNIESPFSKAKISDYLRKIREDLNLERGKEGKARSKKNVRRKVLASKGEDGYKKIFTDFFSKWKEELEGNAMERVLSQSSGDIQWSKKKRIHYTTLVLNINLLDKKGVGNLKYYEIAEKTKILSFDKNENKFWPITIQVLLDGYEIGTEYDEIKQLNEKTSKQFTIYDPNTKIIKIPFTDSKAVPLGMLGINIATLKTVKKTERDIKVSKIFKGGLNSKIVSKIGKGIYAGYFPTVDKEILEEVEEDTLDNEFSSKSQRNIFLKSIIKNYDKMLKEQLFDFYSFLVRNDLGVRFLTDRELQNIEDESFNLEKRFFETDRDRIARWFDNTNTDDGKEKFKKLANEIVDSYKPRLIRLPVVRVIKRIQPVKQREM

SEQ ID NO: 57 - protein, 517 aa, synthetic construct
  REGION 1..517: note = Description of Artificial Sequence: Synthetic polypeptide
KYSTRDFSELNEIQVTACKQDEFFKVIQNAWREIIKKRFLENRENFIEKKIFKNKKGRGKRQESDKTIQRNRASVMKNFQLIENEKIILRAPSGHVACVFPVKVGLDIGGFKTDDLEKNIFPPRTITINVFWKNRDRQRKGRKLEVWGIKARTKLIEKVHKWDKLEEVKKKRLKSLEQKQEKSLDNWSEVNNDSFYKVQIDELQEKIDKSLKGRTMNKILDNKAKESKEAEGLYIEWEKDFEGEMLRRIEASTGGEEKWGKRRQRRHTSLLLDIKNNSRGSKEIINFYSYAKQGKKEKKIEFFPFPLTITLDAEEESPLNIKSIPIEDKNATSKYFSIPFTETRATPLSILGDRVQKFKTKNISGAIKRNLGSSISSCKIVQNAETSAKSILSLPNVKEDNNMEIFINTMSKNYFRAMMKQMESFIFEMEPKTLIDPYKEKAIKWFEVAASSRAKRKLKKLSKADIKKSELLLSNTEEFEKEKQEKLEALEKEIEEFYLPRIVRLQLTKTILETPVM

SEQ ID NO: 58 - protein, 481 aa, synthetic construct
  REGION 1..481: note = Description of Artificial Sequence: Synthetic polypeptide
KKLQLLGHKILLKEYDPNAVNAAANFETSTAELCGQCKMKPFKNKRRFQYTFGKNYHGCLSCIQNVYYAKKRIVQIAKEELKHQLTDSIASIPYKYTSLFSNTNSIDELYILKQERAAFFSNTNSIDELYITGIENNIAFKVISAIWDEIIKKRRQRYAESLTDTGTVKANRGHGGTAYKSNTRQEKIRALQKQTLHMVTNPYISLARYKNNYIVATLPRTIGMHIGAIKDRDPQKKLSDYAINFNVFWSDDRQLIELSTVQYTGDMVRKIEAETGENNKWGENMKRTKTSLLLEILTKKTTDELTFKDWAFSTKKEIDSVTKKTYQGFPIGIIFEGNESSVKFGSQNYFPLPFDAKITPPTAEGFRLDWLRKGSFSSQMKTSYGLAIYSNKVTNAIPAYVIKNMFYKIARAENGKQIKAKFLKKYLDIAGNNYVPFIIMQHYRVLDTFEEMPISQPKVIRLSLTKTQHIIIKKDKTDSKM

SEQ ID NO: 59 - protein, 534 aa, synthetic construct
  REGION 1..534: note = Description of Artificial Sequence: Synthetic polypeptide
NTSNLINLGKKAINISANYDANLEVGCKNCKFLSSNGNFPRQTNVKEGCHSCEKSTYEPSIYLVKIGERKAKYDVLDSLKKFTFQSLKYQSKKSMKSRNKKPKELKEFVIFANKNKAFDVIQKSYNHLILQIKKEINRMNSKKRKKNHKRRLFRDREKQLNKLRLIESSNLFLPRENKGNNHVFTYVAIHSVGRDIGVIGSYDEKLNFETELTYQLYFNDDKRLLYAYKPKQNKIIKIKEKLWNLRKEKEPLDLEYEKPLNKSITFSIKNDNLFKVSKDLMLRRAKFNIQGKEKLSKEERKINRDLIKIKGLVNSMSYGRFDELKKEKNIWSPHIYREVRQKEIKPCLIKNGDRIEIFEQLKKKMERLRRFREKRQKKISKDLIFAERIAYNFHTKSIKNTSNKINIDQEAKRGKASYMRKRIGYETFKNKYCEQCLSKGNVYRNVQKGCSCFENPFDWIKKGDENLLPKKNEDLRVKGAFRDEALEKQIVKIAFNIAKGYEDFYDNLGESTEKDLKLKFKVGTTINEQESLKL

SEQ ID NO: 60 - protein, 537 aa, synthetic construct
  REGION 1..537: note = Description of Artificial Sequence: Synthetic polypeptide
TSNPIKLGKKAINISANYDSNLQIGCKNCKFLSYNGNFPRQTNVKEGCHSCEKSTYEPPVYTVRIGERRSKYDVLDSLKKFIFLSLKYRQSKKMKTRSKGIRGLEEFVISANLKKAMDVIQKSYRHLILNIKNEIVRMNGKKRNKNHKRLLFRDREKQLNKLRLIEGSSFFKPPTVKGDNSIFTCVAIHNIGRDIGIAGDYFDKLEPKIELTYQLYYEYNPKKESEINKRLLYAYKPKQNKIIEIKEKLWNLRKEKSPLDLEYEKPLTKSITFLVKRDGVFRISKDLMLRKAKFIIQGKEKLSKEERKINRDLIKIKSNIISLTYGRFDELKKDKTIWSPHIFRDVKQGKITPCIERKGDRMDIFQQLRKKSERLRENRKKRQKKISKDLIFAERIAYNFHTKSIKNTSNLINIKHEAKRGKASYMRKRIGNETFRIKYCEQCFPKNNVYKNVQKGCSCFEDPFEYIKKGNEDLIPNKNQDLKAKGAFRDDALEKQIIKVAFNIAKGYEDFYENLKKTTEKDIRLKFKVGTIISEEM

SEQ ID NO: 61 - protein, 541 aa, synthetic construct
  REGION 1..541: note = Description of Artificial Sequence: Synthetic polypeptide
NNSINLSKKAINISANYDANLQVRCKNCKFLSSNGNFPRQTDVKEGCHSCEKSTYEPPVYDVKIGEIKAKYEVLDSLKKFTFQSLKYQLSKSMKFRSKKIKELKEFVIFAKESKALNVINRSYKHLILNIKNDINRMNSKKRIKNHKGRLFLDRQKQLSKLKLIEGSSFFVPAKNVGNKSVFTCVAIHSIGRDIGIAGLYDSFTKPVNEITYQIFFSGERRLLYAYKPKQLKILSIKENLWSLKNEKKPLDLLYEKPLGKNLNFNVKGGDLFRVSKDLMIRNAKFNVHGRQRLSDEERLINRNFIKIKGEVVSLSYGRFEELKKDRKLWSPHIFKDVRQNKIKPCLVMQGQRIDIFEQLKRKLELLKKIRKSRQKKLSKDLIFGERIAYNFHTKSIKNTSNKINIDSDAKRGRASYMRKRIGNETFKLKYCDVCFPKANVYRRVQNGCSCSENPYNYIKKGDKDLLPKKDEGLAIKGAFRDEKLNKQIIKVAFNIAKGYEDFYDDLKKRTEKDVDLKFKIGTTVLDQKPMEIFDGIVITWL

SEQ ID NO: 62 - protein, 542 aa, synthetic construct
  REGION 1..542: note = Description of Artificial Sequence: Synthetic polypeptide
LLTTVVETNNLAKKAINVAANFDANIDRQYYRCTPNLCRFIAQSPRETKEKDAGCSSCTQSTYDPKVYVIKIGKLLAKYEILKSLKRFLFMNRYFKQKKTERAQQKQKIGTELNEMSIFAKATNAMEVIKRATKHCTYDIIPETKSLQMLKRRRHRVKVRSLLKILKERRMKIKKIPNTFIEIPKQAKKNKSDYYVAAALKSCGIDVGLCGAYEKNAEVEAEYTYQLYYEYKGNSSTKRILYCYNNPQKNIREFWEAFYIQGSKSHVNTPGTIRLKMEKFLSPITIESEALDFRVWNSDLKIRNGQYGFIKKRSLGKEAREIKKGMGDIKRKIGNLTYGKSPSELKSIHVYRTERENPKKPRAARKKEDNFMEIFEMQRKKDYEVNKKRRKEATDAAKIMDFAEEPIRHYHTNNLKAVRRIDMNEQVERKKTSVFLKRIMQNGYRGNYCRKCIKAPEGSNRDENVLEKNEGCLDCIGSEFIWKKSSKEKKGLWHTNRLLRRIRLQCFTTAKAYENFYNDLFEKKESSLDIIKLKVSITTKSM

SEQ ID NO: 63 - protein, 564 aa, synthetic construct
  REGION 1..564: note = Description of Artificial Sequence: Synthetic polypeptide
ASTMNLAKQAINFAANYDSNLEIGCKGCKFMSTWSKKSNPKFYPRQNNQANKCHSCTYSTGEPEVPIIEIGERAAKYKIFTALKKFVFMSVAYKERRRQRFKSKKPKELKELAICSNREKAMEVIQKSVVHCYGDVKQEIPRIRKIKVLKNHKGRLFYKQKRSKIKIAKLEKGSFFKTFIPKVHNNGCHSCHEASLNKPILVTTALNTIGADIGLINDYSTIAPTETDISWQVYYEFIPNGDSEAVKKRLLYFYKPKGALIKSIRDKYFKKGHENAVNTGFFKYQGKIVKGPIKFVNNELDFARKPDLKSMKIKRAGFAIPSAKRLSKEDREINRESIKIKNKIYSLSYGRKKTLSDKDIIKHLYRPVRQKGVKPLEYRKAPDGFLEFFYSLKRKERRLRKQKEKRQKDMSEIIDAADEFAWHRHTGSIKKTTNHINFKSEVKRGKVPIMKKRIANDSFNTRHCGKCVKQGNAINKYYIEKQKNCFDCNSIEFKWEKAALEKKGAFKLNKRLQYIVKACFNVAKAYESFYEDFRKGEEESLDLKFKIGTTTTLKQYPQNKARAM

SEQ ID NO: 64 - protein, 610 aa, synthetic construct
  REGION 1..610: note = Description of Artificial Sequence: Synthetic polypeptide
HSHNLMLTKLGKQAINFAANYDANLEIGCKNCKFLSYSPKQANPKKYPRQTDVHEDGNIACHSCMQSTKEPPVYIVPIGERKSKYEILTSLNKFTFLALKYKEKKRQAFRAKKPKELQELAIAFNKEKAIKVIDKSIQHLILNIKPEIARIQRQKRLKNRKGKLLYLHKRYAIKMGLIKNGKYFKVGSPKKDGKKLLVLCALNTIGRDIGIIGNIEENNRSETEITYQLYFDCLDANPNELRIKEIEYNRLKSYERKIKRLVYAYKPKQTKILEIRSKFFSKGHENKVNTGSFNFENPLNKSISIKVKNSAFDFKIGAPFIMLRNGKFHIPTKKRLSKEEREINRTLSKIKGRVFRLTYGRNISEQGSKSLHIYRKERQHPKLSLEIRKQPDSFIDEFEKLRLKQNFISKLKKQRQKKLADLLQFADRIAYNYHTSSLEKTSNFINYKPEVKRGRTSYIKKRIGNEGFEKLYCETCIKSNDKENAYAVEKEELCFVCKAKPFTWKKTNKDKLGIFKYPSRIKDFIRAAFTVAKSYNDFYENLKKKDLKNEIFLKFKIGLILSHEKKNHISIAKSVAEDERISGKSIKNILNKSIKLEKNCYSCFFHKEDM

SEQ ID NO: 65 - protein, 552 aa, synthetic construct
  REGION 1..552: note = Description of Artificial Sequence: Synthetic polypeptide
SLERVIDKRNLAKKAINIAANFDANINKGFYRCETNQCMFIAQKPRKTNNTGCSSCLQSTYDPVIYVVKVGEMLAKYEILKSLKRFVFMNRSFKQKKTEKAKQKERIGGELNEMSIFANAALAMGVIKRAIRHCHVDIRPEINRLSELKKTKHRVAAKSLVKIVKQRKTKWKGIPNSFIQIPQKARNKDADFYVASALKSGGIDIGLCGTYDKKPHADPRWTYQLYFDTEDESEKRLLYCYNDPQAKIRDFWKTFYERGNPSMVNSPGTIEFRMEGFFEKMTPISIESKDFDFRVWNKDLLIRRGLYEIKKRKNLNRKAREIKKAMGSVKRVLANMTYGKSPTDKKSIPVYRVEREKPKKPRAVRKEENELADKLENYRREDFLIRNRRKREATEIAKIIDAAEPPIRHYHTNHLRAVKRIDLSKPVARKNTSVFLKRIMQNGYRGNYCKKCIKGNIDPNKDECRLEDIKKCICCEGTQNIWAKKEKLYTGRINVLNKRIKQMKLECFNVAKAYENFYDNLAALKEGDLKVLKLKVSIPALNPEASDPEEDM

SEQ ID NO: 66 - protein, 534 aa, synthetic construct
  REGION 1..534: note = Description of Artificial Sequence: Synthetic polypeptide
NASINLGKRAINLSANYDSNLVIGCKNCKFLSFNGNFPRQTNVREGCHSCDKSTYAPEVYIVKIGERKAKYDVLDSLKKFTFQSLKYQIKKSMRERSKKPKELLEFVIFANKDKAFNVIQKSYEHLILNIKQEINRMNGKKRIKNHKKRLFKDREKQLNKLRLIGSSSLFFPRENKGDKDLFTYVAIHSVGRDIGVAGSYESHIEPISDLTYQLFINNEKRLLYAYKPKQNKIIELKENLWNLKKEKKPLDLEFTKPLEKSITFSVKNDKLFKVSKDLMLRQAKFNIQGKEKLSKEERQINRDFSKIKSNVISLSYGRFEELKKEKNIWSPHIYREVKQKEIKPCIVRKGDRIELFEQLKRKMDKLKKFRKERQKKISKDLNFAERIAYNFHTKSIKNTSNKINIDQEAKRGKASYMRKRIGNESFRKKYCEQCFSVGNVYHNVQNGCSCFDNPIELIKKGDEGLIPKGKEDRKYKGALRDDNLQMQIIRVAFNIAKGYEDFYNNLKEKTEKDLKLKFKIGTTISTQESNNKEM

SEQ ID NO: 67 - protein, 577 aa, synthetic construct
  REGION 1..577: note = Description of Artificial Sequence: Synthetic polypeptide
SNLIKLGKQAINFAANYDANLEVGCKNCKFLSSTNKYPRQTNVHLDNKMACRSCNQSTMEPAIYIVRIGEKKAKYDIYNSLTKFNFQSLKYKAKRSQRFKPKQPKELQELSIAVRKEKALDIIQKSIDHLIQDIRPEIPRIKQQKRYKNHVGKLFYLQKRRKNKLNLIGKGSFFKVFSPKEKKNELLVICALTNIGRDIGLIGNYNTIINPLFEVTYQLYYDYIPKKNNKNVQRRLLYAYKSKNEKILKLKEAFFKRGHENAVNLGSFSYEKPLEKSLTLKIKNDKDDFQVSPSLRIRTGRFFVPSKRNLSRQEREINRRLVKIKSKIKNMTYGKFETARDKQSVHIFRLERQKEKLPLQFRKDEKEFMEEFQKLKRRTNSLKKLRKSRQKKLADLLQLSEKVVYNNHTGTLKKTSNFLNFSSSVKRGKTAYIKELLGQEGFETLYCSNCINKGQKTRYNIETKEKCFSCKDVPFVWKKKSTDKDRKGAFLFPAKLKDVIKATFTVAKAYEDFYDNLKSIDEKKPYIKFKIGLILAHVRHEHKARAKEEAGQKNIYNKPIKIDKNCKECFFFKEEAM

SEQ ID NO: 68 - protein, 613 aa, synthetic construct
  REGION 1..613: note = Description of Artificial Sequence: Synthetic polypeptide
NTTRKKFRKRTGFPQSDNIKLAYCSAIVRAANLDADIQKKHNQCNPNLCVGIKSNEQSRKYEHSDRQALLCYACNQSTGAPKVDYIQIGEIGAKYKILQMVNAYDFLSLAYNLTKLRNGKSRGHQRMSQLDEVVIVADYEKATEVIKRSINHLLDDIRGQLSKLKKRTQNEHITEHKQSKIRRKLRKLSRLLKRRRWKWGTIPNPYLKNWVFTKKDPELVTVALLHKLGRDIGLVNRSKRRSKQKLLPKVGFQLYYKWESPSLNNIKKSKAKKLPKRLLIPYKNVKLFDNKQKLENAIKSLLESYQKTIKVEFDQFFQNRTEEIIAEEQQTLERGLLKQLEKKKNEFASQKKALKEEKKKIKEPRKAKLLMEESRSLGFLMANVSYALFNTTIEDLYKKSNVVSGCIPQEPVVVFPADIQNKGSLAKILFAPKDGFRIKFSGQHLTIRTAKFKIRGKEIKILTKTKREILKNIEKLRRVWYREQHYKLKLFGKEVSAKPRFLDKRKTSIERRDPNKLADQTDDRQAELRNKEYELRHKQHKMAERLDNIDTNAQNLQTLSFWVGEADKPPKLDEKDARGFGVRTCISAWKWFMEDLLKKQEEDPLLKLKLSIM

SEQ ID NO: 69 - protein, 615 aa, synthetic construct
  REGION 1..615: note = Description of Artificial Sequence: Synthetic polypeptide
PKKPKFQKRTGFPQPDNLRKEYCLAIVRAANLDADFEKKCTKCEGIKTNKKGNIVKGRTYNSADKDNLLCYACNISTGAPAVDYVFVGALEAKYKILQMVKAYDFHSLAYNLAKLWKGRGRGHQRMGGLNEVVIVSNNEKALDVIEKSLNHFHDEIRGELSRLKAKFQNEHLHVHKESKLRRKLRKISRLLKRRRWKWDVIPNSYLRNFTFTKTRPDFISVALLHRVGRDIGLVTKTKIPKPTDLLPQFGFQIYYTWDEPKLNKLKKSRLRSEPKRLLVPYKKIELYKNKSVLEEAIRHLAEVYTEDLTICFKDFFETQKRKFVSKEKESLKRELLKELTKLKKDFSERKTALKRDRKEIKEPKKAKLLMEESRSLGFLAANTSYALFNLIAADLYTKSKKACSTKLPRQLSTILPLEIKEHKSTTSLAIKPEEGFKIRFSNTHLSIRTPKFKMKGADIKALTKRKREILKNATKLEKSWYGLKHYKLKLYGKEVAAKPRFLDKRNPSIDRRDPKELMEQIENRRNEVKDLEYEIRKGQHQMAKRLDNVDTNAQNLQTKSFWVGEADKPPELDSMEAKKLGLRTCISAWKWFMKDLVLLQEKSPNLKLKLSLTEM

SEQ ID NO: 70 - protein, 775 aa, synthetic construct
  REGION 1..775: note = Description of Artificial Sequence: Synthetic polypeptide
KFSKRQEGFLIPDNIDLYKCLAIVRSANLDADVQGHKSCYGVKKNGTYRVKQNGKKGVKEKGRKYVFDLIAFKGNIEKIPHEAIEEKDQGRVIVLGKFNYKLILNIEKNHNDRASLEIKNKIKKLVQISSLETGEFLSDLLSGKIGIDEVYGIIEPDVFSGKELVCKACQQSTYAPLVEYMPVGELDAKYKILSAIKGYDFLSLAYNLSRNRANKKRGHQKLGGGELSEVVISANYDKALNVIKRSINHYHVEIKPEISKLKKKMQNEPLKVMKQARIRRELHQLSRKVKRLKWKWGMIPNPELQNIIFEKKEKDFVSYALLHTLGRDIGLFKDTSMLQVPNISDYGFQIYYSWEDPKLNSIKKIKDLPKRLLIPYKRLDFYIDTILVAKVIKNLIELYRKSYVYETFGEEYGYAKKAEDILFDWDSINLSEGIEQKIQKIKDEFSDLLYEARESKRQNFVESFENILGLYDKNFASDRNSYQEKIQSMIIKKQQENIEQKLKREFKEVIERGFEGMDQNKKYYKVLSPNIKGGLLYTDTNNLGFFRSHLAFMLLSKISDDLYRKNNLVSKGGNKGILDQTPETMLTLEFGKSNLPNISIKRKFFNIKYNSSWIGIRKPKFSIKGAVIREITKKVRDEQRLIKSLEGVWHKSTHFKRWGKPRFNLPRHPDREKNNDDNLMESITSRREQIQLLLREKQKQQEKMAGRLDKIDKEIQNLQTANFQIKQIDKKPALTEKSEGKQSVRNALSAWKWFMEDLIKYQKRTPILQLKLAKM

SEQ ID NO: 71 - protein, 777 aa, synthetic construct
  REGION 1..777: note = Description of Artificial Sequence: Synthetic polypeptide
KFSKRQEGFVIPENIGLYKCLAIVRSANLDADVQGHVSCYGVKKNGTYVLKQNGKKSIREKGRKYASDLVAFKGDIEKIPFEVIEEKKKEQSIVLGKFNYKLVLDVMKGEKDRASLTMKNKSKKLVQVSSLGTDEFLLTLLNEKFGIEEIYGIIEPEVFSGKKLVCKACQQSTYAPLVEYMPVGELDSKYKILSAIKGYDFLSLAYNLARHRSNKKRGHQKLGGGELSEVVISANNAKALNVIKRSLNHYYSEIKPEISKLRKKMQNEPLKVGKQARMRRELHQLSRKVKRLKWKWGKIPNLELQNITFKESDRDFISYALLHTLGRDIGMFNKTEIKMPSNILGYGFQIYYDWEEPKLNTIKKSKNTPKRILIPYKKLDFYNDSILVARAIKELVGLFQESYEWEIFGNEYNYAKEAEVELIKLDEESINGNVEKKLQRIKENFSNLLEKAREKKRQNFIESFESIARLYDESFTADRNEYQREIQSFIIEKQKQSIEKKLKNEFKKIVEKKFNEQEQGKKHYRVLNPTIINEFLPKDKNNLGFLRSKIAFILLSKISDDLYKKSNAVSKGGEKGIIKQQPETILDLEFSKSKLPSINIKKKLFNIKYTSSWLGIRKPKFNIKGAKIREITRRVRDVQRTLKSAESSWYASTHFRRWGFPRFNQPRHPDKEKKSDDRLIESITLLREQIQILLREKQKGQKEMAGRLDDVDKKIQNLQTANFQIKQTGDKPALTEKSAGKQSFRNALSAWKWFMENLLKYQNKTPDLKLKIARTVM

SEQ ID NO: 72 - protein, 610 aa, synthetic construct
  REGION 1..610: note = Description of Artificial Sequence: Synthetic polypeptide
KWIEPNNIDFNKCLAITRSANLDADVQGHKMCYGIKTNGTYKAIGKINKKHNTGIIEKRRTYVYDLIVTKEKNEKIVKKTDFMAIDEEIEFDEKKEKLLKKYIKAEVLGTGELIRKDLNDGEKFDDLCSIEEPQAFRRSELVCKACNQSTYASDIRYIPIGEIEAKYKILKAIKGYDFLSLKYNLGRLRDSKKRGHQKMGQGELKEFVICANKEKALDVIKRSLNHYLNEVKDEISRLNKKMQNEPLKVNDQARWRRELNQISRRLKRLKWKWGEIPNPELKNLIFKSSRPEFVSYALIHTLGRDIGLINETELKPNNIQEYGFQIYYKWEDPELNHIKKVKNIPKRFIIPYKNLDLFGKYTILSRAIEGILKLYSSSFQYKSFKDPNLFAKEGEKKITNEDFELGYDEKIKKIKDDFKSYKKALLEKKKNTLEDSLNSILSVYEQSLLTEQINNVKKWKEGLLKSKESIHKQKKIENIEDIISRIEELKNVEGWIRTKERDIVNKEETNLKREIKKELKDSYYEEVRKDFSDLKKGEESEKKPFREEPKPIVIKDYIKFDVLPGENSALGFFLSHLSFNLFDSIQYELFEKSRLSSSKHPQIPETILDL

SEQ ID NO: 73 - protein, 632 aa, synthetic construct
  REGION 1..632: note = Description of Artificial Sequence: Synthetic polypeptide
FRKFVKRSGAPQPDNLNKYKCIAIVRAANLDADIMSNESSNCVMCKGIKMNKRKTAKGAAKTTELGRVYAGQSGNLLCTACTKSTMGPLVDYVPIGRIRAKYTILRAVKEYDFLSLAYNLARTRVSKKGGRQKMHSLSELVIAAEYEIAWNIIKSSVIHYHQETKEEISGLRKKLQAEHIHKNKEARIRREMHQISRRIKRLKWKWHMIPNSELHNFLFKQQDPSFVAVALLHTLGRDIGMINKPKGSAKREFIPEYGFQIYYKWMNPKLNDINKQKYRKMPKRSLIPYKNLNVFGDRELIENAMHKLLKLYDENLEVKGSKFFKTRVVAISSKESEKLKRDLLWKGELAKIKKDFNADKNKMQELFKEVKEPKKANALMKQSRNMGFLLQNISYGALGLLANRMYEASAKQSKGDATKQPSIVIPLEMEFGNAFPKLLLRSGKFAMNVSSPWLTIRKPKFVIKGNKIKNITKLMKDEKAKLKRLETSYHRATHFRPTLRGSIDWDSPYFSSPKQPNTHRRSPDRLSADITEYRGRLKSVEAELREGQRAMAKKLDSVDMTASNLQTSNFQLEKGEDPRLTEIDEKGRSIRNCISSWKKFMEDLMKAQEANPVIKIKIALKDESSVLSEDSM

SEQ ID NO: 74 - protein, 625 aa, synthetic construct
  REGION 1..625: note = Description of Artificial Sequence: Synthetic polypeptide
KFHPENLNKSYCLAIVRAANLDADIQGHINCIGIKSNKSDRNYENKLESLQNVELLCKACTKSTYKPNINSVPVGEKKAKYSILSEIKKYDFNSLVYNLKKYRKGKSRGHQKLNELRELVITSEYKKALDVINKSVNHYLVNIKNKMSKLKKILQNEHIHVGTLARIRRERNRISRKLDHYRKKWKFVPNKILKNYVFKNQSPDFVSVALLHKLGRDIGLITKTAILQKSFPEYSLQLYYKYDTPKLNYLKKSKFKSLPKRILISYKYPKFDINSNYIEESIDKLLKLYEESPIYKNNSKIIEFFKKSEDNLIKSENDSLKRGIMKEFEKVTKNFSSKKKKLKEELKLKNEDKNSKMLAKVSRPIGFLKAYLSYMLFNIISNRIFEFSRKSSGRIPQLPSCIINLGNQFENFKNELQDSNIGSKKNYKYFCNLLLKSSGFNISYEEEHLSIKTPNFFINGRKLKEITSEKKKIRKENEQLIKQWKKLTFFKPSNLNGKKTSDKIRFKSPNNPDIERKSEDNIVENIAKVKYKLEDLLSEQRKEFNKLAKKHDGVDVEAQCLQTKSFWIDSNSPIKKSLEKKNEKVSVKKKMKAIRSCISAWKWFMADLIEAQKETPMIKLKLALM

SEQ ID NO: 75 - protein, 517 aa, synthetic construct
  REGION 1..517: note = Description of Artificial Sequence: Synthetic polypeptide
TTLVPSHLAGIEVMDETTSRNEDMIQKETSRSNEDENYLGVKNKCGINVHKSGRGSSKHEPNMPPEKSGEGQMPKQDSTEMQQRFDESVTGETQVSAGATASIKTDARANSGPRVGTARALIVKASNLDRDIKLGCKPCEYIRSELPMGKKNGCNHCEKSSDIASVPKVESGFRKAKYELVRRFESFAADSISRHLGKEQARTRGKRGKKDKKEQMGKVNLDEIAILKNESLIEYTENQILDARSNRIKEWLRSLRLRLRTRNKGLKKSKSIRRQLITLRRDYRKWIKPNPYRPDEDPNENSLRLHTKLGVDIGVQGGDNKRMNSDDYETSFSITWRDTATRKICFTKPKGLLPRHMKFKLRGYPELILYNEELRIQDSQKFPLVDWERIPIFKLRGVSLGKKKVKALNRITEAPRLVVAKRIQVNIESKKKKVLTRYVYNDKSINGRLVKAEDSNKDPLLEFKKQAEEINSDAKYYENQEIAKNYLWGCEGLHKNLLEEQTKNPYLAFKYGFLNIV

SEQ ID NO: 76 - protein, 410 aa, synthetic construct
  REGION 1..410: note = Description of Artificial Sequence: Synthetic polypeptide
LDFKRTCSQELVLLPEIEGLKLSGTQGVTSLAKKLINKAANVDRDESYGCHHCIHTRTSLSKPVKKDCNSCNQSTNHPAVPITLKGYKIAFYELWHRFTSWAVDSISKALHRNKVMGKVNLDEYAVVDNSHIVCYAVRKCYEKRQRSVRLHKRAYRCRAKHYNKSQPKVGRIYKKSKRRNARNLKKEAKRYFQPNEITNGSSDALFYKIGVDLGIAKGTPETEVKVDVSICFQVYYGDARRVLRVRKMDELQSFHLDYTGKLKLKGIGNKDTFTIAKRNESLKWGSTKYEVSRAHKKFKPFGKKGSVKRKCNDYFRSIASWSCEAASQRAQSNLKNAFPYQKALVKCYKNLDYKGVKKNDMWYRLCSNRIFRYSRIAEDIAQYQSDKGKAKFEFVILAQSVAEYDISAIM

SEQ ID NO: 77 - protein, 602 aa, synthetic construct
  REGION 1..602: note = Description of Artificial Sequence: Synthetic polypeptide
VFLTDDKRKTALRKIRSAFRKTAEIALVRAQEADSLDRQAKKLTIETVSFGAPGAKNAFIGSLQGYNWNSHRANVPSSGSAKDVFRITELGLGIPQSAHEASIGKSFELVGNVVRYTANLLSKGYKKGAVNKGAKQQREIKGKEQLSFDLISNGPISGDKLINGQKDALAWWLIDKMGFHIGLAMEPLSSPNTYGITLQAFWKRHTAPRRYSRGVIRQWQLPFGRQLAPLIHNFFRKKGASIPIVLTNASKKLAGKGVLLEQTALVDPKKWWQVKEQVTGPLSNIWERSVPLVLYTATFTHKHGAAHKRPLTLKVIRISSGSVFLLPLSKVTPGKLVRAWMPDINILRDGRPDEAAYKGPDLIRARERSFPLAYTCVTQIADEWQKRALESNRDSITPLEAKLVTGSDLLQIHSTVQQAVEQGIGGRISSPIQELLAKDALQLVLQQLFMTVDLLRIQWQLKQEVADGNTSEKAVGWAIRISNIHKDAYKTAIEPCTSALKQAWNPLSGFEERTFQLDASIVRKRSTAKTPDDELVIVLRQQAAEMTVAVTQSVSKELMELAVRHSATLHLLVGEVASKQLSRSADKDRGAMDHWKLLSQSM

SEQ ID NO: 78 - protein, 494 aa, synthetic construct
  REGION 1..494: note = Description of Artificial Sequence: Synthetic polypeptide
EDLLQKALNTATNVAAIERHSCISCLFTESEIDVKYKTPDKIGQNTAGCQSCTFRVGYSGNSHTLPMGNRIALDKLRETIQRYAWHSLLFNVPPAPTSKRVRAISELRVAAGRERLFTVITFVQTNILSKLQKRYAANWTPKSQERLSRLREEGQHILSLLESGSWQQKEVVREDQDLIVCSALTKPGLSIGAFCRPKYLKPAKHALVLRLIFVEQWPGQIWGQSKRTRRMRRRKDVERVYDISVQAWALKGKETRISECIDTMRRHQQAYIGVLPFLILSGSTVRGKGDCPILKEITRMRYCPNNEGLIPLGIFYRGSANKLLRVVKGSSFTLPMWQNIETLPHPEPFSPEGWTATGALYEKNLAYWSALNEAVDWYTGQILSSGLQYPNQNEFLARLQNVIDSIPRKWFRPQGLKNLKPNGQEDIVPNEFVIPQNAIRAHHVIEWYHKTNDLVAKTLLGWGSQTTLNQTRPQGDLRFTYTRYYFREKEVPEV

SEQ ID NO: 79 - protein, 649 aa, synthetic construct
  REGION 1..649: note = Description of Artificial Sequence: Synthetic polypeptide
VPKKKLMRELAKKAVFEAIFNDPIPGSFGCKRCTLIDGARVTDAIEKKQGAKRCAGCEPCTFHTLYDSVKHALPAATGCDRTAIDTGLWEILTALRSYNWMSFRRNAVSDASQKQVWSIEELAIWADKERALRVILSALTHTIGKLKNGFSRDGVWKGGKQLYENLAQKDLAKGLFANGEIFGKELVEADHDMLAWTIVPNHQFHIGLIRGNWKPAAVEASTAFDARWLTNGAPLRDTRTHGHRGRRFNRTEKLTVLCIKRDGGVSEEFRQERDYELSVMLLQPKNKLKPEPKGELNSFEDLHDHWWFLKGDEATALVGLTSDPTVGDFIQLGLYIRNPIKAHGETKRRLLICFEPPIKLPLRRAFPSEAFKTWEPTINVFRNGRRDTEAYYDIDRARVFEFPETRVSLEHLSKQWEVLRLEPDRENTDPYEAQQNEGAELQVYSLLQEAAQKMAPKVVIDPFGQFPLELFSTFVAQLFNAPLSDTKAKIGKPLDSGFVVESHLHLLEEDFAYRDFVRVTFMGTEPTFRVIHYSNGEGYWKKTVLKGKNNIRTALIPEGAKAAVDAYKNKRCPLTLEAAILNEEKDRRLVLGNKALSLLAQTARGNLTILEALAAEVLRPLSGTEGVVHLHACVTRHSTLTESTETDNM

SEQ ID NO: 80 - protein, 414 aa, synthetic construct
  REGION 1..414: note = Description of Artificial Sequence: Synthetic polypeptide
VEKLFSERLKRAMWLKNEAGRAPPAETLTLKHKRVSGGHEKVKEELQRVLRSLSGTNQAAWNLGLSGGREPKSSDALKGEKSRVVLETVVFHSGHNRVLYDVIEREDQVHQRSSIMHMRRKGSNLLRLWGRSGKVRRKMREEVAEIKPVWHKDSRWLAIVEEGRQSVVGISSAGLAVFAVQESQCTTAEPKPLEYVVSIWFRGSKALNPQDRYLEFKKLKTTEALRGQQYDPIPFSLKRGAGCSLAIRGEGIKFGSRGPIKQFFGSDRSRPSHADYDGKRRLSLFSKYAGDLADLTEEQWNRTVSAFAEDEVRRATLANIQDFLSISHEKYAERLKKRIESIEEPVSASKLEAYLSAIFETFVQQREALASNFLMRLVESVALLISLEEKSPRVEFRVARYLAESKEGFNRKAM

SEQ ID NO: 81 - protein, 413 aa, synthetic construct
  REGION 1..413: note = Description of Artificial Sequence: Synthetic polypeptide
VVITQSELYKERLLRVMEIKNDRGRKEPRESQGLVLRFTQVTGGQEKVKQKLWLIFEGFSGTNQASWNFGQPAGGRKPNSGDALKGPKSRVTYETVVFHFGLRLLSAVIERHNLKQQRQTMAYMKRRAAARKKWARSGKKCSRMRNEVEKIKPKWHKDPRWFDIVKEGEPSIVGISSAGFAIYIVEEPNFPRQDPLEIEYAISIWFRRDRSQYLTFKKIQKAEKLKELQYNPIPFRLKQEKTSLVFESGDIKFGSRGSIEHFRDEARGKPPKADMDNNRRLTMFSVFSGNLTNLTEEQYARPVSGLLAPDEKRMPTLLKKLQDFFTPIHEKYGERIKQRLANSEASKRPFKKLEEYLPAIYLEFRARREGLASNWVLVLINSVRTLVRIKSEDPYIEFKVSQYLLEKEDNKAL

SEQ ID NO: 82 - protein, 449 aa, synthetic construct
  REGION 1..449: note = Description of Artificial Sequence: Synthetic polypeptide
KQDALFEERLKKAIFIKRQADPLQREELSLLPPNRKIVTGGHESAKDTLKQILRAINGTNQASWNPGTPSGKRDSKSADALAGPKSRVKLETVVFHVGHRLLKKVVEYQGHQKQQHGLKAFMRTCAAMRKKWKRSGKVVGELREQLANIQPKWHYDSRPLNLCFEGKPSVVGLRSAGIALYTIQKSVVPVKEPKPIEYAVSIWFRGPKAMDREDRCLEFKKLKIATELRKLQFEPIVSTLTQGIKGFSLYIQGNSVKFGSRGPIKYFSNESVRQRPPKADPDGNKRLALFSKFSGDLSDLTEEQWNRPILAFEGIIRRATLGNIQDYLTVGHEQFAISLEQLLSEKESVLQMSIEQQRLKKNLGKKAENEWVESFGAEQARKKAQGIREYISGFFQEYCSQREQWAENWVQQLNKSVRLFLTIQDSTPFIEFRVARYLPKGEKKKGKAM

SEQ ID NO: 83 - protein, 711 aa, synthetic construct
  REGION 1..711: note = Description of Artificial Sequence: Synthetic polypeptide
ANHAERHKRLRKEANRAANRNRPLVADCDTGDPLVGICRLLRRGDKMQPNKTGCRSCEQVEPELRDAILVSGPGRLDNYKYELFQRGRAMAVHRLLKRVPKLNRPKKAAGNDEKKAENKKSEIQKEKQKQRRMMPAVSMKQVSVADFKHVIENTVRHLFGDRRDREIAECAALRAASKYFLKSRRVRPRKLPKLANPDHGKELKGLRLREKRAKLKKEKEKQAELARSNQKGAVLHVATLKKDAPPMPYEKTQGRNDYTTFVISAAIKVGATRGTKPLLTPQPREWQCSLYWRDGQRWIRGGLLGLQAGIVLGPKLNRELLEAVLQRPIECRMSGCGNPLQVRGAAVDFFMTTNPFYVSGAAYAQKKFKPFGTKRASEDGAAAKAREKLMTQLAKVLDKVVTQAAHSPLDGIWETRPEAKLRAMIMALEHEWIFLRPGPCHNAAEEVIKCDCTGGHAILWALIDEARGALEHKEFYAVTRAHTHDCEKQKLGGRLAGFLDLLIAQDVPLDDAPAARKIKTLLEATPPAPCYKAATSIATCDCEGKFDKLWAIIDATRAGHGTEDLWARTLAYPQNVNCKCKAGKDLTHRLADFLGLLIKRDGPFRERPPHKVTGDRKLVFSGDKKCKGHQYVILAKAHNEEVVRAWISRWGLKSRTNKAGYAATELNLLLNWLSICRRRWMDMLTVQRDTPYIRMKTGRLVVDDKKERKAM

SEQ ID NO: 84 - protein, 574 aa, synthetic construct
  REGION 1..574: note = Description of Artificial Sequence: Synthetic polypeptide
AKQREALRVALERGIVRASNRTYTLVTNCTKGGPLPEQCRMIERGKARAMKWEPKLVGCGSCAAATVDLPAIEEYAQPGRLDVAKYKLTTQILAMATRRMMVRAAKLSRRKGQWPAKVQEEKEEPPEPKKMLKAVEMRPVAIVDFNRVIQTTIEHLWAERANADEAELKALKAAAAYFGPSLKIRARGPPKAAIGRELKKAHRKKAYAERKKARRKRAELARSQARGAAAHAAIRERDIPPMAYERTQGRNDVTTIPIAAAIKIAATRGARPLPAPKPMKWQCSLYWNEGQRWIRGGMLTAQAYAHAANIHRPMRCEMWGVGNPLKVRAFEGRVADPDGAKGRKAEFRLQTNAFYVSGAAYRNKKFKPFGTDRGGIGSARKKRERLMAQLAKILDKVVSQAAHSPLDDIWHTRPAQKLRAMIKQLEHEWMFLRPQAPTVEGTKPDVDVAGNMQRQIKALMAPDLPPIEKGSPAKRFTGDKRKKGERAVRVAEAHSDEVVTAWISRWGIQTRRNEGSYAAQELELLLNWLQICRRRWLDMTAAQRVSPYIRMKSGRMITDAADEGVAPIPLVENM

SEQ ID NO: 85 - protein, 743 aa, synthetic construct
  REGION 1..743: note = Description of Artificial Sequence: Synthetic polypeptide
KSISGRSIKHMACLKDMLKSEITEIEEKQKKESLRKWDYYSKFSDEILFRRNLNVSANHDANACYGCNPCAFLKEVYGFRIERRNNERIISYRRGLAGCKSCVQSTGYPPIEFVRRKFGADKAMEIVREVLHRRNWGALARNIGREKEADPILGELNELLLVDARPYFGNKSAANETNLAFNVITRAAKKFRDEGMYDIHKQLDIHSEEGKVPKGRKSRLIRIERKHKAIHGLDPGETWRYPHCGKGEKYGVWLNRSRLIHIKGNEYRCLTAFGTTGRRMSLDVACSVLGHPLVKKKRKKGKKTVDGTELWQIKKATETLPEDPIDCTFYLYAAKPTKDPFILKVGSLKAPRWKKLHKDFFEYSDTEKTQGQEKGKRVVRRGKVPRILSLRPDAKFKVSIWDDPYNGKNKEGTLLRMELSGLDGAKKPLILKRYGEPNTKPKNFVFWRPHITPHPLTFTPKHDFGDPNKKTKRRRVFNREYYGHLNDLAKMEPNAKFFEDREVSNKKNPKAKNIRIQAKESLPNIVAKNGRWAAFDPNDSLWKLYLHWRGRRKTIKGGISQEFQEFKERLDLYKKHEDESEWKEKEKLWENHEKEWKKTLEIHGSIAEVSQRCVMQSMMGPLDGLVQKKDYVHIGQSSLKAADDAWTFSANRYKKATGPKWGKISVSNLLYDANQANAELISQSISKYLSKQKDNQGCEGRKMKFLIKIIEPLRENFVKHTRWLHEMTQKDCEVRAQFSRVSM

SEQ ID NO: 86 - protein, 769 aa, synthetic construct
  REGION 1..769: note = Description of Artificial Sequence: Synthetic polypeptide
FPSDVGADALKHVRMLQPRLTDEVRKVALTRAPSDRPALARFAAVAQDGLAFVRHLNVSANHDSNCTFPRDPRDPRRGPCEPNPCAFLREVWGFRIVARGNERALSYRRGLAGCKSCVQSTGFPSVPFHRIGADDCMRKLHEILKARNWRLLARNIGREREADPLLTELSEYLLVDARTYPDGAAPNSGRLAENVIKRAAKKFRDEGMRDIHAQLRVHSREGKVPKGRLQRLRRIERKHRAIHALDPGPSWEAEGSARAEVQGVAVYRSQLLRVGHHTQQIEPVGIVARTLFGVGRTDLDVAVSVLGAPLTKRKKGSKTLESTEDFRIAKARETRAEDKIEVAFVLYPTASLLRDEIPKDAFPAMRIDRFLLKVGSVQADREILLQDDYYRFGDAEVKAGKNKGRTVTRPVKVPRLQALRPDAKFRVNVWADPFGAGDSPGTLLRLEVSGVTRRSQPLRLLRYGQPSTQPANFLCWRPHRVPDPMTFTPRQKFGERRKNRRTRRPRVFERLYQVHIKHLAHLEPNRKWFEEARVSAQKWAKARAIRRKGAEDIPVVAPPAKRRWAALQPNAELWDLYAHDREARKRFRGGRAAEGEEFKPRLNLYLAHEPEAEWESKRDRWERYEKKWTAVLEEHSRMCAVADRTLPQFLSDPLGARMDDKDYAFVGKSALAVAEAFVEEGTVERAQGNCSITAKKKFASNASRKRLSVANLLDVSDKADRALVFQAVRQYVQRQAENGGVEGRRMAFLRKLLAPLRQNFVCHTRWLHM

SEQ ID NO: 87 - protein, 564 aa, synthetic construct
  REGION 1..564: note = Description of Artificial Sequence: Synthetic polypeptide
AARKKKRGKIGITVKAKEKSPPAAGPFMARKLVNVAANVDGVEVHLCVECEADAHGSASARLLGGCRSCTGSIGAEGRLMGSVDVDRERVIAEPVHTETERLGPDVKAFEAGTAESKYAIQRGLEYWGVDLISRNRARTVRKMEEADRPESSTMEKTSWDEIAIKTYSQAYHASENHLFWERQRRVRQHALALFRRARERNRGESPLQSTQRPAPLVLAALHAEAAAISGRARAEYVLRGPSANVRAAAADIDAKPLGHYKTPSPKVARGFPVKRDLLRARHRIVGLSRAYFKPSDVVRGTSDAIAHVAGRNIGVAGGKPKEIEKTFTLPFVAYWEDVDRVVHCSSFKADGPWVRDQRIKIRGVSSAVGTFSLYGLDVAWSKPTSFYIRCSDIRKKFHPKGFGPMKHWRQWAKELDRLTEQRASCVVRALQDDEELLQTMERGQRYYDVFSCAATHATRGEADPSGGCSRCELVSCGVAHKVTKKAKGDTGIEAVAVAGCSLCESKLVGPSKPRVHRQMAALRQSHALNYLRRLQREWEALEAVQAPTPYLRFKYARHLEVRSM

SEQ ID NO: 88 - protein, 565 aa, synthetic construct
  REGION 1..565: note = Description of Artificial Sequence: Synthetic polypeptide
AAKKKKQRGKIGISVKPKEGSAPPADGPFMARKLVNVAANVDGVEVNLCIECEADAHGSAPARLLGGCKSCTGSIGAEGRLMGSVDVDRADAIAKPVNTETEKLGPDVQAFEAGTAETKYALQRGLEYWGVDLISRNRSRTVRRTEEGQPESATMEKTSWDEIAIKSYTRAYHASENHLFWERQRRVRQHALALFKRAKERNRGDSTLPREPGHGLVAIAALACEAYAVGGRNLAETVVRGPTFGTARAVRDVEIASLGRYKTPSPKVAHGSPVKRDFLRARHRIVGLARAYYRPSDVVRGTSDAIAHVAGRNIGVAGGKPRAVEAVFTLPFVAYWEDVDRVVHCSSFQVSAPWNRDQRMKIAGVTTAAGTFSLHGGELKWAKPTSFYIRCSDTRRKFRPKGFGPMKRWRQWAKDLDRLVEQRASCVVRALQDDAALLETMERGQRYYDVFACAVTHATRGEADRLAGCSRCALTPCQEAHRVTTKPRGDAGVEQVQTSDCSLCEGKLVGPSKPRLHRTLTLLRQEHGLNYLRRLQREWESLEAVQVPTPYLRFKYARHLEVRSM

SEQ ID NO: 89 - protein, 499 aa, synthetic construct
  REGION 1..499: note = Description of Artificial Sequence: Synthetic polypeptide
TDSQSESVPEVVYALTGGEVPGRVPPDGGSAEGARNAPTGLRKQRGKIKISAKPSKPGSPASSLARTLVNEAANVDGVQSSGCATCRMRANGSAPRALPIGCVACASSIGRAPQEETVCALPTTQGPDVRLLEGGHALRKYDIQRALEYWGVDLIGRNLDRQAGRGMEPAEGATATMKRVSMDELAVLDFGKSYYASEQHLFAARQRRVRQHAKALKIRAKHANRSGSVKRALDRSRKQVTALAREFFKPSDVVRGDSDALAHVVGRNLGVSRHPAREIPQTFTLPLCAYWEDVDRVISCSSLLAGEPFARDQEIRIEGVSSALGSLRLYRGAIEWHKPTSLYIRCSDTRRKFRPRGGLKKRWRQWAKDLDRLVEQRACCIVRSLQADVELLQTMERAQRFYDVHDCAATHVGPVAVRCSPCAGKQFDWDRYRLLAALRQEHALNYLRRLQREWESLEAQQVKMPYLRFKYARKLEVSGPLIGLEVRREPSMGTAIAEM

SEQ ID NO: 90 - protein, 358 aa, synthetic construct
  REGION 1..358: note = Description of Artificial Sequence: Synthetic polypeptide
AGTAGRRHGSLGARRSINIAGVTDRHGRWGCESCVYTRDQAGNRARCAPCDQSTYAPDVQEVTIGQRQAKYTIFLTLQSFSWTNTMRNNKRAAAGRSKRTTGKRIGQLAEIKITGVGLAHAHNVIQRSLQHNITKMWRAEKGKSKRVARLKKAKQLTKRRAYFRRRMSRQSRGNGFFRTGKGGIHAVAPVKIGLDVGMIASGSSEPADEQTVTLDAIWKGRKKKIRLIGAKGELAVAACRFREQQTKGDKCIPLILQDGEVRWNQNNWQCHPKKLVPLCGLEVSRKFVSQADRLAQNKVASPLAARFDKTSVKGTLVESDFAAVLVNVTSIYQQCHAMLLRSQEPTPSLRVQRTITSM

SEQ ID NO: 91 - protein, 369 aa, synthetic construct
  REGION 1..369: note = Description of Artificial Sequence: Synthetic polypeptide
GVRFSPAQSQVFFRTVIPQSVEARFAINMAAIHDAAGAFGCSVCRFEDRTPRNAKAVHGCSPCTRSTNRPDVFVLPVGAIKAKYDVFMRLLGFNWTHLNRRQAKRVTVRDRIGQLDELAISMLTGKAKAVLKKSICHNVDKSFKAMRGSLKKLHRKASKTGKSQLRAKLSDLRERTNTTQEGSHVEGDSDVALNKIGLDVGLVGKPDYPSEESVEVVVCLYFVGKVLILDAQGRIRDMRAKQYDGFKIPIIQRGQLTVLSVKDLGKWSLVRQDYVLAGDLRFEPKISKDRKYAECVKRIALITLQASLGFKERIPYYVTKQVEIKNASHIAFVTEAIQNCAENFREMTEYLMKYQEKSPDLKVLLTQLM

SEQ ID NO: 92 - protein, 486 aa, synthetic construct
  REGION 1..486: note = Description of Artificial Sequence: Synthetic polypeptide
RAVVGKVFLEQARRALNLATNFGTNHRTGCNGCYVTPGKLSIPQDGEKNAAGCTSCLMKATASYVSYPKPLGEKVAKYSTLDALKGFPWYSLRLNLRPNYRGKPINGVQEVAPVSKFRLAEEVIQAVQRYHFTELEQSFPGGRRRLRELRAFYTKEYRRAPEQRQHVVNGDRNIVVVTVLHELGFSVGMFNEVELLPKTPIECAVNVFIRGNRVLLEVRKPQFDKERLLVESLWKKDSRRHTAKWTPPNNEGRIFTAEGWKDFQLPLLLGSTSRSLRAIEKEGFVQLAPGRDPDYNNTIDEQHSGRPFLPLYLYLQGTISQEYCVFAGTWVIPFQDGISPYSTKDTFQPDLKRKAYSLLLDAVKHRLGNKVASGLQYGRFPAIEELKRLVRMHGATRKIPRGEKDLLKKGDPDTPEWWLLEQYPEFWRLCDAAAKRVSQNVGLLLSLKKQPLWQRRWLESRTRNEPLDNLPLSMALTLHLTNEEAL

SEQ ID NO: 93 - protein, 400 aa, synthetic construct
  REGION 1..400: note = Description of Artificial Sequence: Synthetic polypeptide
AAVYSKFYIENHFKMGIPETLSRIRGPSIIQGFSVNENYINIAGVGDRDFIFGCKKCKYTRGKPSSKKINKCHPCKRSTYPEPVIDVRGSISEFKYKIYNKLKQEPNQSIKQNTKGRMNPSDHTSSNDGIIINGIDNRIAYNVIFSSYKHLMEKQINLLRDTTKRKARQIKKYNNSGKKKHSLRSQTKGNLKNRYHMLGMFKKGSLTITNEGDFITAVRKVGLDISLYKNESLNKQEVETELCLNIKWGRTKSYTVSGYIPLPINIDWKLYLFEKETGLTLRLFGNKYKIQSKKFLIAQLFKPKRPPCADPVVKKAQKWSALNAHVQQMAGLFSDSHLLKRELKNRMHKQLDFKSLWVGTEDYIKWFEELSRSYVEGAEKSLEFFRQDYFCFNYTKQTTM

SEQ ID NO: 94 - protein, 666 aa, synthetic construct
  REGION 1..666: note = Description of Artificial Sequence: Synthetic polypeptide
PQQQRDLMLMAANYDQDYGNGCGPCTVVASAAYRPDPQAQHGCKRHLRTLGASAVTHVGLGDRTATITALHRLRGPAALAARARAAQAASAPMTPDTDAPDDRRRLEAIDADDVVLVGAHRALWSAVRRWADDRRAALRRRLHSEREWLLKDQIRWAELYTLIEASGTPPQGRWRNTLGALRGQSRWRRVLAPTMRATCAETHAELWDALAELVPEMAKDRRGLLRPPVEADALWRAPMIVEGWRGGHSVVVDAVAPPLDLPQPCAWTAVRLSGDPRQRWGLHLAVPPLGQVQPPDPLKATLAVSMRHRGGVRVRTLQAMAVDADAPMQRHLQVPLTLQRGGGLQWGIHSRGVRRREARSMASWEGPPIWTGLQLVNRWKGQGSALLAPDRPPDTPPYAPDAAVAPAQPDTKRARRTLKEACTVCRCAPGHMRQLQVTLTGDGTWRRFRLRAPQGAKRKAEVLKVATQHDERIANYTAWYLKRPEHAAGCDTCDGDSRLDGACRGCRPLLVGDQCFRRYLDKIEADRDDGLAQIKPKAQEAVAAMAAKRDARAQKVAARAAKLSEATGQRTAATRDASHEARAQKELEAVATEGTTVRHDAAAVSAFGSWVARKGDEYRHQVGVLANRLEHGLRLQELMAPDSVVADQQRASGHARVGYRYVLTAM

SEQ ID NO: 95 - protein, 560 aa, synthetic construct
  REGION 1..560: note = Description of Artificial Sequence: Synthetic polypeptide
AVAHPVGRGNAGSPGARGPEELPRQLVNRASNVTRPATYGCAPCRHVRLSIPKPVLTGCRACEQTTHPAPKRAVRGGADAAKYDLAAFFAGWAADLEGRNRRRQVHAPLDPQPDPNHEPAVTLQKIDLAEVSIEEFQRVLARSVKHRHDGRASREREKARAYAQVAKKRRNSHAHGARTRRAVRRQTRAVRRAHRMGANSGEILVASGAEDPVPEAIDHAAQLRRRIRACARDLEGLRHLSRRYLKTLEKPCRRPRAPDLGRARCHALVESLQAAERELEELRRCDSPDTAMRRLDAVLAAAASTDATFATGWTVVGMDLGVAPRGSAAPEVSPMEMAISVFWRKGSRRVIVSKPIAGMPIRRHELIRLEGLGTLRLDGNHYTGAGVTKGRGLSEGTEPDFREKSPSTLGFTLSDYRHESRWRPYGAKQGKTARQFFAAMSRELRALVEHQVLAPMGPPLLEAHERRFETLLKGQDNKSIHAGGGGRYVWRGPPDSKKRPAADGDWFRFGRGHADHRGWANKRHELAANYLQSAFRLWSTLAEAQEPTPYARYKYTRVTM

SEQ ID NO: 96 - protein, 404 aa, synthetic construct
  REGION 1..404: note = Description of Artificial Sequence: Synthetic polypeptide
WDFLTLQVYERHTSPEVCVAGNSTKCASGTRKSDHTHGVGVKLGAQEINVSANDDRDHEVGCNICVISRVSLDIKGWRYGCESCVQSTPEWRSIVRFDRNHKEAKGECLSRFEYWGAQSIARSLKRNKLMGGVNLDELAIVQNENVVKTSLKHLFDKRKDRIQANLKAVKVRMRERRKSGRQRKALRRQCRKLKRYLRSYDPSDIKEGNSCSAFTKLGLDIGISPNKPPKIEPKVEVVFSLFYQGACDKIVTVSSPESPLPRSWKIKIDGIRALYVKSTKVKFGGRTFRAGQRNNRRKVRPPNVKKGKRKGSRSQFFNKFAVGLDAVSQQLPIASVQGLWGRAETKKAQTICLKQLESNKPLKESQRCLFLADNWVVRVCGFLRALSQRQGPTPYIRYRYRCNM

SEQ ID NO: 97 - protein, 392 aa, synthetic construct
  REGION 1..392: note = Description of Artificial Sequence: Synthetic polypeptide
ARNVGQRNASRQSKRESAKARSRRVTGGHASVTQGVALINAAANADRDHTTGCEPCTWERVNLPLQEVIHGCDSCTKSSPFWRDIKVVNKGYREAKEEIMRIASGISADHLSRALSHNKVMGRLNLDEVCILDFRTVLDTSLKHLTDSRSNGIKEHIRAVHRKIRMRRKSGKTARALRKQYFALRRQWKAGHKPNSIREGNSLTALRAVGFDVGVSEGTEPMPAPQTEVVLSVFYKGSATRILRISSPHPIAKRSWKVKIAGIKALKLIRREHDFSFGRETYNASQRAEKRKFSPHAARKDFFNSFAVQLDRLAQQLCVSSVENLWVTEPQQKLLTLAKDTAPYGIREGARFADTRARLAWNWVFRVCGFTRALHQEQEPTPYCRFTWRSKM

SEQ ID NO: 98 - protein, 1228 aa, unidentified
  REGION 1..1228: note = Description of Unknown: Lachnospiraceae bacterium sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 99 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 100 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 101 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 102 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 103 - protein, 1259 aa, Moraxella bovoculi
MGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 104 - protein, 1269 aa, Moraxella bovoculi
MGIHGVPAALFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 105 - protein, 1306 aa, Thiomicrospira sp.
MGIHGVPAATKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 106 - protein, 1214 aa, Butyrivibrio sp.
MGIHGVPAAYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 107 - protein, 1129 aa, unidentified
  REGION 1..1129: note = Description of Unknown: AacCas12b sequence
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 108 - protein, 1224 aa, synthetic construct
  REGION 1..1224: note = Description of Artificial Sequence: Synthetic polypeptide
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 109 - protein, 1297 aa, synthetic construct
  REGION 1..1297: note = Description of Artificial Sequence: Synthetic polypeptide
MATLVSFTKQYQVQKTLRFELIPQGKTQANIDAKGFINDDLKRDENYMKVKGVIDELHKNFIEQTLVNVDYDWRSLATAIKNYRKDRSDTNKKNLEKTQEAARKEIIAWFEGKRGNSAFKNNQKSFYGKLFKKELFSEILRSDDLEYDEETQDAIACFDKFTTYFVGFHENRKNMYSTEAKSTSVAYRVVNENFSKFLSNCEAFSVLEAVCPNVLVEAEQELHLHKAFSDLKLSDVFKVEAYNKYLSQTGIDYYNQIIGGISSAEGVRKIRGVNEVVNNAIQQNDELKVALRNKQFTMVQLFKQILSDRSTLSFVSEQFTSDQEVITVVKQFNDDIVNNKVLAVVKTLFENFNSYDLEKIYINSKELASVSNALLKDWSKIRNAVLENKIIELGANPPKTKISAVEKEVKNKDFSIAELASYNDKYLDKEGNDKEICSIANVVLEAVGALEIMLAESLPADLKTLENKNKVKGILDAYENLLHLLNYFKVSAVNDVDLAFYGAFEKVYVDISGVMPLYNKVRNYATKKPYSVEKFKLNFAMPTLADGWDKNKERDNGSIILLKDGQYYLGVMNPQNKPVIDNAVCNDAKGYQKMVYKMFPEISKMVTKCSTQLNAVKAHFEDNTNDFVLDDTDKFISDLTITKEIYDLNNVLYDGKKKFQIDYLRNTGDFAGYHKALETWIDFVKEFLSKYRSTAIYDLTTLLPTNYYEKLDVFYSDVNNLCYKIDYENISVEQVNEWVEEGNLYLFKIYNKDFATGSTGKPNLHTMYWNAVFAEENLHDVVVKLNGGAELFYRPKSNMPKVEHRVGEKLVNRKNVNGEPIADSVHKEIYAYANGKISKSELSENAQEELPLAIIKDVKHNITKDKRYLSDKYFFHVPITLNYKANGNPSAFNTKVQAFLKNNPDVNIIGIDRGERNLLYVVVIDQQGNIIDKKQVSYNKVNGYDYYEKLNQREKERIEARQSWGAVGKIKELKEGYLSLVVREIADMMVKYNAIVVMENLNAGFKRVRGGIAEKAVYQKFEKMLIDKLNYLVFKDVEAKEAGGVLNAYQLTDKFDSFEKMGNQSGFLFYVPAAYTSKIDPVTGFANVFSTKHITNTEAKKEFICSFNSLRYDEAKDKFVLECDLNKFKIVANSHIKNWKFIIGGKRIVYNSKNKTYMEKYPCEDLKATLNASGIDFSSSEIINLLKNVPANREYGKLFDETYWAIMNTLQMRNSNALTGEDYIISAVADDNEKVFDSRTCGAELPKDADANGAYHIALKGLYLLQRIDISEEGEKVDLSIKNEEWFKFVQQKEYAR

SEQ ID NO: 110 - protein, 1229 aa, synthetic construct
  REGION 1..1229: note = Description of Artificial Sequence: Synthetic polypeptide
MKEQFINRYPLSKTLRFSLIPVGETENNFNKNLLLKKDKQRAENYEKVKCYIDRFHKEYIESVLSKARIEKVNEYANLYWKSNKDDSDIKAMESLENDMRKQISKQLTSTEIYKKRLFGKELICEDLPSFLTDKDERETVECFRSFTTYFKGFNTNRENMYSSDGKSTAIAYRCINDNLPRFLDNVKSFQKVFDNLSDETITKLNTDLYNIFGRNIEDIFSVDYFEFVLTQSGIEIYNSMIGGYTCSDKTKIQGLNECINLYNQQVAKNEKSKKLPLMKPLYKQILSEKDSVSFIPEKFNSDNEVLHAIDDYYTGHIGDFDLLTELLQSLNTYNANGIFVKSGVAITDISNGAFNSWNVLRSAWNEKYEALHPVTSKTKIDKYIEKQDKIYKAIKSFSLFELQSLGNENGNEITDWYISSINESNSKIKEAYLQAQKLLNSDYEKSYNKRLYKNEKATELVKNLLDAIKEFQKLIKPLNGTGKEENKDELFYGKFTSYYDSIADIDRLYDKVRNYITQKPYSKDKIKLNFDNPQLLGGWDKNKESDYRTVLLHKDGLYYLAVMDKSHSKAFVDAPEITSDDKDYYEKMEYKLLPGPNKMLPKVFFASKNIDTFQPSDRILDIRKRESFKKGATFNKAECHEFIDYFKDSIKKHDDWSQFGFKFSPTESYNDISEFYREISDQGYSVRFNKISKNYIDGLVNNGYIYLFQIYNKDFSKYSKGTPNLHTLYFKMLFDERNLSNVVYKLNGEAEMFYREASIGDKEKITHYANQPIKNKNPDNEKKESVFEYDIVKDKRFTKRQFSLHLPITINFKAHGQEFLNYDVRKAVKYKDDNYVIGIDRGERNLIYISVINSNGEIVEQMSLNEIISDNGHKVDYQKLLDTKEKERDKARKNWTSVENIKELKEGYISQVVHKICELVIKYDAVIAMEDLNFGFKRGRFPVEKQVYQKFENMLISKLNLLIDKKAEPTEDGGLLRAYQLTNKFDGVNKAKQNGIIFYVPAWDTSKIDPATGFVNLLKPKCNTSVPEAKKLFETIDDIKYNANTDMFEFYIDYSKFPRCNSDFKKSWTVCTNSSRILTFRNKEKNNKWDNKQIVLTDEFKSLFNEFGIDYKGNLKDSILSISNADFYRRLIKLLSLTLQMRNSITGSTLPEDDYLISPVANKSGEFYDSRNYKGTNAALPCDADANGAYNIARKALWAINVLKDTPDDMLNKAKLSITNAEWLEYTQK

SEQ ID NO: 111 - protein, 1470 aa, synthetic construct
  REGION 1..1470: note = Description of Artificial Sequence: Synthetic polypeptide
MNNPRGAFGGFTNLYSLSKTLRFELKPYLEIPEGEKGKLFGDDKEYYKNCKTYTEYYLKKANKEYYDNEKVKNTDLQLVNFLHDERIEDAYQVLKPVFDTLHEEFITDSLESAEAKKIDFGNYYGLYEKQKSEQNKDEKKKIDKPLETERGKLRKAFTPIYEAEGKNLKNKAGKEKKDKDILKESGFKVLIEAGILKYIKNNIDEFADKKLKNNEGKEITKKDIETALGAENIEGIFDGFFTYFSGFNQNRENYYSTEEKATAVASRIVDENLSKFCDNILLYRKNENDYLKIFNFLKNKGKDLKLKNSKFGKENEPEFIPAYDMKNDEKSFSVADFVNCLSQGEIEKYNAKIANANYLINLYNQNKDGNSSKLSMFKILYKQIGCGEKKDFIKTIKDNAELKQILEKACEAGKKYFIRGKSEDGGVSNIFDFTDYIQSHENYKGVYWSDKAINTISGKYFANWDTLKNKLGDAKVFNKNTGEDKADVKYKVPQAVMLSELFAVLDDNAGEDWREKGIFFKASLFEGDQNKSEIIKNANRPSQALLKMICDDMESLAKNFIDSGDKILKISDRDYQKDENKQKIKNWLDNALWINQILKYFKVKANKIKGDSIDARIDSGLDMLVFSSDNPAEDYDMIRNYLTQKPQDEINKLKLNFENSSLAGGWDENKEKDNSCIILKDEQDKQYLAVMKYENTKVFEQKNSQLYIADNAAWKKMIYKLVPGASKTLPKVFFSKKWTANRPTPSDIVEIYQKGSFKKENVDFNDKKEKDESRKEKNREKIIAELQKTCWMDIRYNIDGKIESAKYVNKEKLAKLIDFYKENLKKYPSEEESWDRLFAFGFSDTKSYKSIDQFYIEVDKQGYKLEFVTINKARLDEYVRDGKIYLFEIRSRDNNLVNGEEKTSAKNLQTIYWNAAFGGDDNKPKLNGEAEIFYRPAIAENKLNKKKDKNGKEIIDGYRFSKEKFIFHCPITLNFCLKETKINDKLNAALAKPENGQGVYFLGIDRGEKHLAYYSLVNQKGEILEQGTLNLPFLDKNGKSRSIKVEKKSFEKDSNGGIIKDKDGNDKIKIEFVECWNYNDLLDARAGDRDYARKNWTTIGTIKELKDGYISQVVRKIVDLSIYKNTETKEFREMPAFIVLEDLNIGFKRGRQKIEKQVYQKLELALAKKLNFLVDKKADIGEIGSVTKAIQLTPPVNNFGDMENRKQFGNMLYIRADYTSQTDPATGWRKSIYLKSGSESNVKEQIEKSFFDIRYESGDYCFEYRDRHGKMWQLYSSKNGVSLDRFHGERNNSKNVWESEKQPLNEMLDILFDEKRFDKSKSLYEQMFKGGVALTRLPKEINKKDKPAWESLRFVIILIQQIRNTGKNGDDRNGDFIQSPVRDEKTGEHFDSRIYLDKEQKGEKADLPTSGDANGAYNIARKGIVVAEHIKRGFDKLYISDEEWDTWLAGDEIWDKWLKENRESLTKTRK

SEQ ID NO: 112 - protein, 1282 aa, synthetic construct
  REGION 1..1282: note = Description of Artificial Sequence: Synthetic polypeptide
MNGNRIIVYREFVGVTPVAKTLRNELRPIGHTQEHIIHNGLIQEDELRQEKSTELKNIMDDYYREYIDKSLSGVTDLDFTLLFELMNLVQSSPSKDNKKALEKEQSKMREQICTHMQSDSNYKNIFNAKFLKEILPDFIKNYNQYDAKDKAGKLETLALFNGFSTYFTDFFEKRKNVFTKEAVSTSIAYRIVHENSLTFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKENDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMSKTQWTVYTNGERLQSEFNNARRTGKTKSINLTETIKLLLEDNEINYADGHDVRIDMEKMDEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 113 - protein, 1225 aa, synthetic construct
  REGION 1..1225: note = Description of Artificial Sequence: Synthetic polypeptide
MKKIDSFVNYYPLSKTLRFSLIPVGKTEDNFNAKLLLEEDEKRAIEYEKVKRYIDRYHKHFIETVLANFHLDDVNEYAELYYKAGKDDKDLKYMEKLEGKMRKSISAAFTKDKKYKEIFGQEIIKNILPEFLENEDEKESVKMFQGFFTYFTGFNDNRKNMYTHEAQTTAISYRCINENLPKFLDNVQSFAKIKESISSDIMNKLDEVCMDLYGVYAQDMFCTDYFSFVLSQSGIDRYNNIIGGYVDDKGVKIQGINEYINLYNQQVDEKNKRLPLMKKLYKQILIEKESISFIPEKFESDNIVINAISDYYHNNVENLFDDFNKLFNEFSEYDDNGIFVTSGLAVTDISNAVFGSWNIISDSWNEEYKDSHPMKKTTNAEKYYEDMKKEYKKNLSFTIAELQRLGEAGCNDECKGDIKEYYKTTVAEKIENIKNAYEISKDLLASDYEKSNDKKLCKNDSAISLLKNLLDSIKDLEKTIKPLLGTGKEENKDDVFYGKFTNLYEMISEIDRLYDKVRNYVTQKPYSKDKIKLNFENPQHLGGWDKNKERDYRSVLLKKEDKYYLAIMDKSNNKAFIDFPDDGECYEKIEYKLLPGPNKMLPKVFFASSNIEYFAPSKKILEIRSRESFKKGDMFNLKDCHEFIDFFKESIKKHEDWSQFGFEFSPTEKYNDISEFYNEVKIQGYSLKYKNVSKKYIDELIECGQLYLFQIYNKDFSVYAKGNPNLHTMYFKMLFDERNLANVVYQLNGGAEMFYRKASIKDSEKIVHHANQPIKNKNADNVKKESVFEYDIIKDKRFTKRQFSIHIPITLNFKAKGQNFINNDVRMALKKADENYVIGIDRGERNLLYICVINSKGEIVEQKSLNEIIGDNGYRVDYHKLLDKKEAERDEARKSWGTIENIKELKEGYLSQIVHEISKLVIKYDAVIAIEDLNSGFKKGRFKVEKQVYQKFENMLCTKLNYLVDKNADANECGGLLKAYQLTNKEDGANRGRQNGIIFSVPAWLTSKIDPVTGFADLLRPKYKSVSESVEFISKIDNIRYNSKEDYFEFDIDYSKFPNSTASYKKKWTVCTYGERIINVRNKEKNNMWDNKTIVLTDEFKKLFADFGVDVSKNIKESVLAIDSKDFYYRFINLLANTLQLRNSEVGNVDVDYLISPVKGVDGSFYDSRLVKEKTLPENADANGAYNIARKALWAIDVLKQTKDEELKNANLSIKNAEWLEYVQK

SEQ ID NO: 114 - protein, 1293 aa, synthetic construct
  REGION 1..1293: note = Description of Artificial Sequence: Synthetic polypeptide
MRTMVTFEDFTKQYQVSKTLRFELIPQGKTLENMKRDGIISVDRQRNEDYQKAKGILDKLYKYILDFTMETVVIDWEALATATEEFRKSKDKKTYEKVQSKIRTALLEHVKKQKVGTEDLFKGMFSSKIITGEVLAAFPEIRLSDEENLILEKFKDFTTYFTGFFENRKNVFTDEALSTSFTYRLVNDNFIKFFDNCIVFKNVVNISPHMAKSLETCASDLGIFPGVSLEEVFSISFYNRLLTQTGIDQFNQLLGGISGKEGEHKQQGLNEIINLAMQQNLEVKEVLKNKAHRFTPLFKQILSDRSTMSFIPDAFADDDEVLSAVDAYRKYLSEKNIGDRAFQLISDMEAYSPELMRIGGKYVSVLSQLLFYSWSEIRDGVKAYKESLITGKKTKKELENIDKEIKYGVTLQEIKEALPKKDIYEEVKKYAMSVVKDYHAGLAEPLPEKIETDDERASIKHIMDSMLGLYRFLEYFSHDSIEDTDPVFGECLDTILDDMNETVPLYNKVRNFSTRKVYSTEKFKLNFNNSSLANGWDKNKEQANGAILLRKEGEYFLGIFNSKNKPKLVSDGGAGIGYEKMIYKQFPDFKKMLPKCTISLKDTKAHFQKSDEDFTLQTDKFEKSIVITKQIYDLGTQTVNGKKKFQVDYPRLTGDMEGYRAALKEWIDFGKEFIQAYTSTAIYDTSLFRDSSDYPDLPSFYKDVDNICYKLTFEWIPDAVIDDCIDDGSLYLFKLHNKDFSSGSIGKPNLHTLYWKALFEEENLSDVVVKLNGQAELFYRPKSLTRPVVHEEGEVIINKTTSTGLPVPDDVYVELSKFVRNGKKGNLTDKAKNWLDKVTVRKMPHAITKDRRFTVDKFFFHVPITLNYKADSSPYRFNDFVRQYIKDCSDVKIIGIDRGERNLIYAVVIDGKGNIIEQRSFNTVGTYNYQEKLEQKEKERQTARQDWATVTKIKDLKKGYLSAVVHELSKMIVKYKAIVALENLNVGFKRMRGGIAERSVYQQFEKALIDKLNYLVFKDEEQSGYGGVLNAYQLTDKFESFSKMGQQTGFLFYVPAAYTSKIDPLTGFINPFSWKHVKNREDRRNFLNLFSKLYYDVNTHDFVLAYHHSNKDSKYTIKGNWEIADWDILIQENKEVFGKTGTPYCVGKRIVYMDDSTTGHNRMCAYYPHTELKKLLSEYGIEYTSGQDLLKIIQEFDDDKLVKGLFYIIKAALQMRNSNSETGEDYISSPIEGRPGICFDSRAEADTLPYDADANGAFHIAMKGLLLTERIRNDDKLAISNEEWLNYIQEMRG

SEQ ID NO: 115 - protein, 1399 aa, synthetic construct
  REGION 1..1399: note = Description of Artificial Sequence: Synthetic polypeptide
MNKDIRKNFTDFVGISEIQKTLRFILIPIGKTAQNIDKYNMFEDDEIRHEYYPILKEACDDFYRNHIDQQFENLELDWSKLDEALASEDRDLINETRATYRQVLFNRLKNSVDIKGDSKKNKTLSLESSDKNLGKKKTKNTFQYNFNDLFKAKLIKAILPLYIEYIYEGEKLENAKKALKMYNRFTSRLSNFWQARANIFTDDEISTGSPYRLVNDNFTIFRINNSIYTKNKPFIEEDILEFEKKLKSKKIIKDFESVDDYFTVNAFNKLCTQNGIDKYNSILGGFTTKEREKVKGLNELFNLAQQSINKGKKGEYRKNIRLGKLTKLKKQILAISDSTSFLIEQIEDDQDLYNKIKDFFELLLKEEIENENIFTQYANLQKLIEQADLSKIYINAKHLNKISHQVTGKWDSLNKGIALLLENININEESKEKSEVISNGQTKDISSEAYKRYLQIQSEEKDIERLRTQIYFSLEDLEKALDLVLIDENMDRSDKSILSYVQSPDLNVNFERDLTDLYSRIMKLEENNEKLLANHSAIDLIKEFLDLIMLRYSRWQILFCDSNYELDQTFYPIYDAVMEILSNIIRLYNLARNYLSRKPDRMKKKKINFNNPTLADGWSESKIPDNSSMLFIKDGMYYLGIIKNRAAYSELLEAESLQSSEKKKSENSSYERMNYHFLPDAFRSIPKSSIAMKAVKEHFEINQKTADLLLDTDKFSKPLRITKEIFDMQYVDLHKNKKKYQVDYLRDTGDKKGYRKALNTWLNFCKDFISKYKGRNLFDYSKIKDADHYETVNEFYNDVDKYSYHIFFTSVAETTVEKFISEGKLYLFQLYNKDFSPHSTGKPNLHTIYWRALFSEENLTSKNIKLNGQAEIFFRPKQIETPFTHKKGSILVNRFDVNGNPIPINVYQEIKGFKNNVIKWDDLNKTTQEGLENDQYLYFESEFEIIKDRRYTEDQLFFHVPISFNWDIGSNPKINDLATQYIVNSNDIHIIGIDRGENHLIYYSVIDLQGAIVEQGSLNTITEYTENKFLNNKTNNLRKIPYKDILQQREDERADARIKWHAIDKIKDLKDGYLGQIVHFLAKLIIKYNAIVILEDLNYGFKRGRFKVERQVYQKFEMALMKKLNVLVFKDYDIDEIGGPLKPWQLTRPIDSYERMGRQNGILFYVPAAYTSAVDPVTGFANLFYLNNVKNSEKFHFFSKFESIKYHSDQDMFSFAFDYNNFGTTTRINDLSKSKWQVFTNHERSVWNNKEKNYVTQNLTDLIKKLLRTYNIEFKNNQNVLDSILKIENNTDKENFARELFRLFRLTIQLRNTTVNENNTEITENELDYIISPVKDKNGNFFDSRDELKNLPDNGDANGAYNIARKGLLYIEQLQESIKTGKLPTLSISTLDWFNYIMK

SEQ ID NO: 116 - protein, 1272 aa, synthetic construct
  REGION 1..1272: note = Description of Artificial Sequence: Synthetic polypeptide
MTPIFCNFVVYQIMLFNNNININVKTMNKKHLSDFTNLFPVSKTLRFRLEPQGKTMENIVKAQTIETDEERSHDYEKTKEYIDDYHRQFIDDTLDKFAFKVESTGNNDSLQDYLDAYLSANDNRTKQTEEIQTNLRKAIVSAFKMQPQFNLLFKKEMVKHLLPQFVDTDDKKRIVAKFNDFTTYFTGFFTNRENMYSDEAKSTSIAYRIVNQNLIKFVENMLTFKSHILPILPQEQLATLYDDFKEYLNVASIAEMFELDHFSIVLTQRQIEVYNSVIGGRKDENNKQIKPGLNQYINQHNQAVKDKSARLPLLKPLFNQILSEKAGVSFLPKQFKSASEVVKSLNEAYAELSPVLAAIQDVVTNITDYDCNGIFIKNDLGLTDIAQRFYGNYDAVKRGLRNQYELETPMHNGQKAEKYEEQVAKHLKSIESVSLAQINQVVTDGGDICDYFKAFGATDDGDIQRENLLASINNAHTAISPVLNKENANDNELRKNTMLIKDLLDAIKRLQWFAKPLLGAGDETNKDQVFYGKFEPLYNQLDETISPLYDKVRSYLTKKPYSLDKFKINFEKSNLLGGWDPGADRKYQYNAVILRKDNDFYLGIMRDEATSKRKCIQVLDCNDEGLDENFEKVEYKQIKPSQNMPRCAFAKKECEENADIMELKRKKNAKSYNTNKDDKNALIRHYQRYLDRTYPEFGFVYKDADEYDTVKAFTDSMDSQDYKLSFLQVSETGLNKLVDEGDLYLFKITNKDFSSYAKGRPNLHTIYWRMLFDPKNLANVVYKLEGKAEVFFRRKSLASTTTHKAKQAIKNKSRYNEAVKPQSTFDYDIIKDRRFTADKFEFHVPIKMNFKAAGWNSTRLTNEVREFIKSQGVRHIIGIDRGERHLLYLTMIDMDGNIVKQCSLNAPAQDNARASEVDYHQLLDSKEADRLAARRNWGTIENIKELKQGYLSQVVHLLATMMVDNDAILVLENLNAGFMRGRQKVEKSVYQKFEKMLIDKLNYIVDKGQSPDKPTGALHAVQLTGLYSDFNKSNMKRANVRQCGFVFYIPAWNTSKIDPVTGFVNLFDTHLSSMGEIKAFFSKFDSIRYNQDKGWFEFKFDYSRFTTRAEGCRTQWTVCTYGERIWTHRSKNQNNQFVNDTVNVTQQMLQLLQDCGIDPNGNLKEAIANIDSKKSLETLLHLFKLTVQMRNSVTGSEVDYMISPVADERGHFFDSRESDEHLPANADANGAFNIARKGLMVVRQIMATDDVSKIKFAVSNKDWLRFAQHIDD

SEQ ID NO: 117 - protein, 1313 aa, synthetic construct
  REGION 1..1313: note = Description of Artificial Sequence: Synthetic polypeptide
MNKGGYVIMEKMTEKNRWENQFRITKTIKEEIIPTGYTKVNLQRVNMLKREMERNEDLKKMKEICDEYYRNMIDVSLRLEQVRTLGWESLIHKYRMLNKDEKEIKALEKEQEDLRKKISKGFGEKKAWTGEQFIKKILPQYLMDHYTGEELEEKLRIVKKFKGCTMFLSTFFKNRENIFTDKPIHTAVGHRITSENAMLFAANINTYEKMESNVTLEIERLQREFWRRGINISEIFTDAYYVNVLTQKQIEAYNKICGDINQHMNEYCQKQKLKFSEFRMRELKKQILAVVGEHFEIPEKIESTKEVYRELNEYYESLKELHGQFEELKSVQLKYSQIYVQKKGYDRISRYIGGQWDLIQECMKKDCASGMKGTKKNHDAKIEEEVAKVKYQSIEHIQKLVCTYEEDRGHKVTDYVDEFIVSVCDLLGADHIITRDGERIELPLQYEPGTDLLKNDTINQRRLSDIKTILDWHMDMLEWLKTFLVNDLVIKDEEFYMAIEELNERMQCVISVYNRIRNYVTQKGYEPEKIRICFDKGTILTGWTTGDNYQYSGFLLMRNDKYYLGIINTNEKSVRKILDGNEECKDENDYIRVGYHLINDASKQLPRIFVMPKAGKKSEILMKDEQCDYIWDGYCHNKHNESKEFMRELIDYYKRSIMNYDKWEGYCFKFSSTESYDNMQDFYKEVREQSYNISFSYINENVLEQLDKDGKIYLFQVYNKDFAAGSTGTPNLHTMYLQNLFSSQNLELKRLRLGGNAELFYRPGTEKDVTHRKGSILVDRTYVREEKDGIEVRDTVPEKEYLEIYRYLNGKQKGDLSESAKQWLDKVHYREAPCDIIKDKRYAQEKYFLHFSVEINPNAEGQTALNDNVRRWLSEEEDIHVIGIDRGERNLIYVSLMDGKGRIKDQKSYNIVNSGNKEPVDYLAKLKVREKERDEARRNWKAIGKIKDIKTGYLSYVVHEIVEMAVREKAIIVMEDLNYGFKRGRFKVERQVYQKFEEMLINKLNYVVDKQLSVDEPGGLLRGYQLAFIPKDKKSSMRQNGIVFYVPAGYTSKIDPTTGFVNIFKFPQFGKGDDDGNGKDYDKIRAFFGKFDEIRYECDEKVTADNTREVKERYRFDFDYSKFETHLVHMKKTKWTVYAEGERIKRKKVGNYWTSEVISDIALRMSNTLNIAGIEYKDGHNLVNEICALRGKQAGIILNELLEIVRLTVQLRNSTTEGDVDERDEIISPVLNEKYGCFYHSTEYKQQNGDVLPKDADANGAYCIGLKGIYEIRQIKNKWKEDMTKGEGKALNEGMRISHDQWFEFIQNMNKGE

SEQ ID NO: 118 - protein, 1134 aa, synthetic construct
  REGION 1..1134: note = Description of Artificial Sequence: Synthetic polypeptide
MNELVKNRCKQTKTICQKLIPIGKTRETIEKYNLMEIDRKIAANKELMNKLFSLIAGKHINDTLSKCTDLDFEPLLTSLSSLNNAKENDRDNLREYYDSVFEEKKTLAEEISSRLTAVKFAGKDFFTKNIPDFLETYEGDDKNEMSELVSLVIENTVTAGYVKKLEKIDRSMEYRLVSGTVVKRVLTDNADIYEKNIEKAKDFDYGVLNIDEASQFTTLVAKDYANYLTADGIAIYNVGIGKINLALNEYCQKNKEYSYNKLALLPLQKMLYGEKLSLFEKLEDFTSDEELINSYNKFAKTVNESGLAEIIKKAVPSYDEIVIKPNKISNYSNSITGHWSLVNRIMKDYLENNGIKNADKYMEKGLTLSEIGDALENKNIKHSDFISNLINDLGHTYTEIKENKESLKKDESVNALIIKKELDMLLSILQNLKVFDIDNEMFDTGFGIEVSKAIEILGYGVPLYNKIRNYITKKPDPKKKFMTKFGSATIGTGITTSVEGSKKATFLKDGDAVFLLLYNTAGCKANNVSVSNLADLINSSLEIENSGKCYQKMIYQTPGDIKKQIPRVFVYKSEDDDLIKDFKAGLHKTDLSFLNGRLIPYLKEAFATHETYKNYTFSYRNSYESYDEFCEHMSEQAYILEWKWIDKKLIDDLVEDGSLLMFRVWNRFMKKKEGKISKHAKIVNELFSDENASNAAIKLLSVFDIFYRDKQIDNPIVHKAGTTLYNKRTKDGEVIVDYTTMVKNKEKRPNVYTTTKKYDIIKDRRYTEEQFEIHLHVNIGKEENKEKLETSKVINEKKNTLVVTRSNEHLLYVVIFDENDNILLKKSLNTVKGMNFKSKLEVVEIQKKENMQSWKTVGSNQALMEGYLSFAIKEIADLVKEYDAILVLEQNSVGKNILNERVYTRFKEMLITNLSLDVDYENKDFYSYTELGGKVASWRDCVTNGICIQVPSAYKYKDPTTSFSTISMYAKTTAEKSKKLKQIKSFKYNRERGLFELVIAKGVGLENNIVCDSFGSRSIIENDISKEVSCTLKIEKYLIDAGIEYNDEKEVLKDLDTAAKTDAVHKAVTLLLKCFNESPDGRYYISPCGEHFTLCDAPEVLSAINYYIRSRYIREQIVEGVKKMEYKKTILLAK

SEQ ID NO: 119 - protein, 1259 aa, synthetic construct
  REGION 1..1259: note = Description of Artificial Sequence: Synthetic polypeptide
MNYKTGLEDFIGKESLSKTLRNALIPTESTKIHMEEMGVIRDDELRAEKQQELKEIMDDYYRTFIEEKLGQIQGIQWNSLFQKMEETMEDISVRKDLDKIQNEKRKEICCYFTSDKRFKDLFNAKLITDILPNFIKDNKEYTEEEKAEKEQTRVLFQRFATAFTNYFNQRRNNFSEDNISTAISFRIVNENSEIHLQNMRAFQRIEQQYPEEVCGMEEEYKDMLQEWQMKHIYSVDFYDRELTQPGIEYYNGICGKINEHMNQFCQKNRINKNDFRMKKLHKQILCKKSSYYEIPFRFESDQEVYDALNEFIKTMKKKEIIRRCVHLGQECDDYDLGKIYISSNKYEQISNALYGSWDTIRKCIKEEYMDALPGKGEKKEEKAEAAAKKEEYRSIADIDKIISLYGSEMDRTISAKKCITEICDMAGQISIDPLVCNSDIKLLQNKEKTTEIKTILDSFLHVYQWGQTFIVSDIIEKDSYFYSELEDVLEDFEGITTLYNHVRSYVTQKPYSTVKFKLHFGSPTLANGWSQSKEYDNNAILLMRDQKFYLGIFNVRNKPDKQIIKGHEKEEKGDYKKMIYNLLPGPSKMLPKVFITSRSGQETYKPSKHILDGYNEKRHIKSSPKFDLGYCWDLIDYYKECIHKHPDWKNYDFHFSDTKDYEDISGFYREVEMQGYQIKWTYISADEIQKLDEKGQIFLFQIYNKDFSVHSTGKDNLHTMYLKNLFSEENLKDIVLKLNGEAELFFRKASIKTPIVHKKGSVLVNRSYTQTVGNKEIRVSIPEEYYTEIYNYLNHIGKGKLSSEAQRYLDEGKIKSFTATKDIVKNYRYCCDHYFLHLPITINFKAKSDVAVNERTLAYIAKKEDIHIIGIDRGERNLLYISVVDVHGNIREQRSFNIVNGYDYQQKLKDREKSRDAARKNWEEIEKIKELKEGYLSMVIHYIAQLVVKYNAVVAMEDLNYGFKTGRFKVERQVYQKFETMLIEKLHYLVFKDREVCEEGGVLRGYQLTYIPESLKKVGKQCGFIFYVPAGYTSKIDPTTGFVNLFSFKNLTNRESRQDFVGKFDEIRYDRDKKMFEFSFDYNNYIKKGTILASTKWKVYTNGTRLKKIVVNGKYTSQSMEVELTDAMEKMLQRAGIEYHDGKDLKGQIVEKGIEAEIIDIFRLTVQMRNSRSESEDREYDRLISPVLNDKGEFFDTATADKTLPQDADANGAYCIALKGLYEVKQIKENWKENEQFPRNKLVQDNKTWFDFMQKKRYL

SEQ ID NO: 120 - protein, 1271 aa, synthetic construct
  REGION 1..1271: note = Description of Artificial Sequence: Synthetic polypeptide
MEDKQFLERYKEFIGLNSLSKTLRNSLIPVGSTLKHIQEYGILEEDSLRAQKREELKGIMDDYYRNYIEMHLRDVHDIDWNELFEALTEVKKNQTDDAKKRLEKIQEKKRKEIYQYLSDDAVFSEMFKEKMISGILPDFIRCNEGYSEEEKEEKLKTVALFHRFTSSFNDFFLNRKNVFTKEAIVTAIGYRVVHENAEIFLENMVAFQNIQKSAESQISIIERKNEHYFMEWKLSHIFTADYYMMLMTQKAIEHYNEMCGVVNQQMREYCQKEKKNWNLYRMKRLHKQILSNASTSFKIPEKYENDAEVYESVNSFLQNVMEKTVMERIAVLKNSTDNFDLSKIYITAPYYEKISNYLCGSWNTITDCLTHYYEQQIAGKGARKDQKVKAAVKADKWKSLSEIEQLLKEYARAEEVKRKPEEYIAEIENIVSLKEAHLLEYHPEVNLIENEKYATEIKDVLDNYMELFHWMKWFYIEEAVEKEVNFYGELDDLYEEIKDIVPLYNKVRNYVTQKPYSDTKIKLNFGTPTLANGWSKSKEYDYNAILLQKDGKYYMGIFNPIQKPEKEIIEGHSQPLEGNEYKKMVYYYLPSANKMLPKVLLSKKGMEIYQPSEYIINGYKERRHIKSEEKFDLQFCHDLIDYFKSGIERNSDWKVFGFDFSDTDTYQDISGFYREVEDQGYKIDWTYIKEADIDRLNEEGKLYLFQIYNKDFSEKSTGRENLHTMYLKNLFSEENVREQVLKLNGEAEIFFRKSSVKKPIIHKKGTMLVNRTYMEEVNGNSVRRNIPEKEYQEIYNYKNHRLKGELSTEAKKYLEKAVCHETKKDIVKDYRYSVDKFFIHLPITINYRASGKETLNSVAQRYIAHQNDMHVIGIDRGERNLIYVSVINMQGEIKEQKSFNIINEFNYKEKLKEREQSRGAARRNWKEIGQIKDLKEGYLSGVIHEIAKMMIKYHAIIAMEDLNYGFKRGRFKVERQVYQKFENMLIQKLNYLVFKDRPADEDGGVLRGYQLAYIPDSVKKMGRQCGMIFYVPAAFTSKIDPTTGFVDIFKHKVYTTEQAKREFILSFDEICYDVERQLFRFTFDYANFVTQNVTLARNNWTIYTNGTRAQKEFGNGRMRDKEDYNPKDKMVELLESEGIEFKSGKNLLPALKKVSNAKVFEELQKIVRFTVQLRNSKSEENDVDYDHVISPVLNEEGNFFDSSKYKNKEEKKESLLPVDADANGAYCIALKGLYIMQAIQKNWSEEKALSPDVLRLNNNDWFDYIQNKRYR

SEQ ID NO: 121 - protein, 1367 aa, synthetic construct
  REGION 1..1367: note = Description of Artificial Sequence: Synthetic polypeptide
MEKSLNDFIGLYSVSKTLRFELKPVSETLENIKKFHFLEEDKKKANDYKDVKKIIDNYHKYFIDDVLKNASFNWKKLEEAIREYNKNKSDDSALVAEQKKLGDAILKLFTSDKRYKALTAATPKELFESILPDWFGEQCNQDLNKAALKTFQKFTSYFTGFQENRKNVYSAEAIPTAVPYRIVNDNFPKFLQNVLIFKTIQEKCPQIIDEVEKELSSYLGKEKLAGIFTLESFNKYLGQGGKENQRGIDFYNQIIGGVVEKEGGINLRGVNQFLNLYWQQHPDFTKEDRRIKMVPLYKQILSDRSSLSFKIESIENDEELKNALLECADKLELKNDEKKSIFEEVCDLFSSVKNLDLSGIYINRKDINSVSRILTGDWSWLQSRMNVYAEEKFTTKAEKARWQKSLDDEGENKSKGFYSLTDLNEVLEYSSENVAETDIRITDYFEHRCRYYVDKETEMFVQGSELVALSLQEMCDDILKKRKAMNTVLENLSSENKLREKTDDVAVIKEYLDAVQELLHRIKPLKVNGVGDSTFYSVYDSIYSALSEVISVYNKTRNYITKKAASPEKYKLNFDNPTLADGWDLNKEQANTSVILRKDGMFYLGIMNPKNKPKFAEKYDCGNESCYEKMIYKQFDATKQIPKCSTQKKEVQKYFLSGATEPYILNDKKSFKSELIITKDIWFMNNHVWDGEKFVPKRDNETRPKKFQIGYFKQTGDFDGYKNALSNWISFCKNFLQSYLSATVYDYNFKNSEEYEGLDEFYNYLNATCYKLNFINIPETEINKMVSEGKLYLFQIYNKDFASGSTGMPNMHTLYWKNLFSDENLKNVCLKLNGEAELFYRPAGIKEPVIHKEGSYLVNRTTEDGESIPEKIYFEIYKNANGKLEKLSDEAQNYISNHEVVIKKAGHEIIKDRHYTEPKFLFHVPLTINFKASGNSYSINENVRKFLKNNPDVNIIGLDRGERHLIYLSLINQKGEIIKQFTFNEVERNKNGRTIKVNYHEKLDQREKERDAARKSWQAIGKIAELKEGYLSAVIHQLTKLMVEYNAVVVMEDLNFGFKRGRFHVEKQVYQKFEHILIDKSNYLVFKDRGLNEPGGVLNGYQIAGQFESFQKLGKQSGMLFYVPAGYTSKIDPKTGFVSMMNFKDLTNVHKKRDFFSKFDNIHYDEANGSFVFTFDYKKFDGKAKEEMKLTKWSVYSRDKRIVYFAKTKSYEDVLPTEKLQKIFESNGIDYKSGNNIQDSVMAIGADLKEGAKPSKEISDFWDGLLSNFKLILQMRNSNARTGEDYIISPVMADDGTFFDSREEFKKGEDAKLPLDADANGAYHIALKGLSLINKINLSKDEELKKFDMKISNADWFKFAQEKNYAK

SEQ ID NO: 122 - protein, 1229 aa, synthetic construct
  REGION 1..1229: note = Description of Artificial Sequence: Synthetic polypeptide
MEEKKMSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 123 - protein, 1261 aa, synthetic construct
  REGION 1..1261: note = Description of Artificial Sequence: Synthetic polypeptide
MIIHNCYIGGSFMKKIDSFTNCYSLSKTLRFKLIPIGATQSNFDLNKMLDEDKKRAENYSKAKSIIDKYHRFFIDKVLSSVTENKAFDSFLEDVRAYAELYYRSNKDDSDKASMKTLESKMRKFIALALQSDEGFKDLFGQNLIKKTLPEFLESDTDKEIIAEFDGFSTYFTGFFNNRKNMYSADDQPTAISYRCINDNLPKFLDNVRTFKNSDVASILNDNLKILNEDFDGIYGTSAEDVFNVDYFPFVLSQKGIEAYNSILGGYTNSDGSKIKGLNEYINLYNQKNENIHRIPKMKQLFKQILSERESVSFIPEKFDSDDDVLSSINDYYLERDGGKVLSIEKTVEKIEKLFSAVTDYSTDGIFVKNAAELTAVCSGAFGYWGTVQNAWNNEYDALNGYKETEKYIDKRKKAYKSIESFSLADIQKYADVSESSETNAEVTEWLRNEIKEKCNLAVQGYESSKDLISKPYTESKKLFNNDNAVELIKNALDSVKELENVLRLLLGTGKEESKDENFYGEFLPCYERICEVDSLYDKVRNYMTQKLYKTDKIKLNFQNPQFLGGWDRNKEADYSAVLLRRNSLYYIAIMPSGYKRVFEKIPAPKADETVYEKVIYKLLPGPNKMLPKVFFSKKGIETFNPPKEILEKYELGTHKTGDGFNLDDCHALIDYFKSALDVHSDWSNFGFRFSDTSTYKNIADFYNEVKNQGYKITFCDVPQSYINELVDEGKLYLFQLYNKDFSEHSKGTPNLHTLYFKMLFDERNLENVVFKLNGEAEMFYREASISKDDMIVHPKNQPIKNKNEQNSRKQSTFEYDIVKDRRYTVDQFMLHIPITLNFTANGGTNINNEVRKALKDCDKNYVIGIDRGERNLLYICVVDSEGRIIEQYSLNEIINEYNGNTYSTDYHALLDKKEKERLESRKAWKTVENIKELKEGYISQVVHKICELVEKYDAVIVMEDLNLGFKQGRSGKFEKSVYQKFEKMLIDKLNYFADKKKSPEEIGSVLNAYQLTNAFESFEKMGKQNGFIFYVPAYLTSKIDPTTGFADLLHPSSKQSKESMRDFVGRFDSITFNKTENYFEFELDYNKFPRCNTDYRKKWTVCTYGSRIKTFRNPEKNSEWDNKTVELTPAFMALFEKYSIDVNGDIKAQIMSVDKKDFFVELIGLLRLTLQMRNSETGKVDRDYLISPVKNSEGVFYNSDDYKGIENASLPKDADANGAYNIARKGLWIIEQIKACENDAELNKIRLAISNAEWLEYAQKK

SEQ ID NO: 124 - protein, 1242 aa, synthetic construct
  REGION 1..1242: note = Description of Artificial Sequence: Synthetic polypeptide
MKEQFVNQYPISKTLRFSLIPIGKTEENFNKNLLLKEDEKKAEEYQKVKGYIDRYHKFFIETALCNINFEGFEEYSLLYYKCSKDDNDLKTMEDIEIKLRKQISKTMTSHKLYKDLFGENMIKTILPNFLDSDEEKNSLEMFRGFYTYFSGFNTNRKNMYTEEAKSTSIAYRCINDNLPKFLDNSKSFEKIKCALNKEELKAKNEEFYEIFQIYATDIFNIDFFNFVLTQPGIDKYNGIIGGYTCSDGTKVQGLNEIINLYNQQIAKDDKSKRLPLLKMLYKQILSDRETVSFIPEKFSSDNEVLESINNYFSKNVSNAIKSLKELFQGFEAYNMNGIFISSGVAITDLSNAVFGDWNAISTAWEKAYFETNPPKKNKSQEKYEEELKANYKKIKSFSLDEIQRLGSIAKSPDSIGSVAEYYKITVTEKIDNITELYDGSKELLNCNYSESYDKKLIKNDTVIEKVKTLLDAVKSLEKLIKPLVGTGKEDKDELFYGTFLPLYTSLSAVDRLYDKVRNYATQKPYSKDKIKLNFNCSSFLSGWATDYSSNGGLIFEKDGLYYLGIVNKKFTTEEIDYLQQNADENPAQRIVYDFQKPDNKNTPRLFIRSKGTNYSPSVKEYNLPVEEIVELYDKRYFTTEYRNKNPELYKASLVKLIDYFKLGFTRHESYRHYDFKWKKSEEYNDISEFYKDVEISCYSLKQEKINYNTLLNFVAENRIYLFQIYNKDFSKYSKGTPNLHTRYFKALFDENNLSDVVFKLNGGSEMFFRKASIKDNEKVVHPANQPIDNKNPDNSKKQSTFDYELIKDKRFTKHQFSIHIPITMNFKARGRDFINNDIRKAIKSEYKPYVIGIDRGERNLIYISVINNNGEIVEQMSLNDIISDNGYKVDYQRLLDRKEKERDNARKSWGTIENIKELKEGYISQVIHKICELVIKYDAVIAMEDLNFGFKRGRFNVEKQVYQKFENMLISKLNYLCDKKSEANSEGGLLKAYQLTNKFDGVNKGKQNGIIFYVPAWLTSKIDPVTGFVDLLHPKYISVEETHSLFEKLDDIRYNFEKDMFEFDIDYSKLPKCNADFKQKWTVCTNADRIMTFRNSEKNNEWDNKRILLSDEFKRLFEEFGIDYCHNLKNKILSISNKDFCYRFIKLFALTMQMRNSITGSTNPEDDYLISPVRDENGVFYDSRNFIGSKAGLPIDADANGAYNIARKGLWAINAIKSTADDMLDKVDLSISNAKWLEYVQK

SEQ ID NO: 125 - protein, 1307 aa, synthetic construct
  REGION 1..1307: note = Description of Artificial Sequence: Synthetic polypeptide
MADLSQFTHKYQVPKTLRFELIPQGKTLENLSAYGMVADDKQRSENYKKLKPVIDRIYKYFIEESLKNTNLDWNPLYEAIREYRKEKTTATITNLKEQQDICRRAIASRFEGKVPDKGDKSVKDFNKKQSKLFKELFGKELFTDSVLEQLPGVSLSDEDKALLKSFDKFTTYFVGFYDNRKNVFSSDDISTGIPHRLVQENFPKFIDNCDDYKRLVLVAPELKEKLEKAAEATKIFEDVSLDEIFSIKFYNRLLQQNQIDQFNQLLGGIAGAPGTPKIQGLNETLNLSMQQDKTLEQKLKSVPHRFSPLYKQILSDRSSLSFIPESFSCDAEVLLAVQEYLDNLKTEHVIEDLKEVFNRLTTLDLKHIYVNSTKVTAFSQALFGDWNLCREQLRVYKMSNGNEKITKKALGELESWLKNSDIAFTELQEALADEALPAKVNLKVQEAISGLNEQMAKSLPKELKIPEEKEELKALLDAIQEVYHTLEWFIVSDDVETDTDFYVPLKETLQIIQPIIPLYNKVRNFATQKPYSVEKFKLNFANPTLADGWDENKEQQNCAVLFQKGNNYYLGILNPKNKPDFDNVDTEKQGNCYQKMVYKQFPDFSKMMPKCTTQLKEVKQHFEGKDSDYILNNKNFIKPLTITREVYDLNNVLYDGKKKFQIDYLRKTKDEDGYYTALHTWIDFAKKFVASYKSTSIYDTSTILPPEKYEKLNEFYGALDNLFYQIKFENIPEEIIDTYVEDGKLFLFQIYNKDFAAGATGAPNLHTIYWKAVFDPENVKDVVVKLNGQAELFYRPKSNMDVIRHKVGEKLVNRTLKDGSILTDELHKELYLYANGSLKKGLSEDAKIILDKNLAVIYDVHHEIVKDRRFTTDKFFFHVPLTLNYKCDKNPVKFNAEVQEYLKENPDTYVIGIDRGERNLIYAVVIDPKGRIVEQKSFNVINGFDYHGKLDQREKERVKARQAWTAVGKIKELKQGYLSLVVHEISKMMVRYQAVVVLENLNVGFKRVRSGIAEKAVYQQFEKMLINKLNYLMFKDAGGTEPGSVLNAYQLTDRFESFAKMGLQTGFLFYIPAAFTSKIDPATGFVDPFRWGAIKTLADKREFLSGFESLKFDSTTGNFILHFDVSKNKNFQKKLEGFVPDWDIIIEANKMKTGKGATYIAGKRIEFVRDNNSQGHYEDYLPCNALAETLRQCDIPYEEGKDILPLILEKNDSKLLHSVFKVVRLTLQMRNSNAETGEDYISSPVEDVSGSCFDSRMENEKLPKDADANGAYHIALKGMLALERLRKDEKMAISNNDWLNYIQEKRA

SEQ ID NO: 126 - protein, 1286 aa, synthetic construct
  REGION 1..1286: note = Description of Artificial Sequence: Synthetic polypeptide
MTNFDNFTKKYVNSKTIRLEAIPVGKTLKNIEKMGFIAADRQRDEDYQKAKSVIDHIYKAFMDDCLKDLFLDWDPLYEAVVACWRERSPEGRQALQIMQADYRKKIADRFRNHELYGSLFTKKIFDGSVAQRLPDLEQSAEEKSLLSNFNKFTSYFRDFFDKRKRLFSDDEKHSAIAYRLINENFLKFVANCEAFRRMTERVPELREKLQNTGSLQVYNGLALDEVFSADFYNQLIVQKQIDLYNQLIGGIAGEPGTPNIQGLNATINLALQGDSSLHEKLAGIPHRFNPLYKQILSDVSTLSFVPSAFQSDGEMLAAVRGFKVQLESGRVLQNVRRLFNGLETEADLSRVYVNNSKLAAFSSMFFGRWNLCSDALFAWKKGKQKKITNKKLTEIKKWLKNSDIAIAEIQEAFGEDFPRGKINEKIQAQADALHSQLALPIPENLKALCAKDGLKSMLDTVLGLYRMLQWFIVGDDNEKDSDFYFGLGKILGSLDPVLVLYNRVRNYITKKPYSLTKFRLNFDNSQLLNGWDENNLDTNCASIFIKDGKYYLGISNKNNRPQFDTVATSGKSGYQRMVYKQFANWGRDLPHSTTQMKKVKKHFSASDADYVLDGDKFIRPLIITKEIFDLNNVKFNGKKKLQVDYLRNTGDREGYTHALHTWINFAKDFCACYKSTSIYDISCLRPTDQYDNLMDFYADLGNLSHRIVWQTIPEEAIDNYVEQGQLFLFQLYNKDFAPGADGKPNLHTLYWKAVFNPENLEDVVVKLNGKAELFYRPRSNMDVVRHKVGEKLVNRKLKNGLTLPSRLHEEIYRYVNGTLNKDLSADARSVLPLAVVRDVQHEIIKDRRFTADKFFFHASLTFNFKSSDKPVGFNEDVREYLREHPDTYVVGVDRGERNLIYIVVIDPQGNIVEQRSFNMINGIDYWSLLDQKEKERVEAKQAWETVGKIKDLKCGYLSFLIHEITKIIIKYHAVVILENLSLGFKRVRTGIAEKAVYQQFERMLVTKLGYVVFKDRAGKAPGGVLNAYQLTDNTRTAENTGIQNGFLFYVPAAFTSRVDPATGFFDFYDWGKIKTATDKKNFIAGFNSVRYERSTGDFIVHVGAKNLAVRRVAEDVRTEWDIVIEANVRKMGIDGNSYISGKRIRYRSGEQGHGQYENHLPCQELIRALQQYGIQYETGKDILPAILQQDDAKLTDTVFDVFRLALQMRNTSAETGEDYFNSVVRDRSGRCFDTRRAEAAMPKEADANDAYHIALKGLFVLEKLRKGESIGIKNTEWLRYVQQRHS

SEQ ID NO: 127 - protein, 1264 aa, synthetic construct
  REGION 1..1264: note = Description of Artificial Sequence: Synthetic polypeptide
MENYGGFTGLYPLQKTLKFELRPQGRTMEHLVSSNFFEEDRDRAEKYKIVKKVIDNYHKDFINECLSKRSFDWTPLMKTSEKYYASKEKNGKKKQDLDQKIIPTIENLSEKDRKELELEQKRMRKEIVSVFKEDKRFKYLFSEKLFSILLKDEDYSKEKLTEKEILALKSFNKFSGYFIGLHKNRANFYSEGDESTAIAYRIVNENFPKFLSNLKKYREVCEKYPEIIQDAEQSLAGLNIKMDDIFPMENFNKVMTQDGIDLYNLAIGGKAQALGEKQKGLNEFLNEVNQSYKKGNDRIRMTPLFKQILSERTSYSYILDAFDDNSQLITSINGFFTEVEKDKEGNTFDRAVGLIASYMKYDLSRVYIRKADLNKVSMEIFGSWERLGGLLRIFKSELYGDVNAEKTSKKVDKWLNSGEFSLSDVINAIAGSKSAETFDEYILKMRVARGEIDNALEKIKCINGNFSEDENSKMIIKAILDSVQRLFHLFSSFQVRADFSQDGDFYAEYNEIYEKLFAIVPLYNRVRNYLTKNNLSMKKIKLNFKNPALANGWDLNKEYDNTAVIFLREGKYYLGIMNPSKKKNIKFEEGSGTGPFYKKMAYKLLPDPNKMLPKVFFAKKNINYYNPSDEIVKGYKAGKYKKGENFDIDFCHKLIDFFKESIQKNEDWRAFNYLFSATESYKDISDFYSEVEDQGYRMYFLNVPVANIDEYVEKGDLFLFQIYNKDFASGAKGNKDMHTIYWNAAFSDENLRNVVVKLNGEAELFYRDKSIIEPICHKKGEMLVNRTCFDKTPVPDKIHKELFDYHNGRAKTLSIEAKGYLDRVGVFQASYEIIKDRRYSENKMYFHVPLKLNFKADGKKNLNKMVIEKFLSDKDVHIIGIDRGERNLLYYSVIDRRGNIIDQDSLNIIDGFDYQKKLGQREIERREARQSWNSIGKIKDLKEGYLSKAVHKVSKMVLEYNAIVVLEDLNFGFKRGRFKVEKQVYQKFEKMLIDKLNYLVFKEVLDSRDAGGVLNAYQLTTQLESFNKLGKQSGILFYVPAAYTSKIDPTTGFVSLFNTSRIESDSEKKDFLSGFDSIVYSAKDGGIFAFKFDYRNRNFQREKTDHKNIWTVYTNGDRIKYKGRMKGYEITSPTKRIKDVLSSSGIRYDDGQELRDSIIQSGNKVLINEVYNSFIDTLQMRNSDGEQDYIISPVKNRNGEFFRTDPDRRELPVDADANGAYHIALRGELLMQKIAEDFDPKSDKFTMPKMEHKDWFEFMQTRGD

SEQ ID NO: 128 - protein, 1232 aa, synthetic construct
  REGION 1..1232: note = Description of Artificial Sequence: Synthetic polypeptide
MLHAFTNQYQLSKTLRFGATLKEDEKKCKSHEELKGFVDISYENMKSSATIAESLNENELVKKCERCYSEIVKFHNAWEKIYYRTDQIAVYKDFYRQLSRKARFDAGKQNSQLITLASLCGMYQGAKLSRYITNYWKDNITRQKSFLKDFSQQLHQYTRALEKSDKAHTKPNLINFNKTFMVLANLVNEIVIPLSNGAISFPNISKLEDGEESHLIEFALNDYSQLSELIGELKDAIATNGGYTPFAKVTLNHYTAEQKPHVFKNDIDAKIRELKLIGLVETLKGKSSEQIEEYFSNLDKFSTYNDRNQSVIVRTQCFKYKPIPFLVKHQLAKYISEPNGWDEDAVAKVLDAVGAIRSPAHDYANNQEGFDLNHYPIKVAFDYAWEQLANSLYTTVTFPQEMCEKYLNSIYGCEVSKEPVFKFYADLLYIRKNLAVLEHKNNLPSNQEEFICKINNTFENIVLPYKISQFETYKKDILAWINDGHDHKKYTDAKQQLGFIRGGLKGRIKAEEVSQKDKYGKIKSYYENPYTKLTNEFKQISSTYGKTFAELRDKFKEKNEITKITHFGIIIEDKNRDRYLLASELKHEQINHVSTILNKLDKSSEFITYQVKSLTSKTLIKLIKNHTTKKGAISPYADFHTSKTGFNKNEIEKNWDNYKREQVLVEYVKDCLTDSTMAKNQNWAEFGWNFEKCNSYEDIEHEIDQKSYLLQSDTISKQSIASLVEGGCLLLPIINQDITSKERKDKNQFSKDWNHIFEGSKEFRLHPEFAVSYRTPIEGYPVQKRYGRLQFVCAFNAHIVPQNGEFINLKKQIENFNDEDVQKRNVTEFNKKVNHALSDKEYVVIGIDRGLKQLATLCVLDKRGKILGDFEIYKKEFVRAEKRSESHWEHTQAETRHILDLSNLRVETTIEGKKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNILRLEETKYKSKPFGVVHFIDPKFTSKKCPVCSKTNVYRDKDDILVCKECGFRSDSQLKERENNIHYIHNGDDNGAYHIALKSVENLIQMK

SEQ ID NO: 129 - protein, 1067 aa, synthetic construct
  REGION 1..1067: note = Description of Artificial Sequence: Synthetic polypeptide
MKNGINLFKTKTTKTKGVDMEKYQITKTIRFKLLPDNAHEIVEKVKSLKTSNVDELMDEVKNVHLKGLELLFALKKYFYFDGNQCKSFKSTLEIKARWLRLYTPDQYYLKKSSKNSYQLKSLSYFKDVFNDWLFNWEESVSELAIIYEKYKICQHQRDSRADIALLIKKLSMKEYFPFISDLIDCVNDKNSNKTFLMKLSEELSVLLEKCNSRALPYQSNGIVVGKASLNYYTVSKSEKMLQNEYEDVCQSLDKNYDITEMKVILYKEKLDNLNFKDVTIANAYNLLKENKALQKRLFSEYVSQGKVLSLIKTELPLFSNINDNDFEKYKEWSNEIKKLADKKNTFCKKTQQDKIKDIQNKISELKKKRGALFQYKFTSFQKHCDNYKKVAVQYGKLKARKKAIEKDEIEANLLRYWSVILEQEDKHSLVLIPKNNAKDAKQYIETINTKGGKYIIHHLDSLTLRALNKLCFNAVDIEKGQMVRENTFYQGIKEEFERNKINCDNQGVLKIQGLYSFKTEGGQINEKEAVEFFKEVLKSNYAREVLNLPYDLESNIFQKEYTNLDQFRQDLEKCCYALHSKIGKDDLDEFTRRFEAQVFDITSIDLKSKKEKTKTTGEMKKHTQLWLEFWKGAIEQNFATRVNPELSIFWRAPKSSREKKYGKGSDLYDPNKNNRYLYEQYTLALTITENAGSHFKDIAFKDTSKIKEAIKEFNMSLSQSKYCFGIDRGNAELVSLCLIKNEKDFPFEKFPVYRLRDLTYQGDFKDKHDQMRYGVAIKNISYFIDQEDLFEKNNLSAIDMTTAKLIKNKIVLNGDVLTYLKLKEETAKHKLTQFFQGSSINKNSRVYFDEDENVFKITTNRNHNPEEIIYFYRGEYGAIKNKNDLEDILNEYLCKMETGESEIVLLNRVNHLRDAISANIVGILSYLIDLFPETIVALENLAKGTIDRHVSQSYENITRRFEWALYRKLLNKQLAPPELKENILLREGDDKIDQFGIIHFVEEKNTSKDCPNCRKTTQQTNDNKFKEKKFVCKSCGFDTSKDRKGMDSLNSPDTVAAYNVARKKFES

SEQ ID NO: 130 - protein, 1309 aa, synthetic construct
  REGION 1..1309: note = Description of Artificial Sequence: Synthetic polypeptide
MAKETKEFKTFDDFTNLYEVQKTLRFELEAVPETEIVLENRGIWYKRDKKRADEKPIVKFYMDILHREFTDEALEKIKESGVLNLSGYFKLFEELRRLQNHGANTKEEKKLKLEEIRAKKREISNELSQIRRVFSVRGFDVVDSDWKKKYTIEGKKIKNDKSKTYLILSENILNFLENRFTSKEVERLRSIDKKHVEDYGNVVNSGGENIFATFKGFFGYFDSLIKNRENFYETDGKAGRVATRSVDENLNFFAENLHIFSTDLPKALKDDLSDTQKAIFERSYYKNCLLQKDIKSYNLIIGDINKEINKHRQQRDTKIKFLNTLFKQILSIEEKEQYKHIEINNDEDLIRAIRDFISLNESKISEGTKIFNQFIQRCLQKEDLGQIYLPKDSVNTIAHRIFKPWDEIMALFDRKYFVSLEEIKDLTESSVWKERVLEESKTKSLIFKDTHIHTIISGQEIFSNFILILEKEYKNQFSGFISETRRGKAAFVGYDESLKNLRATIKWFEGKNLKLSETEKVEWIKAIKDYADAALRIFQMTKYLWLPVVGDEEDKDYLRIKAEIDQLTKDNDFYNKINAFIDGYKPEPFIYRSSFQEYLTRRPFSTDKFKINFENSRLLDGWDKDMIDDRMGILLQRDGDYFLGILNKEDRHCLDNLVDVKSEDKNSYALMQFKQLTGLYRQLPRMAFPKKKQPVLEANAEIKKIKEDFDFLQKQKKEREVNVNVVFDNKKLNLLINHYAEFLKENYKDEKCYDFSLLNKEKVYESLSDFYADVDKITYSLSFIQVSIDQLIKTGKILLFRLKNKDLLKGSLGQNKNLHTYYFHALFERENLSQGRIRLGAQAEIFFRPASIEKEKDKNRSNALKKSPKTRYVKEILKNKRYSEDKVFLHLPIQLNADAYDLPSINQNVFEFIKNRQEKVKIIGIDRGEKNLAYYSVISQNSNGKIKIEEPPRDLNLGYLEPLDELENKRQDERKAWQSISEIKSKRDGYISYAVSKIVELMLKYQAIIVLEDLSGKFKRSRMKFEKAPYQQLELALIKKLNYLVKKNSKSGKPGHYLSAYQLTEPVGSYKEMGKQTGIIFYTQAGYTSRTCPTCGWRKRVQGLYYKDRTSAQRRFDPKTGVKIFYDSVNDRFVFQYHPVYEQKELKEWDKEIYSDVTRIRWNNEEKKNNEYRKGDITLKIKRLFRDRGIDLSRNINEQLVNVGDASFWEELINLLRLITEIRNIDNENNRDFIECPHCHFQSENGFHGVAWNGDANGAYNIARKGLLITKAVCDPEKNVGDITWSDLKVDMKDWDAATDEWAKKNPEK

SEQ ID NO: 131 - protein, 1456 aa, synthetic construct
  REGION 1..1456: note = Description of Artificial Sequence: Synthetic polypeptide
MENEKIFSDLTNRYQVVKTLPFELKPVPRTRVLLGLDNPNKGEIFSKDRERAENFTIIKKYIDRLHSLFINESLKKADIDFSNFYKQYGKNINTKNNKNIDDDNDINDDEKEDSENDNLKKYRQEIANLFNKSKYKSWVNVGKDGDKISGMLFEKGLIDLLRTHFSDNLNEDIEIPELFSNKKIKDTRKLKEIINSFGKDGKDGQNFTTYFSVSFHNNRKNYYKSDGKMGRVSTRIVDENLERFCKNIYLYKEIIGKNEIKEIFSGNWDIYLQKKPNFSNDKTYKKLDEFKNDKYDWEMIFRDVNSYNKYFLQSDIEFYNYIRGKLNQDINEYNGKKRDSKEKINSQFENLRNQVHGEKKNYDDDFEIDEDNIIQFINEIFVRHNQNKMRFSEKLFSDFIDLLMVDNGDKLDKVYFSQKAVENAIARYYFVEETTNEGREPLLISLLLQNAGKDRKKLSNKPIKLGDIKFVLDQANNKPAEDIFKNRYVLSESNNDGIINANDKNHWANLLRLIKKDFYFHKDNLIKSQDKLALETKYNKGSDEGERQIETIKNFAESAKAILRMTKYFDLRKNGVIQNVIGGKDPIHEEVDKYFDGDVLSGEESCRISKYYDALRNFITKKAWSADKIILNFDCSEFLGGWDRSQEQKKRGIILRHRDGDEERYYLAVLGKNGKQYFENRTLFKGCESSDWQKIEYNVIQKPHMSLPKNLITPFFKKDKITNERFIDRSKKGAKALIEIDINPSDEFLNNYNLGKHTKENLDKSFLCDYFKYLMDAIAKYYKGEFNFNFPDVSNFDNTQPFYSFIEKNAYSIKYFGISSKEIEKLIADCYYKEDVYLFQIYCKDFEIDPKIGKAKYGNEFRTKAEIRKSKGEEAGNENLNTKYFKLLFDEKNLKNQNGIVYKLNGGAKMFYRPSSIKKDEKIDGKWRYKEDKYSLNITITCNFSSKKDDLSIDKDINKKIAEVNANSDFRIISIDRGEKNLAYCCVMDENANILDIKSLNRITRYDKNGKAIKEKNMFHEVKDGKLCYGEPVYDFYKDYQNLLDEREIKRLVNRRSWNVIEDIKNLKKGYVALLINYICKAVVIAINEGKYPIIVLESLDKGMLHNRVKIEKQIYRGVEEGLVRKLNYFVDKKTDNVLNAWQLLAKFETVGSSLDRKKQLGIIFYVDPGYTSITCPCCGFRQRKYIKAERAEENFKEIKIKFDGKRYSFAYDYRCIDDNGKEKSKEDIIYSNVKRLLRSGRNGRAVQIEDVTDELTNLFKKHNINIEQDINEQLAGKDNKFWKQLLWWFNAIEQIRNTQSLRRKFNTEENKLEILENNDCDFILCPHCYFDSNKDKFQNKIWNGDANGAFNIGRKGIIDIFEIKKHQRMLSDFMEQWGIDKLPKANGGNQAVIEIVKNDKKYNLCILNNKKIPYYCLRIGKEKIDSIADDRKCNQLPDLMVNWKKWDMWLDKWGK

SEQ ID NO: 132 - protein, 1486 aa, synthetic construct
  REGION 1..1486: note = Description of Artificial Sequence: Synthetic polypeptide
MPEVKNVFQDFTNLYELSKTLRFELKPVPETEKILELNAAKTKKFPKDLYRAENFEIIKKYTDELHRTYIRETLNNVNIDYLKFLEIFRINGKKKNEMTDENEESDENNEKDDIQKIKKELRSKIGNLFNKWNNDKDNKFKDWVKIDVGKKEKEVSGDLFGKELITILKNYFKNKLDSKVNVPMLFFNEQEIKNGEAKKQRKLEAVFENFDKFTTYFTDSFYNNRKNYYKTEGRVGQVATRIIDENLPRFCSNLIAFNEVVSLYSTLLNNFDLGWKEYLNEKKINQTWVEKFELSNYDWKALFNDVNYYNQCLLQEGIDKYNYIIKKLNKDINEYTQNKYKSVEKGNNNNPDINFFQKLHKQIHGERDFKLIEIDIDENNIFTKILPEFILHSDMKLMTKIDEEVGVEEIVGAERIIKIFIKQELKDLEKIYLSRRAIETISAKWFHSWETLKDLILGYLNKDLLESKKRKKVPDFVDFNIIKIVLENNKDDYKDLFKRKYFEADKNEFVDWIDSSGGTKKLEFGGENWINFLNVFEYEFGTLLTEYKKNKNALLYLIDKKIDYDKNNEVGQTAAIKNFADSALGIFRMVSYFALRKKGVMVEPKNGKDEIFYAFVDRYLDGDDNDREEQNKIVQYYNTLRNFVTQKAWSIDKVRLCFDCGEFLKGWDKDKIHERLGIILRNNNKFYLGILNKNHKQIFIKIKSHDNNNFYYVIYDYKQLNNVYRQIPRLAFPSRSVKKGDAYMLRAIQERKKKFFLEDEEFIELQEIKNEYDKIGNDLSKEKLTKLIEYYKKVVISNYSSLYNVSNLNNKKFNSINEFNQYVENLMYSLIPTRISPDFIKEKISKGELYLFQIYNKDFELDESIGKEKFGEDFAPVIMDGKNNLHTEYFKLLFNDSNLKNPNGVVFKLSGGAKMFYRPATENLPIKKDRDGNIIKNKKGENVIVGQRYKEDKYFLHLPIILNFVNKGKNYSINDMVNKAITNASDDQDKFRIIGLDRGEKHLVYYSVINERQEIIEIGSLNNISRKDNKGEIIEEKNWYHDKFGNIEKEPTKEYHKDYHNLLDQREIERLKSRQSWEKIENIKELKEGYISAVINKICNLVIKAIKENKIPIVALENLNSGMKRGRIKIDKQIYQKLELKLAKKLNFLVDKKEKNYLSAWQFTPKIETFSGDIEKKNQVGIIFYVDPAFTSATCPNCGFRKRIKMDPQNAKKKIKDMEITYENGIYKFDYPIENGENDVVYSDVERLKWDNEKKKVIKTKNVSDDFGKLFEDIKDKNNLKKELLSIGEENKEFWKEFSRCFNLLLRIRNSKLIKRKLNDDTGKVEIIADDDLADRDRDFIYCPQCHFHSEGGDVFGEFVKKKYLGKDNFEFNGDANGAYNIARKTIIAVNKIKDYQLGLNHFIEKYRISELPNNGKDKKNIFYNNNSYILSFFEVQDEKFRKVKVYGLKKDGDRQIIQKKEMWYRRYPDIFVNNKEWDKFVQNKS

SEQ ID NO: 133 - protein, 1403 aa, synthetic construct
  REGION 1..1403: note = Description of Artificial Sequence: Synthetic polypeptide
MLFFMSTDITNKPREKGVFDNFTNLYEFSKTLTFGLIPLKWDDNKKMIVEDEDFSVLRKYGVIEEDKRIAESIKIAKFYLNILHRELIGKVLGSLKFEKKNLENYDRLLGEIEKNNKNENISEDKKKEIRKNFKKELSIAQDILLKKVGEVFESNGSGILSSKNCLDELTKRFTRQEVDKLRRENKDIGVEYPDVAYREKDGKEETKSFFAMDVGYLDDFHKNRKQLYSVKGKKNSLGRRILDNFEIFCKNKKLYEKYKNLDIDFSEIERNFNLTLEKVFDFDNYNERLTQEGLDEYAKILGGESNKQERTANIHGLNQIINLYIQKKQSEQKAEQKETGKKKIKFNKKDYPTFTCLQKQILSQVFRKEIIIESDRDLIRELKFFVEESKEKVDKARGIIEFLLNHEENDIDLAMVYLPKSKINSFVYKVFKEPQDFLSVFQDGASNLDFVSFDKIKTHLENNKLTYKIFFKTLIKENHDFESFLILLQQEIDLLIDGGETVTLGGKKESITSLDEKKNRLKEKLGWFEGKVRENEKMKDEEEGEFCSTVLAYSQAVLNITKRAEIFWLNEKQDAKVGEDNKDMIFYKKFDEFADDGFAPFFYFDKFGNYLKRRSRNTTKEIKLHFGNDDLLEGWDMNKEPEYWSFILRDRNQYYLGIGKKDGEIFHKKLGNSVEAVKEAYELENEADFYEKIDYKQLNIDRFEGIAFPKKTKTEEAFRQVCKKRADEFLGGDTYEFKILLAIKKEYDDFKARRQKEKDWDSKFSKEKMSKLIEYYITCLGKRDDWKRFNLNFRQPKEYEDRSDFVRHIQRQAYWIDPRKVSKDYVDKKVAEGEMFLFKVHNKDFYDFERKSEDKKNHTANLFTQYLLELFSCENIKNIKSKDLIESIFELDGKAEIRFRPKTDDVKLKIYQKKGKDVTYADKRDGNKEKEVIQHRRFAKDALTLHLKIRLNFGKHVNLFDFNKLVNTELFAKVPVKILGMDRGENNLIYYCFLDEHGEIENGKCGSLNRVGEQIITLEDDKKVKEPVDYFQLLVDREGQRDWEQKNWQKMTRIKDLKKAYLGNVVSWISKEMLSGIKEGVVTIGVLEDLNSNFKRTRFFRERQVYQGFEKALVNKLGYLVDKKYDNYRNVYQFAPIVDSVEEMEKNKQIGTLVYVPASYTSKICPHPKCGWRERLYMKNSASKEKIVGLLKSDGIKISYDQKNDRFYFEYQWEQEHKSDGKKKKYSGVDKVFSNVSRMRWDVEQKKSIDFVDGTDGSITNKLKSLLKGKGIELDNINQQIVNQQKELGVEFFQSIIFYFNLIMQIRNYDKEKSGSEADYIQCPSCLFDSRKPEMNGKLSAITNGDANGAYNIARKGFMQLCRIRENPQEPMKLITNREWDEAVREWDIYSAAQKIPVLSEEN

SEQ ID NO: 134 - protein, 1356 aa, synthetic construct
  REGION 1..1356: note = Description of Artificial Sequence: Synthetic polypeptide
MTIKKHKPFTNFECLTPVQKTLRFRLIPVGRTTEFVKCRNIIEADRKRSEMYPLLKELADRFYREFMTDQLSNLLFDWSPLVEALLLARNNTDPRENQRIASLVRDEQKKYRTLLLKRLSGQVDRNGTPLPKNTASVNKKYYDDLFKARFVTETLPAYLEHLKNKPDGRISDELFDAYKDALDSYQKFTSRLTNFWQARKNIFTDEDIATGFAYRIVHEIVPDYLFNRRVYEQHKLDFPEPLDLLETELKKKNLIANDESLDALFTIPAINRLLTQKGVDLHNAVIGGFFTDDHTKVQGFNELANLKNQTLKNVSDNSEIKPVGKMTRLKKHILSISESTSFLFEQIESDDDLLARIIEFNNTLSEPDIDGLSIADINDQLYNIMTGVDPSTILVHARNLNKLSHEASLSWNRLRDGLYQMATESPYREDERFKRYIDASEEERDLSKLKNDIYFSLQELQFALDQSIDLEEEATPTEDIFLPFEFPGMDLKSELTVLFRSIEQLISSETKLIGNPDAIATIKKYLDAIMARYSIWNLLSCEAVELQDDLFYPEYDRVMGSLSNIILLYNLARNYLSRKPSSKEKFRLNFDKPTLADGWSESKVPDNFSVLLRKDDLFYLGILKDRKAYRVLSYENCDETAKNIKGYYERMIYHFSPDAYRMIPKCSTARKDVKKHFGEQGETTGYTLYPGASNFVKPFTIPYEIYRLQTELVNDKKRYQADYLKQTEDEEGYRQAVTAWIDFCKSYLESYEGTSTFDYSHLLKSEDYEDVNQFYADVDRASYSIYFEKVSVDLIHTMVDRGDLYLFQLYNKDFSPHSTGKPNLHTMYWRALFSNDNLQNNTIKLNGQAELFYRPKQVEQPTVHLQGSYLLNRFDKHGDVIPAGLYCEIYNHINERHPEGYTLSEEATQGLLDGRFVYREAPFELVKDKRYTEDQLFLHVPLEFNWTASANVPFENLANEYIKKDSDLHIIGIDRGERNLLYYSVINLQGDIVKQGSLNTLIQQTTLKGETVERQIPYQSMLKQREDERAEARQNWQSIDRIKDLKEGYLSHVIYKLSRLIIKYHAIVVMENLNVGFKRGRFKVERQVYQKFEVALINKLNALSFKEYEPNELGGVMRPWQLARRVVSPEDTRSQNGIVFYVPASYTSIVDPVTGFANLFYLNRIRNKDLNSFYGHFQEIRYDHEFDRFIFRFNYADFGVFCRIKNVPSRTWNLVSGERKAFNPKRRMIEKRDTTDEIKKALEAHGIAYQNEQNLLPLLLENENLLARIHRSFRLVLQLRNSDSDRDDIVSPALDKENNTFDSGQQPYESSLPINADANGAYNIARKGLLLVDKVKNDKRAVLSNREWFEYLMAEE

SEQ ID NO: 135 - protein, 1296 aa, synthetic construct
  REGION 1..1296: note = Description of Artificial Sequence: Synthetic polypeptide
MENKDYSLSRFTKQYQNSKTVRFALTPIGRTEEYIIQNQYIEAARRKNQAYKIVKPIIDEKFRSMIDDVLTHCEKQDWVTLDKLILQYQNNKCRENMDALAEQQEEIRKNISEEFTKSDEYKNFFGKEDSKKLFKIFLPEYLNQINASESDKEAVNEFQKFKTYFSNFLIVRADIFKADNKHNTIPYRIVNENFMIFAGNKRTFSNIIRLIPNALEEIAKDGMKKEEWSFYNIQNVDSWFEPDSFQMCMSQKGIQKYNFIIGLVNSYINLYTQQNPQATEVKRSRLKLRMLHKQILSDRVNPSWLPEQFKEGEEGEKQIYEAILALENDLIKNCFDKKYDLWIQSIDIQNPRIYIAASEMARVSSALHMGWNGLNDVRKTILLKSDKKQAKVEKILKQDVSLKDLSDTLNRYADIYKEEQIPSLYQYIEYGSELLQDCAITRKEYHDLLNGNSNTLSLNQNEKLIEGLKAYLDSYQAIVHFLNVFIVGDELDKDTDFYAELDGLVESLSEIVPLYNKVRNYITRKVYSLDKMRIMFERSDFLGGWGQSFDTKEALLFQKDNLYYIGIIEKKYTNMDVEYLHEGIKEGNRAIRFIYNFQKADNKNIPRTFIRSKGTNYAPAVRKYNLPIESIIDIYDVGKFKTNYKKINEKEYYESLEKLIDYFKDGILKNENYKKFHFNWKPSNEYENINEFYNDTNNACFLLEKEEINYDHLKEQANQGKIYLFQISSKDFNEGSKGTPNLQTMYWRELFSNQNCKDGVIKLCGGASIYMRDASIKQPVVHRKNAWLINKWYKVNGQNVVIPDNTYVKFTKIAQERMNEDELTPQERQLWNSGLIQKKKATHDIMKDRRFTKKQYMLHAPLTINYKQQDSPRYFNEKVRSFLKDNPDINIIGIDRGEKNLIYITIIDQKGNILKGMQKSFNQIEEKGKEGRTIDYYSKLESVEARHDAARKNWKQIGTIRELKEGYLSQVVHEITQLMIQYNAVIVMENLNMGFKKGRMKVEKSVYQKFEKMLIDKMNYLAFKRDMQGNAIDPYEVGGVMNGYQLTDRFTSFADMGSQNGFIFYVPAAYTSVIDPVTGFVNVFQKTEFKTNDFLHRFDSISWNDKEQSFVFTFDYQNFKCNGTCYQNKWSLYADVDRIETIIKNNQVDRIEPCNPNQKLIDFFDKKGIIYRDGHNIVDDLEKYDSKTISEIIHNFKLILQLRNSMRNPDTGEIIDYIASPVMHNEERFDSRKRNPELPQDADANGAYHIALKGLMFLQKINEYADSDGNMDNRKLKITNEEWFKYMQTRKEHTYF

SEQ ID NO: 136 - protein, 1250 aa, synthetic construct
  REGION 1..1250: note = Description of Artificial Sequence: Synthetic polypeptide
MSNKTSSITTTNKLSYTGFHNNGKQSKTLMFELKPIGRTTEHLDRKGYLADDIDRAESYKTFKEIADNFHKNLIEESLATFTFSDTLKDYFDLWLSPVRTNEDTPKLRKMEAKLRKELSSALKQHPSFAATSSGKRLIDEALYPNASDKERQCLDRFKGRSSYLDSYTEVRSFIYTDLCKHNTIAYRVVNENLKIYLENILAYEKLMQTAVNGKLETVKEMFHDLYPTFSMDISIFFTSYGFDYCLSQNAITRYNILLGGWSDDNGIHHKGLNNYINEYNQTVPRNKRLPKLNKLQKMILSEENSMSFIIDKFENDVDLANAIRYWLKNCQFDALNLLIWTLDVHYNLDEIHFKNDNQGKNISDLSQALFKNHHVIRDAWDYDYDIVNAKAKSRQKPERYAEKRDKAFKKINSFSLSYLANILSQYDNQYANFVAQFKTRISVHIQNVQQMIADKTLDMRLDPLMLLKSISSDTKLVEDIKRVLDSLKDMQRMLTPLLGEGTEPNRDAMFYSDFEPLMNYVDTLTPLYNKVRNYITKKPYSTKKTSLYFGASNFGSGFDVTKLPVSHTIIMRDKGCYYLAVIDNNKLIDKLYDHNDNDGYEYMVYKQIPSPIKYFSLKNILPQDPPDDIRQLLEDRKNGAKWSHDDETRFIDYIVNEFLPTYPPIHDKNGNPYFSWKFKNPDEYESLNEFFDDVSKQAYQTSFRFVSRDFVDDAVENGDIFFFQIYNQDFSPASHGKPSPHTLWFRALFSDVNLETKDIRLKGNATAYFRPASIFYTDEKWRKGHHYEQLKNKFKFPIIKDKRYALDKFFFHITLEINCNATVEKYFNNRVNEEIRKADRYNILAINRGERNLLYAVVMDQDGTILEQKSFNIIKSELPNKTVKETDYWKKLHAREKERDTARKSWKSIECIKDLKKGYLSYVVKTITDMMFEYNAVLVMENLDIEMKRSRQKIEKNVYAQFQNAIIQKLSMYVNKDIDLHIARTAPGGTLNPYQLTYIPASRTKTPKQNGFVFFLNPWNITEIDPTTGFVDLFQTCFRTKNEYKDFFAKFKDIRYNEAQGWFEFDTDYTYFRDKEKAGKRTRWNICSYGTRLRRFRNPDKNYAEDAMTVYPTQMLKDLFDEYNIPYAPASAKSTSISIKDDIIQIDKLDFYKKLLYILKLIVQLRNTSPSSTEQEDDYIISPVINEDTNWFYDSRDYNEESLLPCNTDANGAYSLALKCNMVIDRIKNTIPGEPVDMYISNADWLDARQ

SEQ ID NO: 137 - protein, 1328 aa, synthetic construct
  REGION 1..1328: note = Description of Artificial Sequence: Synthetic polypeptide
MNSKTSIFDFSNIFGRDITLRFKLTPVTINSKGEVKDANGADPYRPYLSADEELQEQYELLKTAIDAYHQMYIDKKLKHILCLPLTEKGKDGVEHDTAKSKFVKSCLAYIKDYGEKDKKRQTADLRTFISRVFADDNISSLPPYKVKSDFITKTLRQWLEQPDTKVEKKEAILDLIEKNGSKLYANCQGLLEARQRLYEKDGKSTSVPYRCIDRNLPRFSKDYHLFEKILGDCSDVFDFEQLDKDFSEELKGIARLSGIRVESVREVFQPLLYLAYLNQEGIQYLNTIIGTKKEKGTSALGLNEYINQYNQKQGIKKKKDGIPMLNKLNNQILFGDEVFIETLAEHKEAIPVIKKVVSSLGKLGAFDGECHENKLYQFLLSLSSYAGNIYVNTKVVAQISSSLWGDYSILYDAVKHDKNGRLIQKSVTLGELNEKIERLKLEDNRDAFEYFRRSQVKDVVHGSSNVGVFEQLKNCYNDFVEKKILKCSFFSEDQVLVIQRLFDSILSLQRIFKVFCPSLYEVDSDGLFVAKFSDYWNVLRGFDKDYDLLRNLFKRKPYSTDKIRVHFGLSNLMDGFVDSWTDKKDKGTQYNGYILRQAHSFVDENTSKELQEFQRYNYYLVISGNVRLFREKGNALVCEKKKEKLVASDEFSGFERFDYYQSSINNFNREFKRLTGRDRKSFTDEILQNEGKKELKSTYIENLIKVAKSMKRLTALQNLVSDEKVRKYSENLDYETLSAEIGQILATGRERKYVPVSTNEMKNLLKSSKNNKGEEVRTFMFRISNKDLSYAETMQKGERKSHGAENMHTMYFRALLDTLQNTFDIGTGTVYFRKASDKRKMKYDEKNPTHRKGDELAFKNPYNKGKKKSVFGYDLIKDRRYTKDSYLFHLSITQNYQKKGNAEDLNAMVRDYIRTQEDLRVIGIDRGERNLLYATMIDGEGHILAQKSFNVIGYQGTTASGESFQVETDYHQLLNEKAEKMRSLQREWKEMDKIQDMKDGYLSVVVHELAKMVVENNAIIVMEDLNMGFMESRQSQLANVYQKFEEKLRNKLQFYVDKRKRNDEPSGLYHALQLAGTETKDNQNGFIFYIPAWNTSKIDSVTGFVNLFNLKYTNIKDAKAFFSTFEKIEKNVETGHYDFTFSYSSMARKKMAKRMDGTRDSWTISTHGSRIVREQKGNYWEYREIESLTSEFDALFEKYSIDTRCRLKEAIDKCGEAEFFKELIRLMKWTLQLRNYDDRGNDYIVSPVCYRGNEYYCSLDYDNEEGMCISKIPCQMPKDADANGAFNIARKGLMLCERLKKGEKIGVIKGTEWLQYVQNMSERYVGMV

SEQ ID NO: 138 - protein, 1434 aa, synthetic construct
  REGION 1..1434: note = Description of Artificial Sequence: Synthetic polypeptide
MINTMEQPKKSIWDEFTNLYSLQKTLRFELKPQGKTKELVRTLFINPEEHHHKLISDDLELSKNYKKVKKLIDCMHRNIINNVLSKHQFTGEELKKLDKNSNAEDNDTETDNADKKDPFAKIRERLTKALNEESKIMFDNKLLNPKKGKNKGECELKKWMDKAEDKYFELGNNEKIDKEAVKADMERLEGFFTYFGGFNKNRENVYSSKKIATAIPFRIIHDNFPIFKKNIENYKKITEKHPELAKLLNEKGANEIFQLEHFNKCLTQDGIDVYNNEKLGIIAKEQGKEQDKGINQLINEYAQKKNKEIKENAKGGEKPKKIKIAVFDKLKKQILSISKTKSFQFEVFEDTSDIINGINKRYTFLTEAKEGMSIVDEIKKIIGSVGDEKYSLDEIYLKEKFISTLSKKLFNYSRYIEVALEKWYDDRYDDKINKSGTDKRKFISAKQFSITSIQDAINYYLEKYEKDEELSKKYTGKNIIVDYFKNPTITIEHKQKEEVISEEKDLFKELEVRRNVIQHILNGDYKKDLKEEKQQDGDSEKVKAFLDALLEFNYILNPFIIKDKNLRKEQEKDEEFYNEIKKLQESIFEAEILDLYNQTRNYITKKPYKLDKFKLTFGSGYFLSGWSNDMEEREGSILIKYNEDRSKNYYLIIMAKPLTDDDKKQLFSDNGTHSKICIYEFQKMDMKNFPRMFINSKGSNPAPAIEKYNLPIKTIWADYQKYKNLNQKGKDKFLEENPDFRHNLIGYFKICAEKHESLAPFKHQFSSIWKPTKEYENLAQFYKDTLEACYNLKFENVNFDNISQLVSSGKLHLFKIHNKDFNPGSTGKKNLHTLYWEMLFDEKNLQDVIFKLSGGAELFYREASILKNKIIHKIGEKVLKKFFKLPDGKLEPVPAESIKNLSAYFRKELPEHELTEIDRKYIDNYSIIGKKDDKLGIMKDERFTVDKIQFHCPITINFKSKNKNFINDDVLEYLHKRDDVHIIGLDRGERHLIYLTMINKDGKIVDNMQFSLNELQRRYKINGNEEIQKINYQKLLDTREVSRTEARRNWQTIENIKNLKEGYLSLIVHQLAKLMIEKNAIVVMENLNYGFKDSRARVEKQIYQKFESILIKKLQYLVMDKNNLYDSGGVLSAYQLTNQEVPAYKYISKQNGFLFYVPPDYTSKIDPETGFINLLDTRYYSRKNAVALLNKFDKIYYDRDNKYFRFDFDYNSTDSNGNKNFDKLRVDISELTRTKWSVCSHPAKRSITVQINNKWVRQPINDVTDKLIKLFEDKQIGYESGKCLKDEILKVEDAKFFEDLLRYLSVLLALRHTYTENGVEYDLIISSVEKAPGSNEFFVSGKDNNLPANADANGAYNIARKGLWLLRKLDEIDNQELAIKKFNELKHAKEIKKNGEESKEDKGDRKRKKKWVSQWCPNKEWLAFAQSMQDVSEK

SEQ ID NO: 139 - protein, 1263 aa, synthetic construct
  REGION 1..1263: note = Description of Artificial Sequence: Synthetic polypeptide
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 140 - protein, 1283 aa, synthetic construct
  REGION 1..1283: note = Description of Artificial Sequence: Synthetic polypeptide
MSNLNTFISPEFTGKIKMTKSLKVSMIPIGETEHWIAKHKVFEKDRELFDKNLKARPILDEFIKYTVSRALPNLLFDFEAYYLVKKDRTKARAFEKELAKTVTDLILKEMDELKSASLIDSADFVKTTLKKFAGTHDIPGLSRIEAIESLEAASKLTALNGKFNTSRIAIINTLIPKRIIENFDIYLSNMEKIRNVYESGEFGFLFERYPDTLLFMEPANYRTVCSPEAIEDYNRFISGYGDSTESWIKGFNQELSEASNSSKSSNGGVRRYSLIKPLHKQHLFETKKFFTFASISSDDDVRELINSVKGSTEDACLNALAFFSSSDPKTLFVKGSYLHTLSAFLYGSANSYILPERIKEGEKARLTAEYDSVAKKTKAVTTRYNVAMNNISKKINEKIFSLADIDAYCCDISKRRSVREILLGIMQEMYAAVYGENGKWSNIEAEAVLDSKTKIWKAKNGAVAKAVNDYLTAILEIRKFIRPFALRMEELEELGLDTSSALDAGEITNTLFEAVRAQKLVHAYLTRNDADIALSTQVYFGGTQKAAASWWNYETGDIQNRQIALAKKDGMYYFIGTFDERGSYSIEPASPGEDYYEMLDVKKGQDANKQIKKVLFSNKAIREHFADSSNDYVITTKVNSPITVRREIFDKYQAGEFKLTSQKIRKGDLVGEKEMTYYREYMDLLFQMAKGYTEYSRFNMDTLLPIEEYDTENDLLDDVNTNTIDYRWVRISAACIDDGVRNGDIFVFRAQTSSMYGKRENKKGYTGLFLELVSDENLLVTRGMSLNSAMSIYYRAKVHDAITVHKKGDVLVNKFTNARERIPENSYKAICAFYNSGKSIEELTIEDRDWLAKATTRICSGEIIKDRRYTKNQYSISISYNINRSVNNRKRVDLATIVDDTASAGRIISVTRGTKDLVYYTVIDDGGSVIEARSLNVINGINYAKMLAQISEERHDSNANFDIPKRVETIKEAYCAFAVHEIISAALKHNALIVVELISDAIKDKYSLLDNQVFLKFENVLKNCLMSVKVKGARGMEPGSISNPLQLCNADDKSFRNGILYQIPSSYINICPVTGYADIIDYYNIVSAGDIRNFFVRFENIVYNKEKARFEFSFDLKNIPIKLEKCPDRTKWTVLGRGEITTYDPLTKSNHYVFDAAQMLAETVSKEGLDPCANIVEHIDELSAATLKKMFNTFRNIAKGIVSECDEVPVSYYKSPVIDEADIKNKSLDNKSISEIKCYNLDEKARYYLALAKSSSDGENKNRYVSSTAIEWLNYIQEKRTHE

SEQ ID NO: 141 - protein, 707 aa, synthetic construct
  REGION 1..707: note = Description of Artificial Sequence: Synthetic polypeptide
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 142 - protein, 757 aa, synthetic construct
  REGION 1..757: note = Description of Artificial Sequence: Synthetic polypeptide
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 143 - protein, 765 aa, synthetic construct
  REGION 1..765: note = Description of Artificial Sequence: Synthetic polypeptide
MYILEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 144 - protein, 766 aa, synthetic construct
  REGION 1..766: note = Description of Artificial Sequence: Synthetic polypeptide
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 145 - protein, 812 aa, synthetic construct
  REGION 1..812: note = Description of Artificial Sequence: Synthetic polypeptide
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 146 - protein, 812 aa, synthetic construct
  REGION 1..812: note = Description of Artificial Sequence: Synthetic polypeptide
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHKGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 147 - protein, 793 aa, synthetic construct
  REGION 1..793: note = Description of Artificial Sequence: Synthetic polypeptide
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 148 - protein, 441 aa, synthetic construct
  REGION 1..441: note = Description of Artificial Sequence: Synthetic polypeptide
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 149 - protein, 812 aa, synthetic construct
  REGION 1..812: note = Description of Artificial Sequence: Synthetic polypeptide
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 150 - protein, 812 aa, synthetic construct
  REGION 1..812: note = Description of Artificial Sequence: Synthetic polypeptide
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 151 - protein, 761 aa, synthetic construct
  REGION 1..761: note = Description of Artificial Sequence: Synthetic polypeptide
MSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 152 - protein, 717 aa, synthetic construct
  REGION 1..717: note = Description of Artificial Sequence: Synthetic polypeptide
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 153 - protein, 781 aa, synthetic construct
  REGION 1..781: note = Description of Artificial Sequence: Synthetic polypeptide
MRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 154 - protein, 793 aa, synthetic construct; identical to SEQ ID NO: 147
  REGION 1..793: note = Description of Artificial Sequence: Synthetic polypeptide

SEQ ID NO: 155 - protein, 717 aa, synthetic construct; identical to SEQ ID NO: 152
  REGION 1..717: note = Description of Artificial Sequence: Synthetic polypeptide

SEQ ID NO: 156 - protein, 761 aa, synthetic construct; identical to SEQ ID NO: 151
  REGION 1..761: note = Description of Artificial Sequence: Synthetic polypeptide

SEQ ID NO: 157 - protein, 765 aa, synthetic construct
  REGION 1..765: note = Description of Artificial Sequence: Synthetic polypeptide
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 158 - protein, 766 aa, synthetic construct
  REGION 1..766: note = Description of Artificial Sequence: Synthetic polypeptide
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 159 - protein, 870 aa, synthetic construct
  REGION 1..870: note = Description of Artificial Sequence: Synthetic polypeptide
MLVRTSTLVQDNKNSRSASRAFLKKPKMPKNKHIKEPTELAKLIRELFPGQRFTRAINTQAGKILKHKGRDEVVEFLKNKGIDKEQFMDFRPPTKARIVATSGAIEEFSYLRVSMAIQECCFGKYKFPKEKVNGKLVLETVGLTKEELDDFLPKKYYENKKSRDRFFLKTGICDYGYTYAQGLNEIFRNTRAIYEGVFTKVNNRNEKRREKKDKYNEERRSKGLSEEPYDEDESATDESGHLINPPGVNLNIWTCEGFCKGPYVTKLSGTPGYEVILPKVFDGYNRDPNEIISCGITDRFAIPEGEPGHIPWHQRLEIPEGQPGYVPGHQRFADTGQNNSGKANPNKKGRMRKYYGHGTKYTQPGEYQEVFRKGHREGNKRRYWEEDFRSEAHDCILYVIHIGDDWVVCDLRGPLRDAYRRGLVPKEGITTQELCNLFSGDPVIDPKHGVVTFCYKNGLVRAQKTISAGKKSRELLGALTSQGPIALIGVDLGQTEPVGARAFIVNQARGSLSLPTLKGSFLLTAENSSSWNVFKGEIKAYREAIDDLAIRLKKEAVATLSVEQQTEIESYEAFSAEDAKQLACEKFGVDSSFILWEDMTPYHTGPATYYFAKQFLKKNGGNKSLIEYIPYQKKKSKKTPKAVLRSDYNIACCVRPKLLPETRKALNEAIRIVQKNSDEYQRLSKRKLEFCRRVVNYLVRKAKKLTGLERVIIAIEDLKSLEKFFTGSGKRDNGWSNFFRPKKENRWFIPAFHKAFSELAPNRGFYVIECNPARTSITDPDCGYCDGDNRDGIKFECKKCGAKHHTDLDVAPLNIAIVAVTGRPMPKTVSNKSKRERSGGEKSVGASRKRNHRKSKANQEMLDATSSAAE

SEQ ID NO: 160 - protein, 830 aa, synthetic construct
  REGION 1..830: note = Description of Artificial Sequence: Synthetic polypeptide
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPRKRKKNAPPLKKPPKPRELVKRSDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 161 - protein, 790 aa, synthetic construct
  REGION 1..790: note = Description of Artificial Sequence: Synthetic polypeptide
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLNADLDVATTNLVRVALTGKVMPRSERSGDAQTPGPARKARTGKIKGSKPTSAPQGATQTDAKAHLSQTGV

SEQ ID NO: 162 - protein, 790 aa, synthetic construct
  REGION 1..790: note = Description of Artificial Sequence: Synthetic polypeptide
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLHADLDVATTNLVRVALTGKVMPRSERSGDAQTPGPARKARTGKIKGSKPTSAPQGATQTDAKAHLSQTGV

SEQ ID NO: 163 - protein, 782 aa, synthetic construct
  REGION 1..782: note = Description of Artificial Sequence: Synthetic polypeptide
MKTEKPKTALTLLREEVFPGKKYRLDVLKEAGKKLSTKGREATIEFLTGKDEERPQNFQPPAKTSIVAQSRPFDQWPIVQVSLAVQKYIYGLTQSEFEANKKALYGETGKAISTESRRAWFEATGVDNFGFTAAQGINPIFSQAVARYEGVIKKVENRNEKKLKKLTKKNLLRLESGEEIEDFEPEATFNEEGRLLQPPGANPNIYCYQQISPRIYDPSDPKGVILPQIYAGYDRKPEDIISAGVPNRLAIPEGQPGYIPEHQRAGLKTQGRIRCRASVEAKARAAILAVVHLGEDWVVLDLRGLLRNVYWRKLASPGTLTLKGLLDFFTGGPVLDARRGIATFSYTLKSAAAVHAENTYKGKGTREVLLKLTENNSVALVTVDLGQRNPLAAMIARVSRTSQGDLTYPESVEPLTRLFLPDPFLEEVRKYRSSYDALRLSIREAAIASLTPEQQAEIRYIEKFSAGDAKKNVAEVFGIDPTQLPWDAMTPRTTYISDLFLRMGGDRSRVFFEVPPKKAKKAPKKPPKKPAGPRIVKRTDGMIARLREIRPRLSAETNKAFQEARWEGERSNVAFQKLSVRRKQFARTVVNHLVQTAQKMSRCDTVVLGIEDLNVPFFHGRGKYQPGWEGFFRQKKENRWLINDMHKALSERGPHRGGYVLELTPFWTSLRCPKCGHTDSANRDGDDFVCVKCGAKLHSDLEVATANLALVAITGQSIPRPPREQSSGKKSTGTARMKKTSGETQGKGSKACVSEALNKIEQGTARDPVYNPLNSQVSCPAP

SEQ ID NO: 164 - protein, 774 aa, synthetic construct
  REGION 1..774: note = Description of Artificial Sequence: Synthetic polypeptide
VYNPDMKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 165 - protein, 769 aa, synthetic construct
  REGION 1..769: note = Description of Artificial Sequence: Synthetic polypeptide
MKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 166 - protein, 767 aa, synthetic construct
  REGION 1..767: note = Description of Artificial Sequence: Synthetic polypeptide
VIKTHFPAGRFRKDHQKTAGKKLKHEGEEACVEYLRNKVSDYPPNFKPPAKGTIVAQSRPFSEWPIVRASEAIQKYVYGLTVAELDVFSPGTSKPSHAEWFAKTGVENYGYRQVQGLNTIFQNTVNRFKGVLKKVENRNKKSLKRQEGANRRRVEEGLPEVPVTVESATDDEGRLLQPPGVNPSIYGYQGVAPRVCTDLQGFSGMSVDFAGYRRDPDAVLVESLPEGRLSIPKGERGYVPEWQRDPERNKFPLREGSRRQRKWYSNACHKPKPGRTSKYDPEALKKASAKDALLVSISIGEDWAIIDVRGLLRDARRRGFTPEEGLSLNSLLGLFTEYPVFDVQRGLITFTYKLGQVDVHSRKTVPTFRSRALLESLVAKEEIALVSVDLGQTNPASMKVSRVRAQEGALVAEPVHRMFLSDVLLGELSSYRKRMDAFEDAIRAQAFETMTPEQQAEITRVCDVSVEVARRRVCEKYSISPQDVPWGEMTGHSTFIVDAVLRKGGDESLVYFKNKEGETLKFRDLRISRMEGVRPRLTKDTRDALNKAVLDLKRAHPTFAKLAKQKLELARRCVNFIEREAKRYTQCERVVFVIEDLNVGFFHGKGKRDRGWDAFFTAKKENRWVIQALHKAFSDLGLHRGSYVIEVTPQRTSMTCPRCGHCDKGNRNGEKFVCLQCGATLHADLEVATDNIERVALTGKAMPKPPVRERSGDVQKAGTARKARKPLKPKQKTEPSVQEGSSDDGVDKSPGDASRNPVYNPSDTLSI

SEQ ID NO: 167 - protein, 763 aa, synthetic construct
  REGION 1..763: note = Description of Artificial Sequence: Synthetic polypeptide
MAKAKTLAALLRELLPGQHLAPHHRWVANKLLMTSGDAAAFVIGKSVSDPVRGSFRKDVITKAGRIFKKDGPDAAAAFLDGKWEDRPPNFQPPAKAAIVAISRSFDEWPIVKVSCAIQQYLYALPVQEFESSVPEARAQAHAAWFQDTGVDDCNFKSTQGLNAIFNHGKRTYEGVLKKAQNRNDKKNLRLERINAKRAEAGQAPLVAGPDESPTDDAGCLLHPPGINANIYCYQQVSPRPYEQSCGIQLPPEYAGYNRLSNVAIPPMPNRLDIPQGQPGYVPEHHRHGIKKFGRVRKRYGVVPGRNRDADGKRTRQVLTEAGAAAKARDSVLAVIRIGDDWTVVDLRGLLRNAQWRKLVPDGGITVQGLLDLFTGDPVIDPRRGVVTFIYKADSVGIHSEKVCRGKQSKNLLERLCAMPEKSSTRLDCARQAVALVSVDLGQRNPVAARFSRVSLAEGQLQAQLVSAQFLDDAMVAMIRSYREEYDRFESLVREQAKAALSPEQLSEIVRHEADSAESVKSCVCAKFGIDPAGLSWDKMTSGTWRIADHVQAAGGDVEWFFFKTCGKGKEIKTVRRSDFNVAKQFRLRLSPETRKDWNDAIWELKRGNPAYVSFSKRKSEFARRVVNDLVHRARRAVRCDEVVFAIEDLNISFFHGKGQRQMGWDAFFEVKQENRWFIQALHKAFVERATHKGGYVLEVAPARTSTTCPECRHCDPESRRGEQFCCIKCRHTCHADLEVATFNIEQVALTGVSLPKRLSSTLL

SEQ ID NO: 168 - protein, 761 aa, synthetic construct
  REGION 1..761: note = Description of Artificial Sequence: Synthetic polypeptide
MSKEKTPPSAYAILKAKHFPDLDFEKKHKMMAGRMFKNGASEQEVVQYLQGKGSESLMDVKPPAKSPILAQSRPFDEWEMVRTSRLIQETIFGIPKRGSIPKRDGLSETQFNELVASLEVGGKPMLNKQTRAIFYGLLGIKPPTFHAMAQNILIDLAINIRKGVLKKVDNLNEKNRKKVKRIRDAGEQDVMVPAEVTAHDDRGYLNHPPGVNPTIPGYQGVVIPFPEGFEGLPSGMTPVDWSHVLVDYLPHDRLSIPKGSPGYIPEWQRPLLNRHKGRRHRSWYANSLNKPRKSRTEEAKDRQNAGKRTALIEAERLKGVLPVLMRFKEDWLIIDARGLLRNARYRGVLPEGSTLGNLIDLFSDSPRVDTRRGICTFLYRKGRAYSTKPVKRKESKETLLKLTEKSTIALVSIDLGQTNPLTAKLSKVRQVDGCLVAEPVLRKLIDNASEDGKEIARYRVAHDLLRARILEDAIDLLGIYKDEVVRARSDTPDLCKERVCRFLGLDSQAIDWDRMTPYTDFIAQAFVAKGGDPKVVTIKPNGKPKMFRKDRSIKNMKGIRLDISKEASSAYREAQWAIQRESPDFQRLAVWQSQLTKRIVNQLVAWAKKCTQCDTVVLAFEDLNIGMMHGSGKWANGGWNALFLHKQENRWFMQAFHKALTELSAHKGIPTIEVLPHRTSITCTQCGHCHPGNRDGERFKCLKCEFLANTDLEIATDNIERVALTGLPMPKGERSSAKRKPGGTRKTKKSKHSGNSPLAAE

SEQ ID NO: 169 - protein, 746 aa, synthetic construct
  REGION 1..746: note = Description of Artificial Sequence: Synthetic polypeptide
MEKAGPTSPLSVLIHKNFEGCRFQIDHLKIAGRKLAREGEAAAIEYLLDKKCEGLPPNFQPPAKGNVIAQSRPFTEWAPYRASVAIQKYIYSLSVDERKVCDPGSSSDSHEKWFKQTGVQNYGYTHVQGLNLIFKHALARYDGVLKKVDNRNEKNRKKAERVNSFRREEGLPEEVFEEEKATDETGHLLQPPGVNHSIYCYQSVRPKPFNPRKPGGISLPEAYSGYSLKPQDELPIGSLDRLSIPPGQPGYVPEWQRSQLTTQKHRRKRSWYSAQKWKPRTGRTSTFDPDRLNCARAQGAILAVVRIHEDWVVFDVRGLLRNALWRELAGKGLTVRDLLDFFTGDPVVDTKRGVVTFTYKLGKVDVHSLRTVRGKRSKKVLEDLTLSSDVGLVTIDLGQTNVLAADYSKVTRSENGELLAVPLSKSFLPKHLLHEVTAYRTSYDQMEEGFRRKALLTLTEDQQVEVTLVRDFSVESSKTKLLQLGVDVTSLPWEKMSSNTTYISDQLLQQGADPASLFFDGERDGKPCRHKKKDRTWAYLVRPKVSPETRKALNEALWALKNTSPEFESLSKRKIQFSRRCMNYLLNEAKRISGCGQVVFVIEDLNVRVHHGRGKRAIGWDNFFKPKRENRWFMQALHKAASELAIHRGMHIIEACPARSSITCPKCGHCDPENRCSSDREKFLCVKCGAAFHADLEVATFNLRKVALTGTALPKSIDHSRDGLIPKGARNRKLKEPQANDEKACA

SEQ ID NO: 170 - protein, 735 aa, synthetic construct
  REGION 1..735: note = Description of Artificial Sequence: Synthetic polypeptide
MKEQSPLSSVLKSNFPGKKFLSADIRVAGRKLAQLGEAAAVEYLSPRQRDSVPNFRPPAFCTVVAKSRPFEEWPIYKASVLLQEQIYGMTGQEFEERCGSIPTSLSGLRQWASSVGLGAAMEGLHVQGMNLMVKNAINRYKGVLVKVENRNKKLVEANEAKNSSREERGLPPLRPPELGSAFGPDGRLVNPPGIDKSIRLYQGVSPVPVVKTTGRPTVHRLDIPAGEKGHVPLWQREAGLVKEGPRRRRMWYSNSNLKRSRKDRSAEASEARKADSVVVRVSVKEDWVDIDVRGLLRNVAWRGIERAGESTEDLLSLFSGDPVVDPSRDSVVFLYKEGVVDVLSKKVVGAGKSRKQLEKMVSEGPVALVSCDLGQTNYVAARVSVLDESLSPVRSFRVDPREFPSADGSQGVVGSLDRIRADSDRLEAKLLSEAEASLPEPVRAEIEFLRSERPSAVAGRLCLKLGIDPRSIPWEKMGSTTSFISEALSAKGSPLALHDGAPIKDSRFAHAARGRLSPESRKALNEALWERKSSSREYGVISRRKSEASRRMANAVLSESRRLTGLAVVAVNLEDLNMVSKFFHGRGKRAPGWAGFFTPKMENRWFIRSIHKAMCDLSKHRGITVIESRPERTSISCPECGHCDPENRSGERFSCKSCGVSLHADFEVATRNLERVALTGKPMPRRENLHSPEGATASRKTRKKPREATASTFLDLRSVLSSAENEGSGPAARAG

SEQ ID NO: 171 - protein, 725 aa, synthetic construct
  REGION 1..725: note = Description of Artificial Sequence: Synthetic polypeptide
MLPPSNKIGKSMSLKEFINKRNFKSSIIKQAGKILKKEGEEAVKKYLDDNYVEGYKKRDFPITAKCNIVASNRKIEDFDISKFSSFIQNYVFNLNKDNFEEFSKIKYNRKSFDELYKKIANEIGLEKPNYENIQGEIAVIRNAINIYNGVLKKVENRNKKIQEKNQSKDPPKLLSAFDDNGFLAERPGINETIYGYQSVRLRHLDVEKDKDIIVQLPDIYQKYNKKSTDKISVKKRLNKYNVDEYGKLISKRRKERINKDDAILCVSNFGDDWIIFDARGLLRQTYRYKLKKKGLCIKDLLNLFTGDPIINPTKTDLKEALSLSFKDGIINNRTLKVKNYKKCPELISELIRDKGKVAMISIDLGQTNPISYRLSKFTANNVAYIENGVISEDDIVKMKKWREKSDKLENLIKEEAIASLSDDEQREVRLYENDIADNTKKKILEKFNIREEDLDFSKMSNNTYFIRDCLKNKNIDESEFTFEKNGKKLDPTDACFAREYKNKLSELTRKKINEKIWEIKKNSKEYHKISIYKKETIRYIVNKLIKQSKEKSECDDIIVNIEKLQIGGNFFGGRGKRDPGWNNFFLPKEENRWFINACHKAFSELAPHKGIIVIESDPAYTSQTCPKCENCDKENRNGEKFKCKKCNYEANADIDVATENLEKIAKNGRRLIKNFDQLGERLPGAEMPGGARKRKPSKSLPKNGRGAGVGSEPELINQSPSQVIA

SEQ ID NO: 172 - protein, 718 aa, synthetic construct
  REGION 1..718: note = Description of Artificial Sequence: Synthetic polypeptide
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 173 - protein, 708 aa, synthetic construct
  REGION 1..708: note = Description of Artificial Sequence: Synthetic polypeptide
MSKTKELNDYQEALARRLPGVRHQKSVRRAARLVYDRQGEDAMVAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQEAAE

SEQ ID NO: 174 - protein, 491 aa, synthetic construct
  REGION 1..491: note = Description of Artificial Sequence: Synthetic polypeptide
VLLSDRIQYTDPSAPIPAMTVVDRRKIKKGEPGYVPPFMRKNLSTNKHRRMRLSRGQKEACALPVGLRLPDGKDGWDFIIFDGRALLRACRRLRLEVTSMDDVLDKFTGDPRIQLSPAGETIVTCMLKPQHTGVIQQKLITGKMKDRLVQLTAEAPIAMLTVDLGEHNLVACGAYTVGQRRGKLQSERLEAFLLPEKVLADFEGYRRDSDEHSETLRHEALKALSKRQQREVLDMLRTGADQARESLCYKYGLDLQALPWDKMSSNSTFIAQHLMSLGFGESATHVRYRPKRKASERTILKYDSRFAAEEKIKLTDETRRAWNEAIWECQRASQEFRCLSVRKLQLARAAVNWTLTQAKQRSRCPRVVVVVEDLNVRFMHGGGKRQEGWAGFFKARSEKRWFIQALHKAYTELPTNRGIHVMEVNPARTSITCTKCGYCDPENRYGEDFHCRNPKCKVRGGHVANADLDIATENLARVALSGPMPKAPKLK

SEQ ID NO: 175 - protein, 848 aa, synthetic construct
  REGION 1..848: note = Description of Artificial Sequence: Synthetic polypeptide
MTPSFGYQMIIVTPIHHASGAWATLRLLFLNPKTSGVMLGMTKTKSAFALMREEVFPGLLFKSADLKMAGRKFAKEGREAAIEYLRGKDEERPANFKPPAKGDIIAQSRPFDQWPIVQVSQAIQKYIFGLTKAEFDATKTLLYGEGNHPTTESRRRWFEATGVPDFGFTSAQGLNAIFSSALARYEGVIQKVENRNEKRLKKLSEKNQRLVEEGHAVEAYVPETAFHTLESLKALSEKSLVPLDDLMDKIDRLAQPPGINPCLYGYQQVAPYIYDPENPRGVVLPDLYLGYCRKPDDPITACPNRLDIPKGQPGYIPEHQRGQLKKHGRVRRFRYTNPQAKARAKAQTAILAVLRIDEDWVVMDLRGLLRNVYFREVAAPGELTARTLLDTFTGCPVLNLRSNVVTFCYDIESKGALHAEYVRKGWATRNKLLDLTKDGQSVALLSVDLGQRHPVAVMISRLKRDDKGDLSEKSIQVVSRTFADQYVDKLKRYRVQYDALRKEIYDAALVSLPPEQQAEIRAYEAFAPGDAKANVLSVMFQGEVSPDELPWDKMNTNTHYISDLYLRRGGDPSRVFFVPQPSTPKKNAKKPPAPRKPVKRTDENVSHMPEFRPHLSNETREAFQKAKWTMERGNVRYAQLSRFLNQIVREANNWLVSEAKKLTQCQTVVWAIEDLHVPFFHGKGKYHETWDGFFRQKKEDRWFVNVFHKAISERAPNKGEYVMEVAPYRTSQRCPVCGFVDADNRHGDHFKCLRCGVELHADLEVATWNIALVAVQGHGIAGPPREQSCGGETAGTARKGKNIKKNKGLADAVTVEAQDSEGGSKKDAGTARNPVYIPSESQVNCPAP

SEQ ID NO: 176 - protein, 781 aa, synthetic construct
  REGION 1..781: note = Description of Artificial Sequence: Synthetic polypeptide
MKPKTPKPPKTPVAALIDKHFPGKRFRASYLKSVGKKLKNQGEDVAVRFLTGKDEERPPNFQPPAKSNIVAQSRPIEEWPIHKVSVAVQEYVYGLTVAEKEACSDAGESSSSHAAWFAKTGVENFGYTSVQGLNKIFPPTFNRFDGVIKKVENRNEKKRQKATRINEAKRNKGQSEDPPEAEVKATDDAGYLLQPPGINHSVYGYQSITLCPYTAEKFPTIKLPEEYAGYHSNPDAPIPAGVPDRLAIPEGQPGHVPEEHRAGLSTKKHRRVRQWYAMANWKPKPKRTSKPDYDRLAKARAQGALLIVIRIDEDWVVVDARGLLRNVRWRSLGKREITPNELLDLFTGDPVLDLKRGVVTFTYAEGVVNVCSRSTTKGKQTKVLLDAMTAPRDGKKRQIGMVAVDLGQTNPIAAEYSRVGKNAAGTLEATPLSRSTLPDELLREIALYRKAHDRLEAQLREEAVLKLTAEQQAENARYVETSEEGAKLALANLGVDTSTLPWDAMTGWSTCISDHLINHGGDTSAVFFQTIRKGTKKLETIKRKDSSWADIVRPRLTKETREALNDFLWELKRSHEGYEKLSKRLEELARRAVNHVVQEVKWLTQCQDIVIVIEDLNVRNFHGGGKRGGGWSNFFTVKKENRWFMQALHKAFSDLAAHRGIPVLEVYPARTSITCLGCGHCDPENRDGEAFVCQQCGATFHADLEVATRNIARVALTGEAMPKAPAREQPGGAKKRGTSRRRKLTEVAVKSAEPTIHQAKNQQLNGTSRDPVYKGSELPAL

SEQ ID NO: 177 - protein, 767 aa, synthetic construct
  REGION 1..767: note = Description of Artificial Sequence: Synthetic polypeptide
MSEITDLLKANFKGKTFKSADMRMAGRILKKSGAQAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRGVQTTGTARNPGRSLKSNKNPSEPKRVLQSKTRKKITSTETQNEPLATDLKT

SEQ ID NO: 178 - protein, 760 aa, synthetic construct
  REGION 1..760: note = Description of Artificial Sequence: Synthetic polypeptide
MTPKTESPLSALCKKHFPGKRFRTNYLKDAGKILKKHGEDAVVAFLSDKQEDEPANFCPPAKVHILAQSRPFEDWPINLASKAIQTYVYGLTADERKTCEPGTSKESHDRWFKETGVDHHGFTSVQGLNLIFKHTLNRYDGVIKKVETRNEKRRSSVVRINEKKAAEGLPLIAAEAEETAFGEDGRLLQPPGVNHSIYCFQQVSPQPYSSKKHPQVVLPHAVQGVDPDAPIPVGRPNRLDIPKGQPGYVPEWQRPHLSMKCKRVRMWYARANWRRKPGRRSVLNEARLKEASAKGALPIVLVIGDDWLVMDARGLLRSVFWRRVAKPGLSLSELLNVTPTGLFSGDPVIDPKRGLVTFTSKLGVVAVHSRKPTRGKKSKDLLLKMTKPTDDGMPRHVGMVAIDLGQTNPVAAEYSRVVQSDAGTLKQEPVSRGVLPDDLLKDVARYRRAYDLTEESIRQEAIALLSEGHRAEVTKLDQTTANETKRLLVDRGVSESLPWEKMSSNTTYISDCLVALGKTDDVFFVPKAKKGKKETGIAVKRKDHGWSKLLRPRTSPEARKALNENQWAVKRASPEYERLSRRKLELGRRCVNHIIQETKRWTQCEDIVVVLEDLNVGFFHGSGKRPDGWDNFFVSKRENRWFIQVLHKAFGDLATHRGTHVIEVHPARTSITCIKCGHCDAGNRDGESFVCLASACGDRRHADLEVATRNVARVAITGERMPPSEQARDVQKAGGARKRKPSARNVKSSYPAVEPAPASP

SEQ ID NO: 179 - protein, 752 aa, synthetic construct
  REGION 1..752: note = Description of Artificial Sequence: Synthetic polypeptide
MSDNKMKKLSKEEKPLTPLQILIRKYIDKSQYPSGFKTTIIKQAGVRIKSVKSEQDEINLANWIISKYDPTYIKRDFNPSAKCQIIATSRSVADFDIVKMSNKVQEIFFASSHLDKNVFDIGKSKSDHDSWFERNNVDRGIYTYSNVQGMNLIFSNTKNTYLGVAVKAQNKFSSKMKRIQDINNFRITNHQSPLPIPDEIKIYDDAGFLLNPPGVNPNIFGYQSCLLKPLENKEIISKTSFPEYSRLPADMIEVNYKISNRLKFSNDQKGFIQFKDKLNLFKINSQELFSKRRRLSGQPILLVASFGDDWVVLDGRGLLRQVYYRGIAKPGSITISELLGFFTGDPIVDPIRGVVSLGFKPGVLSQETLKTTSARIFAEKLPNLVLNNNVGLMSIDLGQTNPVSYRLSEITSNMSVEHICSDFLSQDQISSIEKAKTSLDNLEEEIAIKAVDHLSDEDKINFANFSKLNLPEDTRQSLFEKYPELIGSKLDFGSMGSGTSYIADELIKFENKDAFYPSGKKKFDLSFSRDLRKKLSDETRKSYNDALFLEKRTNDKYLKNAKRRKQIVRTVANSLVSKIEELGLTPVINIENLAMSGGFFDGRGKREKGWDNFFKVKKENRWVMKDFHKAFSELSPHHGVIVIESPPYCTSVTCTKCNFCDKKNRNGHKFTCQRCGLDANADLDIATENLEKVAISGKRMPGSERSSDERKVAVARKAKSPKGKAIKGVKCTITDEPALLSANSQDCSQSTS

SEQ ID NO: 180 - protein, 747 aa, synthetic construct
  REGION 1..747: note = Description of Artificial Sequence: Synthetic polypeptide
MALSLAEVRERHFKGLRFRSSYLKRAGKILKKEGEAACVAYLTGKDEESPPNFKPPAKCDVVAQSRPFEEWPIVQASVAVQSYVYGLTKEAFEAFNPGTTKQSHEACLAATGIDTCGYSNVQGLNLIFRQAKNRYEGVITKVENRNKKAKKKLTRKNEWRQKNGHSELPEAPEELTFNDEGRLLQPPGINPSLYTYQQISPTPWSPKDSSILPPQYAGYERDPNAPIPFGVAKDRLTIASGCPGYIPEWMRTAGEKTNPRTQKKFMHPGLSTRKNKRMRLPRSVRSAPLGALLVTIHLGEDWLVLDVRGLLRNARWRGVAPKDISTQGLLNLFTGDPVIDTRRGVVTFTYKPETVGIHSRTWLYKGKQTKEVLEKLTQDQTVALVAIDLGQTNPVSAAASRVSRSGENLSIETVDRFFLPDELIKELRLYRMAHDRLEERIREESTLALTEAQQAEVRALEHVVRDDAKNKVCAAFNLDAASLPWDQMTSNTTYLSEAILAQGVSRDQVFFTPNPKKGSKEPVEVMRKDRAWVYAFKAKLSEETRKAKNEALWALKRASPDYARLSKRREELCRRSVNMVINRAKKRTQCQVVIPVLEDLNIGFFHGSGKRLPGWDNFFVAKKENRWLMNGLHKSFSDLAVHRGFYVFEVMPHRTSITCPACGHCDSENRDGEAFVCLSCKRTYHADLDVATHNLTQVAGTGLPMPEREHPGGTKKPGGSRKPESPQTHAPILHRTDYSESADRLGS

SEQ ID NO: 181 - protein, 733 aa, synthetic construct
  REGION 1..733: note = Description of Artificial Sequence: Synthetic polypeptide
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 182 - protein, 702 aa, synthetic construct
  REGION 1..702: note = Description of Artificial Sequence: Synthetic polypeptide
MIKEQSELSKLIEKYYPGKKFYSNDLKQAGKHLKKSEHLTAKESEELTVEFLKSCKEKLYDFRPPAKALIISTSRPFEEWPIYKASESIQKYIYSLTKEELEKYNISTDKTSQENFFKESLIDNYGFANVSGLNLIFQHTKAIYDGVLKKVNNRNNKILKKYKRKIEEGIEIDSPELEKAIDESGHFINPPGINKNIYCYQQVSPTIFNSFKETKIICPFNYKRNPNDIIQKGVIDRLAIPFGEPGYIPDHQRDKVNKHKKRIRKYYKNNENKNKDAILAKINIGEDWVLFDLRGLLRNAYWRKLIPKQGITPQQLLDMFSGDPVIDPIKNNITFIYKESIIPIHSESIIKTKKSKELLEKLTKDEQIALVSIDLGQTNPVAARFSRLSSDLKPEHVSSSFLPDELKNEICRYREKSDLLEIEIKNKAIKMLSQEQQDEIKLVNDISSEELKNSVCKKYNIDNSKIPWDKMNGFTTFIADEFINNGGDKSLVYFTAKDKKSKKEKLVKLSDKKIANSFKPKISKETREILNKITWDEKISSNEYKKLSKRKLEFARRATNYLINQAKKATRLNNVVLVVEDLNSKFFHGSGKREDGWDNFFIPKKENRWFIQALHKSLTDVSIHRGINVIEVRPERTSITCPKCGCCDKENRKGEDFKCIKCDSVYHADLEVATFNIEKVAITGESMPKPDCERLGGEESIG

SEQ ID NO: 183 - protein, 661 aa, synthetic construct
  REGION 1..661: note = Description of Artificial Sequence: Synthetic polypeptide
VAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQ

SEQ ID NO: 184 - protein, 531 aa, synthetic construct
  REGION 1..531: note = Description of Artificial Sequence: Synthetic polypeptide
LEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGIITFSYKEGVVPVFSQKIVSRFKSRDTLEKLTSQGPVALLSVDLGQNEPVAARVCSLKNINDKIALDNSCRIPFLDDYKKQIKDYRDSLDELEIKIRLEAINSLDVNQQVEIRDLDVFSADRAKASTVDMFDIDPNLISWDSMSDARFSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKSFSELSSNRGLCVIEVNPAWTSATCPDCGFCSKENRDGINFTCRKCGVSYHADIDVATLNIARVAVLGKPMSGPADRERLGGTKKPRVARSRKDMKRKDISNGTVEVMVTA

SEQ ID NO: 185 - protein, 479 aa, synthetic construct
  REGION 1..479: note = Description of Artificial Sequence: Synthetic polypeptide
IPSFGYLDRLKIAKGQPGYIPEWQRETINPSKKVRRYWATNHEKIRNAIPLVVFIGDDWVIIDGRGLLRDARRRKLADKNTTIEQLLEMVSNDPVIDSTRGIATLSYVEGVVPVRSFIPIGEKKGREYLEKSTQKESVTLLSVDIGQINPVSCGVYKVSNGCSKIDFLDKFFLDKKHLDAIQKYRTLQDSLEASIVNEALDEIDPSFKKEYQNINSQTSNDVKKSLCTEYNIDPEAISWQDITAHSTLISDYLIDNNITNDVYRTVNKAKYKTNDFGWYKKFSAKLSKEAREALNEKIWELKIASSKYKKLSVRKKEIARTIANDCVKRAETYGDNVVVAMESLTKNNKVMSGRGKRDPGWHNLGQAKVENRWFIQAISSAFEDKATHHGTPVLKVNPAYTSQTCPSCGHCSKDNRSSKDRTIFVCKSCGEKFNADLDVATYNIAHVAFSGKKLSPPSEKSSATKKPRSARKSKKSRKS

SEQ ID NO: 186 - protein, 452 aa, synthetic construct
  REGION 1..452: note = Description of Artificial Sequence: Synthetic polypeptide
SPIEKLLNGLLVKITFGNDWIICDARGLLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 187 - protein, 425 aa, synthetic construct
  REGION 1..425: note = Description of Artificial Sequence: Synthetic polypeptide
LLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 188 - protein, 735 aa, synthetic construct
  REGION 1..735: note = Description of Artificial Sequence: Synthetic polypeptide
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 189 - protein, 1125 aa, synthetic construct
  REGION 1..1125: note = Description of Artificial Sequence: Synthetic polypeptide
MRKKLFKGYILHNKRLVYTGKAAIRSIKYPLVAPNKTALNNLSEKIIYDYEHLFGPLNVASYARNSNRYSLVDFWIDSLRAGVIWQSKSTSLIDLISKLEGSKSPSEKIFEQIDFELKNKLDKEQFKDIILLNTGIRSSSNVRSLRGRFLKCFKEEFRDTEEVIACVDKWSKDLIVEGKSILVSKQFLYWEEEFGIKIFPHFKDNHDLPKLTFFVEPSLEFSPHLPLANCLERLKKFDISRESLLGLDNNFSAFSNYFNELFNLLSRGEIKKIVTAVLAVSKSWENEPELEKRLHFLSEKAKLLGYPKLTSSWADYRMIIGGKIKSWHSNYTEQLIKVREDLKKHQIALDKLQEDLKKVVDSSLREQIEAQREALLPLLDTMLKEKDFSDDLELYRFILSDFKSLLNGSYQRYIQTEEERKEDRDVTKKYKDLYSNLRNIPRFFGESKKEQFNKFINKSLPTIDVGLKILEDIRNALETVSVRKPPSITEEYVTKQLEKLSRKYKINAFNSNRFKQITEQVLRKYNNGELPKISEVFYRYPRESHVAIRILPVKISNPRKDISYLLDKYQISPDWKNSNPGEVVDLIEIYKLTLGWLLSCNKDFSMDFSSYDLKLFPEAASLIKNFGSCLSGYYLSKMIFNCITSEIKGMITLYTRDKFVVRYVTQMIGSNQKFPLLCLVGEKQTKNFSRNWGVLIEEKGDLGEEKNQEKCLIFKDKTDFAKAKEVEIFKNNIWRIRTSKYQIQFLNRLFKKTKEWDLMNLVLSEPSLVLEEEWGVSWDKDKLLPLLKKEKSCEERLYYSLPLNLVPATDYKEQSAEIEQRNTYLGLDVGEFGVAYAVVRIVRDRIELLSWGFLKDPALRKIRERVQDMKKKQVMAVFSSSSTAVARVREMAIHSLRNQIHSIALAYKAKIIYEISISNFETGGNRMAKIYRSIKVSDVYRESGADTLVSEMIWGKKNKQMGNHISSYATSYTCCNCARTPFELVIDNDKEYEKGGDEFIFNVGDEKKVRGFLQKSLLGKTIKGKEVLKSIKEYARPPIREVLLEGEDVEQLLKRRGNSYIYRCPFCGYKTDADIQAALNIACRGYISDNAKDAVKEGERKLDYILEVRKLWEKNGAVLRSAKFL

SEQ ID NO: 190 - protein, 1226 aa, synthetic construct
  REGION 1..1226: note = Description of Artificial Sequence: Synthetic polypeptide
MQKVRKTLSEVHKNPYGTKVRNAKTGYSLQIERLSYTGKEGMRSFKIPLENKNKEVFDEFVKKIRNDYISQVGLLNLSDWYEHYQEKQEHYSLADFWLDSLRAGVIFAHKETEIKNLISKIRGDKSIVDKFNASIKKKHADLYALVDIKALYDFLTSDARRGLKTEEEFFNSKRNTLFPKFRKKDNKAVDLWVKKFIGLDNKDKLNFTKKFIGFDPNPQIKYDHTFFFHQDINFDLERITTPKELISTYKKFLGKNKDLYGSDETTEDQLKMVLGFHNNHGAFSKYFNASLEAFRGRDNSLVEQIINNSPYWNSHRKELEKRIIFLQVQSKKIKETELGKPHEYLASFGGKFESWVSNYLRQEEEVKRQLFGYEENKKGQKKFIVGNKQELDKIIRGTDEYEIKAISKETIGLTQKCLKLLEQLKDSVDDYTLSLYRQLIVELRIRLNVEFQETYPELIGKSEKDKEKDAKNKRADKRYPQIFKDIKLIPNFLGETKQMVYKKFIRSADILYEGINFIDQIDKQITQNLLPCFKNDKERIEFTEKQFETLRRKYYLMNSSRFHHVIEGIINNRKLIEMKKRENSELKTFSDSKFVLSKLFLKKGKKYENEVYYTFYINPKARDQRRIKIVLDINGNNSVGILQDLVQKLKPKWDDIIKKNDMGELIDAIEIEKVRLGILIALYCEHKFKIKKELLSLDLFASAYQYLELEDDPEELSGTNLGRFLQSLVCSEIKGAINKISRTEYIERYTVQPMNTEKNYPLLINKEGKATWHIAAKDDLSKKKGGGTVAMNQKIGKNFFGKQDYKTVFMLQDKRFDLLTSKYHLQFLSKTLDTGGGSWWKNKNIDLNLSSYSFIFEQKVKVEWDLTNLDHPIKIKPSENSDDRRLFVSIPFVIKPKQTKRKDLQTRVNYMGIDIGEYGLAWTIINIDLKNKKINKISKQGFIYEPLTHKVRDYVATIKDNQVRGTFGMPDTKLARLRENAITSLRNQVHDIAMRYDAKPVYEFEISNFETGSNKVKVIYDSVKRADIGRGQNNTEADNTEVNLVWGKTSKQFGSQIGAYATSYICSFCGYSPYYEFENSKSGDEEGARDNLYQMKKLSRPSLEDFLQGNPVYKTFRDFDKYKNDQRLQKTGDKDGEWKTHRGNTAIYACQKCRHISDADIQASYWIALKQVVRDFYKDKEMDGDLIQGDNKDKRKVNELNRLIGVHKDVPIINKNLITSLDINLL

SEQ ID NO: 191 - protein, 1201 aa, synthetic construct
  REGION 1..1201: note = Description of Artificial Sequence: Synthetic polypeptide
MKAKKSFYNQKRKFGKRGYRLHDERIAYSGGIGSMRSIKYELKDSYGIAGLRNRIADATISDNKWLYGNINLNDYLEWRSSKTDKQIEDGDRESSLLGFWLEALRLGFVFSKQSHAPNDFNETALQDLFETLDDDLKHVLDRKKWCDFIKIGTPKTNDQGRLKKQIKNLLKGNKREEIEKTLNESDDELKEKINRIADVFAKNKSDKYTIFKLDKPNTEKYPRINDVQVAFFCHPDFEEITERDRTKTLDLIINRFNKRYEITENKKDDKTSNRMALYSLNQGYIPRVLNDLFLFVKDNEDDFSQFLSDLENFFSFSNEQIKIIKERLKKLKKYAEPIPGKPQLADKWDDYASDFGGKLESWYSNRIEKLKKIPESVSDLRNNLEKIRNVLKKQNNASKILELSQKIIEYIRDYGVSFEKPEIIKFSWINKTKDGQKKVFYVAKMADREFIEKLDLWMADLRSQLNEYNQDNKVSFKKKGKKIEELGVLDFALNKAKKNKSTKNENGWQQKLSESIQSAPLFFGEGNRVRNEEVYNLKDLLFSEIKNVENILMSSEAEDLKNIKIEYKEDGAKKGNYVLNVLARFYARFNEDGYGGWNKVKTVLENIAREAGTDFSKYGNNNNRNAGRFYLNGRERQVFTLIKFEKSITVEKILELVKLPSLLDEAYRDLVNENKNHKLRDVIQLSKTIMALVLSHSDKEKQIGGNYIHSKLSGYNALISKRDFISRYSVQTTNGTQCKLAIGKGKSKKGNEIDRYFYAFQFFKNDDSKINLKVIKNNSHKNIDFNDNENKINALQVYSSNYQIQFLDWFFEKHQGKKTSLEVGGSFTIAEKSLTIDWSGSNPRVGFKRSDTEEKRVFVSQPFTLIPDDEDKERRKERMIKTKNRFIGIDIGEYGLAWSLIEVDNGDKNNRGIRQLESGFITDNQQQVLKKNVKSWRQNQIRQTFTSPDTKIARLRESLIGSYKNQLESLMVAKKANLSFEYEVSGFEVGGKRVAKIYDSIKRGSVRKKDNNSQNDQSWGKKGINEWSFETTAAGTSQFCTHCKRWSSLAIVDIEEYELKDYNDNLFKVKINDGEVRLLGKKGWRSGEKIKGKELFGPVKDAMRPNVDGLGMKIVKRKYLKLDLRDWVSRYGNMAIFICPYVDCHHISHADKQAAFNIAVRGYLKSVNPDRAIKHGDKGLSRDFLCQEEGKLNFEQIGLLI

SEQ ID NO: 192 - protein, 1210 aa, synthetic construct
  REGION 1..1210: note = Description of Artificial Sequence: Synthetic polypeptide
MSKRHPRISGVKGYRLHAQRLEYTGKSGAMRTIKYPLYSSPSGGRTVPREIVSAINDDYVGLYGLSNFDDLYNAEKRNEEKVYSVLDFWYDCVQYGAVFSYTAPGLLKNVAEVRGGSYELTKTLKGSHLYDELQIDKVIKFLNKKEISRANGSLDKLKKDIIDCFKAEYRERHKDQCNKLADDIKNAKKDAGASLGERQKKLFRDFFGISEQSENDKPSFTNPLNLTCCLLPFDTVNNNRNRGEVLFNKLKEYAQKLDKNEGSLEMWEYIGIGNSGTAFSNFLGEGFLGRLRENKITELKKAMMDITDAWRGQEQEEELEKRLRILAALTIKLREPKFDNHWGGYRSDINGKLSSWLQNYINQTVKIKEDLKGHKKDLKKAKEMINRFGESDTKEEAVVSSLLESIEKIVPDDSADDEKPDIPAIAIYRRFLSDGRLTLNRFVQREDVQEALIKERLEAEKKKKPKKRKKKSDAEDEKETIDFKELFPHLAKPLKLVPNFYGDSKRELYKKYKNAAIYTDALWKAVEKIYKSAFSSSLKNSFFDTDFDKDFFIKRLQKIFSVYRRFNTDKWKPIVKNSFAPYCDIVSLAENEVLYKPKQSRSRKSAAIDKNRVRLPSTENIAKAGIALARELSVAGFDWKDLLKKEEHEEYIDLIELHKTALALLLAVTETQLDISALDFVENGTVKDFMKTRDGNLVLEGRFLEMFSQSIVFSELRGLAGLMSRKEFITRSAIQTMNGKQAELLYIPHEFQSAKITTPKEMSRAFLDLAPAEFATSLEPESLSEKSLLKLKQMRYYPHYFGYELTRTGQGIDGGVAENALRLEKSPVKKREIKCKQYKTLGRGQNKIVLYVRSSYYQTQFLEWFLHRPKNVQTDVAVSGSFLIDEKKVKTRWNYDALTVALEPVSGSERVFVSQPFTIFPEKSAEEEGQRYLGIDIGEYGIAYTALEITGDSAKILDQNFISDPQLKTLREEVKGLKLDQRRGTFAMPSTKIARIRESLVHSLRNRIHHLALKHKAKIVYELEVSRFEEGKQKIKKVYATLKKADVYSEIDADKNLQTTVWGKLAVASEISASYTSQFCGACKKLWRAEMQVDETITTQELIGTVRVIKGGTLIDAIKDFMRPPIFDENDTPFPKYRDFCDKHHISKKMRGNSCLFICPFCRANADADIQASQTIALLRYVKEEKKVEDYFERFRKLKNIKVLGQMKKI

SEQ ID NO: 193 - protein, 1287 aa, synthetic construct
  REGION 1..1287: note = Description of Artificial Sequence: Synthetic polypeptide
MKRILNSLKVAALRLLFRGKGSELVKTVKYPLVSPVQGAVEELAEAIRHDNLHLFGQKEIVDLMEKDEGTQVYSVVDFWLDTLRLGMFFSPSANALKITLGKFNSDQVSPFRKVLEQSPFFLAGRLKVEPAERILSVEIRKIGKRENRVENYAADVETCFIGQLSSDEKQSIQKLANDIWDSKDHEEQRMLKADFFAIPLIKDPKAVTEEDPENETAGKQKPLELCVCLVPELYTRGFGSIADFLVQRLTLLRDKMSTDTAEDCLEYVGIEEEKGNGMNSLLGTFLKNLQGDGFEQIFQFMLGSYVGWQGKEDVLRERLDLLAEKVKRLPKPKFAGEWSGHRMFLHGQLKSWSSNFFRLFNETRELLESIKSDIQHATMLISYVEEKGGYHPQLLSQYRKLMEQLPALRTKVLDPEIEMTHMSEAVRSYIMIHKSVAGFLPDLLESLDRDKDREFLLSIFPRIPKIDKKTKEIVAWELPGEPEEGYLFTANNLFRNFLENPKHVPRFMAERIPEDWTRLRSAPVWFDGMVKQWQKVVNQLVESPGALYQFNESFLRQRLQAMLTVYKRDLQTEKFLKLLADVCRPLVDFFGLGGNDIIFKSCQDPRKQWQTVIPLSVPADVYTACEGLAIRLRETLGFEWKNLKGHEREDFLRLHQLLGNLLFWIRDAKLVVKLEDWMNNPCVQEYVEARKAIDLPLEIFGFEVPIFLNGYLFSELRQLELLLRRKSVMTSYSVKTTGSPNRLFQLVYLPLNPSDPEKKNSNNFQERLDTPTGLSRRFLDLTLDAFAGKLLTDPVTQELKTMAGFYDHLFGFKLPCKLAAMSNHPGSSSKMVVLAKPKKGVASNIGFEPIPDPAHPVFRVRSSWPELKYLEGLLYLPEDTPLTIELAETSVSCQSVSSVAFDLKNLTTILGRVGEFRVTADQPFKLTPIIPEKEESFIGKTYLGLDAGERSGVGFAIVTVDGDGYEVQRLGVHEDTQLMALQQVASKSLKEPVFQPLRKGTFRQQERIRKSLRGCYWNFYHALMIKYRAKVVHEESVGSSGLVGQWLRAFQKDLKKADVLPKKGGKNGVDKKKRESSAQDTLWGGAFSKKEEQQIAFEVQAAGSSQFCLKCGWWFQLGMREVNRVQESGVVLDWNRSIVTFLIESSGEKVYGFSPQQLEKGFRPDIETFKKMVRDFMRPPMFDRKGRPAAAYERFVLGRRHRRYRFDKVFEERFGRSALFICPRVGCGNFDHSSEQSAVVLALIGYIADKEGMSGKKLVYVRLAELMAEWKLKKLERSRVEEQSSAQ

SEQ ID NO: 194 - protein, 1192 aa, synthetic construct
  REGION 1..1192: note = Description of Artificial Sequence: Synthetic polypeptide
MAESKQMQCRKCGASMKYEVIGLGKKSCRYMCPDCGNHTSARKIQNKKKRDKKYGSASKAQSQRIAVAGALYPDKKVQTIKTYKYPADLNGEVHDSGVAEKIAQAIQEDEIGLLGPSSEYACWIASQKQSEPYSVVDFWFDAVCAGGVFAYSGARLLSTVLQLSGEESVLRAALASSPFVDDINLAQAEKFLAVSRRTGQDKLGKRIGECFAEGRLEALGIKDRMREFVQAIDVAQTAGQRFAAKLKIFGISQMPEAKQWNNDSGLTVCILPDYYVPEENRADQLVVLLRRLREIAYCMGIEDEAGFEHLGIDPGALSNFSNGNPKRGFLGRLLNNDIIALANNMSAMTPYWEGRKGELIERLAWLKHRAEGLYLKEPHFGNSWADHRSRIFSRIAGWLSGCAGKLKIAKDQISGVRTDLFLLKRLLDAVPQSAPSPDFIASISALDRFLEAAESSQDPAEQVRALYAFHLNAPAVRSIANKAVQRSDSQEWLIKELDAVDHLEFNKAFPFFSDTGKKKKKGANSNGAPSEEEYTETESIQQPEDAEQEVNGQEGNGASKNQKKFQRIPRFFGEGSRSEYRILTEAPQYFDMFCNNMRAIFMQLESQPRKAPRDFKCFLQNRLQKLYKQTFLNARSNKCRALLESVLISWGEFYTYGANEKKFRLRHEASERSSDPDYVVQQALEIARRLFLFGFEWRDCSAGERVDLVEIHKKAISFLLAITQAEVSVGSYNWLGNSTVSRYLSVAGTDTLYGTQLEEFLNATVLSQMRGLAIRLSSQELKDGFDVQLESSCQDNLQHLLVYRASRDLAACKRATCPAELDPKILVLPVGAFIASVMKMIERGDEPLAGAYLRHRPHSFGWQIRVRGVAEVGMDQGTALAFQKPTESEPFKIKPFSAQYGPVLWLNSSSYSQSQYLDGFLSQPKNWSMRVLPQAGSVRVEQRVALIWNLQAGKMRLERSGARAFFMPVPFSFRPSGSGDEAVLAPNRYLGLFPHSGGIEYAVVDVLDSAGFKILERGTIAVNGFSQKRGERQEEAHREKQRRGISDIGRKKPVQAEVDAANELHRKYTDVATRLGCRIVVQWAPQPKPGTAPTAQTVYARAVRTEAPRSGNQEDHARMKSSWGYTWGTYWEKRKPEDILGISTQVYWTGGIGESCPAVAVALLGHIRATSTQTEWEKEEVVFGRLKKFFPS

SEQ ID NO: 195 - protein, 1192 aa, synthetic construct
  REGION 1..1192: note = Description of Artificial Sequence: Synthetic polypeptide
MAESKQMQCRKCGASMKYEVIGLGKKSCRYMCPDCGNHTSARKIQNKKKRDKKYGSASKAQSQRIAVAGALYPDKKVQTIKTYKYPADLNGEVHDRGVAEKIEQAIQEDEIGLLGPSSEYACWIASQKQSEPYSVVDFWFDAVCAGGVFAYSGARLLSTVLQLSGEESVLRAALASSPFVDDINLAQAEKFLAVSRRTGQDKLGKRIGECFAEGRLEALGIKDRMREFVQAIDVAQTAGQRFAAKLKIFGISQMPEAKQWNNDSGLTVCILPDYYVPEENRADQLVVLLRRLREIAYCMGIEDEAGFEHLGIDPGALSNFSNGNPKRGFLGRLLNNDIIALANNMSAMTPYWEGRKGELIERLAWLKHRAEGLYLKEPHFGNSWADHRSRIFSRIAGWLSGCAGKLKIAKDQISGVRTDLFLLKRLLDAVPQSAPSPDFIASISALDRFLEAAESSQDPAEQVRALYAFHLNAPAVRSIANKAVQRSDSQEWLIKELDAVDHLEFNKAFPFFSDTGKKKKKGANSNGAPSEEEYTETESIQQPEDAEQEVNGQEGNGASKNQKKFQRIPRFFGEGSRSEYRILTEAPQYFDMFCNNMRAIFMQLESQPRKAPRDFKCFLQNRLQKLYKQTFLNARSNKCRALLESVLISWGEFYTYGANEKKFRLRHEASERSSDPDYVVQQALEIARRLFLFGFEWRDCSAGERVDLVEIHKKAISFLLAITQAEVSVGSYNWLGNSTVSRYLSVAGTDTLYGTQLEEFLNATVLSQMRGLAIRLSSQELKDGFDVQLESSCQDNLQHLLVYRASRDLAACKRATCPAELDPKILVLPAGAFIASVMKMIERGDEPLAGAYLRHRPHSFGWQIRVRGVAEVGMDQGTALAFQKPTESEPFKIKPFSAQYGPVLWLNSSSYSQSQYLDGFLSQPKNWSMRVLPQAGSVRVEQRVALIWNLQAGKMRLERSGARAFFMPVPFSFRPSGSGDEAVLAPNRYLGLFPHSGGIEYAVVDVLDSAGFKILERGTIAVNGFSQKRGERQEEAHREKQRRGISDIGRKKPVQAEVDAANELHRKYTDVATRLGCRIVVQWAPQPKPGTAPTAQTVYARAVRTEAPRSGNQEDHARMKSSWGYTWSTYWEKRKPEDILGISTQVYWTGGIGESCPAVAVALLGHIRATSTQTEWEKEEVVFGRLKKFFPS

SEQ ID NO: 196 - protein, 1193 aa, synthetic construct
  REGION 1..1193: note = Description of Artificial Sequence: Synthetic polypeptide
MKRIAKFRHDKPVKREAWSKGYRVHKNRIINKVTRSIKYPLVVKDEWKKRLIDDAAHDYRWLVGPINYSDWCRDPNQYSILEFWIDFLCVGGVFQSSHSNICRLAIQLSGGSVFEQEWKDLSPFVRANLIQGIKPAEFIGFLTAEFRSSSNPKNFISKFFEGSNEDLESLTNEFASIVDFIKAKDISLLRKSLPSCKKIAPNLWEKAVGSHSTNELLKLLTKYTRVMLVAEPSHSDRVFSQTVLQSNDQDDPELTGPLPSHKVGKASYLFIPEFIREVNLDKISKLDLSAKSKLAVEQVKKLSELTSDFKQIENQSEAYFGLSTSFNELSNFLGILIRTLRNAPEAILKDQIALCAPLDKDILKITLDWLCDRAQALPENPRFETNWAEYRSYLGGKIKSWFSNYENFFEIPQAASSQQNNNREKKLGNRSAIRALNLKKEAFEKARETFKGDKGTLEKIDLAYRLLGSISPEVLQCDEGLKLYQQFNDELLVLNETINQKFQDAKRDIKAKKEKESFEKLQRNLSSPLPRIPEFFGERAKKGYQKARVSPKLARHLLECLNDWLARFAKVEESAFSEKEFQRILDWLRTSDFLPVFIRKSKDPPSWLRYIARVATGKYYFWVSEYSRKRVQIIDKPIAQNPLKELISWFLLNKDAFSRDNELFKGLSSKMVTLARIMAGILRDRGEGLKELQAMTSKLDNIGLLHPSFSVPVTDSLKDAAFYRAFFSELEGLLNIGRSRLIIERITLQSQQSKNKKTRRPLMPEPFINEDKEVFLAFPKFETKNKVKGTRVVYNSPDEVNWLLSPIRSSKGQLSFMFRCLSEDAKIMTTSGGCSYIVEFKKLLEAQEEVLSIHDCDIIPRAFVSIPFTLERESEETKPDWKPNRFMGVDIGEYAVAYCVIEKGTDSIEILDCGIVRNGAHRVLKEKVDRLKRRQRSMTFGAMDTSIAAARESLVGNYRNRLHAIALKHGAKLVYEYEVSAFESGGNRIKKVYETLKKSDCTGETEADKNARKHIWGETNAVGDQIGAGWTSQTCAKCGRSFGADLKAGNFGVAVPVPEKVEDSKGHYAYHEFPFEDGLKVRGFLKPNKIISDQKELAKAVHAYMRPPLVALGKRKLPKNARYRRGNSSLFRCPFSDCGFTADADIQAAYNIAVKQLYKPKKGYPKERKWQDFVILKPKEPSKLFDKQFYRPN

SEQ ID NO: 197 - protein, 1154 aa, synthetic construct
  REGION 1..1154: note = Description of Artificial Sequence: Synthetic polypeptide
MKDSKINAPININANNVSKNKTPKKKPRRKSGKRGYRLHDERIAYSGGTGSCRSIKYELLNPDATRKNLLRGSGLQHEIISAVRQDNLLLYGPLNFNDYIFDKDAPNLLHFWTLALSLGFVFSNQNSIEREFKDYLGVSTEEAVLFGKLNETLKAVFDEAKFISGFLYRNFRGLASKTREQRIKLLTDTLREPLDGVNGDSVSEIIKPYAEKWAEYDGECDQFVFKCELFSIKSTDKPRENTRLSFAIDPAFEVMKLDDKTVFFDDLITHYKENCSDEAQAKRFLGIGDNGNYFNGIFGGLFELLTDGDEKICETTDHLARIYGFDETKKTEINKRLVRLAEYARQINRRPCLVKRWSEYRSDFNGTIESWYSNRQSKQNDTLKQLDEKLKLLEEMRASFPTDSDLCGIKSLSETIEFIRSLKGERIARKVTDELESYLAVLGSELNQYTQQNKDHALPLGWQKKLSKHIQSSPLFFGENKIALWEKLINLKELIKTEVKELEVVLAEDFDDYEITDKQVDNLAALAGRFSESPDGSGHPLVTERLAKIESTLGVDFTHKNNRAKFYLSGFERGKFGKLDVPNKIKVSHLFELADLSILYNAVANSPEDGYILRDTAQLSKIILSAKLRDADREKQRKTVLAHSTLQGYSALISKREFVSRYPLQAVNGSQNLMAYDANRKYYYAYNSEKFAGTKELTVALRGNNFGPEAFGGKFKKVPALRVQSSKYQIQFLDWFFEKQKKRKTELGAGGSFTIAEISCKVNWDDKTPVIFEKPDPRLFVSQPFTINPPENSAKKDYARYIGIDIGEYGLAWHLVEVFEDANEDIGGAGKNAVRIKSVEKGFFTDPQQISLKEDVKKLRENQVRATFTSPDTKIARVRESLIGSYRNLLEDLAVRKDARLCFEYEVSGFESGGARISKVYDSIKRSSVAKKENKAENKQSWGKLFGPEFSFKAIEITAAGTSQYCTKCKRWASLAIKDNNNYQLLEWDNGETGDKRGSDGLLAVTLDGEGKETNRTVRLFPKDGKKAGDTIKGKDLKSAIYRAMRPNMRPSEDGSISLGAGMEAVRRDLMPEQWEKLTLEFGQGKPRGNMAIYVCPYCGHISDADMQAAFNIAVRGYLANRDKEKKVKLGKEYLTDEQSKLTFDPVGILEHTT

SEQ ID NO: 198 - protein, 1266 aa, synthetic construct
  REGION 1..1266: note = Description of Artificial Sequence: Synthetic polypeptide
MPLMIRNTMNEKKTATQRRNARRRRGERARTKSQELRGYRLHDARIEFSGGLGSMRTVKVELLNPDSSREDPQRGQGLQGKVAKAVFDDYRALYGPMNIEDYLSDPDCPSFLGLWVKAVCLGVIMSRKTATDFGELRGGSKSGQAFDSIPEHLRRQLIKLKWLDWYDKGIRKSSSKASRLKSLTDVFANPKQPDQGVMAAWEQGEKLAESSRDIAALGRREFKDKLFAIPPPTSSVVLDDDVKATKVSRDWQWAVDPQFKLPSTDLDITRALEEVDRQWFERLGNNRGMVQQFFAIGDNGNHLNNGLFGHFFASIRSANLADIVAEMGTAFGFSAEERDIVRQRLETLHEYAQGLPEKPVLASRWAEYRTDMTAKLGSWYSNRTSKGAASITQVWGTINTETGEVKDDGLVRTLENIQSDLPDSCSIKEGILQETLDFIGDRRSSTDRAFTDELELYLATLRSDLNTWCQEQSALWEEKQRQVATPASDEKSKKADNPWAGKGSKTDKWLGALHTRIQSSPLFWGVDKLELWKTLANLKQAIRDEIDKLNEQVEVFGRSAYDEPVGKDADSGEGDRRVDQLSYLSARLGDQAHEEVRQRLDAIALALGVKFSERDDLHRFFVSSRARRRAALLAMPNTITVGKLRELADLTPLWERIKKKPEEPRLLADTVALSKVVNSACASRANPSDQIELTTIHSRLDGYSKNIGHTEFISRATVQSTNGAQNTVALDSLVSPRLFYYNFPNIVESAEPHVSHLEVATRGNLGSFEEFAAKEHRTFDRENPQKDSRNRIDSVNPLAVASSRYQIQFFTWWAGLHRSKETALEVGGSFTIAERQVRLDWSQEKPQAVVSEELRVFVSQPFTIVPDDKKRPATSGTRYIGVAIGEYGLAWSCWEFAPGYWNGSVVNPSKVTCLDYGFLAEPGQRRIVERVKKLRESQATKTFTSPDTYIARLRENVVATYQAQLEALMMAYNAQLVFASEISAFETGGNRVKKIYDAIKRSSVFGRSDAEATDNNQHWGKNGNRSSVKDPDKLRLNEAGQVAARVPWAEPVSAWMTSQTCSACGRVYVRAYRGKNSNEPDSGATGEVRYFDNKQQKILTKTIGADTVWVTDQERKEFERGVYNAMRPNAFMPDGRWTAAGEILEAALKSRGTLDGGRGFAGLHLTSKAQVHEYIEGTGKSHADAHGNSAIFICPYTDCGHIADAALQASYNIALRGFAYAIVRKKHPELFAGSGSSTDGDEGGGKKPQQKQAFIDEIVRAAGRAS

SEQ ID NO: 199 - protein, 18 aa, synthetic construct
  REGION 1..18: note = Description of Artificial Sequence: Synthetic peptide
KRPAATKKAGQAKKKKEF

SEQ ID NO: 200 - protein, 20 aa, synthetic construct
  REGION 1..20: note = Description of Artificial Sequence: Synthetic peptide
  MOD_RES 4: note = Lys, His, or Arg
  MOD_RES 8: note = Lys, His, or Arg
  MOD_RES 11: note = Lys, His, or Arg
  MOD_RES 15: note = Lys, His, or Arg
  MOD_RES 19: note = Lys, His, or Arg
GLFXALLXLLXSLWXLLLXA

SEQ ID NO: 201 - protein, 20 aa, synthetic construct
  REGION 1..20: note = Description of Artificial Sequence: Synthetic peptide
GLFHALLHLLHSLWHLLLHA

SEQ ID NO: 202-203 - skipped (2 entries)

SEQ ID NO: 204 - RNA, 10 nt, synthetic construct
  misc_feature 1..10: note = Description of Artificial Sequence: Synthetic oligonucleotide
  source 1..10: mol_type = other RNA
tttttttttt

SEQ ID NO: 205 - DNA, 10 bp, synthetic construct
  misc_feature 1..10: note = Description of Artificial Sequence: Synthetic oligonucleotide
  misc_feature 1..10: note = Description of Combined DNA/RNA Molecule: Synthetic oligonucleotide
  source 1..10: mol_type = other DNA
  modified_base 5..6: mod_base = OTHER; note = uracil
tttttttttt

SEQ ID NO: 206-212 - skipped (7 entries)

SEQ ID NO: 213 - DNA, 10 bp, synthetic construct
  misc_feature 1..10: note = Description of Artificial Sequence: Synthetic oligonucleotide
  source 1..10: mol_type = other DNA
tttttttttt

SEQ ID NO: 214 - DNA, 12 bp, synthetic construct
  misc_feature 1..12: note = Description of Artificial Sequence: Synthetic oligonucleotide
  source 1..12: mol_type = other DNA
tttttttttttt

SEQ ID NO: 215 - DNA, 14 bp, synthetic construct
  misc_feature 1..14: note = Description of Artificial Sequence: Synthetic oligonucleotide
  source 1..14: mol_type = other DNA
tttttttttttttt

SEQ ID NO: 216-219 - skipped (4 entries)

SEQ ID NO: 220 - RNA, 140 nt, synthetic construct
  misc_feature 1..140: note = Description of Artificial Sequence: Synthetic polynucleotide
  source 1..140: mol_type = other RNA
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcattt

SEQ ID NO: 221 - RNA, 189 nt, synthetic construct
  misc_feature 1..189: note = Description of Artificial Sequence: Synthetic polynucleotide
  source 1..189: mol_type = other RNA
cgattcctccctacagtagttaggtatagccgaaaggtagagactaaatctgtagttggagtgggccgcttgcatcggcctaaagttgagaagtgtcagactctgataaccctcaacgacgatattctttatttcggttcaaagttctgcacaaaacaggtgagtccttataaaccggtgtgcagaacg

SEQ ID NO: 222 - RNA, 103 nt, synthetic construct
  misc_feature 1..103: note = Description of Artificial Sequence: Synthetic polynucleotide
  source 1..103: mol_type = other RNA
gtgaaggtgggctgcttgcatcagcctaatgtcgagaagtgctttcttcggaaagtaaccctcgaaacaaattcatttttcctctccaattctgcacaaaaaa

SEQ ID NO: 223 - RNA, 103 nt, synthetic construct
  misc_feature 1..103: note = Description of Artificial Sequence: Synthetic polynucleotide
  source 1..103: mol_type = other RNA
tttctcagagatggctgatcccaacagccaaatatcgaggtgtctgcacctatatggacgcagtaagtagaaccaccctcgaaacagttctatcggatttttt

SEQ ID NO: 224 - RNA, 103 nt, synthetic construct; identical to SEQ ID NO: 223
  misc_feature 1..103: note = Description of Artificial Sequence: Synthetic polynucleotide
  source 1..103: mol_type = other RNA

SEQ ID NO: 225 - RNA, 103 nt, synthetic construct
  misc_feature 1..103: note = Description of Artificial Sequence: Synthetic polynucleotide
  source 1..103: mol_type = other RNA
ggctgatttcaacagccgaatatcgaggtgccagaatcattttttgatctttggataacaccctcgaaacaactaactattggaagttaatgatgtaaattta

SEQ ID NO: 226 - RNA, 163 nt, synthetic construct
  misc_feature 1..163: note = Description of Artificial Sequence: Synthetic polynucleotide
  source 1..163: mol_type = other RNA
caaagggttggctatagcaggctgttgcttgcagaggataagttgacatgaatttgtcatccagcctgatggaataaggtgcggctgatttcaacagccgaatatcgaggtgccagaatcattttttgatctttggataacaccctcgaaacaactaactatt

SEQ ID NO: 227 - RNA, 103 nt, synthetic construct
  misc_feature 1..103: note = Description of Artificial Sequence: Synthetic polynucleotide
  source 1..103: mol_type = other RNA
gtcccaaaatcgcggataagacgcggttgctttcaacagccgaaaatcgagctaccggcgaacttgtttccgccggatcatagcctcgaaacaacgcgtgctt

SEQ ID NO: 228 - RNA, 132 nt, synthetic construct
  misc_feature 1..132: note = Description of Artificial Sequence: Synthetic polynucleotide
  source 1..132: mol_type = other RNA
cgcggtgatgggtatagcctttgggcagagtcccaaaatcgcggataagacgcggttgctttcaacagccgaaaatcgagctaccggcgaacttgtttccgccggatcatagcctcgaaacaacgcgtgctt

SEQ ID NO: 229 - RNA, 132 nt, synthetic construct; identical to SEQ ID NO: 228
  misc_feature 1..132: note = Description of Artificial Sequence: Synthetic polynucleotide
  source 1..132: mol_type = other RNA

SEQ ID NO: 230 - RNA, 160 nt, synthetic construct
  misc_feature 1..160: note = Description of Artificial Sequence: Synthetic polynucleotide
  source 1..160: mol_type = other RNA
caaaaaggttggctatagcaggctattgcttgcagaggataagttgacaataaatttgtcatccagcctgatggaataaggcgcggctgatttcaacagcctaatatcgaggtgccagaatcatgcatgatctctggaggacaccctcgaaacaattatt

SEQ ID NO: 231 - RNA, 103 nt, synthetic construct
  misc_feature 1..103: note = Description of Artificial Sequence: Synthetic polynucleotide
  source 1..103: mol_type = other RNA
ggctgatttcaacagccaaatatcgaggtgccggaattgttaaataatctccggagtacaccctcgaaacaatttaatggagactgttgaggcaaatttattg

SEQ ID NO: 232 - RNA, 170 nt, synthetic construct
  misc_feature 1..170: note = Description of Artificial Sequence: Synthetic polynucleotide
  source 1..170: mol_type = other RNA
atattgccccgaaatgttggctatagcagattttctaaatctgcagaggataagttggcgtagatgccattcaacaaatcggaaagaggcatggctgatttcaacagccaaatatcgaggtgccggaattgttaaataatctccggagtacaccctcgaaacaatttaat

SEQ ID NO: 233 - RNA, 57 nt, synthetic construct
  misc_feature 1..57: note = Description of Artificial Sequence: Synthetic oligonucleotide
  source 1..57: mol_type = other RNA
gttgcagaacccgaatagacgaatgaaggaatgcaactattaaatactcgtattgct

SEQ ID NO: 234 - RNA, 57 nt, synthetic construct
  misc_feature 1..57: note = Description of Artificial Sequence: Synthetic oligonucleotide
  source 1..57: mol_type = other RNA
gtctcagagcatgttctgatagaagaaggattgggactattaaatactcgtattgct

SEQ ID NO: 235 - RNA, 57 nt, synthetic construct
  misc_feature 1..57: note = Description of Artificial Sequence: Synthetic oligonucleotide
  source 1..57: mol_type = other RNA
gtctcagagatacttccaataaaacaaggattgaaactattaaatactcgtattgct

SEQ ID NO: 236 - RNA, 57 nt, synthetic construct
  misc_feature 1..57: note = Description of Artificial Sequence: Synthetic oligonucleotide
  source 1..57: mol_type = other RNA
gtctgaaagctacttccaataaaacaaggattgaaactattaaatactcgtattgct

SEQ ID NO: 237 - RNA, 52 nt, synthetic construct
  misc_feature 1..52: note = Description of Artificial Sequence: Synthetic oligonucleotide
  source 1..52: mol_type = other RNA
gccgcatctcgcacgcgtacgtggattgaaactattaaatactcgtattgct

SEQ ID NO: 238 - RNA, 57 nt, synthetic construct
  misc_feature 1..57: note = Description of Artificial Sequence: Synthetic oligonucleotide
  source 1..57: mol_type = other RNA
gtctgaatcacaattccattaaaacaaggattgaaactattaaatactcgtattgct

SEQ ID NO: 239 - RNA, 81 nt, synthetic construct
  misc_feature 1..81: note = Description of Artificial Sequence: Synthetic oligonucleotide
  source 1..81: mol_type = other RNA
gtgaaggtgggctgcttgcatcagcctaatgtcgagaagtgctttcttcggaaagtaaccctcgaaacaaattcatttttc

SEQ ID NO: 240 - RNA, 103 nt, synthetic construct
  misc_feature 1..103: note = Description of Artificial Sequence: Synthetic polynucleotide
  source 1..103: mol_type = other RNA
ggctgatcccaacagccaaatatcgaggtgtctgcacctatatggacgcagtaagtagaaccaccctcgaaacagttctatcggatttttttggaaatgattc

SEQ ID NO: 241 - RNA, 103 nt, synthetic construct
  misc_feature 1..103: note = Description of Artificial Sequence: Synthetic polynucleotide
  source 1..103: mol_type = other RNA
ccagcctgatggaataaggtgcggctgatttcaacagccgaatatcgaggtgccagaatcattttttgatctttggataacaccctcgaaacaactaactatt

SEQ ID NO: 242 - RNA, 103 nt, synthetic construct
  misc_feature 1..103: note = Description of Artificial Sequence: Synthetic polynucleotide
  source 1..103: mol_type = other RNA
ggttgctttcaacagccgaaaatcgagctaccggcgaacttgtttccgccggatcatagcctcgaaacaacgcgtgcttgtgtgcctgttgatgcgccgtttc

SEQ ID NO: 243 - RNA, 103 nt, synthetic construct
  misc_feature 1..103: note = Description of Artificial Sequence: Synthetic polynucleotide
  source 1..103: mol_type = other RNA
gtcatccagcctgatggaataaggcgcggctgatttcaacagcctaatatcgaggtgccagaatcatgcatgatctctggaggacaccctcgaaacaattatt

SEQ ID NO: 244 - RNA, 102 nt, synthetic construct
  misc_feature 1..102: note = Description of Artificial Sequence: Synthetic polynucleotide
  source 1..102: mol_type = other RNA
ggctgatttcaacagcccaaaatcgagtagtaccgattcgttatgattcggcaaccctcgaaactattataacttttttcatgtctttataaaagattgaaa

SEQ ID NO: 245 - RNA, 103 nt, synthetic construct
  misc_feature 1..103: note = Description of Artificial Sequence: Synthetic polynucleotide
  source 1..103: mol_type = other RNA
gcgtactccgcaggcgaggtatgtacaactgcacggctgatttcaacagcccaaaatcgagtagtaccgattcgttatgattcggcaaccctcgaaacttatt

SEQ ID NO: 246 - RNA, 103 nt, synthetic construct
  misc_feature 1..103: note = Description of Artificial Sequence: Synthetic polynucleotide
  source 1..103: mol_type = other RNA
cattcaacaaatcggaaagaggcatggctgatttcaacagccaaatatcgaggtgccggaattgttaaataatctccggagtacaccctcgaaacaatttaat

SEQ ID NO: 247 - RNA, 57 nt, synthetic construct
  misc_feature 1..57: note = Description of Artificial Sequence: Synthetic oligonucleotide
  source 1..57: mol_type = other RNA
gtctcagagcatgttctgatagaagaaggattgggacgccgataatgatgtagggat

SEQ ID NO: 248 - RNA, 57 nt, synthetic construct
  misc_feature 1..57: note = Description of Artificial Sequence: Synthetic oligonucleotide
  source 1..57: mol_type = other RNA
gtctcagagatacttccaataaaacaaggattgaaacgccgataatgatgtagggat

SEQ ID NO: 249 - RNA, 52 nt, synthetic construct
  misc_feature 1..52: note = Description of Artificial Sequence: Synthetic oligonucleotide
  source 1..52: mol_type = other RNA
gccgcatctcgcacgcgtacgtggattgaaacgccgataatgatgtagggat

SEQ ID NO: 250 - RNA, 57 nt, synthetic construct
  misc_feature 1..57: note = Description of Artificial Sequence: Synthetic oligonucleotide
  source 1..57: mol_type = other RNA
gtctgaaagctacttccaataaaacaaggattgaaacgccgataatgatgtagggat

SEQ ID NO: 251 - RNA, 57 nt, synthetic construct
  misc_feature 1..57: note = Description of Artificial Sequence: Synthetic oligonucleotide
  source 1..57: mol_type = other RNA
gtctgaatcacaattccattaaaacaaggattgaaacgccgataatgatgtagggat

SEQ ID NO: 252 - RNA, 57 nt, synthetic construct
  misc_feature 1..57: note = Description of Artificial Sequence: Synthetic oligonucleotide
  source 1..57: mol_type = other RNA
gttgcagaacccgaatagacgaatgaaggaatgcaacgccgataatgatgtagggat

SEQ ID NO: 253 - RNA, 57 nt, synthetic construct; identical to SEQ ID NO: 252
  misc_feature 1..57: note = Description of Artificial Sequence: Synthetic oligonucleotide
  source 1..57: mol_type = other RNA

SEQ ID NO: 254 - RNA, 59 nt, synthetic construct
  misc_feature 1..59: note = Description of Artificial Sequence: Synthetic oligonucleotide
  source 1..59: mol_type = other RNA
gttgcagaacccgaatagacgaatgaaggaatgcaactattaaatactcgtattgctgt

SEQ ID NO: 255 - RNA, 17 nt, synthetic construct
  misc_feature 1..17: note = Description of Artificial Sequence: Synthetic oligonucleotide
  source 1..17: mol_type = other RNA
agaagaaggattgggac

SEQ ID NO: 256 - RNA, 17 nt, synthetic construct
  misc_feature 1..17: note = Description of Artificial Sequence: Synthetic oligonucleotide
  source 1..17: mol_type = other RNA
aaaacaaggattgaaac

SEQ ID NO: 257 - RNA, 17 nt, synthetic construct
  misc_feature 1..17: note = Description of Artificial Sequence: Synthetic oligonucleotide
  source 1..17: mol_type = other RNA
cgtacgtggattgaaac

SEQ ID NO: 258 - RNA, 37 nt, synthetic construct
  misc_feature 1..37: note = Description of Artificial Sequence: Synthetic oligonucleotide
  source 1..37: mol_type = other RNA
gtctcagagcatgttctgatagaagaaggattgggac

SEQ ID NO: 259 - RNA, 37 nt, synthetic construct
  misc_feature 1..37: note = Description of Artificial Sequence: Synthetic oligonucleotide
  source 1..37: mol_type = other RNA
gtctcagagatacttccaataaaacaaggattgaaac

SEQ ID NO: 260 - RNA, 32 nt, synthetic construct
  misc_feature 1..32: note = Description of Artificial Sequence: Synthetic oligonucleotide
  source 1..32: mol_type = other RNA
gccgcatctcgcacgcgtacgtggattgaaac

SEQ ID NO: 261 - RNA, 37 nt, synthetic construct
  misc_feature 1..37: note = Description of Artificial Sequence: Synthetic oligonucleotide
  source 1..37: mol_type = other RNA
gtctgaaagctacttccaataaaacaaggattgaaac

SEQ ID NO: 262 - RNA, 37 nt, synthetic construct
  misc_feature 1..37: note = Description of Artificial Sequence: Synthetic oligonucleotide
  source 1..37: mol_type = other RNA
gtctgaatcacaattccattaaaacaaggattgaaac

SEQ ID NO: 263 - RNA, 132 nt, synthetic construct
  misc_feature 1..132: note = Description of Artificial Sequence: Synthetic polynucleotide
  source 1..132: mol_type = other RNA
tttctcagagatggctgatcccaacagccaaatatcgaggtgtctgcacctatatggacgcagtaagtagaaccaccctcgaaacagttctgaaaagaagaaggattgggactattaaatactcgtattgct

SEQ ID NO: 264 - RNA, 172 nt, synthetic construct
  misc_feature 1..172: note = Description of Artificial Sequence: Synthetic polynucleotide
  source 1..172: mol_type = other RNA
ctaaaggctgaccatagcaggtttcctgcaggcggttagttttctcagagatggctgatcccaacagccaaatatcgaggtgtctgcacctatatggacgcagtaagtagaaccaccctcgaaacagttctgaaaagaagaaggattgggactattaaatactcgtattgct

SEQ ID NO: 265 - RNA, 117 nt, synthetic construct
  misc_feature 1..117: note = Description of Artificial Sequence: Synthetic polynucleotide
  source 1..117: mol_type = other RNA
ggctgatttcaacagccgaatatcgaggtgccagaatcattttttgatctttggataacaccctcgaaacaactaagaaaaaaacaaggattgaaactattaaatactcgtattgct

SEQ ID NO: 266 - RNA, 194 nt, synthetic construct
  misc_feature 1..194: note = Description of Artificial Sequence: Synthetic polynucleotide
  source 1..194: mol_type = other RNA
ggttggctatagcaggctgttgcttgcagaggataagttgacatgaatttgtcatccagcctgatggaataaggtgcggctgatttcaacagccgaatatcgaggtgccagaatcattttttgatctttggataacaccctcgaaacaactaagaaaaaaacaaggattgaaactattaaatactcgtattgct

SEQ ID NO: 267 - RNA, 139 nt, synthetic construct
  misc_feature 1..139: note = Description of Artificial Sequence: Synthetic polynucleotide
  source 1..139: mol_type = other RNA
gtcccaaaatcgcggataagacgcggttgctttcaacagccgaaaatcgagctaccggcgaacttgtttccgccggatcatagcctcgaaacaacgcggaaacgtacgtggattgaaactattaaatactcgtattgct

SEQ ID NO: 268 - RNA, 168 nt, synthetic construct
  misc_feature 1..168: note = Description of Artificial Sequence: Synthetic polynucleotide
  source 1..168: mol_type = other RNA
cgcggtgatgggtatagcctttgggcagagtcccaaaatcgcggataagacgcggttgctttcaacagccgaaaatcgagctaccggcgaacttgtttccgccggatcatagcctcgaaacaacgcggaaacgtacgtggattgaaactattaaatactcgtattgct

SEQ ID NO: 269 - RNA, 141 nt, synthetic construct
  misc_feature 1..141: note = Description of Artificial Sequence: Synthetic polynucleotide
  source 1..141: mol_type = other RNA
cggctgatttcaacagcctaatatcgaggtgccagaatcatgcatgatctctggaggacaccctcgaaacaattattggaagctaatgatgcaaatttatgaaaaaaacaaggattgaaactattaaatactcgtattgct

SEQ ID NO: 270 - RNA, 194 nt, synthetic construct
  misc_feature 1..194: note = Description of Artificial Sequence: Synthetic polynucleotide
  source 1..194: mol_type = other RNA
ggttggctatagcaggctattgcttgcagaggataagttgacaataaatttgtcatccagcctgatggaataaggcgcggctgatttcaacagcctaatatcgaggtgccagaatcatgcatgatctctggaggacaccctcgaaacaattatgaaaaaaacaaggattgaaactattaaatactcgtattgct

SEQ ID NO: 271 - RNA, 117 nt, synthetic construct
  misc_feature 1..117: note = Description of Artificial Sequence: Synthetic polynucleotide
  source 1..117: mol_type = other RNA
ggctgatttcaacagccaaatatcgaggtgccggaattgttaaataatctccggagtacaccctcgaaacaatttagaaaaaaacaaggattgaaactattaaatactcgtattgct

SEQ ID NO: 272 - RNA, 168 nt, synthetic construct
  misc_feature 1..168: note = Description of Artificial Sequence: Synthetic polynucleotide
  source 1..168: mol_type = other RNA
tgcagaggataagttggcgtagatgccattcaacaaatcggaaagaggcatggctgatttcaacagccaaatatcgaggtgccggaattgttaaataatctccggagtacaccctcgaaacaatttagaaaaaaacaaggattgaaactattaaatactcgtattgct

SEQ ID NO: 273 - protein, 166 aa, synthetic construct
  REGION 1..166: note = Description of Artificial Sequence: Synthetic polypeptide
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 274 - protein, 32 aa, synthetic construct
  REGION 1..32: note = Description of Artificial Sequence: Synthetic polypeptide
SGGSSGGSSGSETPGTSESATPESSGGSSGGS

SEQ ID NO: 275 - protein, 32 aa, synthetic construct; identical to SEQ ID NO: 274
  REGION 1..32: note = Description of Artificial Sequence: Synthetic polypeptide

SEQ ID NO: 276 - protein, 166 aa, synthetic construct
  REGION 1..166: note = Description of Artificial Sequence: Synthetic polypeptide
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 277 - protein, 364 aa, synthetic construct
  REGION 1..364: note = Description of Artificial Sequence: Synthetic polypeptide
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 278 - protein, 364 aa, synthetic construct
  REGION 1..364: note = Description of Artificial Sequence: Synthetic polypeptide
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 279 - protein, 166 aa, synthetic construct
  REGION 1..166: note = Description of Artificial Sequence: Synthetic polypeptide
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 280 - protein, 198 aa, synthetic construct
  REGION 1..198: note = Description of Artificial Sequence: Synthetic polypeptide
EASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 281 - protein, 228 aa, synthetic construct
  REGION 1..228: note = Description of Artificial Sequence: Synthetic polypeptide
SSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLK

SEQ ID NO: 282 - protein, 224 aa, synthetic construct
  REGION 1..224: note = Description of Artificial Sequence: Synthetic polypeptide
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 283 - protein, 677 aa, synthetic construct
  REGION 1..677: note = Description of Artificial Sequence: Synthetic polypeptide
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 284 - protein, 854 aa, synthetic construct
  REGION 1..854: note = Description of Artificial Sequence: Synthetic polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSPKKKRKVEASGGGSGGGSGGGSGEAAPCDCDGGTQKEKGPYYTHLGAGPSVAAVRELMETRFGQKGKAIRIEKIVFTGKEGKSSQGCPVAKWVIRRSGPEEKLICLVRERVDHHCSTAVIVVLILLWEGIPRLMADRLYKELTENLRSYSGHPTDRRCTLNKKRTCTCQGIDPKTCGASFSFGCSWSMYFNGCKFGRSENPRKFRLAPNYPLHNYYKRITGMSSEGSDVKTGWIIPDRKTLISREEKQLEKNLQELATVLAPLYKQMAPVAYQNQVEYEEVAGDCRLGNEEGRPFSGVTCCMDFCAHSHKDIHNMHNGSTVVCTLIRADGRDTNCPEDEQLHVLPLYRLADTDEFGSVEGMKAKIKSGAIQVNGPTRKRRLRFTEPVPRCGKRAKMKQNHNKSGSHNTKSFSSASSTSHLVKDESTDFCPLQASSAETSTCTYSKTASGGFAETSSILHCTMPSGAHSGANAAAGECTGTVQPAEVAAHPHQSLPTADSPVHAEPLTSPSEQLTSNQSNQQLPLLSNSQKLASCQVEDERHPEADEPQHPEDDNLPQLDEFWSDSEEIYADPSFGGVAIAPIHGSVLIECARKELHATTSLRSPKRGVPFRVSLVFYQHKSLNKPNHGFDINKIKCKCKKVTKKKPADRECPDVSPEANLSHQIPSRVASTLTRDNVVTVSPYSLTHVAGPYNRWVAAADYKDDDDK

SEQ ID NO: 285 - protein, 1006 aa, synthetic construct
  REGION 1..1006: note = Description of Artificial Sequence: Synthetic polypeptide
QSQPGHNQMLRPIKTEPVSKPSSYRYPLSPPQENMSSRIKQEISSPSRDNGQPKSIIETMEQHLKQFQLKSLCDYKALTLKSQKHVKVPTDIQAAESENHARAAEPQATKSTDCSVLDDVSESDTPGEQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETKKKQSPPRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKHSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTVYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 286 - protein, 617 aa, synthetic construct
  REGION 1..617: note = Description of Artificial Sequence: Synthetic polypeptide
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 287 - protein, 523 aa, synthetic construct
  REGION 1..523: note = Description of Artificial Sequence: Synthetic polypeptide
DALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 288 - protein, 322 aa, synthetic construct
  REGION 1..322: note = Description of Artificial Sequence: Synthetic polypeptide
TYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 289 - protein, 380 aa, synthetic construct
  REGION 1..380: note = Description of Artificial Sequence: Synthetic polypeptide
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFST

SEQ ID NO: 290 - protein, 50 aa, synthetic construct
  REGION 1..50: note = Description of Artificial Sequence: Synthetic polypeptide
DALDDFDLDMLGSDALDDFDLDMLGSDALDDFDLDMLGSDALDDFDLDML

SEQ ID NO: 291 - protein, 745 aa, synthetic construct
  REGION 1..745: note = Description of Artificial Sequence: Synthetic polypeptide
GQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 292 - protein, 75 aa, synthetic construct
  REGION 1..75: note = Description of Artificial Sequence: Synthetic polypeptide
MDAKSLTAWSRTLVTFKDVFVDFTREEWKLLDTAQQIVYRNVMLENYKNLVSLGYQLTKPDVILRLEKGEEPWLV

SEQ ID NO: 293 - protein, 99 aa, synthetic construct
  REGION 1..99: note = Description of Artificial Sequence: Synthetic polypeptide
NNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 294 - protein, 479 aa, synthetic construct
  REGION 1..479: note = Description of Artificial Sequence: Synthetic polypeptide
VGTMITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEKLGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIAQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 295 - protein, 340 aa, synthetic construct
  REGION 1..340: note = Description of Artificial Sequence: Synthetic polypeptide
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 296 - protein, 509 aa, synthetic construct
  REGION 1..509: note = Description of Artificial Sequence: Synthetic polypeptide
MPIAPEFLSLAYPGQEFPAYLYGRASRDPKRKGRSVQSQLDEGRATCLDAGWPIAGEFKDVDRSASAYARRTRDEFEEMIAGIQAGECRILVAFEASRYYRDLEAYVRLRRVCREAGVLLCYNGQVYDLSKSADRKATAQDAVNAEGEADDIRERNLRTTRLNAKRGGAHGPVPDGYKRRYDPDSGDLVDQIPHPDRAGLITEIFRRAAAAEPLAAICRDLNERGETTHRGKAWQRHHLHAILRNPAYIGHRRHLGVDTGKGMWAPICDDEDFAETFQAVQEILSLPGRQLSPGPEAQHLQTGIALCGEHPDEPPLRSVTVRGRTNYNCSTRYDVAMREDRMDAFVEESVITWLASDEAVAAFEDNTDDERTRKARIRLKVLEEQLEAAQKQARTLRPDGMGMLLSIDSLAGLEAELTPQIDKARQESRSLHVPALLRDLLGKPRADVDRAWNEALTLPQRRMILRMVVTIRLFKAGSRGVRAIEPGRITLSYVGEPGFKPVGGNRAKQ

SEQ ID NO: 297 - protein, 474 aa, synthetic construct
  REGION 1..474: note = Description of Artificial Sequence: Synthetic polypeptide
MAVGIYIRVSTQEQASEGHSIESQKKKLASYCEIQGWDDYRFYIEEGISGKNTNRPKLKLLMEHIEKGKINILLVYRLDRLTRSVIDLHKLLNFLQEHGCAFKSATETYDTTTANGRMSMGIVSLLAQWETENMSERIKLNLEHKVLVEGERVGAIPYGFDLSDDEKLVKNEKSAILLDMVERVENGWSVNRIVNYLNLTNNDRNWSPNGVLRLLRNPALYGATRWNDKIAENTHEGIISKERFNRLQQILADRSIHHRRDVKGTYIFQGVLRCPVCDQTLSVNRFIKKRKDGTEYCGVLYRCQPCIKQNKYNLAIGEARFLKALNEYMSTVEFQTVEDEVIPKKSEREMLESQLQQIARKREKYQKAWASDLMSDDEFEKLMVETRETYDECKQKLESCEDPIKIDETYLKEIVYMFHQTFNDLESEKQKEFISKFIRTIRYTVKEQQPIRPDKSKTGKGKQKVIITEVEFYQ

SEQ ID NO: 298 - protein, 805 aa, synthetic construct
  REGION 1..805: note = Description of Artificial Sequence: Synthetic polypeptide
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSRSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFKSSKFTRTISPPTLGTLRSCFSWSGGLGDFSRTPSPSPSTALQQFRRKSDSPTSLPENNMSDVSQLKSEESSDDESHPLREGACSSQSQESGEFSLQSSNASKLSQCSSKDSDSEESDCNIKLLDSQSDQTSKLCLSHFSKKDTPLRNKVPGLYKSSSADSLSTTKIKPLGPARASGLSKKPASIQKRKHHNAENKPGLQIKLNELWKNFGFKKFRD

SEQ ID NO: 299 - skipped

SEQ ID NO: 300 - protein, 5 aa, synthetic construct
  REGION 1..5: note = Description of Artificial Sequence: Synthetic peptide
  REGION 1..5: note = See specification as filed for detailed description of substitutions and preferred embodiments
GSGGS

SEQ ID NO: 301 - protein, 6 aa, synthetic construct
  REGION 1..6: note = Description of Artificial Sequence: Synthetic peptide
  REGION 1..6: note = See specification as filed for detailed description of substitutions and preferred embodiments
GGSGGS

SEQ ID NO: 302 - protein, 4 aa, synthetic construct
  REGION 1..4: note = Description of Artificial Sequence: Synthetic peptide
  REGION 1..4: note = See specification as filed for detailed description of substitutions and preferred embodiments
GGGS

SEQ ID NO: 303 - protein, 4 aa, synthetic construct
  REGION 1..4: note = Description of Artificial Sequence: Synthetic peptide
GGSG

SEQ ID NO: 304 - protein, 5 aa, synthetic construct
  REGION 1..5: note = Description of Artificial Sequence: Synthetic peptide
GGSGG

SEQ ID NO: 305 - protein, 5 aa, synthetic construct
  REGION 1..5: note = Description of Artificial Sequence: Synthetic peptide
GSGSG

SEQ ID NO: 306 - protein, 5 aa, synthetic construct
  REGION 1..5: note = Description of Artificial Sequence: Synthetic peptide
GSGGG

SEQ ID NO: 307 - protein, 5 aa, synthetic construct
  REGION 1..5: note = Description of Artificial Sequence: Synthetic peptide
GGGSG

SEQ ID NO: 308 - protein, 5 aa, synthetic construct
  REGION 1..5: note = Description of Artificial Sequence: Synthetic peptide
GSSSG

SEQ ID NO: 309 - DNA, 19 bp, synthetic construct
  misc_feature 1..19: note = Description of Artificial Sequence: Synthetic oligonucleotide
  source 1..19: mol_type = other DNA
acatgaggatcacccatgt

SEQ ID NO: 310 - DNA, 25 bp, synthetic construct
  misc_feature 1..25: note = Description of Artificial Sequence: Synthetic oligonucleotide
  source 1..25: mol_type = other DNA
ggagcagacgatatggcgtcgctcc

SEQ ID NO: 311 - DNA, 15 bp, synthetic construct
  misc_feature 1..15: note = Description of Artificial Sequence: Synthetic oligonucleotide
  source 1..15: mol_type = other DNA
gccctgaagaagggc

SEQ ID NO: 312 - DNA, 140 bp, synthetic construct
  misc_feature 1..140: note = Description of Artificial Sequence: Synthetic polynucleotide
  source 1..140: mol_type = other DNA
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcattt

SEQ ID NO: 313 - DNA, 57 bp, synthetic construct
  misc_feature 1..57: note = Description of Artificial Sequence: Synthetic oligonucleotide
  source 1..57: mol_type = other DNA
gttgcagaacccgaatagacgaatgaaggaatgcaactattaaatactcgtattgct

SEQ ID NO: 314 - DNA, 20 bp, synthetic construct
  misc_feature 1..20: note = Description of Artificial Sequence: Synthetic oligonucleotide
  misc_feature 1..20: note = This sequence may encompass 5-20 nucleotides
  source 1..20: mol_type = other DNA
aaaaaaaaaaaaaaaaaaaa

SEQ ID NO: 315 - DNA, 20 bp, synthetic construct
  misc_feature 1..20: note = Description of Artificial Sequence: Synthetic oligonucleotide
  misc_feature 1..20: note = This sequence may encompass 5-20 nucleotides
  source 1..20: mol_type = other DNA
tttttttttttttttttttt

SEQ ID NO: 316 - DNA, 20 bp, synthetic construct
  misc_feature 1..20: note = Description of Artificial Sequence: Synthetic oligonucleotide
  misc_feature 1..20: note = This sequence may encompass 5-20 nucleotides
  source 1..20: mol_type = other DNA
cccccccccccccccccccc

SEQ ID NO: 317 - DNA, 20 bp, synthetic construct
  misc_feature 1..20: note = Description of Artificial Sequence: Synthetic oligonucleotide
  misc_feature 1..20: note = This sequence may encompass 5-20 nucleotides
  source 1..20: mol_type = other DNA
gggggggggggggggggggg